SEQUENCE LISTING (486 sequences)

SEQ ID NO: 1 - DNA, 39543 bp, Homo sapiens
  source 1..39543: mol_type = genomic DNA
ttttttttaaggttccacatatgagtgagatcatgtagtgtttgtctttctgtgcctggcttattatgctttgtgtaaagtccaccgggttcatctatgttgtggcaaatagcagagtctcaatttttaatactaactaagattgtattgtgtatatatgccccaaactctttatttattcatctgtcaatagacacttgggttgtttgcctatctggaccatggtgaataatgctgctatgaacatgagtgcacagatgtctttgtgaggtggtgattttatttcctttggatacacactcagaagaaggattgctgggtcatacggtagttctatttttaatttctttaggaacctccatattgttttctgcaatggctgcaccaatctgcattcccagcagcaatgtgcaagggttccctgttcttcacgccctagccaatgtttgtctcttgtcattttgctaatagccatcctaacaggtttgaggtggtacctcattgtgggtttggtttgcatttccctgatgattagtgatgttgagcacttttttgtatacatgttgaccatttttgtttcttttttagagaaatgtctattcagttcctttcttcattttaaaaatcagattatttgttttatgctttgagttgtgtgaattccttacatattttgcatattaactacttatctgatatatggcttaaaactattttcttccaatttgttggctaccttttcactctgttgattgtttcttttgctatacaggagctttttactttgatgtattcccacttatttatctttacttttgttcctttgcttttgttgagacattaaaaatatccatgccaaggctaatatcaagaagttttctcctctgttttctattttttatatttagttttttatctgtttagagattgggtctcattctgttacccaggctggaatgcagtagtgtcattatagctcactgtaacctcaaattcctgggctcaagtgatcttcttgcctcggcctcctgagtagctgggctacaggtgcacagcaccatgcctggctaatttttttttttttaattttgtagacacaaagtctcactatgttgcccaggctgactgtgaacttctggcctcaggtgatcctcctgcctcagcctcccagagtgctgggattacaggcatgagccaccatgcctggcttcttctctattttcttctaggagttttacagttttagatcttatgtttagatctttaatccattttgagttggtttttgtgtgtgccaagttttattcttttgcgtgtggaaatccagtttcaccaaaagcttaatttttcaaaatcttatatttttcttgatttttttttcacacttctatagccaacatctttattgaagagattgtcctttctccattgtgtcttcttggtgaccttgtcaaagaattagttgactgcatatacttgagtttatttctgttccactggtctatgggtctgtttttataaaggcatggatttttactgcatatgaattatacctaaatttgtcttttttttttcctttttctggagaatggggtctcactatattgcccaggcaggtctcgaactcctgggctcaagctatcctcccgcctcttgcctccctgagagctgggattacaggcgtgagccactgcgcccggctgaattatacctaaattaacttgactaaacaaacaaaaacaaaaagatcccaggttcagccaatgagctcctttttttttttttgaaatggagtcttgctcttgtcacccaggctggagtgcagtggcacaatctcagctcactgcagcctccacctcccaggttcaagtgattctcttgcctcagcttcctgagtagctgggattacaggtgcccgccaccacacccagctatttttttttatatttttagtagagatggggtttcgtcatgttggccaggctggtctcaaactcctgaccttgtgatctgcctgccttggcctctcaaagtgctgggattacaggcatgagccactgcacccaggctgagctcctttcttttggagcatatttggtctgctttctctctagcttcagcacctgaagtttttgctgctcctgcctcagctccagccccgaatgacacctttcacctggttcatggagactcaggtctgctgtcatgcccctaagcaggactagatgcctctctcatgttgtagagcattctctctgcagttctgtagaaaggtggtagtcatcggaaccaactgcttattctagcttctctgtgccaggctctgttttaaaggcctcacgtttagccattcactcagtgttttctgacagccttctaggataggtaccgttatttgggttgatgtagtgcttggtcaaatgcagtataatgaagggatcaaggagggggcaattccaagcaaaaccacttgttcatccatcctgagtctaggagatttggaagatctaaagaagtagcaggcaattattgtcacccttttacaggtatgaggcacagacagacccagtcatttgctgacatccctccactgaactgggtgctcgtccttttcactcacccagaggctgagcgagtgcctctcacccaccaagctctgttgaacttgtgcctttgccggtctgtggtcctgcggccgtctcatgagctgcagagtaggtctgtgtgtgtctctcctcaagctactcaagctgaggctttgtgtgcatgcctgccacataccagccattccgtgagtgttagtggaacaggtgagtcaatgagtcagggagtaaaatttgtgctttaaaagtagaacatgtcggagagaacctgtactgccttccagccatcttgctgtgagaggacagggcatggactctggagtttgaaggctgggtcaggtcatgctctggattctttactcactcgcatggcatgtccttagtttcacttccctcactctcagtggagccgttgcagcgggcagtgtcagaggcaacatccatttagcactgccgatttgacccccagccccatctcatactggcctcccaccctgcttctgtggggtgcctcagaggagagcaaggtgaaccccagtatggaaccgagacacggttttgacaacactgttgatcccatttggaaaatccagctatgtgattaaccattagtctctctgcctctgctctgatgtaagtggagaccacatccttcctgccccttctggggctgcgactgctataaattcattgcatttcctctctagctgttcctgaggctggcatctgggtaagtccagctccgcggggcagtgtgggcgacaggaccgggagagcagtgggggagattgtgttcagagggcagaacttgtcctggttaatgcactaggaatttcttctggcgtctccaaggttgtgcttcaacaacatttctggaatgttcttttatcattatttgggctctacgtgtgcaagttcgtgtgtgtgtatgtggcggggggaagcggggaggacagcggcagcaaccatagtttacacccacgacttcagcagatttgcccagtatccctcccaggggagaagatggatgggatatacagaaggaaacaaacctccttaaagaccaaagggcagcagatttttgtgctggtgttagggtggaaggagcacagaagaaaggcagaatcctgatgcagatggaaggactgatggggcaggagctgggagggtatgcggagctaatagggctgtctggagagcagtcacctcaggatgggcaggagctgggagggtatgcggagctaatagggctgtctggagagcagtcacctcaggatgcacagggctgggtttgcagggctcaggaggcgaggagtagataggcaggaatggagatgggacgggagagatgagtgagtctgaggcatcttgacattgaactgcagtcacagaaattccaatttggagagctccaccgttttccaggctctctgcctcccttgagtgttatttatggctttaggttgaggtgctttgccaaatagagtataatcaagggatcaaggagggaacaattccaaacaaagccaacttgttcatccatccggactctaggagatatggaagatctaaagatacagcaggtgattgctagtttttgtggaactcacagtctagttgggaagactatgttggtgaatgctgcagtgatacacctgtgataaatgctggggaagtgtgtcttttagtcatctattttgttgtctttgtctttgtctgactgaaacaagtgatggaagacatggtctcatatctctggtcaaaatcctggctctgttccttcctggttctaaacccctcggcaggcttcctagtccccctaagactcagtttatgcatctataaaatggggctaggaataggtgcacctcatagagctctgtgcaggttagatggggaaaaacatcagtgagtgcctagcctgtggaaagctctaaattaatgtgaattataatgataacaattgcgaattttgcaaggagacattaaaggatgtatgtttttatttattttattttattctattttattttttgagatggagttttgctcttattgcccaggctggagtgcagtggtgtgatcttggctcatcgcaacctccgcctcctgggttcaagcgattctcctgcctcagcctcccgagtagctgggattacaggtgcctggtaccatgcccggttaatttttgtatttttagtagagacgggatttctccctgttgtttggtgagcctggtctccaactcctgacctcaggtgatccgcccacctcggcctcccaaagtgctgggattacagggatgtatgtttttattattctccattatgcactcccagcttcatctcacttcaatccactggttgatagaagtgcagtcaaagactgtcccctcctctgcaagcttttatgttctctctcctctctccctagccacgtcctgttctgttatcaacacacttactcacctcctggccttctccttctattgtctctcatccctcttccctcacaaaaacagaagcaaagagccagagccttcagtttggaggaactgaaaacattctcttctgctttctcattttgtagatgaggaaactgaagttgaggaatagtgaagagtttgtccaatgtcatagccccgtaatcaacgggacaaaaattttcttgctgatgggtcaagatggcatcgtgaagtggttgttcaccgtaaactgtaatacaatcctgtttatggatttgtttgcatatttttccctccatagggaaacctttcttccatggctcaggacacactcctggatcgagccaacaggagaactttctggtaagcatttggctaacttttttttttttgagatggagtcttgctgtgtcgcctaggctggagtgcagtggcgtgatcttggctcactgcagcctccacttcccgggttcaatcaattctcctacctcaacttcctgagtagctgggattacaggcgcccgccaccacacccggctcatttttgtacttttagtagagacacagttttgccatgttggccaggctggtcttgaattcctcagctcaggtgatctgcctgccttggcctctcaaagtgctgggattacaggcgtgagccactgtgcccggccttggctaacttttcaaaattaaagattttgacttgttacagtcatgtgacatttttttctttctgtttgctgagtttttgataatttatatctctcaaagtggagactttaaaaaagactcatccgtgtgccgtgttcactgcctggtatcttagtgtggaccgaagcctaaggaccctgaaaacagctgcagatgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggtgagtggaaggaagacttttaaaaaaaattgtggccaagtgcacatagtgtacaattttccatctttatatatatatatatatatatttttttttgagacggagttgctcttgttgcccaggctggagtgcaatggtgagatcttggctcactgcaacctccacctcccgggttcaagcgattttcctgcctcagcctcccaagtagctgggattacaggcattgcacctccacgcccggctaattttgtatttttagtagagatggggtttctccatgttggtcaggttggtctcaaactcccgacctcaggtgatccacccgcctcggcctcgcaaagtgctggaattacaggtgtgagccaccgcgcccggccgtaataatttttaaatgtacagttcagtagtgttaggtacactcacattgtgcaaccagtctccagaactgttttcatcttgcaaaactgaaactcctcacccattgaatgataactcctcattgcctcattctcccagccctggtaaccaccattctatttgccatctctgtgaatcttactactctgggtacctcatataagtggaaacatacagcattttttcctttatgagtagtttatttcactcagcataatgtcctccgggttcaactatgttgtagcatgtgtcggaattcccttcctttaaaaaagactgtataaatactccatggtaccatataccaaattttgtttagttatctgttgatgaacacctggattacttctacctttttgttattgttaatgctgttatgagtgttggtgcacaaatatctcttcaagaccctgctttgaattctctggggtatatactcagaagtgaaatttctgaatcatacggtaattctgttaaatattttgaagaactgctctactgttttagataccatctgcaccatttcccaccaacagcacacaagggttccaatttctccatatcctaagaacacgttatttttatttattttttcttttttatagtagccattctaatgagtgtgaggtggtatctcactgtagttttgatttccgtttccctaatgataagtgatgctgagcacattttcactgcttgttggccatttatagagcttcttcagagaaagttctgttcaagttcttttctcattttcagaccaggtttggcttttgttgttgccttgtaggagttctttatgtattctagatattaaccccatatcagatatatgatttacaaatattttctcctattctgtgggttgtcttttcatttggttaactgtgtcctttgatgcacagaagttagtatgatttttttttttttaaatcatggccgggcatggtggctcatgcctgtaattccagcactttgtgaggctgaggtgggtggatcacctgaggtcaggagttcaacaccagcctggccaacatggcaaaaccccatctctactaaaaatacaaaaaaaattagccgggtgtggtggtgggcacctgtaatcccagctactcaggaggctgaggtaggagaattgcttgaacatggggagtggaggttgcagtgagctgagattgcaccacggcactccagcctgggctaaagagcaagaccctgtttcaaaaaaaaatttttttttcttgtattgtcttggttttttctcttttcccctctcacggtagtgctatgagaaggtggaggcaaactgtttgctttctctaactcctttaaaaagctgatgcagactgtacctaccccctgggcacctgccatgattagtccctctttatctgaatctcagcagtgcctagacctgcctccaattctccatccagcttctcagaccacatgttttgtccagtatctgctgtgtggtcagcaggcttgggcctctgcttcctatggcccagagggcagcctctctccttgaagaaccgtggagtttggggcagaagcctttcctttcaccaccactttgggaattggaatgtaaaagaaattctcttaatccccatgtacacattggggataaggttctttcttttgtcttgtgttctaggcaaaggtagggctcagagaagggcagggtattgcaggatgatgatgtagagctggtggtgatgggaaggaggagtacaaatgagtcagtggatagggagacacatgttaaggcagggcagcagtgtgggtgtaatgacactggggaaacagacagcagtgtgcagccgggggaggtcagaaaccagttgctgcaatggctctcacctgtagtcccagctactcaggaggctaaggcaggaggatcgcttgagcccaggagctcaaaaccagcctgagcaacatagggagacctcatctctaagtaaataaagcaattatcattgtgtcagtacttatatactcacatacccaaatgaaagtgtcataatcacaactgtagaagtactttgctaagcagtttttatggattttatggcacattcaagaaacccatgaaacagcttctggtattatccctcctgcaagtgaatgaggtgagcctggaagagatcctaactggcccatggtttatttctagccataaccagagctggggtaaaaagcagggcatgtctctgatcaaagcctggctcttaaccactacaccgtctttgcttccatatctggattttgaggcagaaaagttataactagggttagaagtttgtttgtttgtttcctggggtggggttaggttttaggtaatagactttggctaatttactgtcatggctcctacctgagtcccagttactcaggtggctgaagtgggtgcatcacttgagctcaggagttccaggctggagtgagctatgaccatgccactgccctccagctttggaaacaaagcaagactctgtctcttaaaaaaaaagaataagattttggttttggcttagattttttggattttattttgtgatatggttaccaagtagacagctgtcagaggtcttttatgggctgggcgcagtggcttatgcctgtaatcccagcacttcaggaggccaagatgggaggattgcttgagcccaggagtttgagaccagcctgggcaacatagtgagaacctgtctctacaaaaaatgcaaacattagttaggtgtggtggcacgtgtcaatagtcccagctgttctggaggctgaggtgggaggatcacttgagcccaggaggttgaggctgcagggagccatgattgtgtcattgcactccagcctgggcaatagagtgaggccctgtctcaaaaaaaaaaaaaaaatcatttatgttacttgtagattcctgcccacctgaagttatctgtcttatatatttcccctagtgactaactcttgttttacctaacctgaatttctcctatagaatgccttcttaagatatgctatctagtgactgtttggatttgtgattctcaacacagatgcatgttggaatcacctgggagcttttagtatgtgatggttcctgggcctcacctcagagggactgaaaaagaacctctgggcgtgcagtcctgaagtccatatttttaaagcttcctgggtgattctaaagtcagccaggattcccaaccactagattaggcacgaaatagactggtcgttctcactcttgaagaaccagacatctagacttgacaatagaagacgtgtgtaaaatggattgcagggaactgcggtcagggaagggtgacctttcttagcccacagccgtgttggggatataggagagcttggagatgccctcacaatcaggctgccaggggcaggcaaaagcaaacgagggtaaaagaaacttagcctgcttcgcatgtttgctgattgatttccaggcctgaggaagagattctgagactggggacagtgggaacacatgcttggcaggtggacagcagaagttgttttgaaactaggagtgcagaaatgctcccaaccagacttttgactggatacctgtcctctgcaactcagagccatgttttgatttgggggatgtttggggtctcctctctcatgccaaatattaggccaggtttcaattgcatcctctgtgataatagttctgggttttgacaccttttttttcccttttaggttcagataatgcacgtgtttcgaatcccactgtgatatgccaggaagacagcattgaagaggagtggatgggtttactggagtacctttcgagaatctctatttgtaaaatgaagaaaggtaagcgactggggtggtgcttctagttgagttttaagacctggttcaggaggctctgggaagctgagcagctgggtaacttggccatactataggttcctgctctttggaatgcaaaggcctgtctcaggaaatccattgtcagttgaagaaacaaattcatgtttgtgtggctcttgcagtcccagagccagcggcggtgattaagcgtttcaagtttgataaggcataggatgcagattacttaaaggaataagattgattcaaatgatctttgggctggtagctacacatagagatgggccttctgctcagctgtgtatcttaaaaacttgacaccagagattctcggcacctttcctacccagtggggaggagctgcaccttccatttctccattccggttaccactcgcttccgatgacaggccccagctgagagcgcatctcaggtggatgtgtgtatactttccccctaacttcctgtctttgccgtagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgagaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatgggtaaggaaactcggcttccaggtgcttcctcctgcttcctcgccagcttcttcttggcgcttgcctcctctcatctcttttcaactatcttccaaatactgttgccacagctacatcataatgccaccactgtctgtttgagactccttcatgagcaaagattgatgtatggtaggtggataaatgggatgaggaaaaaaaaaataaaacaaggaacaaatgtttggggaatgccagtttagcacaaggtattaagtaggatgtaggattgcctacaaatatttgtcaactgaaatttcttgtaagctacttggagcactagtgcctaagagtcagtcaatgtctgtggggcagaacaatgccagagagtctgtgtcccaggagcgaggggacagagggcctcagagctggaggctggggggatgtttcttagaaatgtgcaatctaagcctcctttaaggaatggtgggacctgggcgcatagagaagaaattaaaggaattctgcactgaagactggtatgagccaaatcatgaatgcagtctgagaaaaagtgtgtgcaggtgacagtggagagagacacagtttccttggaaaaaaacatgatgaaggtaaatcaagggaaatagagcagaaatgtggagaccctctttctaggcccccatgttgctctgcactgggcattgccaaaaccccgttttcgggtctgttcacacacttgctgggtggttggaagctagtggccaagtctgtgcaatgaagtggttgagtcacctgacttctgagtcccttttgggtttatcgttcagtgcctcctgcagaaggaccacccacagtggttgtaggaaggtggccaccctgtgatgtgtgagtttagcctggatagaagacacttgacgtggggcaaaattaagggaaacaggaggcttggcccttccacaggcaacaatgagtttcagctttcttgatccgttgacattaccatgaccaggaccaccttcctagtcatggtttggccatgaaggactgactgcccacagggccgcagtggtggtgaagacccaggtcctggtcacagggacttgaggtctggctgtgggacagtccggaaacaagtgcagatgtcggggagggggtcatgggctctgacaaggaaacttgcagagtatccaaagcacaaggggacgaacctgttcaccagcggggcacgggtgggtgtccccgctcagccagcttccggaggatgggcagtgagagagtgagagaaggaaaggggcagccaggctgggggcttacggtgttgagagagagggcaggtgtgggaaagcagggtacggtttctacggcagcaaacagttccgagtgactggaaaaaagacagttgtcttcattcatcaggttgccataacaaaacactgcaggctggttggcttataagcaaaaggaaggttcaccttacggttctggaggctgggaagtccatgatccaggctctggcagatctggtgtttggtgagggcctgttccctggtcgatggtgccttctcactgtgtccttacatggtgaagaggcaaggcagctctctggggtccctttcctaaggacagcaatcccactcctgagggctccaccctcataacttaaccacacctcaaaggccccatcgcctgatacgtcatgttgagggttaggatttcagcgtatggattttggggcacacaaacattcagaccatagaaatgggagaagaagagtaagacagagagaaaagtaggcagaactactcattaagagtgaggtggaaggtggagagggcagggagaccttatgaagatcacacatctgtgaggaaggtttgcacactctcactgaagccccggtaggaggctcagcacccatttctctagatagcaccttccttcctctgaggacagattctgactggagccctggtagggggctcagcacccatccctctagatggcacctgccttactctgaggacagactctcactgaagccccggtaggaggctcagcacccatttctctagatagcaccttccttcctctgaggacagactctgattggagccctggcagggggctcagcacccatttctctagatagcaccttccttcctctgaggacagactctcactgaagccccggtaggaggctcagcacccatttctctagatagcaccttccttcctctgaggacagactctgattggagccctggcagggggctcagcacccatccctctagatggcacctgccttactctgaggacagactctcactgaagccccggtaggaggctcagcaaccatttctctagatagcaccttccttcctctgaggacagactctgattggagccctggcagggggctcagcacccatttctctagatagcaccttccttcctctgaggacagactctcactgaagccccggtaggaggctcagcacccatttctctagatagcaccttccttcctcaggacagactctgactggaaccctggtagggggctcagcacccatccctctagatggcacctgccttactctgaggacagactctgacgggagccctggtagggggctcagcacccatccctctagatggcacctgccttactctgaggacagactctgactggagccctggtagggggctcagcacccatctttctagatggcagctgccttactctgagggcaggagcttgctttgtatgttccagtcccttctgagaaccagaagcctgagcatctattagaaatgcaggattgtggacccaatcttagacctagagagctagactttgcatttgaatgagagctacaggtgattcaaaggtgactgcatgtgtgggtgcagtggctcttgcctgtaatcctaggttttgggaggccaagatggggggattgcttgaagccaggaattcaagtcctacctgagcagcaaagtgagagcacctctctacagaaaaacaaaagcaaaaatgtgactgcatgttggagccacctggctttttttaaataaaaaagatccatatagctggacacagtgactcgtgcctgtaacctcagctactcgggaggctgaggcaagaggattgcttgagcccaggagtttgaggctgcagtgagctatgatcatggcactgcattccagcttgggtttcatctgaaaaaaaatttaaaaaattcatctagttccacctcagagcttctgattttatttccatggatgtggcctgggcttgcgattttctttttcttttttttttttttgagatgaagtcttgctctgttgcccaggctggagtgctgtggcatgatcttggctcactgcaacttccacctcccaggttcaagtgattctcctgcctcagcctccctaatagctgggatgacaggctcgtgccaccacacccaattaatttttgtatttttagtagagagagggtttcaccatgttggccaggctgatcttgaactcctgacctcaggtgatctgcccgcctcggcctcccaaagtgctgggattacaggtgtgagccaccatgcccagcctggcctgaggatttttgtgagcactcctgatgctgttgtgtagccaggagtgaggactgccctctgatcacccagcacagaacgttcagggccatgttgggacagtggactggtaagggcgagaggaggacactggggagcaggctgtagggtgccacagtccgccacagtcacaggctcatgctctccgtccgagaggaggcagaactgcttttaccaaggctcatccagccctcttcatttctctgggaacaatttttggtttcgtgacccatttcttaatccccggaaggcatttctctgaactggtgccaggcaccccggcccccagctccagttagttattattcgaggctgatttcttttctgtctgtcttccttctaattcctagattggtgaacagccacctcacttccagtttttgccggggcctcttttcagttctgagcaccagccagagtctaactgaattggacctcagtgacaattctctgggggacccagggatgagagtgttgtgtgaaacgctccagcatcctggctgtaacattcggagattgtggtgagtccccgtgcatgtgatctgtgtgagtgcaagttcatatgagagagagagagagaaacagactggagaaagatcttcaggaccaggttgctggtgtggtttctttcttttctttttctttttttctttttttgaggcagagttttgctcttattgcccaggctggagtgcaatgccgtgacctcggctcactgcaatctctgcctcctgggttcaagtgattctcctgcctcagcctcctgagtagctgggattacaggcacccaccaccatacccagctaattttttgtatttttagtagagatggggtttcatcatgttgtccaggctagtctcgaagtcctgacctcaggtgatccgcccgcctcggcctcccaaaatgctgggattacaggcatgagccaccgcacccggccggtggtgtggtttctgctgtaacaaagtgccacagactgaatggcttcaacaacaaaaattcattttcttgcagttctgaggccgaaagtctaagatcaaggtgtcagcagggttggtttcttctgaggccgccctccttggtttgtagatgcccccttctccctgtgtactgatgccatcttgccttggggggaagagtggcctagtctcctcttcttacagggacagcagtcctgatggattaggcacagtcaggcatgactacacaagacctaatgttaatctagtcactcctttaaagaccccatttcccaaaacagccacattatgaggtcttgaggattagggcttcaacatgtaaatttttgggggggcacagttcagcccagaacaagtgagaaggtagaatgggttggaggccagctggggaagttttgtgtggggaagaatgaatgaggaactaaaaaaaaagtcatttcttgaaacaaccatgatgctcgatatattccatttcctcatgggccatgttcatagatgagagttggagaatttgccatttacaacaatggcaacctcacctaaaaactatgaaaatactttttttttttttctttgagacaggacctctctctgtcacccaggctggagtgtgtgatcacagcttactgcagcctcgacctcccaggctcaagcaatcctcccacctcagctcccccgagtagctgggactacaggcatgcaccaccatgctggctaatttttgtattttttttgtagagatgtggtctcatcacgttgcccagcctggtcagaatgctttgtatgggttgaaatgtcacagggaatctcctgtgactttattaataatgtgcttgctcacacctgtagtcccagctactcgggaggctgaggcaggagaactgtgtgtacccgggaggcggagatggcagtgagccgagatcgtgccattgcactccagcctgggtgacagagcgagattccatttcaaaataataataataataataataataaaaaatgtgcttgctttctcccacaccatgaatcagattcacacgccccaccgcgctggtgcctgggccgcctcactagctctgtttcattccctgctctgtggacgcatgcaggcgtctgcatttttgaggagctgcacagcaagtgttaaagtccgccactctaggacattttgctacctccctctccggctcaacacactcaccttgtcaccagtcttatcaatttttcctcctacccgagctgttttccacttttcctcgtcctcaccccaccagcccaggtccagcaactgtcaccacttgcctggacacgcagtgacctcctagaccatcagctccccggggatggggtttgcgtttgtttgtcttgctgtcatctttgccacgcctggacagacatcttagtacccatcagtggtcaaaagcacgcactgaatgaatgttggctctttggtctccctgtgtctgcctttttccctcacccgaccattctctgcacaaaagtaagatggtcctttagaaaattctgattcgattgtgctgttcggtggcttaaatgctttctgtctgctctgctctttattttttactttatgtatttatttttgtagatgtagggggcgcaagagcagctttgtttcatggatatgttactgcacagtggtagtgtctgggcttttactgtaactacccctttttttttgagatggactctcgccgtgttgcccaggctggagtacagtggagtgatctcggctcactgcaacatccacctcctgggttcaagcaattcttcgagtagttgggattacaggcatgggccaccacgcccagctgatttttgtatttttagtagggacaaggtttcaccgtattggctaggctggtctcaaactcctgacctcgtgatccacccgcctcggcctcccaaagtgctgggattacaggcgtgagccaccgtgcccggccttagtgtaactcttacccaaatagtgaacattgtacccaataaataattttccagccctcacctggctcccaccctcccacccttctgaggctctagtgtctatcattccatgctctatggatgttaccctttgtttagctcccacttgtaagtgagaacgtgtggtatttgactctgtttctgagttgtttcacttaaggtaatggcctccagttccatctgtgtggcttcaaagatgtgacttcattcttttctatggctgagcagtattccattatgtatatagatcacactcttcatctttgctcttttggtaaatttcaaatcgtagtgccatggagactggttgtttgggacatttaaccttatttctccaacttcatgcccaaattctctccccactgccattgcccctgtatgattctggagaggcagacacaaacagggaagggtaggggacagtgagatgcttcgcagagaagtagagatccatttgtccatttgcttgaagttggtgtaaggatagggacagaaaggaaggtggtctaacaggctccaggacctatgaactattgccctggaaagaagctagaatgttgttcttgcccccatgacagggataaagtatagtttttgttgctggtggtggtggtgacggtggtagtgatgtgcgtgtgtgtttgtgtgcatgtgtgtgacggggaaactaatcaagaatcagagctgcagcgggatgtggaatcccagcactttgagaggctgaggtgggcagatcacctgagcccaggagttcaaaaccagcctgagcaagctggtgagaccttgtctttagaagataataataataataaaaataaaatataaataaaataaaatggctaagcatggtagcacatgcctgtatttgcagccacttgggaggctgaggcaggtggatcccttgagcccaggagttcgaggtggcagtgagctatgatctcgccagtgcactccagcctgtgcaacaaagtgagaccctgtctttaaaaaaagaaaagaaaagaaaagaaaaagaatcagagctgcttttcctttcaatatagatggagggctgggatgaaatcgtcaaacacaatggggtgacaatgttggtgaagacaagaaatagctgagtgcagcactggcgagggcagtgccgactgcgggcacgtcaatggttaggaacacgagggaaggaggtgggtgcacaggcccacaccgcccgcttcacttacaaaccaggagcgcaccattcacatgtctggggggtgatctcaatgcacccggcactgtttacggccgggagttcactgacaggtgatccctctgctgtgactggggagactgggggctgacctcccaatgtgccttgtgctgtattccggagctctctggtcaggtgtattctgatgctttctctattccagagctctctggtcaggtgtgtcctgatgctttctctattccagagctctctggtcaggtgtgtcctgatgctttctctattccggagctctctggtcaggtgtgtcctgatgctttctctattccggagctctctggtcaggtgtgtcctgatgcattctctattccagagctctctggtcaggtgtgtcctgatgctttctctattccagagctctcttgtcaggtgtgtcctgatgctttctctattctggagctctctggtcagatgtgttctgatgctttctctattccggagctctctggtcaggtgtgttctgatgctttctgtattccggagctctctgatcagatgtgttctgatgctttctctattccggagcttcctgatcaggtgtgtcctgatgcttcctctgttctggagctctctggtcaggtgtgttctgatgctttctgcctctgttcttggcatgaaggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggtgagtcgagcccgttcccctaaggaagttctgccagcgaggcgtgctgcgcactctggcttcagtgaggcccggtgggctggggtttgagtgagaatggccttggcggctcgggtgactgcgtgtgcttgtcttggggtgtctagcattgcggtcagtgagtgtttgtcgtggggaaagccacacactcaaccctgacaaggagcataaataataacaaaaagaagatgtctgcctttagggtgcctactgcctagctgacaaaaagaaaaaatcagtgtaatgcattgctttagaggagttttagaacttcatgacaagccctgggtcctcatattgtataagaaaaatggtgctaaagcaatgcagaccctcgatgttgtatgcagcccttccctgagtgcttctgggagacttgcattttgaaggagagaaaaaaccacttaaaaatggaagggccatttctcacccattaggatggctattatcaaacaaaagcaagaaaactagaaaacaacaagtattggtgtgggtgcggagaaatggaagccattgtgtgttgctggtgggaatgtaaatgatgcagccactatggaaaacagcacagcaattcctaaaaaataaatacagaattactactggagccagcatttccactgctgggtatatctgcaaaagagttcaaagcaggaactcagccaggagcggtggctcacacctgtaatcccagcattttgggaggctgaggcaggtggatcactggaggtcgggagttcgagactagcctggccatcatagtaaaaccttgtctctactaaaaatacaaaaattagccaggcatggtggtgcatgcctgtaatcccagttactcggggtggaggcaagagaatcttttgaacctgggaggcgggggttgcagtgagtcaagatcccgtcactgtactcccgcctgggtgacagaggaagcattaaaaaaaacagaaggcaggaactcaaacatgttatttgtacatctatgtccatagcaacactattagaaatagccaaaaggtggaaataaccaaacgtccttcaacggatgaagggtgttgaaaagtgctatatatgtacattcagctttaaaaagaattctagtacacactacaacatggatgagcctcggggacattatgctaagtaagataagctagactcgaaagggcaaatactctgattgtccttataggaggtgtctagaacagacagattcatagagacaggaagaaacgtggttgcaggcaatgggagggcggagagtgggaagacattgtttaatggttatggagtttcagttctgcaagatgaaaggagttctggagatggtggtggtgatggttgcacaacaatgtgaagtgcttaaggccattaattgtgcacttaaaaatggttaaaatggtaaattttattttacacgtattttaccacaataaagctgaatgtagggagctgggaagatgtagtattggtgggagcttggagaatgcttacttgttattccaccaaatggaaaaggaaatcagaagtgtacatagagcttgtgtccactccctttccatgtgtaaactggagtagaggcagtggcaggtacgggtgcttccttgtccatggtggagcgtgggtgagagacatgactgacattctgccatctctatggaaggttggtcagctgctgcctcacatcagcatgttgtcaggatcttgcatcagtattgagcaccagccattccctgaccagactctatgtgggggagaatgccttgggagactcaggagtcgcaattttatgtgaaaaagccaagaatccacagtgtaacctgcagaaactggggtaagtcttcatgggtgtcttaccagaaaagtaacttcttttcagaggtgaattatttggaaaatgtggatgggggttactttggtcaggcagagctgaaggtagagtaaggaggtaaaaacatcagaaatgctttgaaggactggaggcatataatgtatactgctgctaataagataatttatgctaatttatgattatagaactagagtttcttagtgttggaagaaaccttagagatggtgcagcccaattgcctgttgagggaaaaacttgcttccaagacactcacatgaatagtcacccattttatgtgtgcatgcttgcaagcatgcaagttatcgcatagtgcatgcagcctctacactaggaaagagctctcaccctgcagcctctacactaggagaggactcttacactgcagcctctatatcctcaacagtgtcatggctcagcaccttcacctatgtagccatgctctcccaggtacacaggctggtttgttagtgtctgtctttgaaatgtggtgcccagacctgatgatgcatggtggatgttttacctcctgttgcagagaactatggtttcactggtggaatctctgatttccttgttttttttggcagttacataagccttttgattcatgttggaattctgcagtaaacagtagtagggaatacccactaataatcatgcactttccctgtaccactttcctgtatcactttccctgtatcacctgctctttcatgttcttacatggattttctcagttggcttcagaaagatgctatagggtagggtatctgtgtctccaatttaaagaggagaaaataaaattcagggagcctcataactttgtccaaggacactcagagagaaggtagtggaactcagtgtagacaaacgtctgtgacctcccaagccactgccttaattcccagtgcattgccttaggagatgttgctaatgggctctctacagggcttcttcatctggctctcagcttaagggtgggacctccaatttagctttgctaaacctcatcttacctgtcttttgacccattgactaggcagttttgaatcttcattctgacaataatatctacaaatacaataagcaaattggataggtcttcattcattcacattattaatatatataccagccagatcagcattcaagacaaaatttggcaggaaaggtcttcccagaggagtaatccactaatccataattaacaaccaacctctgttcacaaagctctgatacttccttggacctctcccatcagccaatcctcagatgtgaccatatctctttctatgaagtcttcctgcagtgccttcatttcctcgccttagggtattacccttccttagcaaatcgccttagaatattactcttccttggcaatttgacttctgcttctctaagtttgttcaacagacatgattttgtcccttcattttgcaggccctctgggggctctgtggtgggcactgacagcagcagtgagtaaaacagaccaaaatcctgttttcacaaccgttcttttagggaagtgataacagataatagacatgctaaacaatataccctgtgtataagagctataagaaaaactggagtgacggcgaggggtgcttctctgctcacgtttggcgagtgcctctccgagcgggtgacattggagcaggtgctcagtgaagcgtgggatgggctgtgaaggagccggaagagccctccaggcaaagcagatggaaccgaagaggcccaggcgtaggcactcaggtgactctcagtgatcctggttccaaattccacatgacaaacaagccaattacaaatgtgcctgatgcatctgtaatatgctctatgtagttactattgtcaatacctaacctggtagagctgctgtaataaagtatcacaaacagagcggcctaaacaacaagagtttattgcctctcagttcaggaggccaaagtctgagactgaggcatcagcaggctgggtccttataggtcgtacaggtccttcccagtccttactttagttgacatctgctgcctttaattcatttcactcttcctctttggaagccttcttcgctcttatttttaggtcactacttagtctttcctgctaatgttatagcttccccctccctggatcctgcattggcttctcttcctgtggtctggggatttcacgactggtttagttgtgtttttttttttttttttttttttgagttggagtcttactctgtcacccaggctaaaatgcagtggcgcaatcttggctcactgcaacctctgcctcccgggttccaacaattcttctgcctcagccttctgagtagcagggactacaggtgtgcactgccatgcctggctaatttttgtatttttagtagatacggggtttcaccatgtttgccaggctggtctcgaatttctgacctcaggtgatctgcctgccttggcctcccaaagtgttgggattacaggcgtgagccacacacccgcctggtttaccttttttgttgttgttgttactaaatatttctaaactccctgagaattctcatatacctctcaatttcaacaacagtaggtctcaaacccagatatcattcacaaactttttattagaaaagtaacacatgcttggtgtaaacatatcatagtacagacaaagtggaaagtgaaagctccttctcagccctcttctgagagagccatttacaatagtctgatgaacaaccatccagactttttttaatacaatagattgttatataatccattcatctgtccatccatccatccatccatcatccatccctctattttccatccgtcaatccatccatccatccatccatccatccatccatccatccatcaatccatccaagggagtatcagagctttgtgaacatatattggttgctaattatggacaaatggatttcctgtatatgctgatctgggtggtatttatatcagtaatgtgaatgaagacctatgtgattttcatgttgcatttgtaatacattgttatgtagtattttaaatgcctagtctgttagagctactgtaataaagtatcacaaacagagcagcttaaacaacagaaatttattgtccctcagttctggaggccagaagtgtgagattaagatgctagcaggctgggtccttctgagggctgtggggaatctgttcatgcttctctttggcttctggtggaatgtcatcttgctgacacattttgccattccttgacctgtagatgcctctgccacaccattgcctttatctctacgtggtgtcttccctgtgtctgtgtgtgtctttctccatgtcccaagtttccctgtgaaagaacacaagtcatgttagatttgtaacgacctgatcttaactaattacattttcagtaaccgtatcaccaaataaggtcacaatctgaagtactggggcctaggacttccacatataaagtcttagaggaaacaattcaatccataacaccctatctattgaaactagaaccataatataaatatttttctgtgatatccttttttcatttatcatggataaatttccatgctgatttgtgtagatctatcgattggggcataatatcccatagtgtgctttaccataatttatttaatcaaatcatgcattttgggataactgttcttttccactattacaaacaactctgcaaaaagcacatttgtatatatagttttatgcatgtgtatttgctttgagataggtatctataagtggcattaatcagtcgagacttttatgcattttatgtgttaggtacagccatacatgtggctgtgtgttaggtatagcctatgggttaggcatagcctgtgtgttaggtatagcctatgggttaggtatagcctacgtgttaggtatagcctatgtgttaggtatagcctatgtgttaggtatagctacatttttttctctcgaactttgggtcctcatctctggctgcatattgtgtactgcctgtctcaatactgctcaagggtcacacatgctgagcatttgcaacattggtttaattatcataccccaccttccacccccgttctctgacctgctcctgccctcttcctcctgcctcattgaatagtggggctttccaggaggctgctgagtgagtaaacgggggacggtcctaaatgctctgctttcggttccctctaccactcatcactacaccagagggaactttttaagatgcaaatttgaaaatattgaccctttgttcaaaaatcttcaaaagttcttctctgcagtcagagtgacatcttagcttggcacacaaggctgtccctgatactgccctctctcctgccataccttgcactccattttgagtcatattggatgagttttgttttgctgttttagagatggggacttgctatgatgctcaggctgaatttgaattcctgggttcatctacagccataccacccagagtgcctgatctcatctgcactcctgggctcaagcaatcctcccgtctcagccgcccaagtagctgggactataggtggacatcactgtgcccagctatgaattagttttgaaacatcttggtggttcttttaatcttccttattcttgctactcttttccttttgcctggaaggttacccccactttcccactttctactatccatctaagaagctcctacttattttttaattactgtgatcctcattttgtccatttaaaagcagggataaaactaacattcccatcttgaggttgtgatcattcaatgagataaccctgtttaaagtcgtcatccatgtcaagcacacaataaatactcaaaaaatgtttgatcttcttcttcagattccttccttccttccttccttcttcctcctcctcctttctttctctctttttctttttctctttctttctttctttctctctctctctctttctttctttctctttctcccttcccttcccctcccccccccctttccctttcccctttcctttccttattttcctttcttttcttttctttttttaagacagggttttgctctgtggctcagactggagtgcagtggtaggatcatagctcactgtagccttgaactctcctgggctcaagcaatcttcctgcctcagccttctgactagctaggactacaggtgtgtactaccatgcctgggtaatgtttaaaatgtttaaaattttttatgtagagacagggtcttgctatgttgcccaggctggtctcaaactcctggcctcaagtggtcctcccaccttggcctcccaaagcactgggattcagttttcattatgagacagttttgtgacataggcaggtagttaataaataaaaatccccaaccaattaacttaatacagtacatggcaagatatccgtgcttggtaaatgtagagctcagttaggtggattggttgaaatcgagatgtcaatcacattcctctaatccaaggattggttggtgggacatggttgtttgattctttaaacattttattatgtgttcagtgtaaccgttgcttaatagttaccagcattttcacaatttggatatgacgttttcttaacaaaactgcaccgtctgttgcaatttctactactactgtgtagtgctccccaaacatctgttgaataatccattctactcctttttggaaatagtggtaaagcctgcataactttaattacataatttaaaaaatattttctcttttcctctttgaagaccatccccattctactagaatgtccctggcctttgccaggaatgctactaatcccttctgcatgttaattaacaacaaccctctgatagaattcttaccggggatgagagcttagtagaagccactcaaaacttgctttgtttttgtctgtctctcttgggcttcatttgcctattagtcttacttactgtgtcttttcagtttgactcttccttaattgggaagatagaaaccagtggatggataatcactttttcctgctggtcatctgttcatgctttactgtctgaatacactttttctgtatttctatttttcctgcctcgtagacaccaaagagcttaaaaaacatgccattgccaaggactctggcaggaaggaggcaatcacctgcgctattcctggcttcatggaatagaagcatctgtgctcctaggaattacgccaccagtgattttagagctcttatgctcttcctgggtgatagaatgggcttaatttgactcaagaatagtacatcaaaactcttaattccatcgagattgatgcatttttggggttttatgctctaaaaggccttttgctgtgcagaaggaagagagaggtggacagaaagggcagtggggagaagatgagactaaaaggtggaaagtccaggtctggatccctccccaccacacacctgctctgtggccttgagagaaacatgtaatttttataagccttagatttctaatatgtaaaatgtatgtaatagttttagctgcactccttccttgccaggttgatataataatcaaataagactggtctctgggtcaggcacagtggctcatgcctgtaatcccagcactttgggaggccaaggtgggtggattgtctgagcccaggagtttaagactgcagtgagctgtgcacttggcctgggtgactgagcaagagcctggctttttctatttatttatttatttttgagacagagtctcactttgttgcccaggttgaagtgcagtggcaatctcggctcactgcaacctctgcctcccaggttcaagcaattctcctgcctcagcctcccaagtagctgggattacaggtgcccaccaccaccctaactaatttttatatttttagtagagatgggctttcaccatgttggccaggctggttttgaactcctgacctcgtgatccaccctcccaaagtgctggacctcccaaagtgctgggattacaggcataagccactgcacctggccttatttatttatttatttaggcagggtctcactctgtcacccaggctagagtgtggcagcgcaatcatggctcactgcagcctcgacttcctggactcaagcgatcctcccacctctccaacgtggttgagggctgcagcgagaaagatgtgaaaggggcatttgggggcgcaggaaggcgggaggtggcaattgctaagggccccgccgtgttcctgtcacagccctagctacagcttagaaggtcacaatggacgttcacctgttgtctttgatgtttaaatcagcaccgacttcccatgaggaggatttttttttcctgtctctcttaaaaaaaaaaaaaaaggcccctgcagtgtgggtgtctgcagtgttacggggattattagttgaggataaccactgggatgtgtttctggaagaagtggtgctgaggagcactcctggccggaggaaagctgtgtgaggaggaggaggagtatggggtaactggctcagtttgccttggctctttctgtcggactcatacagcaaagcatctgctgagacatcatttgtgtctcctgtgacagccagagacagcaggtcttgctctccccagatcatatctgagatgctggctcctgctctctgagctgaaggctgcagctgctgagagaggacgaggcactgagtcaaagcagctgcacaatgttggggaacagctgggtactgaggactcttctccctgtccttctacaggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggagtaagtcctttggtttattacagcaatgagaacacatggtggccaagggtggagggacgtttaggcagagtggccacaagataatctgtatcttagaagtgaggtgtcttcttagtgtttgccttgttacaggatcatgggactgggaggagtccttccagatgatatagatgtaggaacttgagtccttaccatcacagttttctgtaccacaaaacaaggagggcagtatgacacttacattttatggaaatgtagggaaaggcacagaatttggtaaatcaatgaacaaaccactgttactaatttgatctgtgcaatgcagtggatttgaaaaagagagagaagaataattaccagcatacaaggctgcttgaagctagttagtcctgtgctcctgtgctttttttttttccacctgaaacaagacaggctaagatgctaaaatcttggggagctagggggatggttaaggggacattttctttaaatcaccccctttttgcagattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggtgagtgtgctctgcagagatgcccgtggtgggactctgagttctcaggaaacgttgactgttatcaaaatccaggatggctctcgcttcatttgcagccactcaagattaggttgggcctgggtcagttgtggtggatattttaaaatagagaatatggggttaaactacacattccacttcactgagatggttagaatcaccaaggctaagctggatttcacccacattccccaaagaaacacaagccctcatgcgatcatggccaaggaggattctgttctacacaaggaaccgaacttcgtagaactcgtctatgggtcatccaaattggaagggtctttacatagtggggatcttgttgttttgtttctgaggatttattttttttttccttgagacagagtcttgctctgtcgcccaggctggagtgcagtggcatgatctcggctcactgcaagctccgcctcccgggttcacgccattctcctgcctcggcctcccaagtagctgggaccacaggcgcctgccaccacacccggctaattttttgtatttttagtagagttggggtttcactgtgttagccaggatggtctccatctcctgacctcgtgatctgcctgcctcggcctccccaagtgctgggattacaggtgtgagcctccgtgcctggcggtttctgaggatttctaaatgggaaaagagtgctccaggaatagcaagatgggaaattacacgaaattcgacagtgtgtcaggatattgccagcccacctctgtgatactactgaggtagtcgtctcctctctagcaagagcttcctggcgagtcccacggttctgtcagccgtgatgattgcacttttccctgaagacgttttcactgcttggctttaaggccaacatgctctcctgggttttctcctatttcatcagatccttctactcagtctcctttctggcctcttcttcatcatcctgacccttatgtgttgaatcctcagagcttagtcgctgggctaatttttcttccctacactttctccctcatttaaattatctggtcttctggcttcatgatatgtgtgctgtggcctccagattatatgtccataaattctgtgtctccgtgcttggctgatctttcttcttgaaggtttaataaacctctcaaacgtcacatctagatccacacccttgctactctcttctacccgaaacctgattcttccccatttcagtcgatggtcacttcctccaagttgctcgggccaaaacatttctggttctgtcgtcactgtctctttttccttataatcatgtccaatgtgtcagcacatcctgtgagctctgccttcccagcttggccactccctgaccacttcaggccagcttcacggcttcttccttggctcaagccaccatcacctggtgtctggattgtcatggtggcattctgacaggtcaccctgcatctgttattctgattttagcagacaagggattcagctaaaacgtaactcgcatcatatcactcttgtcaacatcgtcttcttatcactttcataatcagagcaacaaactcatggtggcttccatgccccacataatccagcctctcctgcttccgaagattccgcttctcccaacccttgcttattctgccccagtacagtgatctccttgctgttttgcaagaacactgtcatctctggatttttgcacttgcttttcttctggcctagaactctctccccttcagtatccacctgcattattccctgtaccttcctaaattctctgcttaaaggtcttctctgcgaggctgtctttgactattctactgaaaatagaagcactttttcttctcttctcattctcctctgcagtgtagcacatcttatgtgctatgtcattttcttgtttatttattatctatctctgctcccaaaatataaattccattagtccagggacttctgtccatattaatgatctcaaggctgagaacttgttggaatgcaggtttgaataaatacaagtttgaatcagccttgcatccttgtgccaacataatggagcacatttgctgcaggacaagccaatattcccagagctaagagagatgggggaacaaacaaaggattggtcaggtcaaagctggggacactgctacatgtagcatcactgacctagccctgagggacattctggacccaggctgagggacaggctcaaaatagattgaagaatccatgaatgcaatcaacccgttttctcaggctgcgataacaagctgctttagactgtgtgactcaaacacagatttattatctcacaatactggaggctagaaagtccaaaatcaagtctctgatgaatttggttgctggcgagagtcctcttcctgatttgcagatggctaccttcttgttatgtgattgcatggagggaggggaggcaagggagagaagacaggggagaaagaacaaactctctgatatctctcctgtaagggcactcaccccatactgagggccccaccctcacaacttcatctaactctaactacttcccaagggcctcacctttaagtaccgtacattaggggttcgggcttcatcatgggaattgttaaggaagacaaacattcagtatataacaccaccaaattgagatggttataaatggtgcaggacaaatgggtggatttattagttaagttgcaagggaaactttattaaagaggaggaagaacttacatcgaagatgtgagaggcatcctgggcaaatgcaatgtgtagaattgatcaggttagtgactaaatgaacataaggtaaaaaaatttacaagacagttgaggaagcgtaaatgctgactggacatacagtgatactaagaaattattgttaaacgattttggtgtgatagtgacattgtaattatattttagtgtcacaaagtgaattattgatagatgcattaaatgcagtatttaaaataatccatgggtgggaggagtgggtgggcaatagaagaaacaagttgacatgacctgtttaattgtcgaacttgggtaatggacacagggggttcattatactattttctatacttttatgtatgcttaacattttcccaactagatagttaaaaatataacaggaaccacattgggcacttagagcttttttttttttttccccacatttttattgggagccgtgggaggggcctcctctgtcattggaggtgctcacagtttcttcagccactccaggctggggtcctgggggtctgaggctcccagtgacgaaggacacgacgagcaccggctccttggcccaggcattcttgaagaaggtgcggagtctcgcggccatcttggtctcggcggcggcgacggcggcgaggacgcggagcactctgggagttgtggtccctcttttttttttttttttttttttacattttagaagtgtgtggagtttagggaaatgaagagatgagacttttaaggttaacgactctgacagagttatctgaagagtgcaacccaggctttctatttgcttttacaggttgtctgaaatgtatttcaattatgagacaaaaagtgcgttagaaacacttcaagaagaaaagcctgagctgaccgtcgtctttgagccttcttggtaggagtggaaacggggctgccagacgccagtgttctccggtccctccagctgggggccctcaggtggagagagctgcgatccatccaggccaagaccacagctctgtgatccttccggtggagtgtcggagaagagagcttgccgacgatgccttcctgtgcagagcttgggcatctcctttacgccagggtgaggaagacaccaggacaatgacagcatcgggtgttgttgtcatcacagcgcctcagttagaggatgttcctcttggtgacctcatgtaattagctcattcaataaagcactttctttatttttctcttctctgtctaactttctttttcctatcttttttcttctttgttctgtttacttttgctcatatcatcattcccgctatctttctattaactgaccataacacagaactagttgactatatattatgttgaaattttatggcagctatttatttatttaaattttttgtaacagttttgttttctaataagaaaaatccatgctttttgtagctggttgaaaattcaggaatatgtaaaactttttggtatttaattaaattgattccttttcttaattttaaaaaagagtacctagaaatttttcaaattatttctctaaaccactaaagataaattaaattgtttggtgcattttaaaagtcactttggcaaatgattccattgtagataatttttaaaacgttacctcactctaactgtaatgagtggaatttcatctttgataggataggagattgaaaatcgtggagggaaaggtttgagagtatcagagcctgcgctcctaactagaagtattatgtagaagatgcatcttgctttcagttgataacagcttattttggatttatcccatccgaggaacagttcatctattatacttcacagagcattaagttataaggaaatatttactaattgcttctctacacatggacaactcttaaaccgcatggaattgctcaggatggaaatactatttcctctgtacaattagccatcaaagtagccacactttgctactcaaagtgtagtctatgaactgtcgatataaggagcttgtataagaatgtaaaacaatcacattattaaacataccttttagatgagctgacatttaaaaaaaaccctgaagactatttttataagggaaacagtatgagatttacattctgttgcaaactctgtcttgttagcacttctgagcagcaccaactggagactgggtttctgcatcttggtgaactattaccgactgccttcctgctacaaacactttgataacctatataaataatgtaatcagaaacacatagctgagcttgaaagagagaaagagaacctttgggcattaggacattaaaactggccaggttgctgagaatgccattatttcattcctttttatggctgaatagtatttttccatggtatatatctcacattttctttttcttttttttttgagatggagtcttgctctgtcgcccagtctggagtgcagaggcgcgatctcggctcactgcaacctccgcctcccgggttcaagcaattctctgcctcagcctcctgagtagctgggattacaggcgtgtgccaccatgcccagctaatttttgtatttttagtagagatggggtttcaccatcttggccaggctggtcttgaactcctgaccttgtgattcacccgcctcggcctcccaaagtgctgggattacaggcatgagccactgcgcccagatggttccatatttttccaattgcaaattgtgctgctataaacacgcatgagcaagtatctttttcgtataatgacttattttcctctgggtagatgccccgtagtgggattgctggatcaaatggtagatctactttttgttctttaaggatccacaatgttttccatcgtggttgtactagtgtttgtactagtttacatttcatggaaaaactttctatagaagaaaatgtgaactacagagaaaaaaaaatcaaaatcacaggaagaagatagcaatctacttcattcctggaattgtctgaaaaagtcgtgtaaaatttgtggagatttaagtatatggagaaaaacacttattcattgtaagtaaaataagattgtatttcagcttcatcttgtgtatacttaggagtaatagaaaagagacaaatggggagagaataaaagaaatataggaagaggaaaagagaaaagaatcaggaaattggaagtgaaataattccacacatacttattgaagtctatttaaacaatgcttaaaaatatatggcctcttagaattatacgtttaaaatgaactttgtcgctatgggatttggggcattataacaggaagtgagatgtttgaagacagggtttttgagctctctgggtattaactccttaagtgaaagatgctctgagtgcacaatagacttgtgctgtgttctttaattgatggagatgctacatctcatgtcctcatcatccacagagcctccagatcctggccagaagtctcctcagagcccccttcatatctttatttctcagggtgtaggtgaagggattgagagtgggggtgattatggaatagaagagagaaataaatttgccctgctcttgggagtagctggaagggggctgcagatacatgtaaatcgcaggtaggtagaagagggagacgatcatcaggtgggaggaacacgtcccaaaggccttgtgtcgtcccttggaggactggatcctgagcactgcccgggcaataaagccataggagagaaggatgagagccaggggcaccaacacgaagaaggccaccagcacagccagtatggtgtcattcacagcggtgtcagcacacgacagcttgatcacggccggcacctcacagaaaaagttgttcagcacctgccgcccgcagaatggcaattgcaccgtcaggaccacctgcacgaaggagttgccgaagccactgagccaggccagagccacgagctgctgacagagagcacggtgcatgagaacggcatagtgcaggggcttgcagatggccacgtagcggtccagggccatggcggccaggacgatgcactccgtgcatcccagccagtggaagactgcatattgcacagtgcagcctccatagctgatggtcttctgggaactgcccatgttgaccagcatctgagggactgtcgtggtggtgtagcagaggtccaggaaggacaggtgactgaggaagatgtacatggggctgtggagttgaggatccacccgggatgccaggatgatggcgacgttccccaacatggccagcacataggacagcaggaggaccacaaagagagggagttccagccacggcctgtcagacacacccagaaggatgaaggctttaggggagtcccctaagaagctatggttgtcacttttcatgttgcggcattttctggcacttgtggcaaattgagaaaattggaatgaataatacctgagaagaggaattgatcactttcctccctcttgcatccactcttcctttcccaggtgatagcctgtttgattctccttacctctgtggagacgctgggatgcggaagggtcagaaccaggagccccagtagcagcctctgtgtctttgctctccttcctactcagtggccgcaactaaaccatttaatattcctgaacttctgcatctgtaaaatggggagaaatacctccttcaattgccaagacttggggacgatagagacattaagtatgtgagaagaatacctcacgaatgacaattatgttccaggtagagcgaagcgtgctgcagctcccatcaccgtcagagtgtttgatttctctc

SEQ ID NO: 2 - DNA, 4470 bp, Homo sapiens
  source 1..4470: mol_type = genomic DNA
gtagatgaggaaactgaagttgaggaatagtgaagagtttgtccaatgtcatagccccgtaatcaacgggacaaaaattttcttgctgatgggtcaagatggcatcgtgaagtggttgttcaccgtaaactgtaatacaatcctgtttatggatttgtttgcatatttttccctccatagggaaacctttcttccatggctcaggacacactcctggatcgagccaacaggagaactttctggtaagcatttggctaacttttttttttttgagatggagtcttgctgtgtcgcctaggctggagtgcagtggcgtgatcttggctcactgcagcctccacttcccgggttcaatcaattctcctacctcaacttcctgagtagctgggattacaggcgcccgccaccacacccggctcatttttgtacttttagtagagacacagttttgccatgttggccaggctggtcttgaattcctcagctcaggtgatctgcctgccttggcctctcaaagtgctgggattacaggcgtgagccactgtgcccggccttggctaacttttcaaaattaaagattttgacttgttacagtcatgtgacatttttttctttctgtttgctgagtttttgataatttatatctctcaaagtggagactttaaaaaagactcatccgtgtgccgtgttcactgcctggtatcttagtgtggaccgaagcctaaggaccctgaaaacagctgcagatgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgggaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatggattggtgaacagccacctcacttccagtttttgccggggcctcttttcagttctgagcaccagccagagtctaactgaattggacctcagtgacaattctctgggggacccagggatgagagtgttgtgtgaaacgctccagcatcctggctgtaacattcggagattgtggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtcagctgctgcctcacatcagcatgttgtcaggatcttgcatcagtattgagcaccagccattccctgaccagactctatgtgggggagaatgccttgggagactcaggagtcgcaattttatgtgaaaaagccaagaatccacagtgtaacctgcagaaactggggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtaggagtggaaacggggctgccagacgccagtgttctccggtccctccagctgggggccctcaggtggagagagctgcgatccatccaggccaagaccacagctctgtgatccttccggtggagtgtcggagaagagagcttgccgacgatgccttcctgtgcagagcttgggcatctcctttacgccagggtgaggaagacaccaggacaatgacagcatcgggtgttgttgtcatcacagcgcctcagttagaggatgttcctcttggtgacctcatgtaattagctcattcaataaagcactttctttatttttctcttctctgtctaactttctttttcctatcttttttcttctttgttctgtttacttttgctcatatcatcattcccgctatctttctattaactgaccataacacagaactagttgactatatattatgttgaaattttatggcagctatttatttatttaaattttttgtaacagttttgttttctaataagaaaaatccatgctttttgtagctggttgaaaattcaggaatatgtaaaactttttggtatttaattaaattgattccttttcttaattttaaaaaaaaaaaaaaa

SEQ ID NO: 3 - DNA, 30466 bp, Mus musculus
  source 1..30466: mol_type = genomic DNA
acatttattgtctttttggtaatatttattctaccaggcttgagctaatgtttcattatggtttgtgtttgcttgtctgaggcagagatgtatgactgaggctggcctgaactcactgtgtagctcaggctggccataatcttgtgaggaccttcctgtctcaaactccagaatgctaggattacaaaaaagggcttctgtgcttggcttggttccaaataaaaagatgatgatgatgatggtggtggtgatgatggtgatgatgatctacagtactgaccaaccaggcaggaggagtctactggtgctatcgtgttgtaggaagaaaccaaccaatttcctattggatttgagccctgaacataagagggaattcatatttagtattataaacatgatcaaactccagtttcaggggtttgacctcttccgacctccctgggcactaagcacatggcaggtatatctatatacatgcaagcaaaacactttttatttattaatttatttatttaagcaaaaacacttgtacacataaaataaataaatctaaaaatatgtttgtttgtttgtttgttttaaaaaaacacagtacccattccatacccaggagacagcgttttatatctccatctccttccctgcctcttcattcctttctgcccccgcctgttcacaatgtttcccaagcctcacgtgagtgaaatgcatgtcccacctaggactgacgtacaatagtctctcctttaaagatgttttcaggccaggcgtggtagtgtacacctgcaatcccagcgctcaggaggcacagacaggcagatctctgtgagtttgaagccagcctggtctacatagcaagtttcaggatagccagggctacaaagagaggaggctctgtcaataatagtaataataataataacaattttatagtcaattttattgatgaacattttacagttccttgaaattatgtctgtttgtttgcttttttggagggggcgaggcatgccacagcacaagtcagaggacagcttgtaggagctgtttctctctctcaaccatgcgggtttcagagattggactggggccatcttttacttgctgagccatcttgctgacccttcaccaactcttatgtgggactggggaccaaattgagggcttcatacattccaggaaagtactctatctacgctctgtctacagcttcttgtcattttgatttgctattatctgaggactggtgacgttgaacacacactcctgtgggcttttgacttccttttttgagtggtgtttgctcatcctcctatgatggagttgaatgagagtcttgcatatttttgctattaactgcttatctaacctgtggtttacaaatgtcttcttcaaggtcattagtggcttctttgagccactcttgtgtcctcagagcttttgagttcgattgattggtcttggtagcttattttgcctcttgctgtgttgctttttgggagatgcctaaaaaccttcactactgtatcaacaggctttccctgtcttctaggcttttggggcttcagatctgcttaggcctttagcactctgagtggatttaaatgtggtgtgagatcagatagttttattcttcagcatgtaactatccagatttctggagaccaacctagtaatgctttttgcgttttgcagtacttgggacccagtcgttgggcatacccagggtcttgggcacgctcagtaaggcagggggctcctgtcttcctgagtctggctggtctgcttctcttcagccttagcatttgcattcctgttgcttctgtccctgaatcagcagagtctctgccctttcacctgtagagtagcatgaggagctgcctcattcaggacactgagagagtacctcttgctcagagttggttggagtctctgccatggtcagacagtggttctacagtgaccaagggtggcagagaagcaggtctgagcggccatcagttcccttgtccttcggggtgtaaagaaagccaagcactttggctttttcctgggacacagccacctcagcctgacaggtgagaccaaaagtcatggagtaagacttatgcttttaaaactaacaggagatggagaacacttggagctgcgtgctgaccacctccatgccaggagaaagaaattagaatctagggtttcaaagcctgattctgtcctcagctctccacacactggttctgcttgccactgcttatgtccccctctctgggatcctaggcctcactgagctgctcgctgcctgtgctgagccctgaggtttcactttttcccattgtttctaaaggcttctgcagggaacccccgggaattctggtctgcagcttaggcttacactcagaactctattgcttgggtttggaaaatccatccagatgagtaactgccaatccgtctttgacagtgatgcaagcttgttcacgtcttgaagccacatccttcctgtctccagtgtcacagtgacccctatatatttatctcatttcccctttatttgtacccaaggctgctatctgggtgagtacaatgtcatggagcagagtggataaaaggtccagggtatttttaatggcatgatttatcttagtcaatagacaaggaatttcttttagactcatctaacattaaggtatttctgggatgttctgtttgctgttacgtggttcatgcatgttagggaggtgtgtgtgcgcgtgagcgctcacagaagaagcaattatagctccctaggacttaaacagcatctactcagggtggcagagggtggactgcctgtgagggggggaatgtgctggaagcccagggggcagctaatgtaaagagcaaatctgttgtggcaggtcttagcagcagggggcagagggctgagaactcctgggaaagactaggaactgaggggtcaaaagtctaggaggatctgtggagctggggcagcagtcagcaagaacactagagatgacagaaagatgtgaggtgcggcaggagacagggagaggagatgcaagagtggacacgagacaggtacatcgagcttggggcaccttcaatgtcatcttgctgtcacatgaatatcaattcggaggccatcaccacctttcaggcactttcctctaccatctatggcttcagtctgagaaagccacttagtgtctctaccactcagcagaaggaacctacacacctcagtggagaaactagatgagaagagtaaaaagaatcagggcagcatctaggcccagagttctgactaaatgtgaatcacgatgctcgttatagatgtgagatgttaaagagccgttaaagcgatgtacgcatgtagtcttcctctttatatgcacacaattccacctgatcgggttctgtctgtcagtgggaaatagaggaacactgaagggttagctgtcagcccgtctgggctgtgagtcctcaccttggtgctctggggttattatagcttattttctcctggcttcctccttcctttgaaaataaaagcaagaaaccagggctttccattaaaagaaatcgggaagcaatctcttctgctttctcagtgtctgaaggaggaagctgaggttgagaaacagtgaaggctgtgcccaggtcctagcctcgtcaccatgggttctggtcagacacgagtcctggtgactttgtatatgcgtgttctctgtataccacatctgattgtgttaatggctttcttatttttatctctacagaggaacttttcttccatggctcaggacatacgtctggatcaagctaagagaactttctggtaaacatttggcttactcagttcttactaagtcagagatggtgattcattaaaaacctgtaccaatctatggggtacccttagaggtttcaatattagcatctagtttcctgtgtttctgataacttcgttttctggaagtggagacttttaataccattcttctttactcccatggggttttcattcctgcactgccagtgtggacctaagccccgagaccctcgaaagggctgctgctgaagatgacgagtgtccgttgcaagctggctcagtatctagaggaccttgaagatgtggacctcaagaaattcaaaatgcatttggaagattacccgcccgagaaaggctgtatcccagtccccaggggccagatggagaaggcagatcacttggatctagccacactcatgattgacttcaatggcgaggagaaggcctgggccatggctgtgtggatctttgctgcgatcaacaggcgagacctctgggaaaaagctaagaaggaccagccagagtggagtgagtaaacagaagggtttttggttttgtttgtttgtttgtttgtttcttacagtaaggtacacgtgacataaaaggtggaattttagtaattttacatatatggcttagcaacaagaaaacatatggttgtgcaaccaacttctcatcttgcacaactgaagctcccaacacactagatgataagtccccattacctaaccccatctcctagcaaccaccgctgttttctgtccctgtgaactttacctctatggattcctcgtgcaggtagattcatggagggcttgtttcttttggctggcttacttcacttagcataatgtgttcaggtacatcagaatttccttcttaaaaaaggttaatgttgaatattcatgtgtgtctattacattgtgcttgtctcatctgtgcatatccatttgctatggataactatgtctattcaacatcttcagttctctggttttgagttcgagtctcaagtatcccaggctggctccaaacacagaatcctcttttctctgaccatgggttttccttctctcctgcaggggtacgggagggtgagggtcagagggaggggagcagtgtggttttcttaattctgtaaaaactccggtctcagtcttcactcactcgcaggtgactgttgtggctggttccaacctcgtgagttggctcagtgctgaggtcctcctctggttccccaaccagctcatagcagatgctcgtctgctctaaaccagggaagcagcagaagggatttggaataaaccagggaagtcccaggcagaagatgctgagtgggagctgcggaaggaagtagtcaccggagataaggatgctggggtggtgtgactcagcagtagagcacctccctgttatgcacagggatctgggttccactgggccagttggagatgatgatgaggggtgtggcaaggcaatggcaatccgggagaaggagacggagctaggtggcaatccaattgcacatcacaatgcagccgtaattgttcctgacctgagaatgtgtacaacaggagtgaagtctcagcaatacaagtcctgactgagcaattttagattttgtttttcaaattaaagcattaaacgtttttagctatttatgttgtgtgcctgtatgtgtgtgctacggcacctatgtggaggttagaggacaatatgcaggggttgattctttttaccatgtaggccttggggatcaaacttcaggtcatctggttcgtgcctttactccctgaaccagctctctccggctctgtaccaggtagttgggtttgtttgtctgttttttataagatttattttgaattggatgtatgtagtagcacaccagaagagggagtcatatctcgttacggatggttgtgagccaccatgtggttgctaggatttgaactcaggaccttcggaagagcagtcagtgctcttaaccactgagccatctcaccagccccagcacatgtctttatccccagtacttgaaaagcagaggcaggcagatctatgggagtttgaggccagcctggtctacagaggacattgttccaggacaatcaggactatataacaaagagatcaggtctcaaagaaaacccaaaacacaagaaccagaaaacaacaaaaacttcacgtgtgtgagtgttttcctgcacgtctattatgtgcactgtgcatgtgttggcacctgaggaggctagaagagggtatcatattccctgcagctggagctacagacagttgtgagctaccacgtgggtgctgggaactgaatccaggtcctctggaagagcatcaagtgctcttagtcactgagccaccgctccaggcaccctgtacaaagcagttttaatgggttttgcttttctttcaataaccccatgatgtagttcctattactatccgtgctttcaaatgaaggactcgagccagggagcggctctaattacccagggctccccagtcagggccggtcatagtaagagtaaaaggccaggtaagactatacaagtatagccttgtgatgttgcttttatatctgcattttaggcataaacattgtcatcagaatttgaagttctttttggagctgaagtgagacattacctgagctttgggctgaggttcgcggttgcacgaatgtttagatttttgttttgtgactccattgccagtggcactttcatttcttttctcttccctcccttctctcctgcctgtccttctcaagctcagtgtgtggctgaggaagaccttgaactcctgttccttctgccttcacttcctgggtgctgggtctgtgtattaacaccatccctggcttagatccatgccaggaaggcactttaccaactgagctacatccctgacattatcttccagaataattctacatccacaggagtatgcccatgtctttgatctgttgtatgtcttttcctgttggggactttgctatattcctgaatttcccctaggaggaaagtccatattattcggatacccagttcttggctgtaggcacctgttgggatcacctcggactttcccatggataactatgcctaagccttggcacagagtaaaaagaactgctttgagtaaagtgttacccagcattttcaaacttttccacgtgaatttcgtaagccattgctctgaactacttagactgactgacgatgttcccttctgaagacctagaaacctagaggtgacagcataggatatggtccacatgggctacgtacatccaactgagctcaggggagggtgaacctgtcaggagaacattgtggatcgtgcacatggaccctaagtcagggtgccggggacaactagggacagatgacaatggcaaagaaacaccatttgctctcatatgttctctgacagatttctgggcctgggaaagacgttctgagcctggggacacagttctgtctgatgattgacagctcccctttcaaagtgaggccagcagaatcatgcctattaggcttttgatgccataactatcaacagtctgcaacccttgagccatgtttactcccctctcccctgttttttttctttttttcttttcttttttcctctttgagacatgatctcatgtagcctaggctggccttgagctcactatgtagctgaggataactctgaagttctggttttcctgcctccatctcccaagtgctgagcttagagatatatgctctcatgcctggctaagccacatttaattcagaaggaataggcagtaggggccattgacttaccccatagactaggctcaattctatcctctgtgttaggacttttttttgatgtactttgtgttttttcttttgtgcttagatgacacgtgtacatcacattcctctatggtatgccaggaggacagccttgaagaagagtggatgggtttgctgggatatctctcccgcatctccatttgtaaaaagaagaaaggtaagcgtcaagcttctgccttcatctttgagacttgggatagggggctaaggaactggaaagcgggggagcttggccactctacagggagcaagccctgcctcaggaaacccgccttcactgaaggcacagaggcatattcacatggcttcccaaagtcccagggaaggggttaactataggtagatatgggcttcagactaaaggtataaggcttattaagtaacagatttttctggctggaggccacagtgctggctttctgccctatcatgaatggaaagtaattgacaccagaattttgggagcctgatacactcgagcagtggagacaaattatacctcctatttttcatatatatatttcatatatatatttcatatatatatttcacatatatatttcatatatatatatatatatgtatatatatatatatatatatatatatatatatatttgtttggcttttcaagacagggtttctctgtatagctctggctgtcctggaactcactttgtaaaccagactggcctcgaactcagaaatccacctgcctctgcctcccgagtgctgggattaaaggcatgcgtcaccacgcctggctcttttcttatatttttgagccctctgtccggtcataccacctatttttctaagccaatccttacttcctctagagtataaaccactggctttcctttccttttgttaaaacacagccatgttttaatatccacttgcattgtatgttgcctccatacaaatttgggaaagagtaatagatattaggagatctctgcagaatacattgctgccaaggcttacagatcacaaccataggcttcaaaccaatcataaaatggttggctgtgccactactctgtatcaatgacgacagctcgctctatacatttgaatgtttagtgagctcattgcttctagttgacagtcagcaccctgcattttgttgttgttgttcgtaaatcctttcaattcttaatgttccccctgtaacagcagaggtccgctagcagcttattgttggggaatggatgtgtgggcttttcctggatcccgcctttctgtctctgctgcagattactgtaagatgtacagacgacatgtgagaagcaggttctactctatcaaggacaggaacgcgcgtctaggtgagagtgtggacctcaacagtcgctacacgcagctccaactggtcaaggagcatccaagcaagcaggagcgggagcatgaactcctgaccatcggccggactaaaatgcgggacagccccatgagttcccttaagctggagctgctgtttgagcccgaggacgggcactcggagcctgtgcacacagtggtgttccagggagcagcaggcatcgggaaaaccatcctagccaggaagattatgttggactgggcactgggaaagctcttcaaagacaaatttgactatttgttctttatccactgccgagaggtgagcctcaggacgccaaggagtctagcagacctgattgtcagctgctggcctgacccaaacccaccagtgtgcaagatcctgcgcaagccttccaggatcctcttcctcatggatggctttgatgagctacaaggggcctttgacgagcacattggggaggtctgcacagactggcaaaaggctgtgcggggagacattctgctaagcagcctcatccgaaagaaactgctgcccaaggcctctctgctcataacgacgaggccggtagccttggagaaactgcagcatctcctggaccacccccgccatgtggagatcctaggtttctctgaggccaaaaggaaggagtatttctttaagtatttctccaacgagctgcaggcccgggaggccttcaggctgatccaagagaatgaggtcctctttaccatgtgcttcatccccctggtctgctggattgtgtgcacggggctaaagcaacagatggagaccgggaagagcctggcccagacctccaagaccactacggccgtctacgtcttcttcctttccagcctgctgcaatcccgggggggcattgaggagcatctcttctctgactacctacaggggctctgttcactggctgcggatggaatttggaaccagaaaatcctatttgaggagtgtgatctgcggaagcacggcctgcagaagactgacgtctccgctttcctgaggatgaacgtgttccagaaggaagtggactgcgagagattctacagcttcagccacatgactttccaggagttcttcgctgctatgtactatttgctggaagaggaggcagagggggagaccgtgaggaaaggaccaggaggttgttcagatcttctgaaccgagacgtgaaggtcctactagaaaattacggcaagtttgaaaaaggctatctgatttttgttgtccgattcctctttggccttgtaaaccaggagagaacctcttatttggagaagaaactaagttgcaagatctctcagcaagtcagactggaactactgaagtggattgaagtgaaagccaaggccaagaagctgcagtggcagcccagccaactggaactgttctactgcctgtacgagatgcaggaggaagactttgtgcagagtgccatggaccactttcccaaaattgagatcaacctctctaccagaatggaccacgtggtttcctccttttgtattaagaactgtcatagggtcaaaacgctttccctgggtttttttcacaactcgcccaaggaggaagaagaagagaggagaggaggtcgacccttggaccaggttcagtgtgttttcccagacactcatgttgcctgttcttccaggtaatttggcttcgggcagatgatagcatgtgccttatgcttttggctattttttctttcttccttccttccttccttccttccttccttccttcctccctccctccctccctccctccctccctccctctcttccttccttcctttctttcctctcctctatttctgaatattttccaaatataattagcaagcttcatgataatgtgactgccaccctctcgttggtagagagagactttggctaggtggatgaatgggattgagaacaaaacccaatgaactgagaagtaagcgtttgggagaatatcgcttccatgcacagcattgggtggactgtggcactgtgtagatgtctgtagacatctgttgcttgtattctagggtagctctagcacactgttcttcaacccatgcagctgtgatggatgccagattgaagacaaaaggcttagttagttattacagttgtggaggatgggaagtctaaagacagggccctccccacaggctggttttacccggtggagagaagaacgaagctccacaggtctcccttgcacaagatgctgacattctctatgagggtctgtcagcctggccaatcgtcctcctcacctcctgattccgtcgttctgagacgatggtgtctatctgagttttttaagggtagggtgaacgtgcagactgtgccagtgggagagggggagagagaggaggtaagagaaggcagacttgttctttaaggatcgttggagaaacaatcaacagcctgacaaggaccacacactgatgaggaaggacagggactctgctaccacccctgatgagaactgagctcctacctcaggtatcagccagttttcttacaggatacaggctaagtttatcccagaaccttctaagatgactagctaagtgcatacccccatcagaggtccagagtcacatactcagtcctgcatctatacaaagaagctcaactctttttgaaagctagggtttcgtgtagcccaggctaaccttgaacctgaactcatcctgtaactgagaatgaccttgaataattcttcttcttcttcttcttcttcttcttcttcttcttcttcttcttcttcttcttcttcttcttcttcttctttcctagacagggtttttctgtgtagccctggctgtcctggaactcactctgtagaccaggctggcctcgaactcagaaattcgcctgcctctgcctcccgagtgctgggaataaaggtgtgcgccaccgtgcttggcttgaactctttatcttctaacctccgtgttctgagtactaggacttcgggtatcggcaaacatgctctgttgttatgtagtgttaggggccaaatccagggtctcaaacatgccagtcaagcctctacccactaagctacgtcccatccgcaggctctctgcattttaataagtgctacagggttttctaatgtgactccctcaaaatgactataagaattaaaaactgccagtgtagctgtgtcactcttttttttttaagagagtgatttatttatttattatatgtaagtacattgtagctgtcttcagacactccagaagagggagtcagatctcgttacggatggttgtgagccaccatgtagttgctgggatttgaactctggaccttcggaagagcagtcgggtgctctaacccactgagccatctcaccagccctgtgtcactcttttaagactcagacttcacttctgtggttgcagcctggccttgggcattttgacagcactcctgctgcatctgggcagcctgagtcaaagatgctccttggccacttaagctgcaaacaggccagggaaacaacggtgtgactgggaaggaggggcctggacagtaggtctgtgactggtggcttaacacttaggagacggctatgaacaggtgccaggatccctgtcctgagctccagtttgttgttcctggaaactaatcctttcttctggcctcctcctttgccattctagactggtgaactgctgcctcacttctagcttctgccgtggtctcttctcaagtctaagcaccaaccggagcctcactgaactggacctcagtgacaatactctgggagacccgggcatgagggtgctgtgtgaggcactccagcacccaggctgtaacattcagagactgtggtaagtctgtgtgtgtgtgtgtgtgtgtgtgtgtgtgtgtgtagaatgattgggagaagacagtagagaaagatgattaatgaatgggattgagaacaaaacccaatgaactaagaagtaagcgtttgggagaatatcgcttccatgcacagtattgggtggactgtggccactcaggtttggctgttaggagttcaaaatcacatatccattggttgatttgttctgtgtctctctccctggcttgtaggtggccattttctccttgaatctttgcatcatcttctagctgtgtgtctgtcctccaacctctaacaacctttccttgtactgataccagtccaacggcactagggctaacactgaattctgttcaacgacctgtttgaagcacctgtttccagtatgtgttaaataaagctagagggagggtccaggggcccagctggagggagggtccaggggcccagctggggactcagatgctcagagtacagcatataaatcacagtaggctgaacacgtgggtagtactagtgaggccaacctgaggagataaacttttcatccccataacagcatctgtgatgaaacttcccagttctcacccagaagcctggtccatgaagaaatggaaaacacaacctttgttttgccaccaacaaatagctcctttcagctcttcttcatcttccctgtctacagctttctaaacttacctccagtccattcagccagcacacttataatagaatcaaatgtgctgatacgtgaagctttggagggacacatttgggcctttaacacatactctggattcatgtagcatctgaatttttaacaagttacatagctctttaaatttcatttgtttgtttatttacttatttaaaagacaggacctcactatgttgcccaagctggcctactttctggctccagcactctcccttcctcagcttcttttgtgggtctaagagcgtgaggtggcgtcctgccattacgttcacttcttttgagtatttctctatctccctattgctcgtactatatatctttccaacaccatttgtaaaaactcaattttattttctgtataagctgctgttcacagtctctcatcctcacttggccagcccagggccaggagcactgtgctggatgttacagtgagcttagcttatgactctggggaagaattcctgttatttcgctgtggtctttggaacaccagggcacaccggtctttaagagtgtgtgtgcctcagaaggatgttgggtgttttgtctctccacatctgctttttcttttcttttagatttatttattttatgtgtctgaatgttttgcctgtacatatgtctgtgtaccatgtgcatgcctggtgcccttggaggccagaagaaggtactggagtccctggaactgtagtaatggatagctgtaagctaccatgtggtgttgtgaaccaaactcgaggtgttggagaaatagcttggcagctaagagctcttgctgtactggcagaggacccaagtttggtaaacagacatatatgcaggcaaaacattcataacacataaaaataaataattctcccattctgctctcccagtggctttatattattgagaattattttattatttctgagtgttattgaaaactaatgattactccatctttctttgttggcttgttagctgtagatttttgtgctgttattttgatgaattttcttttggatgtattccctacattagcttaactgtagcattgccacaaagtccattccatagtatattactttctttcttttttaaaaaaagatttacttatttattttatgtatatgagtacaattatagctgccagccccacttgctccggcctaaagatttttttattattatatgtaagtacattgtagctgtcttcagacacaccaggagagagcatcagatcccattacagatggttgtgagccactatgtggttgctgggatttgaactcacgacctttggaagagcagtcagtgctcttacccactgagccatctctccggccccatagtatatttcaatgtctccccttctagcctgtgagctatgactgtcttatgcttctgaatttcacacactccattttgtgctttattttgtttaactccaagtatatggaacaaataaaattttaaattaaaaaacccatacacttaaaaaagaacatttctatttttcttttctttaaaaacttatggtatgtgcatgtgtacgcgcgcgcgcgcgtgtgtgtatgtgtgtgttgtgttaccgatgtctgttgtatgtgggcctgcacacgtcacaacatgcatgtggaagtcagtgggcaagtttggaaattggttcctgacacagacagagatgaggtcataagttagggtggcaagagcattgacccgctgagtcacttccctggccttactgtggagaactaagtggatcctgagaaaactcctctcactcctttggagagccgtgcttacagcagcatcattattatggacttagcctcagctcttaaaggtttcagcccctctcccatcactacgctgaagaccaagcttctgactgtggcctcctggggacaaaccacagccaaacaatagtgcaaaaaaggactcacaaatgggggtgaggggtgcacgctgacccagccctgagtgttgcctagctgacatttccccacagctcttctctgtggctcacagagatctgttcaccaagaaggaaacggagtgaaggccacatgtaccaagaggttggcactgtgggaagcacagagagaccctaacttcccccatccttcctatcggtgtcttcctgagtgtggctttggtgtccctgacccacccagcagaataggaactttatgagatcaccggtcagttctgcttcctaaatttctcctcggtgtgaagagagcacggggatcacattctcctaacgcacggaaagacaaaaatgtcatgggaacgagaactctagccacacgcatgcctcgtgaggggacttcaaccattagaaacgctggaaggcaggggctccaggctcaccacccttgtcatttacaaaccaggatgtaccctttgtgcacccgaagtgcagaccaacacgagcttggtgcattcaccatgaggccccctcccacttctaaggtgtcctgtgtgctgctcaggaattctggttaccccagaacacatcgggctcctgctgaatacagaaactgcatctaaactgaagaacgatagcagggaaggaggctgtgcgagccggctctccatcatgaaataagcacctgagacagtcaatttaaaaggaggaaaactttgttttggctcatggttctagcctatggtcagtcaagtctgttgtgatgagccagaatatcgtggtagcattatatggtcggggagtctatgcacatcatgaggctaaggaacaaaggaggaagagagcaagagtgagggggaaagagacagacacacagacacacatacagacagagacagagagacagagagagacagagacagacagagagacagagagagatagagagagacaaaacagtatttcagtatctccctaaggtcacccccttcagtgtccatacatcccatgtgtcttcctctaggctccacttcccagaggtttcagtccccgccccccatagtgtgagcgtgtaggcgtgggacacgcataggatctggatcctgagagaggcctctctctgatgtctgattctgttctcgctgtgaaggttggggcgctgcggactgtcccatcaatgctgcttcgacatctcctctgtcctgagcagcagccagaagctggtggagctggacctcagtgacaatgccctgggggactttggaatcagattgctgtgtgtgggactgaagcacctgctctgcaacctccagaaactgtggtgagtctggcctacctcccttaggcagctctgctgtggaggcccaattcaggaactctgcttcagggagacacagcgagccatcatcagctcctgtgtgaggatgagaatattctcagcggcctggagggcttcctgcgctcccttctgctagtttgccagtcgttcagtcagccagtgattgctgtgcggggagttaatgggaaaatatagcagtggtgtgtatgtctgtgcgtgtgtgtatgtttataaatcatcaaataaactgtctttaagatatccgaggaaacagaagcactgctaagcatcatggtagaacggttttgagaggtcataataaatcctcacactaaatagaaaaaaagcagcgcttaagcaatggaatatttattgtgtggggtccttactgaacgcttctaggagacgagaattctaaaaagttaagagagtgcttgcaggctaggaatgcagctcagttgacggagtgcttgcctaacatatgtgaagcccaaagtttgatccttagcaccaaatcaaaccagcaaaccagatgtggtggcagaaagatcaaaggctcaagctcatttttggttacatatttttggccaggctgggctacatgagaccctgtctcaaaacaaaaacacaagcaagcaaacaaacaaacaaaaacaaaaaacaaacaaacaaacaaacaaaaaggaacaaaaaccccacttgatcaggtggtggtggctcatagttcatcaccatgattatagaaaacaaacgcacaacaaacaaataagaaactggactagtgttggtaaggacttggagaactggaaccttttgtctctttaatgcagttgttctcaacttgtggattgagaccctgttgggggtcaaacaaccctttcacaggaattgcttaagtacatcagaaaatacagatatttacagaaggaaaatcacagccatggagtagaaacgaaaataatgttgtggttggggtcaccacaacatgaggatttggattaaagagtcacagcactaggaaggttgagaacccctgctctagtaggaacgtaaaacaacagcaagcaggaacataaaccacggcaaccagcacagtgattctcacacgagtatggacctgtacttgatccatgtgttttcgggtgcatctgccagctatgcaaccgtgtttgcaacatcaagtccagagcatgtcagtaacggcagccagaggttagaaacttctccgtgtctgttgatggacactggtcagaaggtggcatctgcacatacgtagctttgaacaaatcaggacacttgcttaaatcttcacgagcctcgagggtgcaatactaagtgagataaatctgtcacaaaaggcaaacactatgattacacttacataacgtttcttaaattcataatgtagggcataacggtagcttccagggactgggaaggaatgagtgggaagttttcgtttgtttgcttgttttgttttgttttgttttaacaggtgtttcagttttataaaataaagataatcagaaatggatagtgacggtggctgctcttaaaaaacactgagttctttttctttctcttcttcttctttctcttcttctttctcttcttcttctttctcttttccttctccttctccttctcctcctcctcctcctcctcctcctcctcctcctccttcttcttcttcttcttttctcttcctcctcctcctttttctcttcctcctcccccctcctcccctccttcctctccttcttcttcttctggtgctgggggctggatctaggttctctcacgtgctaggaaagcactctacctcaccccctttcctttttcatactgagcagggtcttgctaagttgcccaggtactccctagcttttgatcctcttgctttcgcctcctgagtaactgggactgcaggcctctgctaccaggcctcaataaaattctgctctgaaagtggttaggatgacagattttaccacacatacattaaaaccctggttttatattattattattattattattattattatcattatcattaccattattattagtgtgtgtgtgtgttccatagagtttgtgtgaaggtcagaggacaactttttagagtcagctctctcctacttcgtgggttctgggattaaattcaggttgtcaggctccttagcaagtgcttttactcgataagccatctcccttgaccccacatttacattttatttcaataaaatagatgaagggaaaaggaaagatgttcttgggaattggcttactgtctgttactccttcagacggggaagaggataagaactgtgtggagctaaggtgcaggttgtagctgaggagggcagtagcaggtacaggtctcctagcccacggtgcagagtgcatgagagatggcactgaccttgttccctctgcatggaaggttggtgagctgctgtctcacatccgcgtgttgtcaggatctcgcattggttctgagctccaaccattctctgaccagactgtacattggagaaaatgccttgggagactcaggagtccaagttttgtgtgaaaagatgaaggacccacagtgtaacttgcagaagctggggtaagtaagtcaaattcttctcagagatcgcttatttgggaagcacgcatgggtggggcccctctagagaatgttcatctgctggagatgaaggtgggaaccgtagcatcagcatccgaagtggtttgaagctctagcttcatgaggcacacagctaggattctgtacttcagattcaaacagccatgggtttagccagctgttggttaaaaatattaggaaaaaattatttctgtgcttaacatatgtgagccttcctctttgtcattattctctgaacaacagagtatagctacataccatgtagcacacatgccatgacagatagcacaggtaacctaaagataagtacagtccacgggaggatgcgcagaggtgatatgtaaacactctgcatttcacatgagggacatgaccatgtgcaaattttagcacctgtgggggtgtcttggggccagtcactaaggacactgggggacagttgtattactgctgatgagatagttctactgagttgggagacagaggcaggtagatttctgtgagttcgaggccagcctggtctatatattgagttctaggacagctacaactacatatagggccttgcttcagtaaaagataaatacaacaaaatgagtctggaaatgtggttcaattgttagtttgattagcatgtattatgccttccatccttaggatagaataaagtgggttttatgctacactcatgtaatcctagcacttgagagggaacggtgggatgatcagaagttcaaggtcgtactcagtaacatagggagtttgagactagcttgggctacataagaccatgtctttaaaaaaaaaaaaatcaaaaccaaaacaaaacaacaaaacctaacactgacaagagactttttctgagggaaaattaattatccatgattattaaccaatattagttgtaaactctaatactttcttgggtttgtcctgttagcacagcctcagacgttaccaaattgctttcctagaatgttaaggtgctgccacggctctgccagagcagtgtggcttccgatgctttgtttgatcaacagatgtgatttggcctctgagcttgccaggtgccctagactctgttgtgggtgcggatggcagaagtggccttgtttacacacacagcactatagggaggggtgacccactagagcacaaaacagtatgtaatacgtgttgcttctgacagtccttagcttggctcttgccttggctttagtccatccatttttcctcgagttcctttccccatgtttttcttgatttgataacttcctcttctataagttctgcattggcttctcttcctgttatctttgggttttgtcgttgattttctattgtttctattttctctgagatatcttatgtacccctaaacaccaatgaccactcataggcaaatgtcttcccaagtagatctcacacccagataccacttgggaggtgttttgtttttgctacaaaaacaacataaaaaaatctttctgcctctagaaaaagccacttactaacctggcaaacaaaatttgagaaactgtattgtacacacaaactcccattcccccatatgcccatacattgccaattgctttatcaatctacatattaatccatgaatttactcactattatatttatccaactatccatctttttacccaaacattctgtattgttggggccggcctgtggctctcatgtccaggttcgagcctgggaggcaacctggagctgaaagaaaagagggaacttaggtgagtggagaaataatggaaccaagacatgctagtctgttcaaggttcaaatgtttaatggtgactgcacttataaaaagggggggagaaggcccattcccaccaattcatccttggagtctggaaccagctgcaggtgatgacgtgcagaatagggcatagtctccagtatcttgaagaggaacagtgactctgcctgaacaatagagtgatctagggaggaaggctccaccctaggtaatctccttagtggcaccaaggtcaggttctggctcagcctgcttcaggctgagggaaggttacactgtatgtctgagtttactaaggttgctatataacacagtgcccaaaacataatggtgtagacaacacaaactgtctctcagttctcgaggagagggttaaggaattagcagagctcgtgcctctgagggccatgagcaagagtacattctttcgtctcccttcctggcagtctttggtaccttgtcttggagaggcatcacctgattgtggcctccatcttcacacaagtttctctctgtgcctctgcaggtaacactgagtcccaagttctggtactaagattgtatttatttgtttttaacggtgtgtttgagtgtgggttggggagaccattgcatagaagtgcagtgtcaagagaggagtcactagaccccctggagttagcatcccaggtggctgtggtctgtctgacatgaatgctgggtaccaaactcgggtcttctggaagagcagcatgctctcttaactgctgagtcatctttccagtcagacgctccagttctgatgaggatgtcagtcataccagatttgggcccacctagagtcatcttaactgactatatctgtgaggttgtgaggttgtgaggttgtgaggttgtgaggtttggtgttcagttatggtcacattttaaagaactggaattttaatatgtgaagtttttggaagaacaaaattcaacccaacactgtagttagctattcaaactacaaacactatagaagtatttttctttgacttgtttttcatttatctggatacatttccatatgacttctagtttggagcacaaaaacccatagtacccaccaccagcatttattcagctactcattaagtcaagaactttttgggtaagtttctacatttttactgttacaaatcactctgtaaaatacatgcttgtttatggaactcaatgcatgtgtgtgcatgcgtgtgtgtgtgtgtgtgtgtgtgtgtgtgtgtgtgtgtgtgtatttctttaaactaggttccctaatgtgtagcatgaatgaactaaaaattttagccatcttatatattagtatcttcttctcttgaactttagcataatatgttctagtctcttgttctataatatgttctaatctcacttctgtgctacagattcgtcacacatggacacataaaactgggcttagctactataacaccccttccatctgccactgttagatccctccctgcccctgctcaaacttgctgagcagaggcctgtccagaggttcgaagcaggttcctagagggcttcacacacttcactacgctcagttccttctttgtctgcctgtctttccttcctcctttccttcggagaaaagccagccttcgttccactttcctctggtctgtgttctattgaattagttttatgttccaaaggcctggcctgttctgccacaatctctcttatggaagtttagatcatcgacaagcaggaagccctcattatttttcaagtaattattctggttggcaattttatgatatttatttatttattttctatgatatgtctctgtgtgtgtgtgtgtattggtgggtgtgcattgccgtgccatgcatgtagaggtcagatgacaacttgtcatgccttattctcttctttctttcatcttgtgggccatggggttcaaatttagatcatgaggtttgggagtaagagttttttcctaccgattcatcttgccaccctaaataatcattctgatctccattttcctaactaaaagtggagctatgctggctagggatgtagtttagtcagcagagtgtttgtctatcttgaacaaagccctcaatttgatcactagcatgtaagtagggtgtggcggcacatgctggtaatccctgcactccagaggcaggtggatcagaagttcaaggtcatgccagataataccgcaagttagacaccattctaggttacaagagattatgtctggaagagaaacaaggaaaggaataaaaataacactcctatcttgagtttgcataatgaaataaaacattcacactcgatgccattatcccacaaagtaagtagccaacaccattagaagttcggttttcatccgaaggcagttttagggaacaacgaggattgctgacgtagaaaagccactcggagttaagtgaatgcagcaaatagcatgtgttcaagacacacagaacaaagccacccagtgattagtggcttttaccggacagcaaacttaaggaattagggaaggaaggtttactgtggctcatactgtgaaggtacagctcatctcatcgcaaggcgaggggcgtgggggggtggggagggggggtggttgccgaggaacagcagcagcaacagcagcagcgtcatgaagcagccatcacgctgtgtctgcaaacgggaagcagagagaagagtgtggtgctcacttccttttccccattaattcagcctgggacgccagcccatgaagagttttccactttaacctaatctagaaaattcctcacagatatgcccaaagacttgtcttgagagtgagtctagattttgtcaagttgacagtaaacacataaaccatcatagctggattggctgggactgagctgttaccagtgggcgggctgactggatgtgtttgatttgctaagcatcgcttctcggccttttggctaagatcaagtgtgatttgctaagcattatatcatgcattaaacatacttatccctcagcctcaatgggtaccaatatcctaatcacataactttgatgtcgtcttcttttctgaggtgctaggaatggaacttaggcctgaaagaacctagtggggaaacccccactcaattcttgttcggttcgcacccaagaatcacgaacagacgaccatcttgatgtaaaagcatgaggtagtttaatgacggagctcgggaccgacacatatctcccgcaggagacagaggtgtcaaccacatggtggagccataatggcggagctccggttcgaaacgtatctcacgcaggagacagtggtttcgaccccaaggcttggaagctagggacttttatagaaaaggggtggggctgggggaggaattggcacggttttacatgattggtccatttaaacatcaacagactgtcagaagggcgggagatagggaggcactaggccagtcaggacatgtaacgacgggcctgctcgggcacgtcctggcctgttctgctatgttctcagccccaggtttcaaagctcacaaacaactctttgggctatttgacataaattacatgaataacatgtctcaagttttatttccttttaggccctcacatatgataggcttggattcaggtgaccccatgacactgagagattgttcatctctgggagggtctggaagcagagcttggatctgaactgggttacaaggccctccctccggtggtctcttcccctttagtctggccacatctggaggttccacaccctggctaagcagcaccacaagcgttcactgtagacagtgttcaacttgacctcagaatagtaaatcaaaaattcttggactgttctgaggttccagaggtaaagacttctgccatcaaggctgacaacctgagttcaattcctgggcctcacagagtggaagatgtcttagttaaggtttccattgctgtaaagagatgaccaaggcaactcttaaaaaggtaaacatttgcatattccactccaggggagtttgctgatagaatattaaaaaaaatattacaaaaaaaaaaggtaaacatttaattggggctggtttacacattcagaggttcggtccgttatcatcaaggcctgaagcatggcagtttccaggcagacatggtgctggagaaggagttgagattttcacatcttgatagagtagcggccaggagactgcttccaggcagctagagggtctccaaacctacccctccaacaagaccaaacctactccaacaaggccacacccactctaataagtccacaccccctcatagtgccactacctgggccaaacatattcaaaccaccaccgaaggaaatagccaaagttgtcctctgatctccaaagaatgttcttgctctctctctctctctctctctctctctctctctctctcgtggatccaagcaagtatgctgagaccattgtccatctctcctgtgggatccagagacgtggtccatctctcctgtgggatccagagacgtggagccatgcttcagattatgttaaagatgctggcttctgaccttttaagttggaaggatgcagctcgcagtgacacacaatgtctgagagagcctgggcactgaggactccttctcttcttcctataggttggtgaattccggccttacttcaatctgttgttcagctctgacctctgtgctcaaaaccaaccagaacttcacacacctctatctacgaagcaatgcccttggagacacaggactcaggctcctctgtgaggggcttctgcacccggactgtaaactacagatgctggagtgagttcatgggctgcctgaggtgatgggaacatggggtgtgaggcaggccctggagcaaccctgatgcaaatctgtgtctgaggagtgaggcgttcttggtgtccattagtgagaaatgggaagtttctagatggtatacagcttaaaggctaagcccctgccaccacagtcctgccaactgcaaacattcatttggtccattgatactttgtggaaatctgaggggaacataatgtttagaaaaagtcaattgacaaatttctgttactattttggtctgttcaacccagtaccttgggaaaagaaacttccatctaaagaggggagaaaagaaaccatatcatatgaagcagcttcacgctggtcagtctgtggtttgttgttgttgttgttttcaagacagggtttatctgtgtagccctggcttgcctggaactcactctgtagaccaggttggcttcgaactcagaaatccgcctgcttctgcctcccaagtgctgggattaaaggtgtgcaccaccactgcccagcccaatctgcgctttttatgcaaatgaaactgcaggtttggagacccggaagtatggatggtcaaggctattttctttatatcacccttctcttctcaaagattagacaactgcagcctcacctcacacagctgctggaatctctccacaattctgacccacaaccacagccttcggaagctgaacctgggcaacaatgatcttggcgatctgtgcgtggtgaccctctgtgaggtgctgaaacagcagggctgcctcctgcagagcctacagtgagtgtggtttgcctagagcttctcatggggtaggcgagcggggtgctgaggggagggtgaccacgggacaaaagtcagagtttctctggattaatttgcagttttctgaagagtccaactcaaagcttcttttctgtgttcacaggttgggtgaaatgtacttaaatcgtgaaacaaaacgtgccttagaagcgctccaggaagaaaagcctgagctgactatagtcttcgagatttcctggtaggcgtggaagcaggaccaccaggtgcctcggtcctgccccaagtcctgccccaagccccagtgcgcactgctcttcactgctatcaagccctccttcaccatcaggatcacagccgaggctcttctggtatagggtctggagcaaaggcttgtgtgggaccaaatatttttcctcacatcgataacgtgaaactgccagaggctgcccttcccatcatatcctcagtgggcaaggtgttccctcttggtgacttcatggaaacagcttcaagaaaacgccttttctgtcctcccccgccctcctcttactcctgcccctcctcctcctcctcctcccctccccccccctcctcctccgcttctccccccacctgtctttctctctctgggcctctggttttttgacctttgcccataccttcagtcttgtcttcctgttaactgaccatcccgcataaggagctgcccgtgggctagatggaaggtttgtggcagcctctcagctacattgtttgtttttattttttaatagttatgatttctctttagctacctgaaaactcagagatttataaaacccatttttgtatttattgtatgtttgtactgctttcttaatttaaaaatgtatctagaattcttttaagttatttatccaaactactaaaaataaatcagtttacacatttaaaatttctctttgcaaaatgattcaacgttggttaatgagggggaaaaagtcacgtcctttggattttagaggagggatttcagctgtagtagcatagagggtaacagcatgcaggggaaggttagctcacatgcagaaatgaatcctacagcattctcaaacagaggcataacctggtttcctcttgcctgggtgacaaaagcttaccttaggtttatccatttgaagagcagctcatctcctaagtgacactgagtgctaagtcatgaataaatactaactgcccacagtacacagaaacaactcttaaacataaaacattgctcagagtggaactattgtttcatatcgtctggtgaggctgctagacctttgctatggagacggactgatgaactgttgttgtctttaaatttttaaaaagatttatttatgtttatgtatatgagcacactgtagctgtcttaagacagcaccagaagagggcattggatccattacaggtgactgtgagccaccatgtggttgctgggatttgaactcaggacctctggaagaacagtcagtgctcttaaccactgagccatctctccagcttctgatgaacttttaatataaggagattatgcaaggcaaagcaaccatgccactaaacaatctctttacataaactgtgttttttaaaaatagagcctatctttccctttgaacgtctgatcctccacgccccatctctccagtgctgggattacaggcatgtgccagcatgcagcttatgagacgctggagctcaaacccagggcactgaggatgattgtcaggcatcccaccaactgggctctttctagtaaggctaatttatttaataatttaaattaaaccaattgaaaaaccatgtggtgcacaattctgtgcaaactctctgcctgcttaggagtgtgagtgtgtttgtgtgtgtgtgtgtgtgtgtgtgtgtgtgtgtgtgtgtgcaggtatgcatgtgtatgcatgtgtgtctctctatgtgtatgcttgtgtatgcatgtgtgtctgcatgtgttcagtagtagcagcagcagcagtacttatggtgattaaactctggtactgagccccaagtctgggtttagggctttgaatatttatctatacatttgctttctgtgtcatggtaagctgaattgtgacatgttgtctccctgttgtaagtttttgacacgcattataaaaatctattttttcctatcacacatcaatttgtcgtgtcaaaatgcttgaatttaattgagagtttaatcccttagatactttataactctgagcaacagaagttctcgggactggagagatggatcggtgggtaagagcaccggctgttcttccagaggacctgggtttgattcccagcaaccacatggtggctcacaatcatctgtaactccagttccagagaactgaacacctctttgactcccccaggcaaaacacccataaaataaattaaagatgacttaaaaaaaatagaagttctatatgcttatcctgtagcattacctgtctatgacaatgaatattctaagggtccctcagtgctccaagcacctttgctgcttgacagcccatgccaacacccaggacttgggaaccctgactatttacttgatttcatcctctgagggccatggattcattcatggttctgctgggtgtttcttctgtcccatattgaggctgccttgctggaggatccagtcccgttcaacagtcacagagctggctgcaagcctggctcacatggctctgcagttacagtctccccagcatggccagtcttacagatgatgggcctcttggagacttggggtacctgagaatatcactaactaaaagttccaggtgctttacaactgtgtccaggaagcactagagaatctctcactgagttttagaggtcaagcaagtcactaggcatagcctttcacgaagttaagagaatttgagattgtcagttagcacagctgttttgggaaacacagttctcaaatgtttcctagaattagatcggctcattacactgatatatatatactgattacctacctctcttggtgattctcatcatgttgattttactgactgtggcttggatttgtcccattgcttaagggctcgtctatgttactctgggatataaagggctttgagctgctctgctgtgatagctctatggaaatccattggaacaagcactgtggtcctggactggcatggtcattatttttcacatttcttacatcaaattcttcagctctgtgatttgtcttgccttcacagttagaaacactttcttaatttccctctttttgtaatttccattcatatattgttaacttgctagtcttggttacattaataggcggtaggatagccacaaagaactaaatataaaagggttttcagtgcgggggtggaggtgggggtacaaatgcaaggtgctggagatgagttcaattccaactttagtggatctgatgccctctgc

SEQ ID NO: 4 - DNA, 4033 bp, Mus musculus
  source 1..4033: mol_type = genomic DNA
ccaggtcctagcctcgtcaccatgggttctggtcagacacgagtcctggtgactttgtatatgcgtgttctctgtataccacatctgattgtgttaatggctttcttatttttatctctacagaggaacttttcttccatggctcaggacatacgtctggatcaagctaagagaactttctgtgtggacctaagccccgagaccctcgaaagggctgctgctgaagatgacgagtgtccgttgcaagctggctcagtatctagaggaccttgaagatgtggacctcaagaaattcaaaatgcatttggaagattacccgcccgagaaaggctgtatcccagtccccaggggccagatggagaaggcagatcacttggatctagccacactcatgattgacttcaatggcgaggagaaggcctgggccatggctgtgtggatctttgctgcgatcaacaggcgagacctctgggaaaaagctaagaaggaccagccagagtggaatgacacgtgtacatcacattcctctatggtatgccaggaggacagccttgaagaagagtggatgggtttgctgggatatctctcccgcatctccatttgtaaaaagaagaaagattactgtaagatgtacagacgacatgtgagaagcaggttctactctatcaaggacaggaacgcgcgtctaggtgagagtgtggacctcaacagtcgctacacgcagctccaactggtcaaggagcatccaagcaagcaggagcgggagcatgaactcctgaccatcggccggactaaaatgcgggacagccccatgagttcccttaagctggagctgctgtttgagcccgaggacgggcactcggagcctgtgcacacagtggtgttccagggagcagcaggcatcgggaaaaccatcctagccaggaagattatgttggactgggcactgggaaagctcttcaaagacaaatttgactatttgttctttatccactgccgagaggtgagcctcaggacgccaaggagtctagcagacctgattgtcagctgctggcctgacccaaacccaccagtgtgcaagatcctgcgcaagccttccaggatcctcttcctcatggatggctttgatgagctacaaggggcctttgacgagcacattggggaggtctgcacagactggcaaaaggctgtgcggggagacattctgctaagcagcctcatccgaaagaaactgctgcccaaggcctctctgctcataacgacgaggccggtagccttggagaaactgcagcatctcctggaccacccccgccatgtggagatcctaggtttctctgaggccaaaaggaaggagtatttctttaagtatttctccaacgagctgcaggcccgggaggccttcaggctgatccaagagaatgaggtcctctttaccatgtgcttcatccccctggtctgctggattgtgtgcacggggctaaagcaacagatggagaccgggaagagcctggcccagacctccaagaccactacggccgtctacgtcttcttcctttccagcctgctgcaatcccgggggggcattgaggagcatctcttctctgactacctacaggggctctgttcactggctgcggatggaatttggaaccagaaaatcctatttgaggagtgtgatctgcggaagcacggcctgcagaagactgacgtctccgctttcctgaggatgaacgtgttccagaaggaagtggactgcgagagattctacagcttcagccacatgactttccaggagttcttcgctgctatgtactatttgctggaagaggaggcagagggggagaccgtgaggaaaggaccaggaggttgttcagatcttctgaaccgagacgtgaaggtcctactagaaaattacggcaagtttgaaaaaggctatctgatttttgttgtccgattcctctttggccttgtaaaccaggagagaacctcttatttggagaagaaactaagttgcaagatctctcagcaagtcagactggaactactgaagtggattgaagtgaaagccaaggccaagaagctgcagtggcagcccagccaactggaactgttctactgcctgtacgagatgcaggaggaagactttgtgcagagtgccatggaccactttcccaaaattgagatcaacctctctaccagaatggaccacgtggtttcctccttttgtattaagaactgtcatagggtcaaaacgctttccctgggtttttttcacaactcgcccaaggaggaagaagaagagaggagaggaggtcgacccttggaccaggttcagtgtgttttcccagacactcatgttgcctgttcttccagactggtgaactgctgcctcacttctagcttctgccgtggtctcttctcaagtctaagcaccaaccggagcctcactgaactggacctcagtgacaatactctgggagacccgggcatgagggtgctgtgtgaggcactccagcacccaggctgtaacattcagagactgtggttggggcgctgcggactgtcccatcaatgctgcttcgacatctcctctgtcctgagcagcagccagaagctggtggagctggacctcagtgacaatgccctgggggactttggaatcagattgctgtgtgtgggactgaagcacctgctctgcaacctccagaaactgtggttggtgagctgctgtctcacatccgcgtgttgtcaggatctcgcattggttctgagctccaaccattctctgaccagactgtacattggagaaaatgccttgggagactcaggagtccaagttttgtgtgaaaagatgaaggacccacagtgtaacttgcagaagctggggttggtgaattccggccttacttcaatctgttgttcagctctgacctctgtgctcaaaaccaaccagaacttcacacacctctatctacgaagcaatgcccttggagacacaggactcaggctcctctgtgaggggcttctgcacccggactgtaaactacagatgctggaattagacaactgcagcctcacctcacacagctgctggaatctctccacaattctgacccacaaccacagccttcggaagctgaacctgggcaacaatgatcttggcgatctgtgcgtggtgaccctctgtgaggtgctgaaacagcagggctgcctcctgcagagcctacagttgggtgaaatgtacttaaatcgtgaaacaaaacgtgccttagaagcgctccaggaagaaaagcctgagctgactatagtcttcgagatttcctggtaggcgtggaagcaggaccaccaggtgcctcggtcctgccccaagtcctgccccaagccccagtgcgcactgctcttcactgctatcaagccctccttcaccatcaggatcacagccgaggctcttctggtatagggtctggagcaaaggcttgtgtgggaccaaatatttttcctcacatcgataacgtgaaactgccagaggctgcccttcccatcatatcctcagtgggcaaggtgttccctcttggtgacttcatggaaacagcttcaagaaaacgccttttctgtcctcccccgccctcctcttactcctgcccctcctcctcctcctcctcccctccccccccctcctcctccgcttctccccccacctgtctttctctctctgggcctctggttttttgacctttgcccataccttcagtcttgtcttcctgttaactgaccatcccgcataaggagctgcccgtgggctagatggaaggtttgtggcagcctctcagctacattgtttgtttttattttttaatagttatgatttctctttagctacctgaaaactcagagatttataaaacccatttttgtatttattgtatgtttgtactgctttcttaatttaaaaatgtatctagaattcttttaagttatttatccaaactactaaaaataaatcagtttacacatttaaaaaaaaaaaaaaaaa

SEQ ID NO: 5 - DNA, 17 bp, synthetic construct
  source 1..17: mol_type = other DNA
acgatgccatcttgacc

SEQ ID NO: 6 - DNA, 17 bp, synthetic construct
  source 1..17: mol_type = other DNA
acggtgaacaaccactt

SEQ ID NO: 7 - DNA, 16 bp, synthetic construct
  source 1..16: mol_type = other DNA
tacggtgaacaaccac

SEQ ID NO: 8 - DNA, 17 bp, synthetic construct
  source 1..17: mol_type = other DNA
tcgatccaggagtgtgt

SEQ ID NO: 9 - DNA, 17 bp, synthetic construct
  source 1..17: mol_type = other DNA
acacggcacacggatga

SEQ ID NO: 10 - DNA, 17 bp, synthetic construct
  source 1..17: mol_type = other DNA
tcggtccacactaagat

SEQ ID NO: 11 - DNA, 17 bp, synthetic construct
  source 1..17: mol_type = other DNA
cttaggcttcggtccac

SEQ ID NO: 12 - DNA, 17 bp, synthetic construct
  source 1..17: mol_type = other DNA
atcattagcgtggctag

SEQ ID NO: 13 - DNA, 17 bp, synthetic construct
  source 1..17: mol_type = other DNA
gcagcgaagatccacac

SEQ ID NO: 14 - DNA, 16 bp, synthetic construct
  source 1..16: mol_type = other DNA
ttgatcgcagcgaaga

SEQ ID NO: 15 - DNA, 17 bp, synthetic construct
  source 1..17: mol_type = other DNA
tgttgatcgcagcgaag

SEQ ID NO: 16 - DNA, 17 bp, synthetic construct
  source 1..17: mol_type = other DNA
acgtgcattatctgaac

SEQ ID NO: 17 - DNA, 16 bp, synthetic construct
  source 1..16: mol_type = other DNA
ttcgaaacacgtgcat

SEQ ID NO: 18 - DNA, 17 bp, synthetic construct
  source 1..17: mol_type = other DNA
attctcgaaaggtactc

SEQ ID NO: 19 - DNA, 17 bp, synthetic construct
  source 1..17: mol_type = other DNA
cgtgtgtagcgtttgtt

SEQ ID NO: 20 - DNA, 16 bp, synthetic construct
  source 1..16: mol_type = other DNA
cgtgtgtagcgtttgt

SEQ ID NO: 21 - DNA, 17 bp, synthetic construct
  source 1..17: mol_type = other DNA
gcagtcgtgtgtagcgt

SEQ ID NO: 22 - DNA, 17 bp, synthetic construct
  source 1..17: mol_type = other DNA
ctctcacacgtcttggt

SEQ ID NO: 23 - DNA, 17 bp, synthetic construct
  source 1..17: mol_type = other DNA
agagcggtcctatgtgc

SEQ ID NO: 24 - DNA, 16 bp, synthetic construct
  source 1..16: mol_type = other DNA
agagcggtcctatgtg

SEQ ID NO: 25 - DNA, 17 bp, synthetic construct
  source 1..17: mol_type = other DNA
agaagacgtacaccgcg

SEQ ID NO: 26 - DNA, 17 bp, synthetic construct
  source 1..17: mol_type = other DNA
aacgttcgtccttcctt

SEQ ID NO: 27 - DNA, 17 bp, synthetic construct
  source 1..17: mol_type = other DNA
ggaagcttcaaacgact

SEQ ID NO: 28 - DNA, 16 bp, synthetic construct
  source 1..16: mol_type = other DNA
ggactgtcacgtctcg

SEQ ID NO: 29 - DNA, 17 bp, synthetic construct
  source 1..17: mol_type = other DNA
tcgaatttgccatagtt

SEQ ID NO: 30 - DNA, 16 bp, synthetic construct
  source 1..16: mol_type = other DNA
tcgaatttgccatagt

SEQ ID NO: 31 - DNA, 17 bp, synthetic construct
  source 1..17: mol_type = other DNA
ctcgtacaaacagtaga

SEQ ID NO: 32 - DNA, 17 bp, synthetic construct
  source 1..17: mol_type = other DNA
aggtccaattcagttag

SEQ ID NO: 33 - DNA, 17 bp, synthetic construct
  source 1..17: mol_type = other DNA
agcgtttcacacaacac

SEQ ID NO: 34 - DNA, 17 bp, synthetic construct
  source 1..17: mol_type = other DNA
atctccgaatgttacag

SEQ ID NO: 35 - DNA, 17 bp, synthetic construct
  source 1..17: mol_type = other DNA
gtcgaagcagcactcat

SEQ ID NO: 36 - DNA, 17 bp, synthetic construct
  source 1..17: mol_type = other DNA
ggaccaaggagatgtcg

SEQ ID NO: 37 - DNA, 17 bp, synthetic construct
  source 1..17: mol_type = other DNA
attccgaagtcaccgag

SEQ ID NO: 38 - DNA, 17 bp, synthetic construct
  source 1..17: mol_type = other DNA
tcacataaaattgcgac

SEQ ID NO: 39 - DNA, 17 bp, synthetic construct
  source 1..17: mol_type = other DNA
agactgacgtaaggcca

SEQ ID NO: 40 - DNA, 17 bp, synthetic construct
  source 1..17: mol_type = other DNA
gtaaaggtgcgtgagat

SEQ ID NO: 41 - DNA, 16 bp, synthetic construct
  source 1..16: mol_type = other DNA
gtgtgacgtgaggttg

SEQ ID NO: 42 - DNA, 17 bp, synthetic construct
  source 1..17: mol_type = other DNA
cagtgtgacgtgaggtt

SEQ ID NO: 43 - DNA, 17 bp, synthetic construct
  source 1..17: mol_type = other DNA
ctaacgcactttttgtc

SEQ ID NO: 44 - DNA, 17 bp, synthetic construct
  source 1..17: mol_type = other DNA
ctcaaagacgacggtca

SEQ ID NO: 45 - DNA, 17 bp, synthetic construct
  source 1..17: mol_type = other DNA
cggagaacactggcgtc

SEQ ID NO: 46 - DNA, 17 bp, synthetic construct
  source 1..17: mol_type = other DNA
atggatcgcagctctct

SEQ ID NO: 47 - DNA, 17 bp, synthetic construct
  source 1..17: mol_type = other DNA
cgacactccaccggaag

SEQ ID NO: 48 - DNA, 17 bp, synthetic construct
  source 1..17: mol_type = other DNA
tctccgacactccaccg

SEQ ID NO: 49 - DNA, 17 bp, synthetic construct
  source 1..17: mol_type = other DNA
cgtcggcaagctctctt

SEQ ID NO: 50 - DNA, 16 bp, synthetic construct
  source 1..16: mol_type = other DNA
cgtcggcaagctctct

SEQ ID NO: 51 - DNA, 17 bp, synthetic construct
  source 1..17: mol_type = other DNA
aaggcatcgtcggcaag

SEQ ID NO: 52 - DNA, 17 bp, synthetic construct
  source 1..17: mol_type = other DNA
ctaactgaggcgctgtg

SEQ ID NO: 53 - DNA, 17 bp, synthetic construct
  source 1..17: mol_type = other DNA
tagcactaccgtgagag

SEQ ID NO: 54 - DNA, 16 bp, synthetic construct
  source 1..16: mol_type = other DNA
cgtgcctaatctagtg

SEQ ID NO: 55 - DNA, 17 bp, synthetic construct
  source 1..17: mol_type = other DNA
acgaccagtctatttcg

SEQ ID NO: 56 - DNA, 17 bp, synthetic construct
  source 1..17: mol_type = other DNA
gcttaatcaccgccgct

SEQ ID NO: 57 - DNA, 17 bp, synthetic construct
  source 1..17: mol_type = other DNA
tcggaagcgagtggtaa

SEQ ID NO: 58 - DNA, 17 bp, synthetic construct
  source 1..17: mol_type = other DNA
catcggaagcgagtggt

SEQ ID NO: 59 - DNA, 16 bp, synthetic construct
  source 1..16: mol_type = other DNA
gtaatgtcaacggatc

SEQ ID NO: 60 - DNA, 16 bp, synthetic construct
  source 1..16: mol_type = other DNA
ccgtagaaaccgtacc

SEQ ID NO: 61 - DNA, 17 bp, synthetic construct
  source 1..17: mol_type = other DNA
ccgtaaggtgaaccttc

SEQ ID NO: 62 - DNA, 17 bp, synthetic construct
  source 1..17: mol_type = other DNA
gacgtatcaggcgatgg

SEQ ID NO: 63 - DNA, 16 bp, synthetic construct
  source 1..16: mol_type = other DNA
tgacgtatcaggcgat

SEQ ID NO: 64 - DNA, 16 bp, synthetic construct
  source 1..16: mol_type = other DNA
tcggacggagagcatg

SEQ ID NO: 65 - DNA, 17 bp, synthetic construct
  source 1..17: mol_type = other DNA
ctagagtggcggacttt

SEQ ID NO: 66 - DNA, 17 bp, synthetic construct
  source 1..17: mol_type = other DNA
gagttacactaaggccg

SEQ ID NO: 67 - DNA, 16 bp, synthetic construct
  source 1..16: mol_type = other DNA
ccggccgtaaacagtg

SEQ ID NO: 68 - DNA, 15 bp, synthetic construct
  source 1..15: mol_type = other DNA
tccgttgaaggacgt

SEQ ID NO: 69 - DNA, 16 bp, synthetic construct
  source 1..16: mol_type = other DNA
tcgagtctagcttatc

SEQ ID NO: 70 - DNA, 17 bp, synthetic construct
  source 1..17: mol_type = other DNA
tcgccgtcactccagtt

SEQ ID NO: 71 - DNA, 17 bp, synthetic construct
  source 1..17: mol_type = other DNA
gaggcactcgccaaacg

SEQ ID NO: 72 - DNA, 17 bp, synthetic construct
  source 1..17: mol_type = other DNA
ggacctgtacgacctat

SEQ ID NO: 73 - DNA, 16 bp, synthetic construct
  source 1..16: mol_type = other DNA
tagcgcaggtgattgc

SEQ ID NO: 74 - DNA, 17 bp, synthetic construct
  source 1..17: mol_type = other DNA
ggtggcgtaattcctag

SEQ ID NO: 75 - DNA, 16 bp, synthetic construct
  source 1..16: mol_type = other DNA
tctacgaagttcggtt

SEQ ID NO: 76 - DNA, 17 bp, synthetic construct
  source 1..17: mol_type = other DNA
tagacgagttctacgaa

SEQ ID NO: 77 - DNA, 17 bp, synthetic construct
  source 1..17: mol_type = other DNA
atgatgcgagttacgtt

SEQ ID NO: 78 - DNA, 15 bp, synthetic construct
  source 1..15: mol_type = other DNA
tgatgcgagttacgt

SEQ ID NO: 79 - DNA, 17 bp, synthetic construct
  source 1..17: mol_type = other DNA
cctaatgtacggtactt

SEQ ID NO: 80 - DNA, 17 bp, synthetic construct
  source 1..17: mol_type = other DNA
gctcgtcgtgtccttcg

SEQ ID NO: 81 - DNA, 15 bp, synthetic construct
  source 1..15: mol_type = other DNA
gctcgtcgtgtcctt

SEQ ID NO: 82 - DNA, 17 bp, synthetic construct
  source 1..17: mol_type = other DNA
agccggtgctcgtcgtg

SEQ ID NO: 83 - DNA, 17 bp, synthetic construct
  source 1..17: mol_type = other DNA
atggccgcgagactccg

SEQ ID NO: 84 - DNA, 17 bp, synthetic construct
  source 1..17: mol_type = other DNA
cgccgccgagaccaaga

SEQ ID NO: 85 - DNA, 17 bp, synthetic construct
  source 1..17: mol_type = other DNA
ttcgaatttgccatagt

SEQ ID NO: 86 - DNA, 17 bp, synthetic construct
  source 1..17: mol_type = other DNA
tttcgaatttgccatag

SEQ ID NO: 87 - DNA, 17 bp, synthetic construct
  source 1..17: mol_type = other DNA
ttgatcgcagcgaagat

SEQ ID NO: 88 - DNA, 17 bp, synthetic construct
  source 1..17: mol_type = other DNA
tcgaaaggtactccagt

SEQ ID NO: 89 - DNA, 17 bp, synthetic construct
  source 1..17: mol_type = other DNA
gcttctcacgtactttc

SEQ ID NO: 90 - DNA, 17 bp, synthetic construct
  source 1..17: mol_type = other DNA
tgtagcgtttgttgagg

SEQ ID NO: 91 - DNA, 17 bp, synthetic construct
  source 1..17: mol_type = other DNA
cagtcgtgtgtagcgtt

SEQ ID NO: 92 - DNA, 17 bp, synthetic construct
  source 1..17: mol_type = other DNA
acgcagtcgtgtgtagc

SEQ ID NO: 93 - DNA, 17 bp, synthetic construct
  source 1..17: mol_type = other DNA
tgagacgcagtcgtgtg

SEQ ID NO: 94 - DNA, 17 bp, synthetic construct
  source 1..17: mol_type = other DNA
tgatgagacgcagtcgt

SEQ ID NO: 95 - DNA, 17 bp, synthetic construct
  source 1..17: mol_type = other DNA
gtgcagagcggtcctat

SEQ ID NO: 96 - DNA, 17 bp, synthetic construct
  source 1..17: mol_type = other DNA
atctcgtacaaacagta

SEQ ID NO: 97 - DNA, 17 bp, synthetic construct
  source 1..17: mol_type = other DNA
ctccgaatgttacagcc

SEQ ID NO: 98 - DNA, 17 bp, synthetic construct
  source 1..17: mol_type = other DNA
cataaaattgcgactcc

SEQ ID NO: 99 - DNA, 17 bp, synthetic construct
  source 1..17: mol_type = other DNA
aaggctcaaagacgacg

SEQ ID NO: 100 - DNA, 17 bp, synthetic construct
  source 1..17: mol_type = other DNA
gcatcgtcggcaagctc

SEQ ID NO: 101 - DNA, 17 bp, synthetic construct
  source 1..17: mol_type = other DNA
ggtaatgtcaacggatc

SEQ ID NO: 102 - DNA, 16 bp, synthetic construct
  source 1..16: mol_type = other DNA
ggtaatgtcaacggat

SEQ ID NO: 103 - DNA, 17 bp, synthetic construct
  source 1..17: mol_type = other DNA
acgcttaatcaccgccg

SEQ ID NO: 104 - DNA, 16 bp, synthetic construct
  source 1..16: mol_type = other DNA
cgagtggtaaccggaa

SEQ ID NO: 105 - DNA, 17 bp, synthetic construct
  source 1..17: mol_type = other DNA
tgtcatcggaagcgagt

SEQ ID NO: 106 - DNA, 16 bp, synthetic construct
  source 1..16: mol_type = other DNA
acgtatcaggcgatgg

SEQ ID NO: 107 - DNA, 16 bp, synthetic construct
  source 1..16: mol_type = other DNA
gacgtatcaggcgatg

SEQ ID NO: 108 - DNA, 17 bp, synthetic construct
  source 1..17: mol_type = other DNA
cctagagtggcggactt

SEQ ID NO: 109 - DNA, 15 bp, synthetic construct
  source 1..15: mol_type = other DNA
agacgagttctacga

SEQ ID NO: 110 - DNA, 16 bp, synthetic construct
  source 1..16: mol_type = other DNA
gatgcgagttacgttt

SEQ ID NO: 111 - DNA, 16 bp, synthetic construct
  source 1..16: mol_type = other DNA
gtgatatgatgcgagt

SEQ ID NO: 112 - DNA, 17 bp, synthetic construct
  source 1..17: mol_type = other DNA
ttctcctgttggctcga

SEQ ID NO: 113 - DNA, 17 bp, synthetic construct
  source 1..17: mol_type = other DNA
accaggcagtgaacacg

SEQ ID NO: 114 - DNA, 17 bp, synthetic construct
  source 1..17: mol_type = other DNA
ttaggcttcggtccaca

SEQ ID NO: 115 - DNA, 17 bp, synthetic construct
  source 1..17: mol_type = other DNA
ggtccttaggcttcggt

SEQ ID NO: 116 - DNA, 17 bp, synthetic construct
  source 1..17: mol_type = other DNA
tcgatcattagcgtggc

SEQ ID NO: 117 - DNA, 17 bp, synthetic construct
  source 1..17: mol_type = other DNA
cgaagatccacacggcc

SEQ ID NO: 118 - DNA, 17 bp, synthetic construct
  source 1..17: mol_type = other DNA
tccttgatgagacgcag

SEQ ID NO: 119 - DNA, 17 bp, synthetic construct
  source 1..17: mol_type = other DNA
ttgccgatggccagaag

SEQ ID NO: 120 - DNA, 17 bp, synthetic construct
  source 1..17: mol_type = other DNA
tcttggtcttgccgatg

SEQ ID NO: 121 - DNA, 17 bp, synthetic construct
  source 1..17: mol_type = other DNA
cacacgtcttggtcttg

SEQ ID NO: 122 - DNA, 17 bp, synthetic construct
  source 1..17: mol_type = other DNA
gctctcacacgtcttgg

SEQ ID NO: 123 - DNA, 17 bp, synthetic construct
  source 1..17: mol_type = other DNA
gcagctcatcgaagccg

SEQ ID NO: 124 - DNA, 17 bp, synthetic construct
  source 1..17: mol_type = other DNA
gctcgtcaaaggcacct

SEQ ID NO: 125 - DNA, 17 bp, synthetic construct
  source 1..17: mol_type = other DNA
cagagcggtcctatgtg

SEQ ID NO: 126 - DNA, 17 bp, synthetic construct
  source 1..17: mol_type = other DNA
cggccttctgccagtca

SEQ ID NO: 127 - DNA, 17 bp, synthetic construct
  source 1..17: mol_type = other DNA
cacatgccgaggatggt

SEQ ID NO: 128 - DNA, 17 bp, synthetic construct
  source 1..17: mol_type = other DNA
ttggcctcggagaaacc

SEQ ID NO: 129 - DNA, 17 bp, synthetic construct
  source 1..17: mol_type = other DNA
gtgaagaggacctcgtt

SEQ ID NO: 130 - DNA, 16 bp, synthetic construct
  source 1..16: mol_type = other DNA
gaagacgtacaccgcg

SEQ ID NO: 131 - DNA, 17 bp, synthetic construct
  source 1..17: mol_type = other DNA
aggaagaagacgtacac

SEQ ID NO: 132 - DNA, 17 bp, synthetic construct
  source 1..17: mol_type = other DNA
gctgtagaacttctcgc

SEQ ID NO: 133 - DNA, 17 bp, synthetic construct
  source 1..17: mol_type = other DNA
gactgtcacgtctcggc

SEQ ID NO: 134 - DNA, 17 bp, synthetic construct
  source 1..17: mol_type = other DNA
tgcacgaagtcctcctc

SEQ ID NO: 135 - DNA, 17 bp, synthetic construct
  source 1..17: mol_type = other DNA
tctccgaatgttacagc

SEQ ID NO: 136 - DNA, 17 bp, synthetic construct
  source 1..17: mol_type = other DNA
tcatgcgagaggccaca

SEQ ID NO: 137 - DNA, 17 bp, synthetic construct
  source 1..17: mol_type = other DNA
cactcatgcgagaggcc

SEQ ID NO: 138 - DNA, 17 bp, synthetic construct
  source 1..17: mol_type = other DNA
agaagtctgattccgaa

SEQ ID NO: 139 - DNA, 17 bp, synthetic construct
  source 1..17: mol_type = other DNA
ggtgcgtgagattctga

SEQ ID NO: 140 - DNA, 17 bp, synthetic construct
  source 1..17: mol_type = other DNA
ctcgcaggtaaaggtgc

SEQ ID NO: 141 - DNA, 17 bp, synthetic construct
  source 1..17: mol_type = other DNA
cctgaagcttgcagtcg

SEQ ID NO: 142 - DNA, 17 bp, synthetic construct
  source 1..17: mol_type = other DNA
cgtgaggttgcagttgt

SEQ ID NO: 143 - DNA, 17 bp, synthetic construct
  source 1..17: mol_type = other DNA
gtgtgacgtgaggttgc

SEQ ID NO: 144 - DNA, 17 bp, synthetic construct
  source 1..17: mol_type = other DNA
caggctcagctttcgca

SEQ ID NO: 145 - DNA, 17 bp, synthetic construct
  source 1..17: mol_type = other DNA
atcgcagctctctccac

SEQ ID NO: 146 - DNA, 17 bp, synthetic construct
  source 1..17: mol_type = other DNA
tggcctggatggatcgc

SEQ ID NO: 147 - DNA, 17 bp, synthetic construct
  source 1..17: mol_type = other DNA
gcaagctctcttctccg

SEQ ID NO: 148 - DNA, 17 bp, synthetic construct
  source 1..17: mol_type = other DNA
gaaggcatcgtcggcaa

SEQ ID NO: 149 - DNA, 17 bp, synthetic construct
  source 1..17: mol_type = other DNA
aggaaggcatcgtcggc

SEQ ID NO: 150 - DNA, 17 bp, synthetic construct
  source 1..17: mol_type = other DNA
tgaggcgctgtgatgac

SEQ ID NO: 151 - DNA, 16 bp, synthetic construct
  source 1..16: mol_type = other DNA
acgtgttcttaggata

SEQ ID NO: 152 - DNA, 16 bp, synthetic construct
  source 1..16: mol_type = other DNA
gcactaccgtgagagg

SEQ ID NO: 153 - DNA, 17 bp, synthetic construct
  source 1..17: mol_type = other DNA
cacggttcttcaaggag

SEQ ID NO: 154 - DNA, 16 bp, synthetic construct
  source 1..16: mol_type = other DNA
agacggtgtagtggtt

SEQ ID NO: 155 - DNA, 16 bp, synthetic construct
  source 1..16: mol_type = other DNA
gtcactagatagcata

SEQ ID NO: 156 - DNA, 16 bp, synthetic construct
  source 1..16: mol_type = other DNA
tatttcgtgcctaatc

SEQ ID NO: 157 - DNA, 16 bp, synthetic construct
  source 1..16: mol_type = other DNA
agaacgaccagtctat

SEQ ID NO: 158 - DNA, 16 bp, synthetic construct
  source 1..16: mol_type = other DNA
gaacctatagtatggc

SEQ ID NO: 159 - DNA, 16 bp, synthetic construct
  source 1..16: mol_type = other DNA
taatcaccgccgctgg

SEQ ID NO: 160 - DNA, 16 bp, synthetic construct
  source 1..16: mol_type = other DNA
gaaacgcttaatcacc

SEQ ID NO: 161 - DNA, 16 bp, synthetic construct
  source 1..16: mol_type = other DNA
tgtgtagctaccagcc

SEQ ID NO: 162 - DNA, 16 bp, synthetic construct
  source 1..16: mol_type = other DNA
ccgagaatctctggtg

SEQ ID NO: 163 - DNA, 17 bp, synthetic construct
  source 1..17: mol_type = other DNA
cgagtggtaaccggaat

SEQ ID NO: 164 - DNA, 16 bp, synthetic construct
  source 1..16: mol_type = other DNA
tgtcatcggaagcgag

SEQ ID NO: 165 - DNA, 17 bp, synthetic construct
  source 1..17: mol_type = other DNA
ccacctgagatgcgctc

SEQ ID NO: 166 - DNA, 16 bp, synthetic construct
  source 1..16: mol_type = other DNA
gtcaacggatcaagaa

SEQ ID NO: 167 - DNA, 17 bp, synthetic construct
  source 1..17: mol_type = other DNA
ccttcatggccaaacca

SEQ ID NO: 168 - DNA, 16 bp, synthetic construct
  source 1..16: mol_type = other DNA
gcacttgtttccggac

SEQ ID NO: 169 - DNA, 17 bp, synthetic construct
  source 1..17: mol_type = other DNA
cgctggtgaacaggttc

SEQ ID NO: 170 - DNA, 16 bp, synthetic construct
  source 1..16: mol_type = other DNA
gccgtagaaaccgtac

SEQ ID NO: 171 - DNA, 16 bp, synthetic construct
  source 1..16: mol_type = other DNA
actgtttgctgccgta

SEQ ID NO: 172 - DNA, 17 bp, synthetic construct
  source 1..17: mol_type = other DNA
aggcaccatcgaccagg

SEQ ID NO: 173 - DNA, 17 bp, synthetic construct
  source 1..17: mol_type = other DNA
tatgaacttgcactcac

SEQ ID NO: 174 - DNA, 16 bp, synthetic construct
  source 1..16: mol_type = other DNA
ctagagtggcggactt

SEQ ID NO: 175 - DNA, 17 bp, synthetic construct
  source 1..17: mol_type = other DNA
actgcgtgtccaggcaa

SEQ ID NO: 176 - DNA, 17 bp, synthetic construct
  source 1..17: mol_type = other DNA
cattcagtgcgtgcttt

SEQ ID NO: 177 - DNA, 16 bp, synthetic construct
  source 1..16: mol_type = other DNA
taagccaccgaacagc

SEQ ID NO: 178 - DNA, 17 bp, synthetic construct
  source 1..17: mol_type = other DNA
tgtttgacgatttcatc

SEQ ID NO: 179 - DNA, 16 bp, synthetic construct
  source 1..16: mol_type = other DNA
agagagctccggaata

SEQ ID NO: 180 - DNA, 17 bp, synthetic construct
  source 1..17: mol_type = other DNA
ggacacacctgacaaga

SEQ ID NO: 181 - DNA, 16 bp, synthetic construct
  source 1..16: mol_type = other DNA
cgcctcgctggcagaa

SEQ ID NO: 182 - DNA, 16 bp, synthetic construct
  source 1..16: mol_type = other DNA
ccgttgaaggacgttt

SEQ ID NO: 183 - DNA, 16 bp, synthetic construct
  source 1..16: mol_type = other DNA
atccgttgaaggacgt

SEQ ID NO: 184 - DNA, 16 bp, synthetic construct
  source 1..16: mol_type = other DNA
ttgtagtgtgtactag

SEQ ID NO: 185 - DNA, 17 bp, synthetic construct
  source 1..17: mol_type = other DNA
tgtgagtgtcttggaag

SEQ ID NO: 186 - DNA, 16 bp, synthetic construct
  source 1..16: mol_type = other DNA
aatattctaaggcgat

SEQ ID NO: 187 - DNA, 16 bp, synthetic construct
  source 1..16: mol_type = other DNA
cgccgtcactccagtt

SEQ ID NO: 188 - DNA, 16 bp, synthetic construct
  source 1..16: mol_type = other DNA
cctgtacgacctataa

SEQ ID NO: 189 - DNA, 16 bp, synthetic construct
  source 1..16: mol_type = other DNA
acatggatgacgactt

SEQ ID NO: 190 - DNA, 16 bp, synthetic construct
  source 1..16: mol_type = other DNA
acggatatcttgccat

SEQ ID NO: 191 - DNA, 16 bp, synthetic construct
  source 1..16: mol_type = other DNA
caacagacggtgcagt

SEQ ID NO: 192 - DNA, 17 bp, synthetic construct
  source 1..17: mol_type = other DNA
ggtgtctacgaggcagg

SEQ ID NO: 193 - DNA, 16 bp, synthetic construct
  source 1..16: mol_type = other DNA
atgcatcaatctcgat

SEQ ID NO: 194 - DNA, 16 bp, synthetic construct
  source 1..16: mol_type = other DNA
gaacgtccattgtgac

SEQ ID NO: 195 - DNA, 17 bp, synthetic construct
  source 1..17: mol_type = other DNA
agccttgtatgctggta

SEQ ID NO: 196 - DNA, 16 bp, synthetic construct
  source 1..16: mol_type = other DNA
ggactaactagcttca

SEQ ID NO: 197 - DNA, 17 bp, synthetic construct
  source 1..17: mol_type = other DNA
ccttggccatgatcgca

SEQ ID NO: 198 - DNA, 16 bp, synthetic construct
  source 1..16: mol_type = other DNA
tcggttccttgtgtag

SEQ ID NO: 199 - DNA, 16 bp, synthetic construct
  source 1..16: mol_type = other DNA
agacgagttctacgaa

SEQ ID NO: 200 - DNA, 16 bp, synthetic construct
  source 1..16: mol_type = other DNA
gtgaccatcgactgaa

SEQ ID NO: 201 - DNA, 16 bp, synthetic construct
  source 1..16: mol_type = other DNA
agtgatatgatgcgag

SEQ ID NO: 202 - DNA, 16 bp, synthetic construct
  source 1..16: mol_type = other DNA
gataagaagacgatgt

SEQ ID NO: 203 - DNA, 16 bp, synthetic construct
  source 1..16: mol_type = other DNA
agcggaatcttcggaa

SEQ ID NO: 204 - DNA, 17 bp, synthetic construct
  source 1..17: mol_type = other DNA
tgcctctcacatcttcg

SEQ ID NO: 205 - DNA, 16 bp, synthetic construct
  source 1..16: mol_type = other DNA
gctcgtcgtgtccttc

SEQ ID NO: 206 - DNA, 16 bp, synthetic construct
  source 1..16: mol_type = other DNA
tgctcgtcgtgtcctt

SEQ ID NO: 207 - DNA, 16 bp, synthetic construct
  source 1..16: mol_type = other DNA
cgccgccgagaccaag

SEQ ID NO: 208 - DNA, 17 bp, synthetic construct
  source 1..17: mol_type = other DNA
gctccgcgtcctcgccg

SEQ ID NO: 209 - DNA, 18 bp, synthetic construct
  source 1..18: mol_type = other DNA
accaggcagtgaacacgg

SEQ ID NO: 210 - DNA, 18 bp, synthetic construct
  source 1..18: mol_type = other DNA
ggtgcttgccatcttcat

SEQ ID NO: 211 - DNA, 19 bp, synthetic construct
  source 1..19: mol_type = other DNA
gatagtcctctaagtgcat

SEQ ID NO: 212 - DNA, 18 bp, synthetic construct
  source 1..18: mol_type = other DNA
tagtcctctaagtgcatc

SEQ ID NO: 213 - DNA, 19 bp, synthetic construct
  source 1..19: mol_type = other DNA
agcgtggctagatccacat

SEQ ID NO: 214 - DNA, 18 bp, synthetic construct
  source 1..18: mol_type = other DNA
gctagatccacatggtct

SEQ ID NO: 215 - DNA, 18 bp, synthetic construct
  source 1..18: mol_type = other DNA
cattagcgtggctagatc

SEQ ID NO: 216 - DNA, 19 bp, synthetic construct
  source 1..19: mol_type = other DNA
acacgtgcattatctgaac

SEQ ID NO: 217 - DNA, 18 bp, synthetic construct
  source 1..18: mol_type = other DNA
tctcgaaaggtactccag

SEQ ID NO: 218 - DNA, 18 bp, synthetic construct
  source 1..18: mol_type = other DNA
aatctgcttctcacgtac

SEQ ID NO: 219 - DNA, 19 bp, synthetic construct
  source 1..19: mol_type = other DNA
gcgtttgttgaggctcaca

SEQ ID NO: 220 - DNA, 19 bp, synthetic construct
  source 1..19: mol_type = other DNA
gtgtagcgtttgttgaggc

SEQ ID NO: 221 - DNA, 19 bp, synthetic construct
  source 1..19: mol_type = other DNA
cgtgtgtagcgtttgttga

SEQ ID NO: 222 - DNA, 19 bp, synthetic construct
  source 1..19: mol_type = other DNA
gcagtcgtgtgtagcgttt

SEQ ID NO: 223 - DNA, 18 bp, synthetic construct
  source 1..18: mol_type = other DNA
tgtagcgtttgttgaggc

SEQ ID NO: 224 - DNA, 18 bp, synthetic construct
  source 1..18: mol_type = other DNA
agtcgtgtgtagcgtttg

SEQ ID NO: 225 - DNA, 18 bp, synthetic construct
  source 1..18: mol_type = other DNA
gcagtcgtgtgtagcgtt

SEQ ID NO: 226 - DNA, 18 bp, synthetic construct
  source 1..18: mol_type = other DNA
ctccggtgctccttgatg

SEQ ID NO: 227 - DNA, 19 bp, synthetic construct
  source 1..19: mol_type = other DNA
gtcttggtcttgccgatgg

SEQ ID NO: 228 - DNA, 19 bp, synthetic construct
  source 1..19: mol_type = other DNA
tagtcaaacctgtcttggt

SEQ ID NO: 229 - DNA, 19 bp, synthetic construct
  source 1..19: mol_type = other DNA
tagaacagatagtcaaacc

SEQ ID NO: 230 - DNA, 19 bp, synthetic construct
  source 1..19: mol_type = other DNA
cgacagtggatatagaaca

SEQ ID NO: 231 - DNA, 18 bp, synthetic construct
  source 1..18: mol_type = other DNA
agaacagatagtcaaacc

SEQ ID NO: 232 - DNA, 18 bp, synthetic construct
  source 1..18: mol_type = other DNA
cgacagtggatatagaac

SEQ ID NO: 233 - DNA, 19 bp, synthetic construct
  source 1..19: mol_type = other DNA
tcgtcaaaggcaccttgca

SEQ ID NO: 234 - DNA, 19 bp, synthetic construct
  source 1..19: mol_type = other DNA
cagtgcagagcggtcctat

SEQ ID NO: 235 - DNA, 18 bp, synthetic construct
  source 1..18: mol_type = other DNA
tcgtcaaaggcaccttgc

SEQ ID NO: 236 - DNA, 19 bp, synthetic construct
  source 1..19: mol_type = other DNA
cggccttctgccagtcagt

SEQ ID NO: 237 - DNA, 18 bp, synthetic construct
  source 1..18: mol_type = other DNA
agagcggtcctatgtgct

SEQ ID NO: 238 - DNA, 19 bp, synthetic construct
  source 1..19: mol_type = other DNA
ggtctcgtggtgatgagca

SEQ ID NO: 239 - DNA, 19 bp, synthetic construct
  source 1..19: mol_type = other DNA
cgaggatggtccagcaagt

SEQ ID NO: 240 - DNA, 18 bp, synthetic construct
  source 1..18: mol_type = other DNA
aggatctccacatgccga

SEQ ID NO: 241 - DNA, 19 bp, synthetic construct
  source 1..19: mol_type = other DNA
cacgatccagcagaccagg

SEQ ID NO: 242 - DNA, 19 bp, synthetic construct
  source 1..19: mol_type = other DNA
gacacatccgccttctgca

SEQ ID NO: 243 - DNA, 18 bp, synthetic construct
  source 1..18: mol_type = other DNA
gcagacacatccgccttc

SEQ ID NO: 244 - DNA, 19 bp, synthetic construct
  source 1..19: mol_type = other DNA
tacatggcggcaaagaact

SEQ ID NO: 245 - DNA, 18 bp, synthetic construct
  source 1..18: mol_type = other DNA
gtacatggcggcaaagaa

SEQ ID NO: 246 - DNA, 18 bp, synthetic construct
  source 1..18: mol_type = other DNA
ggtagtacatggcggcaa

SEQ ID NO: 247 - DNA, 18 bp, synthetic construct
  source 1..18: mol_type = other DNA
tcgaatttgccatagttt

SEQ ID NO: 248 - DNA, 19 bp, synthetic construct
  source 1..19: mol_type = other DNA
ctcgtacaaacagtagaac

SEQ ID NO: 249 - DNA, 18 bp, synthetic construct
  source 1..18: mol_type = other DNA
tcgtacaaacagtagaac

SEQ ID NO: 250 - DNA, 18 bp, synthetic construct
  source 1..18: mol_type = other DNA
ttgcacgaagtcctcctc

SEQ ID NO: 251 - DNA, 18 bp, synthetic construct
  source 1..18: mol_type = other DNA
gcatgttatggagaaacc

SEQ ID NO: 252 - DNA, 19 bp, synthetic construct
  source 1..19: mol_type = other DNA
gcgtttcacacaacactct

SEQ ID NO: 253 - DNA, 19 bp, synthetic construct
  source 1..19: mol_type = other DNA
cacaatctccgaatgttac

SEQ ID NO: 254 - DNA, 19 bp, synthetic construct
  source 1..19: mol_type = other DNA
gtcgaagcagcactcatgc

SEQ ID NO: 255 - DNA, 19 bp, synthetic construct
  source 1..19: mol_type = other DNA
gaccaaggagatgtcgaag

SEQ ID NO: 256 - DNA, 18 bp, synthetic construct
  source 1..18: mol_type = other DNA
tgtcgaagcagcactcat

SEQ ID NO: 257 - DNA, 18 bp, synthetic construct
  source 1..18: mol_type = other DNA
ggaccaaggagatgtcga

SEQ ID NO: 258 - DNA, 18 bp, synthetic construct
  source 1..18: mol_type = other DNA
ttcttcagattgcacaac

SEQ ID NO: 259 - DNA, 19 bp, synthetic construct
  source 1..19: mol_type = other DNA
gcgtgagattctgattagt

SEQ ID NO: 260 - DNA, 19 bp, synthetic construct
  source 1..19: mol_type = other DNA
aggtgcgtgagattctgat

SEQ ID NO: 261 - DNA, 19 bp, synthetic construct
  source 1..19: mol_type = other DNA
gtaaaggtgcgtgagattc

SEQ ID NO: 262 - DNA, 19 bp, synthetic construct
  source 1..19: mol_type = other DNA
ttgtctccgagagtgttgc

SEQ ID NO: 263 - DNA, 18 bp, synthetic construct
  source 1..18: mol_type = other DNA
cgtgagattctgattagt

SEQ ID NO: 264 - DNA, 19 bp, synthetic construct
  source 1..19: mol_type = other DNA
gtgaggttgcagttgtcta

SEQ ID NO: 265 - DNA, 19 bp, synthetic construct
  source 1..19: mol_type = other DNA
tgacgtgaggttgcagttg

SEQ ID NO: 266 - DNA, 18 bp, synthetic construct
  source 1..18: mol_type = other DNA
cctcgcaggtaaaggtgc

SEQ ID NO: 267 - DNA, 18 bp, synthetic construct
  source 1..18: mol_type = other DNA
ttgtctccgagagtgttg

SEQ ID NO: 268 - DNA, 19 bp, synthetic construct
  source 1..19: mol_type = other DNA
tttcgcaggctctggctgg

SEQ ID NO: 269 - DNA, 18 bp, synthetic construct
  source 1..18: mol_type = other DNA
acgtgaggttgcagttgt

SEQ ID NO: 270 - DNA, 19 bp, synthetic construct
  source 1..19: mol_type = other DNA
tcttgaagtgtttctaacg

SEQ ID NO: 271 - DNA, 18 bp, synthetic construct
  source 1..18: mol_type = other DNA
tcttgaagtgtttctaac

SEQ ID NO: 272 - DNA, 18 bp, synthetic construct
  source 1..18: mol_type = other DNA
tacatgaggtcaccaaga

SEQ ID NO: 273 - DNA, 19 bp, synthetic construct
  source 1..19: mol_type = other DNA
tgcacaatgtgagtgtacc

SEQ ID NO: 274 - DNA, 19 bp, synthetic construct
  source 1..19: mol_type = other DNA
caccttctcatagcactac

SEQ ID NO: 275 - DNA, 19 bp, synthetic construct
  source 1..19: mol_type = other DNA
tgagtatataagtactgac

SEQ ID NO: 276 - DNA, 19 bp, synthetic construct
  source 1..19: mol_type = other DNA
agtacttctacagttgtga

SEQ ID NO: 277 - DNA, 19 bp, synthetic construct
  source 1..19: mol_type = other DNA
agacagataacttcaggtg

SEQ ID NO: 278 - DNA, 19 bp, synthetic construct
  source 1..19: mol_type = other DNA
tataggagaaattcaggtt

SEQ ID NO: 279 - DNA, 18 bp, synthetic construct
  source 1..18: mol_type = other DNA
aaggcattctataggaga

SEQ ID NO: 280 - DNA, 19 bp, synthetic construct
  source 1..19: mol_type = other DNA
aacaacttctgctgtccac

SEQ ID NO: 281 - DNA, 19 bp, synthetic construct
  source 1..19: mol_type = other DNA
acatggctctgagttgcag

SEQ ID NO: 282 - DNA, 19 bp, synthetic construct
  source 1..19: mol_type = other DNA
attgaaacctggcctaata

SEQ ID NO: 283 - DNA, 19 bp, synthetic construct
  source 1..19: mol_type = other DNA
aatctctggtgtcaagttt

SEQ ID NO: 284 - DNA, 18 bp, synthetic construct
  source 1..18: mol_type = other DNA
agaagaagctggcgagga

SEQ ID NO: 285 - DNA, 19 bp, synthetic construct
  source 1..19: mol_type = other DNA
gactgactcttaggcacta

SEQ ID NO: 286 - DNA, 19 bp, synthetic construct
  source 1..19: mol_type = other DNA
cagacttggccactagctt

SEQ ID NO: 287 - DNA, 18 bp, synthetic construct
  source 1..18: mol_type = other DNA
tcattgcacagacttggc

SEQ ID NO: 288 - DNA, 18 bp, synthetic construct
  source 1..18: mol_type = other DNA
gactcaaccacttcattg

SEQ ID NO: 289 - DNA, 19 bp, synthetic construct
  source 1..19: mol_type = other DNA
cagtccttcatggccaaac

SEQ ID NO: 290 - DNA, 18 bp, synthetic construct
  source 1..18: mol_type = other DNA
gtgctttggatactctgc

SEQ ID NO: 291 - DNA, 19 bp, synthetic construct
  source 1..19: mol_type = other DNA
ttcaaatgcaaagtctagc

SEQ ID NO: 292 - DNA, 18 bp, synthetic construct
  source 1..18: mol_type = other DNA
tcacctgtagctctcatt

SEQ ID NO: 293 - DNA, 19 bp, synthetic construct
  source 1..19: mol_type = other DNA
ggtcctgaagatctttctc

SEQ ID NO: 294 - DNA, 19 bp, synthetic construct
  source 1..19: mol_type = other DNA
acaaagcattctgaccagg

SEQ ID NO: 295 - DNA, 18 bp, synthetic construct
  source 1..18: mol_type = other DNA
ttgataagactggtgaca

SEQ ID NO: 296 - DNA, 18 bp, synthetic construct
  source 1..18: mol_type = other DNA
ggtgactattcatgtgag

SEQ ID NO: 297 - DNA, 19 bp, synthetic construct
  source 1..19: mol_type = other DNA
catagttctctgcaacagg

SEQ ID NO: 298 - DNA, 19 bp, synthetic construct
  source 1..19: mol_type = other DNA
agtgcatgattattagtgg

SEQ ID NO: 299 - DNA, 19 bp, synthetic construct
  source 1..19: mol_type = other DNA
ctgagttccactaccttct

SEQ ID NO: 300 - DNA, 18 bp, synthetic construct
  source 1..18: mol_type = other DNA
cgtttgtctacactgagt

SEQ ID NO: 301 - DNA, 19 bp, synthetic construct
  source 1..19: mol_type = other DNA
gaatgctgatctggctggt

SEQ ID NO: 302 - DNA, 19 bp, synthetic construct
  source 1..19: mol_type = other DNA
tcttccggctccttcacag

SEQ ID NO: 303 - DNA, 19 bp, synthetic construct
  source 1..19: mol_type = other DNA
gttccatctgctttgcctg

SEQ ID NO: 304 - DNA, 19 bp, synthetic construct
  source 1..19: mol_type = other DNA
tacaccaagcatgtgttac

SEQ ID NO: 305 - DNA, 19 bp, synthetic construct
  source 1..19: mol_type = other DNA
actattgtaaatggctctc

SEQ ID NO: 306 - DNA, 18 bp, synthetic construct
  source 1..18: mol_type = other DNA
cacaaagctctgatactc

SEQ ID NO: 307 - DNA, 19 bp, synthetic construct
  source 1..19: mol_type = other DNA
gacttgtgttctttcacag

SEQ ID NO: 308 - DNA, 19 bp, synthetic construct
  source 1..19: mol_type = other DNA
attaatgccacttatagat

SEQ ID NO: 309 - DNA, 18 bp, synthetic construct
  source 1..18: mol_type = other DNA
ataggctatacctaacac

SEQ ID NO: 310 - DNA, 18 bp, synthetic construct
  source 1..18: mol_type = other DNA
acataggctatacctaac

SEQ ID NO: 311 - DNA, 18 bp, synthetic construct
  source 1..18: mol_type = other DNA
gccaagctaagatgtcac

SEQ ID NO: 312 - DNA, 18 bp, synthetic construct
  source 1..18: mol_type = other DNA
cagtgatgtccacctata

SEQ ID NO: 313 - DNA, 18 bp, synthetic construct
  source 1..18: mol_type = other DNA
gtaggagcttcttagatg

SEQ ID NO: 314 - DNA, 18 bp, synthetic construct
  source 1..18: mol_type = other DNA
gatatcttgccatgtact

SEQ ID NO: 315 - DNA, 19 bp, synthetic construct
  source 1..19: mol_type = other DNA
caggctttaccactatttc

SEQ ID NO: 316 - DNA, 18 bp, synthetic construct
  source 1..18: mol_type = other DNA
tgagtggcttctactaag

SEQ ID NO: 317 - DNA, 18 bp, synthetic construct
  source 1..18: mol_type = other DNA
tttaagctctttggtgtc

SEQ ID NO: 318 - DNA, 19 bp, synthetic construct
  source 1..19: mol_type = other DNA
tttgatgtactattcttga

SEQ ID NO: 319 - DNA, 19 bp, synthetic construct
  source 1..19: mol_type = other DNA
gtttctctcaaggccacag

SEQ ID NO: 320 - DNA, 18 bp, synthetic construct
  source 1..18: mol_type = other DNA
tgaccttctaagctgtag

SEQ ID NO: 321 - DNA, 18 bp, synthetic construct
  source 1..18: mol_type = other DNA
ccgtaacactgcagacac

SEQ ID NO: 322 - DNA, 19 bp, synthetic construct
  source 1..19: mol_type = other DNA
acacctcacttctaagata

SEQ ID NO: 323 - DNA, 19 bp, synthetic construct
  source 1..19: mol_type = other DNA
gaagtggaatgtgtagttt

SEQ ID NO: 324 - DNA, 19 bp, synthetic construct
  source 1..19: mol_type = other DNA
ccttaaagccaagcagtga

SEQ ID NO: 325 - DNA, 18 bp, synthetic construct
  source 1..18: mol_type = other DNA
acgtttgagaggtttatt

SEQ ID NO: 326 - DNA, 18 bp, synthetic construct
  source 1..18: mol_type = other DNA
aaagagacagtgacgaca

SEQ ID NO: 327 - DNA, 18 bp, synthetic construct
  source 1..18: mol_type = other DNA
gagtttgttgctctgatt

SEQ ID NO: 328 - DNA, 19 bp, synthetic construct
  source 1..19: mol_type = other DNA
ggagaggctggattatgtg

SEQ ID NO: 329 - DNA, 19 bp, synthetic construct
  source 1..19: mol_type = other DNA
gatgacagtgttcttgcaa

SEQ ID NO: 330 - DNA, 19 bp, synthetic construct
  source 1..19: mol_type = other DNA
gatcattaatatggacaga

SEQ ID NO: 331 - DNA, 19 bp, synthetic construct
  source 1..19: mol_type = other DNA
gcttgttatcgcagcctga

SEQ ID NO: 332 - DNA, 18 bp, synthetic construct
  source 1..18: mol_type = other DNA
cagcttgttatcgcagcc

SEQ ID NO: 333 - DNA, 19 bp, synthetic construct
  source 1..19: mol_type = other DNA
accaaattcatcagagact

SEQ ID NO: 334 - DNA, 19 bp, synthetic construct
  source 1..19: mol_type = other DNA
ctcctctttaataaagttt

SEQ ID NO: 335 - DNA, 19 bp, synthetic construct
  source 1..19: mol_type = other DNA
tagtatcactgtatgtcca

SEQ ID NO: 336 - DNA, 19 bp, synthetic construct
  source 1..19: mol_type = other DNA
ggtcatgtcaacttgtttc

SEQ ID NO: 337 - DNA, 18 bp, synthetic construct
  source 1..18: mol_type = other DNA
tcagagtcgttaacctta

SEQ ID NO: 338 - DNA, 19 bp, synthetic construct
  source 1..19: mol_type = other DNA
ccacaatctccgaatgtta

SEQ ID NO: 339 - DNA, 18 bp, synthetic construct
  source 1..18: mol_type = other DNA
cgtttaggctatgtactt

SEQ ID NO: 340 - DNA, 16 bp, synthetic construct
  source 1..16: mol_type = other DNA
tacgcgcggttgttta

SEQ ID NO: 341 - DNA, 17 bp, synthetic construct
  source 1..17: mol_type = other DNA
gatcattcgcggacaac

SEQ ID NO: 342 - DNA, 16 bp, synthetic construct
  source 1..16: mol_type = other DNA
cttgatccagacgtat

SEQ ID NO: 343 - DNA, 17 bp, synthetic construct
  source 1..17: mol_type = other DNA
aacggacactcgtcatc

SEQ ID NO: 344 - DNA, 17 bp, synthetic construct
  source 1..17: mol_type = other DNA
ttgcaacggacactcgt

SEQ ID NO: 345 - DNA, 17 bp, synthetic construct
  source 1..17: mol_type = other DNA
ctcgcctgttgatcgca

SEQ ID NO: 346 - DNA, 16 bp, synthetic construct
  source 1..16: mol_type = other DNA
ctcgcctgttgatcgc

SEQ ID NO: 347 - DNA, 15 bp, synthetic construct
  source 1..15: mol_type = other DNA
ctcgcctgttgatcg

SEQ ID NO: 348 - DNA, 16 bp, synthetic construct
  source 1..16: mol_type = other DNA
tctcgcctgttgatcg

SEQ ID NO: 349 - DNA, 16 bp, synthetic construct
  source 1..16: mol_type = other DNA
agacgcgcgttcctgt

SEQ ID NO: 350 - DNA, 17 bp, synthetic construct
  source 1..17: mol_type = other DNA
cacctagacgcgcgttc

SEQ ID NO: 351 - DNA, 17 bp, synthetic construct
  source 1..17: mol_type = other DNA
cgtgtagcgactgttga

SEQ ID NO: 352 - DNA, 16 bp, synthetic construct
  source 1..16: mol_type = other DNA
cgtgtagcgactgttg

SEQ ID NO: 353 - DNA, 17 bp, synthetic construct
  source 1..17: mol_type = other DNA
gcgtgtagcgactgttg

SEQ ID NO: 354 - DNA, 16 bp, synthetic construct
  source 1..16: mol_type = other DNA
gcgtgtagcgactgtt

SEQ ID NO: 355 - DNA, 17 bp, synthetic construct
  source 1..17: mol_type = other DNA
tgcgtgtagcgactgtt

SEQ ID NO: 356 - DNA, 17 bp, synthetic construct
  source 1..17: mol_type = other DNA
ggagctgcgtgtagcga

SEQ ID NO: 357 - DNA, 16 bp, synthetic construct
  source 1..16: mol_type = other DNA
tagtccggccgatggt

SEQ ID NO: 358 - DNA, 17 bp, synthetic construct
  source 1..17: mol_type = other DNA
ttagtccggccgatggt

SEQ ID NO: 359 - DNA, 17 bp, synthetic construct
  source 1..17: mol_type = other DNA
accggcctcgtcgttat

SEQ ID NO: 360 - DNA, 17 bp, synthetic construct
  source 1..17: mol_type = other DNA
ctaccggcctcgtcgtt

SEQ ID NO: 361 - DNA, 16 bp, synthetic construct
  source 1..16: mol_type = other DNA
ctaccggcctcgtcgt

SEQ ID NO: 362 - DNA, 17 bp, synthetic construct
  source 1..17: mol_type = other DNA
aaggctaccggcctcgt

SEQ ID NO: 363 - DNA, 17 bp, synthetic construct
  source 1..17: mol_type = other DNA
tagacggccgtagtggt

SEQ ID NO: 364 - DNA, 16 bp, synthetic construct
  source 1..16: mol_type = other DNA
tagacggccgtagtgg

SEQ ID NO: 365 - DNA, 17 bp, synthetic construct
  source 1..17: mol_type = other DNA
gtagacggccgtagtgg

SEQ ID NO: 366 - DNA, 16 bp, synthetic construct
  source 1..16: mol_type = other DNA
gtagacggccgtagtg

SEQ ID NO: 367 - DNA, 16 bp, synthetic construct
  source 1..16: mol_type = other DNA
acgtagacggccgtag

SEQ ID NO: 368 - DNA, 16 bp, synthetic construct
  source 1..16: mol_type = other DNA
agaagacgtagacggc

SEQ ID NO: 369 - DNA, 17 bp, synthetic construct
  source 1..17: mol_type = other DNA
agcggagacgtcagtct

SEQ ID NO: 370 - DNA, 17 bp, synthetic construct
  source 1..17: mol_type = other DNA
ggaccttcacgtctcgg

SEQ ID NO: 371 - DNA, 17 bp, synthetic construct
  source 1..17: mol_type = other DNA
tagtaggaccttcacgt

SEQ ID NO: 372 - DNA, 16 bp, synthetic construct
  source 1..16: mol_type = other DNA
tagtaggaccttcacg

SEQ ID NO: 373 - DNA, 17 bp, synthetic construct
  source 1..17: mol_type = other DNA
ctagtaggaccttcacg

SEQ ID NO: 374 - DNA, 17 bp, synthetic construct
  source 1..17: mol_type = other DNA
acaacacgcggatgtga

SEQ ID NO: 375 - DNA, 17 bp, synthetic construct
  source 1..17: mol_type = other DNA
gcacgttttgtttcacg

SEQ ID NO: 376 - DNA, 17 bp, synthetic construct
  source 1..17: mol_type = other DNA
acgttatcgatgtgagg

SEQ ID NO: 377 - DNA, 16 bp, synthetic construct
  source 1..16: mol_type = other DNA
acgttatcgatgtgag

SEQ ID NO: 378 - DNA, 16 bp, synthetic construct
  source 1..16: mol_type = other DNA
ttcacgttatcgatgt

SEQ ID NO: 379 - DNA, 17 bp, synthetic construct
  source 1..17: mol_type = other DNA
tttcacgttatcgatgt

SEQ ID NO: 380 - DNA, 16 bp, synthetic construct
  source 1..16: mol_type = other DNA
gtttcacgttatcgat

SEQ ID NO: 381 - DNA, 17 bp, synthetic construct
  source 1..17: mol_type = other DNA
agtttcacgttatcgat

SEQ ID NO: 382 - DNA, 17 bp, synthetic construct
  source 1..17: mol_type = other DNA
acgtgtaccttactgta

SEQ ID NO: 383 - DNA, 17 bp, synthetic construct
  source 1..17: mol_type = other DNA
ctacctgcacgaggaat

SEQ ID NO: 384 - DNA, 16 bp, synthetic construct
  source 1..16: mol_type = other DNA
gaacaattacggctgc

SEQ ID NO: 385 - DNA, 17 bp, synthetic construct
  source 1..17: mol_type = other DNA
ccacataggtgccgtag

SEQ ID NO: 386 - DNA, 17 bp, synthetic construct
  source 1..17: mol_type = other DNA
gcacggatagtaatagg

SEQ ID NO: 387 - DNA, 16 bp, synthetic construct
  source 1..16: mol_type = other DNA
cttactatgaccggcc

SEQ ID NO: 388 - DNA, 17 bp, synthetic construct
  source 1..17: mol_type = other DNA
tactcttactatgaccg

SEQ ID NO: 389 - DNA, 16 bp, synthetic construct
  source 1..16: mol_type = other DNA
gtgcaaccgcgaacct

SEQ ID NO: 390 - DNA, 17 bp, synthetic construct
  source 1..17: mol_type = other DNA
cgtgcaaccgcgaacct

SEQ ID NO: 391 - DNA, 16 bp, synthetic construct
  source 1..16: mol_type = other DNA
acattcgtgcaaccgc

SEQ ID NO: 392 - DNA, 17 bp, synthetic construct
  source 1..17: mol_type = other DNA
aacattcgtgcaaccgc

SEQ ID NO: 393 - DNA, 17 bp, synthetic construct
  source 1..17: mol_type = other DNA
ctcagttggatgtacgt

SEQ ID NO: 394 - DNA, 16 bp, synthetic construct
  source 1..16: mol_type = other DNA
ctcagttggatgtacg

SEQ ID NO: 395 - DNA, 17 bp, synthetic construct
  source 1..17: mol_type = other DNA
cactgctcgagtgtatc

SEQ ID NO: 396 - DNA, 17 bp, synthetic construct
  source 1..17: mol_type = other DNA
tggtatgaccggacaga

SEQ ID NO: 397 - DNA, 16 bp, synthetic construct
  source 1..16: mol_type = other DNA
atagagcgagctgtcg

SEQ ID NO: 398 - DNA, 17 bp, synthetic construct
  source 1..17: mol_type = other DNA
tatagagcgagctgtcg

SEQ ID NO: 399 - DNA, 16 bp, synthetic construct
  source 1..16: mol_type = other DNA
aggatttacgaacaac

SEQ ID NO: 400 - DNA, 17 bp, synthetic construct
  source 1..17: mol_type = other DNA
taagctgctagcggacc

SEQ ID NO: 401 - DNA, 17 bp, synthetic construct
  source 1..17: mol_type = other DNA
gcatggaagcgatattc

SEQ ID NO: 402 - DNA, 17 bp, synthetic construct
  source 1..17: mol_type = other DNA
tctcagaacgacggaat

SEQ ID NO: 403 - DNA, 17 bp, synthetic construct
  source 1..17: mol_type = other DNA
gagcatgtttgccgata

SEQ ID NO: 404 - DNA, 17 bp, synthetic construct
  source 1..17: mol_type = other DNA
ccgttggactggtatca

SEQ ID NO: 405 - DNA, 16 bp, synthetic construct
  source 1..16: mol_type = other DNA
ttcacgtatcagcaca

SEQ ID NO: 406 - DNA, 17 bp, synthetic construct
  source 1..17: mol_type = other DNA
gtgaacgtaatggcagg

SEQ ID NO: 407 - DNA, 17 bp, synthetic construct
  source 1..17: mol_type = other DNA
ttaaagaccggtgtgcc

SEQ ID NO: 408 - DNA, 17 bp, synthetic construct
  source 1..17: mol_type = other DNA
acctcgagtttggttca

SEQ ID NO: 409 - DNA, 17 bp, synthetic construct
  source 1..17: mol_type = other DNA
gaccggtgatctcataa

SEQ ID NO: 410 - DNA, 16 bp, synthetic construct
  source 1..16: mol_type = other DNA
gaactgaccggtgatc

SEQ ID NO: 411 - DNA, 17 bp, synthetic construct
  source 1..17: mol_type = other DNA
ccgtgcgttaggagaat

SEQ ID NO: 412 - DNA, 16 bp, synthetic construct
  source 1..16: mol_type = other DNA
tctttccgtgcgttag

SEQ ID NO: 413 - DNA, 17 bp, synthetic construct
  source 1..17: mol_type = other DNA
tgaatgcaccaagctcg

SEQ ID NO: 414 - DNA, 17 bp, synthetic construct
  source 1..17: mol_type = other DNA
ccgaccatataatgcta

SEQ ID NO: 415 - DNA, 17 bp, synthetic construct
  source 1..17: mol_type = other DNA
agcgttcagtaaggacc

SEQ ID NO: 416 - DNA, 17 bp, synthetic construct
  source 1..17: mol_type = other DNA
tacgttcctactagagc

SEQ ID NO: 417 - DNA, 17 bp, synthetic construct
  source 1..17: mol_type = other DNA
acggttgcatagctggc

SEQ ID NO: 418 - DNA, 17 bp, synthetic construct
  source 1..17: mol_type = other DNA
tgttactgagtacgacc

SEQ ID NO: 419 - DNA, 17 bp, synthetic construct
  source 1..17: mol_type = other DNA
agcaatttggtaacgtc

SEQ ID NO: 420 - DNA, 17 bp, synthetic construct
  source 1..17: mol_type = other DNA
ccgtggcagcaccttaa

SEQ ID NO: 421 - DNA, 17 bp, synthetic construct
  source 1..17: mol_type = other DNA
aactgagcgtagtgaag

SEQ ID NO: 422 - DNA, 17 bp, synthetic construct
  source 1..17: mol_type = other DNA
taacttgcggtattatc

SEQ ID NO: 423 - DNA, 16 bp, synthetic construct
  source 1..16: mol_type = other DNA
gtctaacttgcggtat

SEQ ID NO: 424 - DNA, 16 bp, synthetic construct
  source 1..16: mol_type = other DNA
caatcctcgttgttcc

SEQ ID NO: 425 - DNA, 17 bp, synthetic construct
  source 1..17: mol_type = other DNA
tacgtcagcaatcctcg

SEQ ID NO: 426 - DNA, 17 bp, synthetic construct
  source 1..17: mol_type = other DNA
aagtttgctgtccggta

SEQ ID NO: 427 - DNA, 17 bp, synthetic construct
  source 1..17: mol_type = other DNA
cgagaagcgatgcttag

SEQ ID NO: 428 - DNA, 17 bp, synthetic construct
  source 1..17: mol_type = other DNA
gcttggatccacgagag

SEQ ID NO: 429 - DNA, 16 bp, synthetic construct
  source 1..16: mol_type = other DNA
cactgcgagctgcatc

SEQ ID NO: 430 - DNA, 17 bp, synthetic construct
  source 1..17: mol_type = other DNA
cgcatatacaaagtcac

SEQ ID NO: 431 - DNA, 17 bp, synthetic construct
  source 1..17: mol_type = other DNA
cttgatccagacgtatg

SEQ ID NO: 432 - DNA, 16 bp, synthetic construct
  source 1..16: mol_type = other DNA
ttgatccagacgtatg

SEQ ID NO: 433 - DNA, 17 bp, synthetic construct
  source 1..17: mol_type = other DNA
gcttgatccagacgtat

SEQ ID NO: 434 - DNA, 17 bp, synthetic construct
  source 1..17: mol_type = other DNA
cggacactcgtcatctt

SEQ ID NO: 435 - DNA, 17 bp, synthetic construct
  source 1..17: mol_type = other DNA
agctcgttggagaaata

SEQ ID NO: 436 - DNA, 17 bp, synthetic construct
  source 1..17: mol_type = other DNA
ctgtagaatctctcgca

SEQ ID NO: 437 - DNA, 17 bp, synthetic construct
  source 1..17: mol_type = other DNA
atagtacatagcagcga

SEQ ID NO: 438 - DNA, 16 bp, synthetic construct
  source 1..16: mol_type = other DNA
tagtacatagcagcga

SEQ ID NO: 439 - DNA, 17 bp, synthetic construct
  source 1..17: mol_type = other DNA
caaacttgccgtaattt

SEQ ID NO: 440 - DNA, 17 bp, synthetic construct
  source 1..17: mol_type = other DNA
tagaacagttccagttg

SEQ ID NO: 441 - DNA, 17 bp, synthetic construct
  source 1..17: mol_type = other DNA
tgcttcgtagatagagg

SEQ ID NO: 442 - DNA, 17 bp, synthetic construct
  source 1..17: mol_type = other DNA
cattgcttcgtagatag

SEQ ID NO: 443 - DNA, 17 bp, synthetic construct
  source 1..17: mol_type = other DNA
cgccaagatcattgttg

SEQ ID NO: 444 - DNA, 17 bp, synthetic construct
  source 1..17: mol_type = other DNA
gcacagatcgccaagat

SEQ ID NO: 445 - DNA, 17 bp, synthetic construct
  source 1..17: mol_type = other DNA
cgttttgtttcacgatt

SEQ ID NO: 446 - DNA, 17 bp, synthetic construct
  source 1..17: mol_type = other DNA
gaaatctcgaagactat

SEQ ID NO: 447 - DNA, 17 bp, synthetic construct
  source 1..17: mol_type = other DNA
cgttatcgatgtgagga

SEQ ID NO: 448 - DNA, 17 bp, synthetic construct
  source 1..17: mol_type = other DNA
gcagtttcacgttatcg

SEQ ID NO: 449 - DNA, 16 bp, synthetic construct
  source 1..16: mol_type = other DNA
gagccagcttgcaacg

SEQ ID NO: 450 - DNA, 17 bp, synthetic construct
  source 1..17: mol_type = other DNA
gtctcgcctgttgatcg

SEQ ID NO: 451 - DNA, 16 bp, synthetic construct
  source 1..16: mol_type = other DNA
gtctcgcctgttgatc

SEQ ID NO: 452 - DNA, 17 bp, synthetic construct
  source 1..17: mol_type = other DNA
gtcgtctgtacatctta

SEQ ID NO: 453 - DNA, 17 bp, synthetic construct
  source 1..17: mol_type = other DNA
agcgactgttgaggtcc

SEQ ID NO: 454 - DNA, 17 bp, synthetic construct
  source 1..17: mol_type = other DNA
agttggagctgcgtgta

SEQ ID NO: 455 - DNA, 17 bp, synthetic construct
  source 1..17: mol_type = other DNA
gtccaacataatcttcc

SEQ ID NO: 456 - DNA, 17 bp, synthetic construct
  source 1..17: mol_type = other DNA
gtcgttatgagcagaga

SEQ ID NO: 457 - DNA, 16 bp, synthetic construct
  source 1..16: mol_type = other DNA
taccggcctcgtcgtt

SEQ ID NO: 458 - DNA, 17 bp, synthetic construct
  source 1..17: mol_type = other DNA
acgtagacggccgtagt

SEQ ID NO: 459 - DNA, 17 bp, synthetic construct
  source 1..17: mol_type = other DNA
ccgtgcttccgcagatc

SEQ ID NO: 460 - DNA, 17 bp, synthetic construct
  source 1..17: mol_type = other DNA
gccgtgcttccgcagat

SEQ ID NO: 461 - DNA, 17 bp, synthetic construct
  source 1..17: mol_type = other DNA
ctctcgcagtccacttc

SEQ ID NO: 462 - DNA, 17 bp, synthetic construct
  source 1..17: mol_type = other DNA
atctctcgcagtccact

SEQ ID NO: 463 - DNA, 17 bp, synthetic construct
  source 1..17: mol_type = other DNA
gaaccaatgcgagatcc

SEQ ID NO: 464 - DNA, 16 bp, synthetic construct
  source 1..16: mol_type = other DNA
agtaaggccggaattc

SEQ ID NO: 465 - DNA, 17 bp, synthetic construct
  source 1..17: mol_type = other DNA
ggactcgtgtctgacca

SEQ ID NO: 466 - DNA, 17 bp, synthetic construct
  source 1..17: mol_type = other DNA
cgcgttcctgtccttga

SEQ ID NO: 467 - DNA, 17 bp, synthetic construct
  source 1..17: mol_type = other DNA
tagacgcgcgttcctgt

SEQ ID NO: 468 - DNA, 16 bp, synthetic construct
  source 1..16: mol_type = other DNA
tagacgcgcgttcctg

SEQ ID NO: 469 - DNA, 16 bp, synthetic construct
  source 1..16: mol_type = other DNA
ctcacctagacgcgcg

SEQ ID NO: 470 - DNA, 16 bp, synthetic construct
  source 1..16: mol_type = other DNA
ctctcacctagacgcg

SEQ ID NO: 471 - DNA, 17 bp, synthetic construct
  source 1..17: mol_type = other DNA
gctgcgtgtagcgactg

SEQ ID NO: 472 - DNA, 17 bp, synthetic construct
  source 1..17: mol_type = other DNA
cggccgatggtcaggag

SEQ ID NO: 473 - DNA, 17 bp, synthetic construct
  source 1..17: mol_type = other DNA
cattttagtccggccga

SEQ ID NO: 474 - DNA, 16 bp, synthetic construct
  source 1..16: mol_type = other DNA
gcctcgtcgttatgag

SEQ ID NO: 475 - DNA, 17 bp, synthetic construct
  source 1..17: mol_type = other DNA
caaggctaccggcctcg

SEQ ID NO: 476 - DNA, 16 bp, synthetic construct
  source 1..16: mol_type = other DNA
cggccgtagtggtctt

SEQ ID NO: 477 - DNA, 15 bp, synthetic construct
  source 1..15: mol_type = other DNA
agacgtagacggccg

SEQ ID NO: 478 - DNA, 17 bp, synthetic construct
  source 1..17: mol_type = other DNA
agaagacgtagacggcc

SEQ ID NO: 479 - DNA, 17 bp, synthetic construct
  source 1..17: mol_type = other DNA
gcaggccgtgcttccgc

SEQ ID NO: 480 - DNA, 17 bp, synthetic construct
  source 1..17: mol_type = other DNA
tcggttcagaagatctg

SEQ ID NO: 481 - DNA, 17 bp, synthetic construct
  source 1..17: mol_type = other DNA
ggttggtgcttagactt

SEQ ID NO: 482 - DNA, 17 bp, synthetic construct
  source 1..17: mol_type = other DNA
tgtcgaagcagcattga

SEQ ID NO: 483 - DNA, 17 bp, synthetic construct
  source 1..17: mol_type = other DNA
cgcttctaaggcacgtt

SEQ ID NO: 484 - DNA, 17 bp, synthetic construct
  source 1..17: mol_type = other DNA
tcacgttatcgatgtga

SEQ ID NO: 485 - DNA, 17 bp, synthetic construct
  source 1..17: mol_type = other DNA
gccggaattcaccaacc

SEQ ID NO: 486 - DNA, 17 bp, synthetic construct
  source 1..17: mol_type = other DNA
ttatgtccggttatttc

(19) United States
(12) Patent Application Publication
(10) Pub. No.: US 2025/0059547 A1
(43) Pub. Date: Feb. 20, 2025

(54) NLRP3 oligonucleotide for use in preventing and/or treating an inflammatory disease

(71) Applicant: Secarna Pharmaceuticals GmbH & Co. KG, Marburg (DE)

(72) Inventors: Marta Lucia DE LOS REYES, Munich (DE); Frank JASCHINSKI, Puchheim (DE); Sven MICHEL, Bernried (DE); Anna URI, Munich (DE)

(21) Appl. No.: 18/722,941

(22) PCT Filed: Dec. 23, 2022
(86) PCT No.: PCT/EP2022/087670

§ 371 Date: Jun. 21, 2024

(30) Foreign Application Priority Data
Dec. 23, 2021 (EP) 21217418.9

(51) Int. Cl. C12N 15/113; A61K 45/06; A61P 37/06

(52) U.S. Cl. CPC C12N 15/1138; A61K 45/06; A61P 37/06; C12N 2310/11; C12N 2310/316; C12N 2310/3231

(57) ABSTRACT

The present invention refers to oligonucleotide consisting of 12 to 20 nucleotides comprising at least one modified nucleotide hybridizing with a nucleic acid sequence of NLR family pyrin domain containing 3 (NLRP3) and a pharmaceutical composition 5 comprising such oligonucleotide together with a pharmaceutically acceptable carrier, excipient and/or dilutant to inhibit the expression of NLRP3 for example for preventing and/or treating an inflammatory disease.

The present invention refers to an oligonucleotide hybridizing with a nucleic acid sequence of NLR family pyrin domain containing 3 (NLRP3) and a pharmaceutical composition comprising such oligonucleotide together with a pharmaceutically acceptable carrier, excipient and/or dilutant to inhibit the expression of NLRP3 for example for use in preventing and/or treating an inflammatory disease.

### TECHNICAL BACKGROUND

NLR family pyrin domain containing 3 (NLRP3) is primarily expressed in cells of the innate immune system like macrophages and dendritic cells. Upon activation, it forms a proteolytic complex with Apoptosis-associated speck-like protein containing a CARD (ASC) and caspase-1, the NLRP3 inflammasome, which cleaves pro-IL-1β and pro-IL-18 to the mature forms of these pro-inflammatory cytokines. In addition, NLRP3 activation leads to a form of cell death called pyroptosis by cleaving gasdermin D into its pore-forming active form (Anders, H.-J., and Muruve, D. A. (2011), J Am Soc Nephrol 22, 1007-18; Masood, H., Che, R., and Zhang, A. (2015), Kidney Dis (Basel) 1, 187-93).

Activation of the NLRP3-caspase-1-IL-1β/IL-18 axis requires two signals: Signal 1 is derived from pattern recognition receptors, e.g., toll like receptor (TLR) 4 recognizing lipopolysaccharide (LPS) and increases the transcription and translation of pro-IL-1β, pro-IL-18 and NLRP3 through nuclear factor “kappa-light-chain-enhancer” of activated B-cells (NFκB). Signal 2 is then required for activation of caspase-1 via NLRP3. Various danger associated molecular patterns (DAMPs) and pathogen associated molecular patterns (PAMPs) can act as signal 2, i.e., ATP or uric acid that are released by necroptotic cells or reactive oxygen species (ROS) as a result of cellular stress (Sutterwala, F. S., Haasken, S., and Cassel, S. L. (2014), Ann N Y Acad Sci 1319, 82-95).

Dysregulation of the NLRP3 inflammasome by activating mutations in the NLRP3 gene is the underlying cause of cryopyrin-associated periodic syndromes (CAPS): Muckle-Wells syndrome (MWS), familial cold autoinflammatory syndrome (FCAS), and chronic infantile neurologic cutaneous and articular syndrome (NOMID) (Kuemmerle-Deschner, J. B. (2015), Semin Immunopathol 37, 377-85). Moreover, NLRP3 activation and its downstream effects are involved in various inflammatory diseases of the kidney, arthritis, neuroinflammation and metabolic disorders. Blocking IL-1β signalling with antibodies, i.e., Canakinumab, is an efficient treatment for many of the above mentioned diseases. However, NLRP3 has inflammasome-independent effects, presumably through enhancing SMAD signalling downstream of transforming growth factor beta (TGFβ) via inducing mitochondrial ROS production (Bracey, N. A. et al., (2014), Biol Chem 289, 19571-84; Wang, W. et al., (2013), J Immunol 190, 1239-49). Thus, targeting NLRP3 instead of IL-1β and/or IL-18 is a superior approach to treat NLRP3-mediated diseases.

Oligonucleotides of the present invention are very successful in the inhibition of the expression and activity of NLRP3, respectively. The mode of action of an oligonucleotide differs from the mode of action of an antibody or small molecule, and oligonucleotides are highly advantageous regarding for example
(i) the penetration of tissue,
(ii) the blocking of multiple functions and activities, respectively, of a target by prevention of its expression,
(iii) the combination of oligonucleotides with each other or an antibody, a polypeptide or a small molecule, and
(iv) the inhibition of intracellular targets which are not accessible for an antibody or inhibitable via a small molecule.

### SUMMARY

The present invention refers to an oligonucleotide comprising or consisting of a sequence selected from the group consisting of SEQ ID NO. 59, SEQ ID NO. 133, SEQ ID NO. 297 and SEQ ID NO. 335 comprising at least one modified nucleotide, hybridizing with a nucleic acid sequence of a NLR family pyrin domain containing 3 (NLRP3) of SEQ ID NO. 1 and/or SEQ ID NO. 2 (human), SEQ ID NO. 3 and/or SEQ ID NO. 4 (mouse) resulting in a reduction of the level of NLRP3, NLRP3 mRNA, NLRP3 pre-mRNA or a combination thereof. The reduction of the level of NLRP3, NLRP3 mRNA, NLRP3 pre-mRNA or a combination thereof is 30 to 99% compared to an untreated control.

The modified nucleotide is for example selected from the group consisting of a bridged nucleic acid such as LNA, cET, ENA, a 2′Fluoro modified nucleotide, a 2′O-Methyl modified nucleotide, a 2 O-Methoxy modified nucleotide, a FANA and a combination thereof.

The oligonucleotide of the present invention, wherein the oligonucleotide comprises for example a modification or consists of a sequence selected from the group consisting of

(A31109Hi; SEQ ID NO. 59)
+G*+T*+A*A*T*G*T*C*A*A*C*G*G*A*+T*+C,
(A31055Hi, SEQ ID NO. 59)
+G*+T*+A*A*T*G*T*C*A*A*C*G*G*+A*+T*+C,
(A31110Hi, SEQ ID NO. 59)
+G*+T*A*A*T*G*T*C*A*A*C*G*G*+A*+T*+C,
(A31111Hi, SEQ ID NO. 59)
+G*+T*+A*+A*T*G*T*C*A*A*C*G*G*+A*+T*+C,
(A31112Hi, SEQ ID NO. 59)
+G*T*+A*A*T*G*T*C*A*A*C*G*G*+A*+T*+C,
(A31368Hi, SEQ ID NO. 59)
+G*+T*+A*A*+T*G*T*C*A*A*C*G*G*+A*+T*+C,
(A31369Hi, SEQ ID NO. 59)
+G*+T*+A*A*T*+G*T*C*A*A*C*G*G*+A*+T*+C,
(A31370Hi, SEQ ID NO. 59)
+G*+T*+A*A*T*G*+T*C*A*A*C*G*G*+A*+T*+C,
(A31371Hi, SEQ ID NO. 59)
+G*+T*+A*A*T*G*T*C*A*A*C*+G*G*+A*+T*+C,
(A31372Hi, SEQ ID NO. 59)
+G*+T*+A*A*T*G*T*C*A*A*+C*G*G*+A*+T*+C,
(A31373Hi, SEQ ID NO. 59)
+G*+T*+A*+A*T*G*T*C*A*A*C*G*+G*+A*+T*+C,
(A31149H, SEQ ID NO. 133)
+G*+A*C*T*G*T*C*A*C*G*T*C*T*C*+G*+G*+C,
(A31314Hi, SEQ ID NO. 297)
+C*+A*+T*A*G*T*T*C*T*C*T*G*C*A*A*C*+A*+G*+G,
(A31352Hi, SEQ ID NO. 335)
+T*+A*+G*T*A*T*C*A*C*T*G*T*A*T*G*T*+C*+C*+A

and a combination thereof, wherein + indicates an LNA nucleotide and * indicates a phosphorothioate (PTO) linkage between the nucleotides.

The oligonucleotide comprises for example the modification or consists of the sequence selected from the group consisting of +G*+T*+A*A*T*G*T*C*A*A*C*G*G*A*+T*+C (A31109Hi; SEQ ID NO. 59), +G*+A*C*T*G*T*C*A*C*G*T*C*T*C*+G*+G*+C (A31149H, SEQ ID NO. 133), +C*+A*+T*A*G*T*T*C*T*C*T*G*C*A*A*C*+A*+G*+G (A31314Hi, SEQ ID NO. 297), +T*+A*+G*T*A*T*C*A*C*T*G*T*A*T*G*T*+C*+C*+A (A31352Hi, SEQ ID NO. 335) and a combination thereof, wherein + indicates an LNA nucleotide and * indicates a phosphorothioate (PTO) linkage between the nucleotides.

Further, the oligonucleotide hybridizes for example with at least one exon or intron of SEQ ID NO. 1 and/or with the mRNA of SEQ ID NO. 2.

The oligonucleotide inhibits the expression of NLRP3, NLRP3 mRNA, NLRP3 pre-mRNA or a combination thereof for example at a nanomolar or micromolar concentration.

The present invention further refers to a pharmaceutical composition comprising an oligonucleotide of the present invention and a pharmaceutically acceptable carrier, excipient, dilutant or a combination thereof. The pharmaceutical composition further comprises for example another active agent selected from the group consisting of an oligonucleotide, an antibody, a small molecule, a polypeptide, a lipid, a sugar and a combination thereof. The oligonucleotide and the other active agent inhibit for example the same target or a different target. The other active agent for example modulates, e.g., inhibits or stimulates the target selected from the group consisting of NLRP3, CD39, CD73, IL-1β, IL-1 receptor, IL-1R accessory protein, IL-18, IL-18 receptor, ASC, NLRC4, AIM2, Caspase-1, RIPK3, Gasdermin D, MLKL, TLR4, Caspase-8, P2X7, NFκB, RORγt, TGF-β, IL-21, IL-17, IL-22, IL-23, IL-6, TNF-α, CCR6, CCL20, STAT3, MMP-1, MMP-8, ADAMTS-5, HMG-CoA, Myd-88, HMGB-1, ROS, TAK-1, Chop, FPR1, LIMCH1, caspase inhibitor and a combination thereof.

The oligonucleotide of the present invention or the pharmaceutical composition of the present invention is for example for use in a method of preventing and/or treating a disorder, where an NLRP3 imbalance is involved.

The disorder is for example selected from the group consisting of a hyperproliferative disorder such as cancer, an inflammatory or autoimmune disorder, neurodegenerative disease, a neurological disorder, cardiovascular, metabolic disorder, renal disorder, liver disorder, lung disorder, skin disorder, ocular disorder, disorder of the gastro-intestinal tract, joint inflammation, organ transplantation, fibrotic disorder and a combination thereof.

Further, the disorder is for example selected from the group consisting of Alzheimer's disease, multiple sclerosis, autoimmune encephalitis, stroke, traumatic brain injury, atherosclerosis, nonalcoholic fatty liver disease, nonalcoholic steatohepatitis, hypertension, myocardial infarction, acute kidney injury, ischemia reperfusion injury, chronic kidney diseases, crystal-induced nephropathies, glomerulonephritis, silicosis, asthma, allergic airway inflammation, inflammatory bowel disease, colitis ulcerosa, osteoarthritis, rheumatoid arthritis, juvenile idiopathic arthritis, transplantation of kidney, lung, liver and/or heart, fibrotic disorder of kidney, lung, liver and/or heart, hyperinflammation following influenza infection, graft-versus-host disease, interstitial cystitis, uveitis, sinusitis, periodontal disease, optic neuritis, myelodysplastic syndrome, cryopyrin-associated periodic syndromes (CAPS) including familial cold autoinflammatory syndrome (FCAS), the Muckle-Wells syndrome (MWS) and neonatal-onset multisystem inflammatory disease (NOMID), gout, obesity-induced inflammation, insulin resistance, type 1 and type 2 diabetes, contact hypersensitivity and a combination thereof, and/or a cancer selected from the group consisting of breast cancer, lung cancer, malignant melanoma, lymphoma, skin cancer, bone cancer, prostate cancer, liver cancer, brain cancer, cancer of the larynx, gall bladder, pancreas, testicular, rectum, parathyroid, thyroid, adrenal, neural tissue, head and neck, colon, stomach, bronchi, kidneys, basal cell carcinoma, squamous cell carcinoma, metastatic skin carcinoma, osteo sarcoma, Ewing's sarcoma, reticulum cell sarcoma, liposarcoma, myeloma, giant cell tumor, small-cell lung tumor, islet cell tumor, primary brain tumor, meningioma, acute and chronic lymphocytic and granulocytic tumors, acute and chronic myeloid leukemia, hairy-cell tumor, adenoma, hyperplasia, medullary carcinoma, intestinal ganglioneuromas, Wilm's tumor, seminoma, ovarian tumor, leiomyomater tumor, cervical dysplasia, retinoblastoma, soft tissue sarcoma, malignant carcinoid, topical skin lesion, rhabdomyosarcoma, Kaposi's sarcoma, osteogenic sarcoma, malignant hypercalcemia, renal cell tumor, polycythermia vera, adenocarcinoma, anaplastic astrocytoma, glioblastoma multiforma, leukemia, epidermoid carcinoma and a combination thereof.

The oligonucleotide or the pharmaceutical composition of the present invention is for example administered locally or systemically.

Further, the present invention refers to a kit comprising an oligonucleotide and/or a pharmaceutical composition of the present invention and an instruction manual. The oligonucleotide and/or the pharmaceutical composition of the kit is for example for use in preventing and/or treating a disease caused by an imbalanced NLRP3 expression.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

More specifically, all referenced documents are incorporated by reference to the same extent as if each individual document was specifically and individually indicated to be incorporated by reference.

### DESCRIPTION OF FIGURES

FIG. 1 is a schematic presentation of the signal cascade of the NLRP3 activation (see McAllister et al., Osteoarthritis and Cartilage, 2018). NLRP3 is part of the NLRP3 inflammasome comprising NLRP3, adaptor protein ASC and (pro-) caspase-1. Two signals are necessary for the activation of NLRP3: signal 1 induces transcription and translation of pro-IL-1β, pro-IL-18 and NLRP3 via NFκB. Signal 2 leads to the activation of the proteolytic activity of the inflammasome complex. Activated caspase-1 induces cleaving of pro-IL-1β and pro-IL-18 to mature IL-1β and IL-18 which are secreted into the extracellular space. NLRP3 induced Caspase-1 activity results additionally in a specific form of cell death which is pyroptosis.

FIG. 2 shows a single concentration efficacy screen in human THP-1 cells in a first screening round for human NLRP3-specific antisense oligonucleotides (ASOs) after three days treatment.

FIG. 3 depicts a single concentration efficacy screen in human U87MG cells in a first screening round for human NLRP3-specific ASOs after three days treatment.

FIG. 4 depicts a single concentration efficacy screen in human PBMC-derived macrophages of selected human NLRP3-specific ASOs after six days treatment.

FIG. 5 depicts a single concentration efficacy screen of human NLRP3-specific ASOs in human U87MG cells in a second screening round for human NLRP3-specific ASOs after three days treatment.

FIG. 6 shows a single concentration efficacy screen of human NLRP3-specific ASOs in human PBMC-derived macrophages in a second screening round for human NLRP3-specific ASOs after six days treatment.

FIG. 7 depicts concentration-dependent NLRP3 mRNA knockdown by selected human NLRP3-specific ASOs from the first and second screening round in human PBMC-derived macrophages after six days treatment.

FIG. 8 shows a single concentration efficacy screen in human THP-1-derived macrophages in a third screening round for human NLRP3-specific ASOs after three days treatment.

FIG. 9 shows a single concentration efficacy screen in human PBMC-derived macrophages in a third screening round for human NLRP3-specific ASOs after six days treatment FIG. 10 shows a single concentration efficacy screen in human THP-1-derived macrophages in a fourth screening round for human NLRP3-specific ASOs after three days treatment.

FIG. 11 shows a single concentration efficacy screen in human PBMC-derived macrophages in a fourth screening round for human NLRP3-specific ASOs after six days treatment.

FIG. 12 depicts concentration-dependent NLRP3 mRNA knockdown by selected human NLRP3-specific ASOs from the first, second, third and fourth screening round in human THP-1-derived macrophages after three days treatment.

FIG. 13 shows the efficacy of selected human NLRP3-specific ASOs in human microglial HMC3 cells after three days treatment.

FIG. 14 shows a single concentration screen in human TPH-1-derived macrophages in a fifth screening round after three days treatment with human NLRP3-specific ASOs.

FIG. 15 shows a single concentration screen in human PBMC-derived macrophages in a fifth screening round after six days treatment with human NLRP3-specific ASOs.

FIG. 16 depicts functional assays investigating selected human NLRP3-specific ASOs in differentiated human THP-1-derived macrophages after six days treatment. FIG. 16A shows residual NLRP3 mRNA expression in ASO-treated THP-1-derived macrophages compared to mock-treated cells. FIG. 16B shows the inhibitory effects of the ASOs on caspase-1 activity. FIG. 16C shows IL-1β protein expression of pro-IL-1β and mature-IL-1β in cell lysates and cell culture supernatants of ASO-treated THP-1-derived macrophages analyzed by Western Blot.

FIGS. 17A and 17B show single concentration efficacy screens in mouse 4T1 cells in a first screening round for mouse NLRP3-specific ASOs after three days treatment. FIG. 17A depicts residual NLRP3 mRNA expression in ASO-treated 4T1 cells compared to mock-treated cells after three days treatment. FIG. 17B depicts cell viability of 4T1 cells after three days ASO treatment assessed by cell titer blue assay.

FIG. 18 depicts a single concentration efficacy screen in mouse Raw246.7 cells in a first screening round for mouse NLRP3-specific ASOs after three days treatment

FIGS. 19A and 19 B show single concentration screens in mouse Raw246.7 cells in a second screening round for mouse NLRP3-specific ASOs after three days treatment.

FIG. 20 shows a single concentration screen in mouse 4T1 cells for selected mouse NLRP3-specific from the second screening round after three days treatment.

FIG. 21 depicts concentration-dependent NLRP3 mRNA knockdown by selected NLRP3-specific ASOs from the first and second screening round in mouse Raw246.7 cells after three days treatment.

FIG. 22 depicts functional assays investigating the mouse NLRP3-specific ASOs A31087Mi (SEQ ID NO. 419) in mouse bone marrow derived macrophages (BMDM) after six days treatment. FIG. 22 A depicts residual NLRP3 mRNA expression in ASO-treated BMDM compared to mock-treated cells. FIG. 22 B shows NLRP3 protein expression and IL-1β protein expression of pro-IL-1β and mature-IL-1β in cell lysates and cell culture supernatants of ASO-treated BMDM cells analyzed by Western Blot.

FIG. 23 shows the efficacy of selected mouse NLRP3-specific ASOs in mouse Raw246.7 cells after three days treatment.

FIG. 24 shows the efficacy of selected mouse NLRP3-specific antisense oligonucleotides in mouse microglial IMG cells after three days treatment.

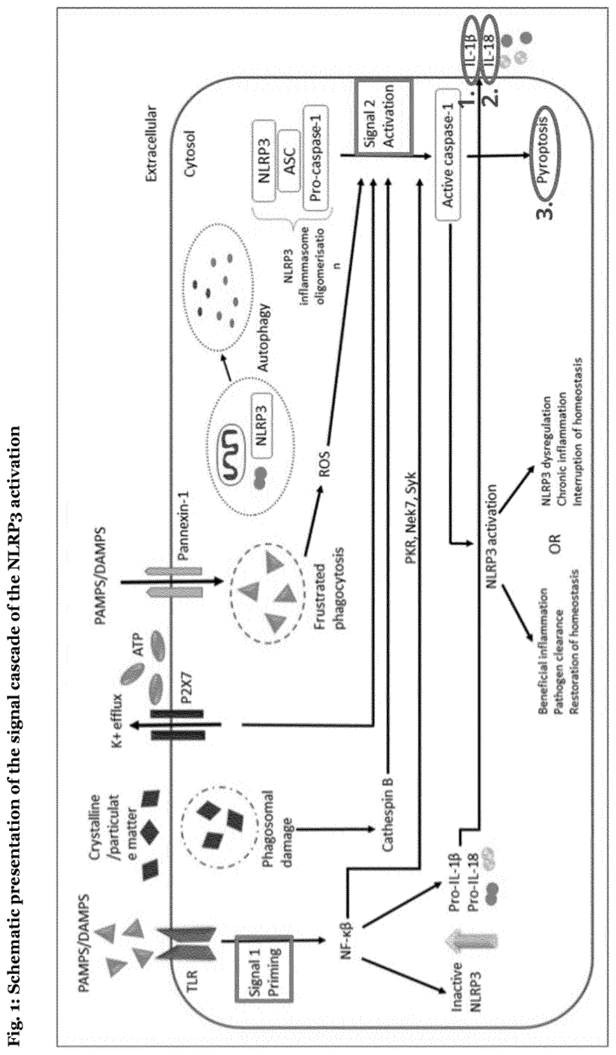

### DETAILED DESCRIPTION

The present invention provides for the first time human and murine oligonucleotides which hybridize with mRNA sequences of NLR family pyrin domain containing 3 (NLRP3) and inhibit the expression and activity, respectively, of NLRP3 of human SEQ ID NO. 1 (GRCh38_Chr1: 247412861-247452403, in particular GRCh38.p12_Chr1: 247412861-247452403, comprising 39543 nucleotides) and/or human SEQ ID NO. 2 (RefSeq ID NM_004895.4), murine SEQ ID NO. 3 (GRmCh38_Chr11: 59539030-59569495, in particular GRmCh38.p4_Chr11: 59539030-59569495, comprising 30466 nucleotides), and/or SEQ ID NO. 4 (RefSeq ID NM_145827). Thus, the oligonucleotides of the present invention represent an interesting and highly efficient tool for use in a method of preventing and/or treating a disorder, where the NLRP3 expression and activity, respectively, is imbalanced, e.g., increased or decreased for example in an inflammatory disease.

In the following, the features of the present invention will be described in more detail. These features are listed with specific embodiments, however, it should be understood that they may be combined in any manner and in any number to create additional embodiments. The variously described examples and embodiments should not be construed to limit the present invention to only the explicitly described embodiments. This description should be understood to support and encompass embodiments which combine the explicitly described embodiments with any number of the disclosed elements. Furthermore, any permutations and combinations of all described features in this application should be considered disclosed by the description of the present application unless the context indicates otherwise.

Throughout this specification and the claims, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by the context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”), provided herein is intended merely to better illustrate the invention and does not pose a limitation on the scope of the invention otherwise claimed. No language in the specification should be construed as indicating any non-claimed element essential to the practice of the invention.

Oligonucleotides of the present invention are for example antisense oligonucleotides (ASO) consisting of or comprising 10 to 25 nucleotides, 12 to 20 nucleotides, 11 to 15 nucleotides, 13 to 18 nucleotides, or 14 to 17 nucleotides. The oligonucleotides for example consist of or comprise 10, 11, 12, 13, 14, 15, 16, 17, 18, 19, 20 or 25 nucleotides. The oligonucleotides of the present invention comprise at least one nucleotide which is modified. The modified nucleotide is for example a bridged nucleotide such as a locked nucleic acid (LNA, e.g., 2′,4′-LNA), cET, ENA, a 2′Fluoro modified nucleotide, a 2′O-Methyl modified nucleotide, a 2 O-Methoxy modified nucleotide, a FANA or a combination thereof. The oligonucleotide of the present invention comprises nucleotides having for example one or more, two or more, three or more, four or more, five or more or six or more, for example six of the same or different modifications. Further, the oligonucleotide of the present invention comprises a phosphate backbone, optionally a modified phosphate backbone, wherein the phosphate is for example a phosphorothioate or methylphosphonate or a combination thereof, or where sulfur, amines or hydrocarbons are substituted for the bridging of non-bridging atoms in the phosphodiester bond. Modified nucleotide backbones include for example further chiral phosphorothioates, phosphorodithioates, phosphotriesters, aminoalkylphosphotriester, methyl and other alkyl phosphonates including 3′-alkylene phosphonate and/or chiral phosphonate, phosphinate, phosphoramidate including 3′-amino phosphoramidate and/or aminoalkylphosphoramidate, thionoalkylphosphonate, thionoalkylphosphotriester, and borano phosphate having 3′-5′-linkage and/or 2′-5′-linkages.

The oligonucleotide of the present invention comprises one or more modified nucleotide at the 3′- and/or 5′-end of the oligonucleotide and/or at any position within the oligonucleotide, wherein modified nucleotides follow in a row of 1, 2, 3, 4, 5, or 6 modified nucleotides, or a modified nucleotide is combined with one or more unmodified nucleotides.

The following Tables 1 and 2 present embodiments of oligonucleotides comprising modified nucleotides for example LNA which are indicated by (+) and phosphorothioate (PTO) indicated by (*). The oligonucleotides consisting of or comprising the sequences of Tables 1 and 2 (mRNA (Antisense) sequence 5′-3′), respectively, may comprise any other modified nucleotide and any other combination of modified and unmodified nucleotides. Some oligonucleotides are exon spanning, i.e., the oligonucleotide hybridizes with one or more different exons. Oligonucleotides of Table 1 hybridize with human NLRP3 mRNA:

TABLE 1
List of human NLRP3-specific ASOs hybridizing with human NLRP3 for
example of SEQ ID NO .1; Neg1, R01002 and R01011 are antisense oligonucleotides
representing a negative control which is not hybridizing with NLRP3 of SEQ ID NO.1 or
SEQ ID NO. 2. An “H” after the ASO ID indicates a human NLRP3-specific sequence that
binds to an exonic region of the pre-mRNA and a “Hi” after the ASO ID indicates a human
NLRP3-specific sequence that binds to an intronic region of the pre-mRNA. ** = exon
spanning oligo, position depicted in Table 1 indicates positions on mRNA of SEQ ID NO.
2 (RefSeq ID NM_004895.4) for exon spanning oligonucleotides.

| | | | | Position on |
|---|---|---|---|---|
| | | | | pre-mRNA |
| | | | | (GR Ch38, |
| | | | | Chr1: |
| | | Name of | | 247412861- |
| SEQ | | the LNA- | | 247452403; |
| ID | Antisense Sequence | modified | Antisense Sequence | SEQ ID |
| NO. | 5′-3′ | ASO | 5′-3′ with PTO (*) and LNA (+) | NO. 1) |
| 5 | ACGATGCCATCTTGACC | A31001H | +A*+C*+G*A*T*G*C*C*A*T*C*T*T*G*A*+C*+C | 5280 |
| 6 | ACGGTGAACAACCACTT | A31002H | +A*+C*+G*G*T*G*A*A*C*A*A*C*C*A*+C*+T*+T | 5298 |
| 7 | TACGGTGAACAACCAC | A31003H | +T*+A*+C*G*G*T*G*A*A*C*A*A*C*+C*+A*+C | 5300 |
| 8 | TCGATCCAGGAGTGTGT | A31004H | +T*+C*+G*A*T*C*C*A*G*G*A*G*T*G*+T*+G*+T | 5394 |
| 9 | ACACGGCACACGGATGA | A31005H | +A*+C*+A*C*G*G*C*A*C*A*C*G*G*A*+T*+G*+A | 5861 |
| 10 | TCGGTCCACACTAAGAT | A31006H | +T*+C*+G*G*T*C*C*A*C*A*C*T*A*A*+G*+A*+T | 5890 |
| 11 | CTTAGGCTTCGGTCCAC | A31007H | +C*+T*+T*A*G*G*C*T*T*C*G*G*T*C*+C*+A*+C | 5898 |
| 12 | ATCATTAGCGTGGCTAG | A31008H | +A*+T*+C*A*T*T*A*G*C*G*T*G*G*C*+T*+A*+G | 6094 |
| 13 | GCAGCGAAGATCCACAC | A31009H | +G*+C*+A*G*C*G*A*A*G*A*T*C*C*A*+C*+A*+C | 6148 |
| 14 | TTGATCGCAGCGAAGA | A31010H | +T*+T*+G*A*T*C*G*C*A*G*C*G*A*+A*+G*+A | 6155 |
| 15 | TGTTGATCGCAGCGAAG | A31011H | +T*+G*+T*T*G*A*T*C*G*C*A*G*C*G*+A*+A*+G | 6156 |
| 16 | ACGTGCATTATCTGAAC | A31012H | +A*+C*+G*T*G*C*A*T*T*A*T*C*T*G*+A*+A*+C | 10370 |
| 17 | TTCGAAACACGTGCAT | A31013H | +T*+T*+C*G*A*A*A*C*A*C*G*T*G*+C*+A*+T | 10379 |
| 18 | ATTCTCGAAAGGTACTC | A31014H | +A*+T*+T*C*T*C*G*A*A*A*G*G*T*A*+C*+T*+C | 10450 |
| 19 | CGTGTGTAGCGTTTGTT | A31015H | +C*+G*+T*G*T*G*T*A*G*C*G*T*T*T*+G*+T*+T | 11076 |
| 20 | CGTGTGTAGCGTTTGT | A31016H | +C*+G*+T*G*T*G*T*A*G*C*G*T*T*+T*+G*+T | 11077 |
| 21 | GCAGTCGTGTGTAGCGT | A31017H | +G*+C*+A*G*T*C*G*T*G*T*G*T*A*G*+C*+G*+T | 11081 |
| 22 | CTCTCACACGTCTTGGT | A31018H | +C*+T*+C*T*C*A*C*A*C*G*T*C*T*T*G*+G*+T | 11160 |
| 23 | AGAGCGGTCCTATGTGC | A31019H | +A*+G*+A*G*C*G*G*T*C*C*T*A*T*G*+T*+G*+C | 11522 |
| 24 | AGAGCGGTCCTATGTG | A31020H | +A*+G*+A*G*C*G*G*T*C*C*T*A*T*+G*+T*+G | 11523 |
| 25 | AGAAGACGTACACCGCG | A31021H | +A*+G*+A*A*G*A*C*G*T*A*C*A*C*C*+G*C*+G | 11903 |
| 26 | AACGTTCGTCCTTCCTT | A31022H | +A*+A*+C*G*T*T*C*G*T*C*C*T*T*C*+C*+T*+T | 12205 |
| 27 | GGAAGCTTCAAACGACT | A31023H | +G*+G*A*A*G*C*T*T*C*A*A*A*C*G*+A*+C*+T | 12228 |
| 28 | GGACTGTCACGTCTCG | A31024H | +G*+G*+A*C*T*G*T*C*A*C*G*T*C*+T*+C*+G | 12249 |
| 29 | TCGAATTTGCCATAGTT | A31025H | +T*+C*+G*A*A*T*T*T*G*C*C*A*T*A*+G*+T*+T | 12273 |
| 30 | TCGAATTTGCCATAGT | A31026H | +T*+C*+G*A*A*T*T*T*G*C*C*A*T*+A*+G*+T | 12274 |
| 31 | CTCGTACAAACAGTAGA | A31027H | +C*+T*+C*G*T*A*C*A*A*A*C*A*G*T*+A*+G*+A | 12475 |
| 32 | AGGTCCAATTCAGTTAG | A31028H | +A*+G*+G*+T*C*C*A*A*T*T*C*A*G*T*+T*+A*+G | 16795 |
| 33 | AGCGTTTCACACAACAC | A31029H | +A*+G*+C*G*T*T*T*C*A*C*A*C*A*A*+C*+A*+C | 16846 |
| 34 | ATCTCCGAATGTTACAG | A31030H | +A*+T*+C*T*C*C*G*A*A*T*G*T*T*A*+C*+A*+G | 16875 |
| 35 | GTCGAAGCAGCACTCAT | A31031H | +G*+T*C*G*A*A*G*C*A*G*C*A*C*T*+C*+A*+T | 21266 |
| 36 | GGACCAAGGAGATGTCG | A31032H | +G*+G*+A*C*C*A*A*G*G*A*G*A*T*G*+T*+C*+G | 21279 |
| 37 | ATTCCGAAGTCACCGAG | A31033H | +A*+T*+T*C*C*G*A*A*G*T*C*A*C*C*+G*+A*+G | 21343 |
| 38 | TCACATAAAATTGCGAC | A31034H | +T*+C*+A*C*A*T*A*A*A*A*T*T*G*C*+G*+A*+C | 23225 |
| 39 | AGACTGACGTAAGGCCA | A31035H | +A*+G*+A*C*T*G*A*C*G*T*A*A*G*G*C*+C*+A | 31124 |
| 40 | GTAAAGGTGCGTGAGAT | A31036H | +G*+T*+A*A*A*G*G*T*G*C*G*T*G*A*+G*+A*+T | 31180 |
| 41 | GTGTGACGTGAGGTTG | A31037H | +G*+T*+G*T*G*A*C*G*T*G*A*G*G*+T*+T*+G | 31803 |
| 42 | CAGTGTGACGTGAGGTT | A31038H | +C*+A*+G*T*G*T*G*A*C*G*T*G*A*G*+G*+T*+T | 31804 |
| 43 | CTAACGCACTTTTTGTC | A31039H | +C*+T*+A*A*C*G*C*A*C*T*T*T*T*T*+G*+T*+C | 35572 |
| 44 | CTCAAAGACGACGGTCA | A31040H | +C*+T*+C*A*A*A*G*A*C*G*A*C*G*G*+T*+C*+A | 35616 |
| 45 | CGGAGAACACTGGCGTC | A31041H | +C*+G*+G*A*G*A*A*C*A*C*T*G*G*C*G*+T*+C | 35665 |
| 46 | ATGGATCGCAGCTCTCT | A31042H | +A*+T*+G*G*A*T*C*G*C*A*G*C*T*C*T*+C*+T | 35710 |
| 47 | CGACACTCCACCGGAAG | A31043H | +C*+G*+A*C*A*C*T*C*C*A*C*C*G*G*+A*+A*+G | 35754 |
| 48 | TCTCCGACACTCCACCG | A31044H | +T*+C*+T*C*C*G*A*C*A*C*T*C*C*A*+C*+C*+G | 35758 |
| 49 | CGTCGGCAAGCTCTCTT | A31045H | +C*+G*+T*C*G*G*C*A*A*G*C*T*C*T*+C*+T*+T | 35774 |
| 50 | CGTCGGCAAGCTCTCT | A31046H | +C*+G*+T*C*G*G*C*A*A*G*C*T*C*+T*+C*+T | 35775 |
| 51 | AAGGCATCGTCGGCAAG | A31047H | +A*+A*+G*G*C*A*T*C*G*T*C*G*G*C*+A*+A*+G | 35781 |
| 52 | CTAACTGAGGCGCTGTG | A31048H | +C*+T*+A*A*C*T*G*A*G*G*C*G*C*T*+G*+T*+G | 35879 |
| 53 | TAGCACTACCGTGAGAG | A31049Hi | +T*+A*+G*C*A*C*T*A*C*C*G*T*G*A*+G*+A*+G | 7906 |
| 54 | CGTGCCTAATCTAGTG | A31050Hi | +C*+G*+T*G*C*C*T*A*A*T*C*T*+A*+G*+T*+G | 9849 |
| 55 | ACGACCAGTCTATTTCG | A31051Hi | +A*+C*G*A*C*C*A*G*T*C*T*A*T*T*+T*+C*+G | 9863 |
| 56 | GCTTAATCACCGCCGCT | A31052Hi | +G*+C*+T*T*A*A*T*C*A*C*C*G*C*C*+G*+C*+T | 10682 |
| 57 | TCGGAAGCGAGTGGTAA | A31053Hi | +T*+C*+G*G*A*A*G*C*G*A*G*T*G*G*+T*+A*+A | 10894 |
| 58 | CATCGGAAGCGAGTGGT | A31054Hi | +C*+A*+T*C*G*G*A*A*G*C*G*A*G*T*+G*+G*+T | 10896 |
| 59 | GTAATGTCAACGGATC | A31055Hi | +G*+T*+A*A*T*G*T*C*A*A*C*G*G*+A*+T*+C | 13706 |
| 60 | CCGTAGAAACCGTACC | A31056Hi | +C*+C*+G*T*A*G*A*A*A*C*C*G*T*A*+C*+C | 14070 |
| 61 | CCGTAAGGTGAACCTTC | A31057Hi | +C*+C*+G*T*A*A*G*G*T*G*A*A*C*C*T*+T*+C | 14184 |
| 62 | GACGTATCAGGCGATGG | A31058Hi | +G*+A*+C*G*T*A*T*C*A*G*G*C*G*A*+T*+G*+G | 14401 |
| 63 | TGACGTATCAGGCGAT | A31059Hi | +T*+G*+A*C*G*T*A*T*C*A*G*G*C*+G*+A*+T | 14403 |
| 64 | TCGGACGGAGAGCATG | A31060Hi | +T*+C*+G*G*A*C*G*G*A*G*A*G*C*+A*+T*+G | 16512 |
| 65 | CTAGAGTGGCGGACTTT | A31061Hi | +C*+T*+A*G*A*G*T*G*G*C*G*G*A*C*+T*+T*+T | 18501 |
| 66 | GAGTTACACTAAGGCCG | A31062Hi | +G*+A*+G*T*T*A*C*A*C*T*A*A*G*G*+C*+C*+G | 19318 |
| 67 | CCGGCCGTAAACAGTG | A31063Hi | +C*+C*G*G*C*C*G*T*A*A*A*C*A*+G*+T*+G | 20629 |
| 68 | TCCGTTGAAGGACGT | A31064Hi | +T*+C*C*G*T*T*G*A*A*G*G*A*+C*+G*+T | 22478 |
| 69 | TCGAGTCTAGCTTATC | A31065Hi | +T*+C*+G*A*G*T*C*T*A*G*C*T*T*+A*+T*+C | 22594 |
| 70 | TCGCCGTCACTCCAGTT | A31066Hi | +T*+C*+G*C*C*G*T*C*A*C*T*C*C*A*+G*T*+T | 24910 |
| 71 | GAGGCACTCGCCAAACG | A31067Hi | +G*+A*+G*G*C*A*C*T*C*G*C*C*A*A*A*+C*+G | 24945 |
| 72 | GGACCTGTACGACCTAT | A31068Hi | +G*+G*+A*C*C*T*G*T*A*C*G*A*C*C*+T*+A*+T | 25310 |
| 73 | TAGCGCAGGTGATTGC | A31069Hi | +T*+A*+G*C*G*C*A*G*G*T*G*A*T*T*+G*+C | 29495 |
| 74 | GGTGGCGTAATTCCTAG | A31070Hi | +G*+G*+T*G*G*C*G*T*A*A*T*T*C*C*+T*+A*+G | 29545 |
| 75 | TCTACGAAGTTCGGTT | A31071Hi | +T*+C*+T*A*C*G*A*A*G*T*T*C*G*+G*+T*+T | 32254 |
| 76 | TAGACGAGTTCTACGAA | A31072Hi | +T*+A*+G*A*C*G*A*G*T*T*C*T*A*C*+G*+A*+A | 32262 |
| 77 | ATGATGCGAGTTACGTT | A31073Hi | +A*+T*+G*A*T*G*C*G*A*G*T*T*A*C*+G*+T*+T | 33476 |
| 78 | TGATGCGAGTTACGT | A31074Hi | +T*+G*+A*T*G*C*G*A*G*T*T*A*+C*+G*+T | 33477 |
| 79 | CCTAATGTACGGTACTT | A31075Hi | +C*+C*+T*A*A*T*G*T*A*C*G*G*T*A*+C*+T*+T | 34555 |
| 80 | GCTCGTCGTGTCCTTCG | A31076Hi | +G*+C*+T*C*G*T*C*G*T*G*T*C*C*T*+T*+C*+G | 35277 |
| 81 | GCTCGTCGTGTCCTT | A31077Hi | +G*+C*+T*C*G*T*C*G*T*G*T*C*+C*+T*+T | 35279 |
| 82 | AGCCGGTGCTCGTCGTG | A31078Hi | +A*+G*+C*C*G*G*T*G*C*T*C*G*T*C*+G*+T*+G | 35284 |
| 83 | ATGGCCGCGAGACTCCG | A31079Hi | +A*+T*+G*G*C*C*G*C*G*A*G*A*C*T*C*+C*+G | 35330 |
| 84 | CGCCGCCGAGACCAAGA | A31080Hi | +C*+G*+C*C*G*C*C*G*A*G*A*C*C*A*+A*+G*+A | 35346 |
| 29 | TCGAATTTGCCATAGTT | A31081H | +T*+C*G*A*A*T*T*T*G*C*C*A*T*A*+G*+T*+T | 12273 |
| 29 | TCGAATTTGCCATAGTT | A31082H | +T*+C*+G*A*A*T*T*T*G*C*C*A*T*A*G*+T*+T | 12273 |
| 29 | TCGAATTTGCCATAGTT | A31083H | +T*C*+G*A*A*T*T*T*G*C*C*A*T*A*+G*+T*+T | 12273 |
| 29 | TCGAATTTGCCATAGTT | A31084H | +T*+C*+G*A*A*T*T*T*G*C*C*A*T*A*+G*T*+T | 12273 |
| 30 | TCGAATTTGCCATAGT | A31085H | +T*+C*G*A*A*T*T*T*G*C*C*A*T*+A*+G*+T | 12274 |
| 30 | TCGAATTTGCCATAGT | A31086H | +T*+C*+G*A*A*T*T*T*G*C*C*A*T*A*+G*+T | 12274 |
| 30 | TCGAATTTGCCATAGT | A31087H | +T*+C*+G*A*A*T*T*T*G*C*C*A*T*+A*G*+T | 12274 |
| 30 | TCGAATTTGCCATAGT | A31088H | +T*C*+G*A*A*T*T*T*G*C*C*A*T*+A*+G*+T | 12274 |
| 85 | TTCGAATTTGCCATAGT | A31089H | +T*+T*+C*G*A*A*T*T*T*G*C*C*A*T*+A*+G*+T | 12274 |
| 85 | TTCGAATTTGCCATAGT | A31090H | +T*+T*C*G*A*A*T*T*T*G*C*C*A*T*+A*+G*+T | 12274 |
| 85 | TTCGAATTTGCCATAGT | A31091H | +T*+T*+C*G*A*A*T*T*T*G*C*C*A*T*A*+G*+T | 12274 |
| 86 | TTTCGAATTTGCCATAG | A31092H | +T*+T*+T*C*G*A*A*T*T*T*G*C*C*A*+T*+A*+G | 12275 |
| 86 | TTTCGAATTTGCCATAG | A31093H | +T*+T*+T*+C*G*A*A*T*T*T*G*C*C*A*+T*+A*+G | 12275 |
| 86 | TTTCGAATTTGCCATAG | A31094H | +T*+T*+T*C*G*A*A*T*T*T*G*C*C*+A*+T*+A*+G | 12275 |
| 87 | TTGATCGCAGCGAAGAT | A31095H | +T*+T*+G*A*T*C*G*C*A*G*C*G*A*A*+G*+A*+T | 6154 |
| 88 | TCGAAAGGTACTCCAGT | A31096H | +T*+C*+G*A*A*A*G*G*T*A*C*T*C*C*+A*+G*+T | 10446 |
| 89 | GCTTCTCACGTACTTTC | A31097H | +G*+C*+T*T*C*T*C*A*C*G*T*A*C*T*+T*+T*+C | 11005 |
| 90 | TGTAGCGTTTGTTGAGG | A31098H | +T*+G*+T*A*G*C*G*T*T*T*G*T*T*G*+A*+G*+G | 11072 |
| 91 | CAGTCGTGTGTAGCGTT | A31099H | +C*+A*+G*T*C*G*T*G*T*G*T*A*G*C*+G*+T*+T | 11080 |
| 92 | ACGCAGTCGTGTGTAGC | A31100H | +A*+C*+G*C*A*G*T*C*G*T*G*T*G*T*+A*+G*+C | 11083 |
| 93 | TGAGACGCAGTCGTGTG | A31101H | +T*+G*+A*G*A*C*G*C*A*G*T*C*G*T*+G*+T*+G | 11087 |
| 94 | TGATGAGACGCAGTCGT | A31102H | +T*+G*+A*T*G*A*G*A*C*G*C*A*G*T*+C*+G*+T | 11090 |
| 95 | GTGCAGAGCGGTCCTAT | A31103H | +G*+T*+G*C*A*G*A*G*C*G*G*T*C*C*+T*+A*+T | 11526 |
| 96 | ATCTCGTACAAACAGTA | A31104H | +A*+T*+C*T*C*G*T*A*C*A*A*A*C*A*+G*+T*+A | 12477 |
| 97 | CTCCGAATGTTACAGCC | A31105H | +C*+T*+C*C*G*A*A*T*G*T*T*A*C*A*+G*+C*+C | 16873 |
| 98 | CATAAAATTGCGACTCC | A31106H | +C*+A*+T*A*A*A*A*T*T*G*C*G*A*C*+T*+C*+C | 23222 |
| 99 | AAGGCTCAAAGACGACG | A31107H | +A*+A*+G*G*C*T*C*A*A*A*G*A*C*G*+A*+C*+G | 35620 |
| 100 | GCATCGTCGGCAAGCTC | A31108H | +G*+C*+A*T*C*G*T*C*G*G*C*A*A*G*+C*+T*+C | 35778 |
| 59 | GTAATGTCAACGGATC | A31109Hi | +G*+T*+A*A*T*G*T*C*A*A*C*G*G*A*+T*+C | 13706 |
| 59 | GTAATGTCAACGGATC | A31110Hi | +G*+T*A*A*T*G*T*C*A*A*C*G*G*+A*+T*+C | 13706 |
| 59 | GTAATGTCAACGGATC | A31111Hi | +G*+T*+A*+A*T*G*T*C*A*A*C*G*G*+A*+T*+C | 13706 |
| 59 | GTAATGTCAACGGATC | A31112Hi | +G*T*+A*A*T*G*T*C*A*A*C*G*G*+A*+T*+C | 13706 |
| 101 | GGTAATGTCAACGGATC | A31113Hi | +G*+G*+T*A*A*T*G*T*C*A*A*C*G*G*+A*+T*+C | 13706 |
| 101 | GGTAATGTCAACGGATC | A31114Hi | +G*+G*+T*A*A*T*G*T*C*A*A*C*G*G*A*+T*+C | 13706 |
| 101 | GGTAATGTCAACGGATC | A31115Hi | +G*+G*T*A*A*T*G*T*C*A*A*C*G*G*+A*+T*+C | 13706 |
| 101 | GGTAATGTCAACGGATC | A31116Hi | +G*+G*+T*+A*A*T*G*T*C*A*A*C*G*G*+A*+T*+C | 13706 |
| 102 | GGTAATGTCAACGGAT | A31117Hi | +G*+G*+T*A*A*T*G*T*C*A*A*C*G*+G*+A*+T | 13707 |
| 102 | GGTAATGTCAACGGAT | A31118Hi | +G*+G*+T*+A*A*T*G*T*C*A*A*C*G*+G*+A*+T | 13707 |
| 103 | ACGCTTAATCACCGCCG | A31119Hi | +A*+C*+G*C*T*T*A*A*T*C*A*C*C*G*+C*+C*+G | 10684 |
| 104 | CGAGTGGTAACCGGAA | A31120Hi | +C*+G*+A*G*T*G*G*T*A*A*C*C*G*+G*+A*+A | 10888 |
| 105 | TGTCATCGGAAGCGAGT | A31121Hi | +T*+G*+T*C*A*T*C*G*G*A*A*G*C*G*+A*+G*+T | 10899 |
| 106 | ACGTATCAGGCGATGG | A31122Hi | +A*+C*+G*T*A*T*C*A*G*G*C*G*A*+T*+G*+G | 14401 |
| 107 | GACGTATCAGGCGATG | A31123Hi | +G*+A*+C*G*T*A*T*C*A*G*G*C*G*+A*+T*+G | 14402 |
| 108 | CCTAGAGTGGCGGACTT | A31124Hi | +C*+C*+T*A*G*A*G*T*G*G*C*G*G*A*+C*+T*+T | 18502 |
| 109 | AGACGAGTTCTACGA | A31125Hi | +A*+G*+A*C*G*A*G*T*T*C*T*A*+C*+G*+A | 32263 |
| 110 | GATGCGAGTTACGTTT | A31126Hi | +G*+A*+T*G*C*G*A*G*T*T*A*C*G*+T*+T*+T | 33475 |
| 111 | GTGATATGATGCGAGT | A31127Hi | +G*+T*+G*A*T*A*T*G*A*T*G*C*G*+A*+G*+T | 33482 |
| 112 | TTCTCCTGTTGGCTCGA | A31128H | +T*+T*C*T*C*C*T*G*T*T*G*G*C*T*+C*+G*+A | 5407 |
| 113 | ACCAGGCAGTGAACACG | A31129H | +A*+C*C*A*G*G*C*A*G*T*G*A*A*C*+A*+C*+G | 5873 |
| 114 | TTAGGCTTCGGTCCACA | A31130H | +T*+T*+A*G*G*C*T*T*C*G*G*T*C*C*+A*+C*+A | 5897 |
| 115 | GGTCCTTAGGCTTCGGT | A31131H | +G*+G*T*C*C*T*T*A*G*G*C*T*T*C*+G*+G*+T | 5902 |
| 116 | TCGATCATTAGCGTGGC | A31132H | +T*+C*G*A*T*C*A*T*T*A*G*C*G*T*+G*+G*+C | 6097 |
| 117 | CGAAGATCCACACGGCC | A31133H | +C*+G*+A*A*G*A*T*C*C*A*C*A*C*G*G*+C*+C | 6144 |
| 118 | TCCTTGATGAGACGCAG | A31134H | +T*+C*C*T*T*G*A*T*G*A*G*A*C*G*+C*+A*+G | 11094 |
| 119 | TTGCCGATGGCCAGAAG | A31135H | +T*+T*+G*C*C*G*A*T*G*G*C*C*A*G*+A*+A*+G | 11142 |
| 120 | TCTTGGTCTTGCCGATG | A31136H | +T*+C*+T*T*G*G*T*C*T*T*G*C*C*G*+A*+T*+G | 11150 |
| 121 | CACACGTCTTGGTCTTG | A31137H | +C*+A*+C*A*C*G*T*C*T*T*G*G*T*C*+T*T*+G | 11156 |
| 122 | GCTCTCACACGTCTTGG | A31138H | +G*C*+T*C*T*C*A*C*A*C*G*T*C*T*+T*+G*+G | 11161 |
| 123 | GCAGCTCATCGAAGCCG | A31139H | +G*C*+A*G*C*T*C*A*T*C*G*A*A*G*+C*+C*+G | 11489 |
| 124 | GCTCGTCAAAGGCACCT | A31140H | +G*+C*+T*C*G*T*C*A*A*A*G*G*C*A*+C*C*+T | 11507 |
| 125 | CAGAGCGGTCCTATGTG | A31141H | +C*A*+G*A*G*C*G*G*T*C*C*T*A*T*+G*+T*+G | 11523 |
| 126 | CGGCCTTCTGCCAGTCA | A31142H | +C*+G*+G*C*C*T*T*C*T*G*C*C*A*G*+T*+C*+A | 11543 |
| 127 | CACATGCCGAGGATGGT | A31143H | +C*+A*C*A*T*G*C*C*G*A*G*G*A*T*+G*+G*+T | 11671 |
| 128 | TTGGCCTCGGAGAAACC | A31144H | +T*+T*+G*G*C*C*T*C*G*G*A*G*A*A*+A*C*+C | 11697 |
| 129 | GTGAAGAGGACCTCGTT | A31145H | +G*+T*+G*A*A*G*A*G*G*A*C*C*T*C*+G*T*+T | 11787 |
| 130 | GAAGACGTACACCGCG | A31146H | +G*+A*+A*G*A*C*G*T*A*C*A*C*C*+G*C*+G | 11903 |
| 131 | AGGAAGAAGACGTACAC | A31147H | +A*+G*+G*A*A*G*A*A*G*A*C*G*T*A*+C*+A*+C | 11907 |
| 132 | GCTGTAGAACTTCTCGC | A31148H | +G*C*+T*G*T*A*G*A*A*C*T*T*C*T*+C*+G*+C | 12124 |
| 133 | GACTGTCACGTCTCGGC | A31149H | +G*+A*C*T*G*T*C*A*C*G*T*C*T*C*+G*+G*+C | 12247 |
| 134 | TGCACGAAGTCCTCCTC | A31150H | +T*+G*+C*A*C*G*A*A*G*T*C*C*T*C*+C*+T*+C | 12498 |
| 135 | TCTCCGAATGTTACAGC | A31151H | +T*+C*+T*C*C*G*A*A*T*G*T*T*A*C*+A*+G*+C | 16874 |
| 136 | TCATGCGAGAGGCCACA | A31152H | +T*+C*+A*T*G*C*G*A*G*A*G*G*C*C*A*+C*+A | 21253 |
| 137 | CACTCATGCGAGAGGCC | A31153H | +C*+A*+C*T*C*A*T*G*C*G*A*G*A*G*G*+C*+C | 21256 |
| 138 | AGAAGTCTGATTCCGAA | A31154H | +A*+G*+A*A*G*T*C*T*G*A*T*T*C*C*+G*+A*+A | 21352 |
| 139 | GGTGCGTGAGATTCTGA | A31155H | +G*+G*+T*G*C*G*T*G*A*G*A*T*T*C*+T*+G*+A | 31175 |
| 140 | CTCGCAGGTAAAGGTGC | A31156H | +C*+T*+C*G*C*A*G*G*T*A*A*A*G*G*+T*+G*+C | 31187 |
| 141 | CCTGAAGCTTGCAGTCG | A31157H | +C*+C*+T*G*A*A*G*C*T*T*G*C*A*G*+T*+C*+G | 31259 |
| 142 | CGTGAGGTTGCAGTTGT | A31158H | +C*+G*+T*G*A*G*G*T*T*G*C*A*G*T*+T*+G*+T | 31796 |
| 143 | GTGTGACGTGAGGTTGC | A31159H | +G*+T*G*T*G*A*C*G*T*G*A*G*G*T*+T*+G*+C | 31802 |
| 144 | CAGGCTCAGCTTTCGCA | A31160H | +C*+A*+G*G*C*T*C*A*G*C*T*T*T*C*+G*+C*+A | 31862 |
| 145 | ATCGCAGCTCTCTCCAC | A31161H | +A*+T*C*G*C*A*G*C*T*C*T*C*T*C*+C*+A*+C | 35706 |
| 146 | TGGCCTGGATGGATCGC | A31162H | +T*+G*+G*C*C*T*G*G*A*T*G*G*A*T*C*+G*+C | 35718 |
| 147 | GCAAGCTCTCTTCTCCG | A31163H | +G*+C*+A*A*G*C*T*C*T*C*T*T*C*T*+C*+C*+G | 35769 |
| 148 | GAAGGCATCGTCGGCAA | A31164H | +G*+A*+A*G*G*C*A*T*C*G*T*C*G*G*+C*+A*+A | 35782 |
| 149 | AGGAAGGCATCGTCGGC | A31165H | +A*+G*+G*A*A*G*G*C*A*T*C*G*T*C*+G*+G*+C | 35784 |
| 150 | TGAGGCGCTGTGATGAC | A31166H | +T*+G*A*G*G*C*G*C*T*G*T*G*A*T*+G*+A*+C | 35874 |
| 151 | ACGTGTTCTTAGGATA | A31167Hi | +A*+C*+G*T*G*T*T*C*T*T*A*G*G*+A*+T*+A | 7195 |
| 152 | GCACTACCGTGAGAGG | A31168Hi | +G*C*+A*C*T*A*C*C*G*T*G*A*G*+A*+G*+G | 7905 |
| 153 | CACGGTTCTTCAAGGAG | A31169Hi | +C*+A*+C*G*G*T*T*C*T*T*C*A*A*G*+G*+A*+G | 8158 |
| 154 | AGACGGTGTAGTGGTT | A31170Hi | +A*G*+A*C*G*G*T*G*T*A*G*T*G*+G*+T*+T | 8871 |
| 155 | GTCACTAGATAGCATA | A31171Hi | +G*+T*+C*A*C*T*A*G*A*T*A*G*C*+A*+T*+A | 9654 |
| 156 | TATTTCGTGCCTAATC | A31172Hi | +T*+A*+T*T*T*C*G*T*G*C*C*T*A*+A*+T*+C | 9854 |
| 157 | AGAACGACCAGTCTAT | A31173Hi | +A*+G*+A*A*C*G*A*C*C*A*G*T*C*+T*+A*+T | 9867 |
| 158 | GAACCTATAGTATGGC | A31174Hi | +G*+A*+A*C*C*T*A*T*A*G*T*A*T*+G*G*+C | 10573 |
| 159 | TAATCACCGCCGCTGG | A31175Hi | +T*+A*+A*T*C*A*C*C*G*C*C*G*C*+T*G*+G | 10680 |
| 160 | GAAACGCTTAATCACC | A31176Hi | +G*+A*+A*A*C*G*C*T*T*A*A*T*C*+A*+C*+C | 10688 |
| 161 | TGTGTAGCTACCAGCC | A31177Hi | +T*+G*+T*G*T*A*G*C*T*A*C*C*A*+G*+C*+C | 10767 |
| 162 | CCGAGAATCTCTGGTG | A31178Hi | +C*+C*G*A*G*A*A*T*C*T*C*T*G*G*+T*+G | 10826 |
| 163 | CGAGTGGTAACCGGAAT | A31179Hi | +C*+G*+A*G*T*G*G*T*A*A*C*C*G*G*+A*+A*+T | 10887 |
| 164 | TGTCATCGGAAGCGAG | A31180Hi | +T*+G*+T*C*A*T*C*G*G*A*A*G*C*+G*+A*+G | 10900 |
| 165 | CCACCTGAGATGCGCTC | A31181Hi | +C*+C*+A*C*C*T*G*A*G*A*T*G*C*G*+C*T*+C | 10928 |
| 166 | GTCAACGGATCAAGAA | A31182Hi | +G*+T*+C*A*A*C*G*G*A*T*C*A*A*+G*+A*+A | 13701 |
| 167 | CCTTCATGGCCAAACCA | A31183Hi | +C*+C*+T*T*C*A*T*G*G*C*C*A*A*A*+C*+C*+A | 13748 |
| 168 | GCACTTGTTTCCGGAC | A31184Hi | +G*+C*+A*C*T*T*G*T*T*T*C*C*G*+G*+A*+C | 13843 |
| 169 | CGCTGGTGAACAGGTTC | A31185Hi | +C*+G*C*T*G*G*T*G*A*A*C*A*G*G*T*+T*+C | 13926 |
| 170 | GCCGTAGAAACCGTAC | A31186Hi | +G*+C*+C*G*T*A*G*A*A*A*C*C*G*+T*+A*+C | 14071 |
| 171 | ACTGTTTGCTGCCGTA | A31187Hi | +A*+C*+T*G*T*T*T*G*C*T*G*C*C*G*+T*+A | 14081 |
| 172 | AGGCACCATCGACCAGG | A31188Hi | +A*+G*+G*C*A*C*C*A*T*C*G*A*C*C*+A*+G*+G | 14266 |
| 173 | TATGAACTTGCACTCAC | A31189Hi | +T*+A*+T*G*A*A*C*T*T*G*C*A*C*T*+C*+A*+C | 16921 |
| 174 | CTAGAGTGGCGGACTT | A31190Hi | +C*+T*+A*G*A*G*T*G*G*C*G*G*A*+C*+T*+T | 18502 |
| 175 | ACTGCGTGTCCAGGCAA | A31191Hi | +A*+C*T*G*C*G*T*G*T*C*C*A*G*G*C*+A*+A | 18654 |
| 176 | CATTCAGTGCGTGCTTT | A31192Hi | +C*+A*+T*T*C*A*G*T*G*C*G*T*G*C*+T*+T*+T | 18775 |
| 177 | TAAGCCACCGAACAGC | A31193Hi | +T*+A*+A*G*C*C*A*C*C*G*A*A*C*+A*+G*+C | 18893 |
| 178 | TGTTTGACGATTTCATC | A31194Hi | +T*+G*+T*T*T*G*A*C*G*A*T*T*T*C*+A*+T*+C | 20418 |
| 179 | AGAGAGCTCCGGAATA | A31195Hi | +A*+G*+A*G*A*G*C*T*C*C*G*G*A*+A*+T*+A | 20719 |
| | | | | 20845 |
| | | | | 20887 |
| | | | | 21055 |
| | | | | 21097 |
| 180 | GGACACACCTGACAAGA | A31196Hi | +G*+G*+A*C*A*C*A*C*C*T*G*A*C*A*+A*+G*+A | 20984 |
| 181 | CGCCTCGCTGGCAGAA | A31197Hi | +C*+G*+C*C*T*C*G*C*T*G*G*C*A*+G*+A*+A | 21442 |
| 182 | CCGTTGAAGGACGTTT | A31198Hi | +C*+C*+G*T*T*G*A*A*G*G*A*C*G*+T*+T*+T | 22476 |
| 183 | ATCCGTTGAAGGACGT | A31199Hi | +A*+T*+C*C*G*T*T*G*A*A*G*G*A*+C*+G*+T | 22478 |
| 184 | TTGTAGTGTGTACTAG | A31200Hi | +T*+T*+G*T*A*G*T*G*T*G*T*A*C*+T*+A*+G | 22544 |
| 185 | TGTGAGTGTCTTGGAAG | A31201Hi | +T*+G*+T*G*A*G*T*G*T*C*T*T*G*G*+A*+A*+G | 23557 |
| 186 | AATATTCTAAGGCGAT | A31202Hi | +A*+A*+T*A*T*T*C*T*A*A*G*G*C*+G*+A*+T | 24672 |
| 187 | CGCCGTCACTCCAGTT | A31203Hi | +C*+G*+C*C*G*T*C*A*C*T*C*C*A*+G*+T*+T | 24910 |
| 188 | CCTGTACGACCTATAA | A31204Hi | +C*+C*+T*G*T*A*C*G*A*C*C*T*A*+T*+A*+A | 25308 |
| 189 | ACATGGATGACGACTT | A31205Hi | +A*+C*+A*T*G*G*A*T*G*A*C*G*A*+C*+T*+T | 28147 |
| 190 | ACGGATATCTTGCCAT | A31206Hi | +A*+C*+G*G*A*T*A*T*C*T*T*G*C*+C*+A*+T | 28750 |
| 191 | CAACAGACGGTGCAGT | A31207Hi | +C*+A*+A*C*A*G*A*C*G*G*T*G*C*+A*+G*+T | 28958 |
| 192 | GGTGTCTACGAGGCAGG | A31208Hi | +G*+G*+T*G*T*C*T*A*C*G*A*G*G*C*+A*+G*+G | 29430 |
| 193 | ATGCATCAATCTCGAT | A31209Hi | +A*+T*+G*C*A*T*C*A*A*T*C*T*C*+G*+A*+T | 29648 |
| 194 | GAACGTCCATTGTGAC | A31210Hi | +G*+A*+A*C*G*T*C*C*A*T*T*G*T*+G*+A*+C | 30615 |
| 195 | AGCCTTGTATGCTGGTA | A31211Hi | +A*+G*+C*C*T*T*G*T*A*T*G*C*T*G*+G*+T*+A | 31637 |
| 196 | GGACTAACTAGCTTCA | A31212Hi | +G*+G*+A*C*T*A*A*C*T*A*G*C*T*+T*+C*+A | 31657 |
| 197 | CCTTGGCCATGATCGCA | A31213Hi | +C*+C*+T*T*G*G*C*C*A*T*G*A*T*C*+G*+C*+A | 32216 |
| 198 | TCGGTTCCTTGTGTAG | A31214Hi | +T*+C*+G*G*T*T*C*C*T*T*G*T*G*+T*+A*+G | 32244 |
| 199 | AGACGAGTTCTACGAA | A31215Hi | +A*+G*+A*C*G*A*G*T*T*C*T*A*C*+G*+A*+A | 32262 |
| 200 | GTGACCATCGACTGAA | A31216Hi | +G*+T*+G*A*C*C*A*T*C*G*A*C*T*+G*+A*+A | 33199 |
| 201 | AGTGATATGATGCGAG | A31217Hi | +A*+G*+T*G*A*T*A*T*G*A*T*G*C*+G*+A*+G | 33483 |
| 202 | GATAAGAAGACGATGT | A31218Hi | +G*+A*+T*A*A*G*A*A*G*A*C*G*A*+T*+G*+T | 33506 |
| 203 | AGCGGAATCTTCGGAA | A31219Hi | +A*+G*+C*G*G*A*A*T*C*T*T*C*G*+G*+A*+A | 33588 |
| 204 | TGCCTCTCACATCTTCG | A31220Hi | +T*+G*+C*C*T*C*T*C*A*C*A*T*C*T*+T*+C*+G | 34727 |
| 205 | GCTCGTCGTGTCCTTC | A31221Hi | +G*+C*+T*C*G*T*C*G*T*G*T*C*C*+T*+T*+C | 35278 |
| 206 | TGCTCGTCGTGTCCTT | A31222Hi | +T*+G*+C*T*C*G*T*C*G*T*G*T*C*+C*+T*+T | 35279 |
| 207 | CGCCGCCGAGACCAAG | A31223Hi | +C*+G*+C*C*G*C*C*G*A*G*A*C*C*+A*+A*+G | 35347 |
| 208 | GCTCCGCGTCCTCGCCG | A31224Hi | +G*+C*+T*C*C*G*C*G*T*C*C*T*C*G*+C*+C*+G | 35370 |
| 209 | ACCAGGCAGTGAACACGG | A31225H | +A*+C*+C*A*G*G*C*A*G*T*G*A*A*C*A*+C*+G*+G | 5872 |
| 210 | GGTGCTTGCCATCTTCAT | A31226H | +G*+G*+T*G*C*T*T*G*C*C*A*T*C*T*T*+C*+A*+T | 5935 |
| 211 | GATAGTCCTCTAAGTGCAT | A31227H | +G*+A*+T*A*G*T*C*C*T*C*T*A*A*G*T*G*+C*+A*+T | 6013 |
| 212 | TAGTCCTCTAAGTGCATC | A31228H | +T*+A*+G*T*C*C*T*C*T*A*A*G*T*G*C*+A*+T*+C | 6012 |
| 213 | AGCGTGGCTAGATCCACAT | A31229H | +A*+G*+C*G*T*G*G*C*T*A*G*A*T*C*C*A*+C*+A*+T | 6086 |
| 214 | GCTAGATCCACATGGTCT | A31230H | +G*+C*+T*A*G*A*T*C*C*A*C*A*T*G*G*+T*+C*+T | 6081 |
| 215 | CATTAGCGTGGCTAGATC | A31231H | +C*+A*+T*T*A*G*C*G*T*G*G*C*T*A*G*+A*+T*+C | 6091 |
| 216 | ACACGTGCATTATCTGAAC | A31232H | +A*+C*+A*C*G*T*G*C*A*T*T*A*T*C*T*G*+A*+A*+C | 10370 |
| 217 | TCTCGAAAGGTACTCCAG | A31233H | +T*+C*+T*C*G*A*A*A*G*G*T*A*C*T*C*+C*+A*+G | 10447 |
| 218 | AATCTGCTTCTCACGTAC | A31234H | +A*+A*+T*C*T*G*C*T*T*C*T*C*A*C*G*+T*+A*+C | 11009 |
| 219 | GCGTTTGTTGAGGCTCACA | A31235H | +G*+C*+G*T*T*T*G*T*T*G*A*G*G*C*T*C*+A*+C*+A | 11066 |
| 220 | GTGTAGCGTTTGTTGAGGC | A31236H | +G*+T*+G*T*A*G*C*G*T*T*T*G*T*T*G*A*+G*+G*+C | 11071 |
| 221 | CGTGTGTAGCGTTTGTTGA | A31237H | +C*+G*+T*G*T*G*T*A*G*C*G*T*T*T*G*T*+T*+G*+A | 11074 |
| 222 | GCAGTCGTGTGTAGCGTTT | A31238H | +G*+C*+A*G*T*C*G*T*G*T*G*T*A*G*C*G*+T*+T*+T | 11079 |
| 223 | TGTAGCGTTTGTTGAGGC | A31239H | +T*+G*+T*A*G*C*G*T*T*T*G*T*T*G*A*+G*+G*+C | 11071 |
| 224 | AGTCGTGTGTAGCGTTTG | A31240H | +A*+G*+T*C*G*T*G*T*G*T*A*G*C*G*T*+T*+T*+G | 11078 |
| 225 | GCAGTCGTGTGTAGCGTT | A31241H | +G*+C*+A*G*T*C*G*T*G*T*G*T*A*G*C*+G*+T*+T | 11080 |
| 226 | CTCCGGTGCTCCTTGATG | A31242H | +C*+T*+C*C*G*G*T*G*C*T*C*C*T*T*G*+A*+T*+G | 11102 |
| 227 | GTCTTGGTCTTGCCGATGG | A31243H | +G*+T*+C*T*T*G*G*T*C*T*T*G*C*C*G*A*+T*+G*+G | 11149 |
| 228 | TAGTCAAACCTGTCTTGGT | A31244H | +T*+A*+G*T*C*A*A*A*C*C*T*G*T*C*T*T*+G*+G*+T | 11329 |
| 229 | TAGAACAGATAGTCAAACC | A31245H | +T*+A*+G*A*A*C*A*G*A*T*A*G*T*C*A*A*+A*+C*+C | 11338 |
| 230 | CGACAGTGGATATAGAACA | A31246H | +C*+G*+A*C*A*G*T*G*G*A*T*A*T*A*G*A*+A*+C*+A | 11350 |
| 231 | AGAACAGATAGTCAAACC | A31247H | +A*+G*+A*A*C*A*G*A*T*A*G*T*C*A*A*+A*+C*+C | 11338 |
| 232 | CGACAGTGGATATAGAAC | A31248H | +C*+G*+A*C*A*G*T*G*G*A*T*A*T*A*G*+A*+A*+C | 11351 |
| 233 | TCGTCAAAGGCACCTTGCA | A31249H | +T*+C*+G*T*C*A*A*A*G*G*C*A*C*C*T*T*+G*+C*+A | 11503 |
| 234 | CAGTGCAGAGCGGTCCTAT | A31250H | +C*+A*+G*T*G*C*A*G*A*G*C*G*G*T*C*C*+T*+A*+T | 11526 |
| 235 | TCGTCAAAGGCACCTTGC | A31251H | +T*+C*+G*T*C*A*A*A*G*G*C*A*C*C*T*+T*+G*+C | 11504 |
| 236 | CGGCCTTCTGCCAGTCAGT | A31252H | +C*+G*+G*C*C*T*T*C*T*G*C*C*A*G*T*C*+A*+G*+T | 11541 |
| 237 | AGAGCGGTCCTATGTGCT | A31253H | +A*+G*+A*G*C*G*G*T*C*C*T*A*T*G*T*+G*+C*+T | 11521 |
| 238 | GGTCTCGTGGTGATGAGCA | A31254H | +G*+G*+T*C*T*C*G*T*G*G*T*G*A*T*G*A*+G*+C*+A | 11620 |
| 239 | CGAGGATGGTCCAGCAAGT | A31255H | +C*+G*+A*G*G*A*T*G*G*T*C*C*A*G*C*A*+A*+G*+T | 11662 |
| 240 | AGGATCTCCACATGCCGA | A31256H | +A*+G*+G*A*T*C*T*C*C*A*C*A*T*G*C*+C*+G*+A | 11678 |
| 241 | CACGATCCAGCAGACCAGG | A31257H | +C*+A*+C*G*A*T*C*C*A*G*C*A*G*A*C*C*+A*+G*+G | 11819 |
| 242 | GACACATCCGCCTTCTGCA | A31258H | +G*+A*+C*A*C*A*T*C*C*G*C*C*T*T*C*T*+G*+C*+A | 12064 |
| 243 | GCAGACACATCCGCCTTC | A31259H | +G*+C*+A*G*A*C*A*C*A*T*C*C*G*C*C*+T*+T*+C | 12068 |
| 244 | TACATGGCGGCAAAGAACT | A31260H | +T*+A*+C*A*T*G*G*C*G*G*C*A*A*A*G*A*+A*+C*+T | 12163 |
| 245 | GTACATGGCGGCAAAGAA | A31261H | +G*+T*+A*C*A*T*G*G*C*G*G*C*A*A*A*+G*+A*+A | 12165 |
| 246 | GGTAGTACATGGCGGCAA | A31262H | +G*+G*+T*A*G*T*A*C*A*T*G*G*C*G*G*+C*+A*+A | 12169 |
| 247 | TCGAATTTGCCATAGTTT | A31263H | +T*+C*+G*A*A*T*T*T*G*C*C*A*T*A*G*+T*+T*+T | 12272 |
| 248 | CTCGTACAAACAGTAGAAC | A31264H | +C*+T*+C*G*T*A*C*A*A*A*C*A*G*T*A*G*+A*+A*+C | 12473 |
| 249 | TCGTACAAACAGTAGAAC | A31265H | +T*+C*+G*T*A*C*A*A*A*C*A*G*T*A*G*+A*+A*+C | 12473 |
| 250 | TTGCACGAAGTCCTCCTC | A31266H | +T*+T*+G*C*A*C*G*A*A*G*T*C*C*T*C*+C*+T*+C | 12498 |
| 251 | GCATGTTATGGAGAAACC | A31267H | +G*+C*+A*T*G*T*T*A*T*G*G*A*G*A*A*+A*+C*+C | 12628 |
| 252 | GCGTTTCACACAACACTCT | A31268H | +G*+C*+G*T*T*T*C*A*C*A*C*A*A*C*A*C*+T*+C*+T | 16843 |
| 253 | CACAATCTCCGAATGTTAC | A31269H | +C*+A*+C*A*A*T*C*T*C*C*G*A*A*T*G*T*+T*+A*+C | 16877 |
| 254 | GTCGAAGCAGCACTCATGC | A31271H | +G*+T*+C*G*A*A*G*C*A*G*C*A*C*T*C*A*+T*+G*+C | 21264 |
| 255 | GACCAAGGAGATGTCGAAG | A31272H | +G*+A*+C*C*A*A*G*G*A*G*A*T*G*T*C*G*+A*+A*+G | 21276 |
| 256 | TGTCGAAGCAGCACTCAT | A31273H | +T*+G*+T*C*G*A*A*G*C*A*G*C*A*C*T*+C*+A*+T | 21266 |
| 257 | GGACCAAGGAGATGTCGA | A31274H | +G*+G*+A*C*C*A*A*G*G*A*G*A*T*G*T*+C*+G*+A | 21278 |
| 258 | TTCTTCAGATTGCACAAC | A31275H | +T*+T*+C*T*T*C*A*G*A*T*T*G*C*A*C*+A*+A*+C | 21390 |
| 259 | GCGTGAGATTCTGATTAGT | A31276H | +G*+C*+G*T*G*A*G*A*T*T*C*T*G*A*T*T*+A*+G*+T | 31170 |
| 260 | AGGTGCGTGAGATTCTGAT | A31277H | +A*+G*+G*T*G*C*G*T*G*A*G*A*T*T*C*T*+G*+A*+T | 31174 |
| 261 | GTAAAGGTGCGTGAGATTC | A31278H | +G*+T*+A*A*A*G*G*T*G*C*G*T*G*A*G*A*+T*+T*+C | 31178 |
| 262 | TTGTCTCCGAGAGTGTTGC | A31279H | +T*+T*+G*T*C*T*C*C*G*A*G*A*G*T*G*T*+T*+G*+C | 31204 |
| 263 | CGTGAGATTCTGATTAGT | A31280H | +C*+G*+T*G*A*G*A*T*T*C*T*G*A*T*T*+A*+G*+T | 31170 |
| 264 | GTGAGGTTGCAGTTGTCTA | A31281H | +G*+T*+G*A*G*G*T*T*G*C*A*G*T*T*G*T*+C*+T*+A | 31793 |
| 265 | TGACGTGAGGTTGCAGTTG | A31282H | +T*+G*+A*C*G*T*G*A*G*G*T*T*G*C*A*G*+T*+T*+G | 31797 |
| 266 | CCTCGCAGGTAAAGGTGC | A31283H | +C*+C*+T*C*G*C*A*G*G*T*A*A*A*G*G*+T*+G*+C | 31187 |
| 267 | TTGTCTCCGAGAGTGTTG | A31284H | +T*+T*+G*T*C*T*C*C*G*A*G*A*G*T*G*+T*+T*+G | 31205 |
| 268 | TTTCGCAGGCTCTGGCTGG | A31285H | +T*+T*+T*C*G*C*A*G*G*C*T*C*T*G*G*C*+T*+G*+G | 31850 |
| 269 | ACGTGAGGTTGCAGTTGT | A31286H | +A*+C*+G*T*G*A*G*G*T*T*G*C*A*G*T*+T*+G*+T | 31796 |
| 270 | TCTTGAAGTGTTTCTAACG | A31287H | +T*+C*+T*T*G*A*A*G*T*G*T*T*T*C*T*A*+A*+C*+G | 35583 |
| 271 | TCTTGAAGTGTTTCTAAC | A31288H | +T*+C*+T*T*G*A*A*G*T*G*T*T*T*C*T*+A*+A*+C | 35584 |
| 272 | TACATGAGGTCACCAAGA | A31289H | +T*+A*+C*A*T*G*A*G*G*T*C*A*C*C*A*+A*+G*+A | 35906 |
| 273 | TGCACAATGTGAGTGTACC | A31290Hi | +T*+G*+C*A*C*A*A*T*G*T*G*A*G*T*G*T*+A*+C*+C | 6630 |
| 274 | CACCTTCTCATAGCACTAC | A31291Hi | +C*+A*+C*C*T*T*C*T*C*A*T*A*G*C*A*C*+T*+A*+C | 7914 |
| 275 | TGAGTATATAAGTACTGAC | A31292Hi | +T*+G*+A*G*T*A*T*A*T*A*A*G*T*A*C*T*+G*+A*+C | 8621 |
| 276 | AGTACTTCTACAGTTGTGA | A31293Hi | +A*+G*+T*A*C*T*T*C*T*A*C*A*G*T*T*G*+T*+G*+A | 8664 |
| 277 | AGACAGATAACTTCAGGTG | A31294Hi | +A*+G*+A*C*A*G*A*T*A*A*C*T*T*C*A*G*+G*+T*+G | 9563 |
| 278 | TATAGGAGAAATTCAGGTT | A31295Hi | +T*+A*+T*A*G*G*A*G*A*A*A*T*T*C*A*G*+G*+T*+T | 9619 |
| 279 | AAGGCATTCTATAGGAGA | A31296Hi | +A*+A*+G*G*C*A*T*T*C*T*A*T*A*G*G*+A*+G*+A | 9629 |
| 280 | AACAACTTCTGCTGTCCAC | A31297Hi | +A*+A*+C*A*A*C*T*T*C*T*G*C*T*G*T*C*+C*+A*+C | 10160 |
| 281 | ACATGGCTCTGAGTTGCAG | A31298Hi | +A*+C*+A*T*G*G*C*T*C*T*G*A*G*T*T*G*+C*+A*+G | 10236 |
| 282 | ATTGAAACCTGGCCTAATA | A31299Hi | +A*+T*+T*G*A*A*A*C*C*T*G*G*C*C*T*A*+A*+T*+A | 10297 |
| 283 | AATCTCTGGTGTCAAGTTT | A31300Hi | +A*+A*+T*C*T*C*T*G*G*T*G*T*C*A*A*G*+T*+T*+T | 10818 |
| 284 | AGAAGAAGCTGGCGAGGA | A31301Hi | +A*+G*+A*A*G*A*A*G*C*T*G*G*C*G*A*+G*+G*+A | 12776 |
| 285 | GACTGACTCTTAGGCACTA | A31302Hi | +G*+A*+C*T*G*A*C*T*C*T*T*A*G*G*C*A*+C*+T*+A | 13059 |
| 286 | CAGACTTGGCCACTAGCTT | A31303Hi | +C*+A*+G*A*C*T*T*G*G*C*C*A*C*T*A*G*+C*+T*+T | 13473 |
| 287 | TCATTGCACAGACTTGGC | A31304Hi | +T*+C*+A*T*T*G*C*A*C*A*G*A*C*T*T*+G*+G*+C | 13482 |
| 288 | GACTCAACCACTTCATTG | A31305Hi | +G*+A*+C*T*C*A*A*C*C*A*C*T*T*C*A*+T*+T*+G | 13494 |
| 289 | CAGTCCTTCATGGCCAAAC | A31306Hi | +C*+A*+G*T*C*C*T*T*C*A*T*G*G*C*C*A*+A*+A*+C | 13750 |
| 290 | GTGCTTTGGATACTCTGC | A31307Hi | +G*+T*+G*C*T*T*T*G*G*A*T*A*C*T*C*+T*+G*+C | 13900 |
| 291 | TTCAAATGCAAAGTCTAGC | A31308Hi | +T*+T*+C* A*A*A*T*G*C*A*A*A*G*T*C*T*+A*+G*+C | 15571 |
| 292 | TCACCTGTAGCTCTCATT | A31309Hi | +T*+C*+A*C*C*T*G*T*A*G*C*T*C*T*C*+A*+T*+T | 15588 |
| 293 | GGTCCTGAAGATCTTTCTC | A31310Hi | +G*+G*+T*C*C*T*G*A*A*G*A*T*C*T*T*T*+C*+T*+C | 16964 |
| 294 | ACAAAGCATTCTGACCAGG | A31311Hi | +A*+C*+A*A*A*G*C*A*T*T*C*T*G*A*C*C*+A*+G*+G | 18120 |
| 295 | TTGATAAGACTGGTGACA | A31312Hi | +T*+T*+G*A*T*A*A*G*A*C*T*G*G*T*G*+A*+C*+A | 18559 |
| 296 | GGTGACTATTCATGTGAG | A31313Hi | +G*+G*+T*G*A*C*T*A*T*T*C*A*T*G*T*+G*+A*+G | 23568 |
| 297 | CATAGTTCTCTGCAACAGG | A31314Hi | +C*+A*+T*A*G*T*T*C*T*C*T*G*C*A*A*C*+A*+G*+G | 23845 |
| 298 | AGTGCATGATTATTAGTGG | A31315Hi | +A*+G*+T*G*C*A*T*G*A*T*T*A*T*T*A*G*+T*+G*+G | 23964 |
| 299 | CTGAGTTCCACTACCTTCT | A31316Hi | +C*+T*+G*A*G*T*T*C*C*A*C*T*A*C*C*T*+T*+C*+T | 24162 |
| 300 | CGTTTGTCTACACTGAGT | A31317Hi | +C*+G*+T*T*T*G*T*C*T*A*C*A*C*T*G*+A*+G*+T | 24175 |
| 301 | GAATGCTGATCTGGCTGGT | A31318Hi | +G*+A*+A*T*G*C*T*G*A*T*C*T*G*G*C*T*+G*+G*+T | 24450 |
| 302 | TCTTCCGGCTCCTTCACAG | A31319Hi | +T*+C*+T*T*C*C*G*G*C*T*C*C*T*T*C*A*+C*+A*+G | 25010 |
| 303 | GTTCCATCTGCTTTGCCTG | A31320Hi | +G*+T*+T*C*C*A*T*C*T*G*C*T*T*T*G*C*+C*+T*+G | 25035 |
| 304 | TACACCAAGCATGTGTTAC | A31321Hi | +T*+A*+C*A*C*C*A*A*G*C*A*T*G*T*G*T*+T*+A*+C | 25951 |
| 305 | ACTATTGTAAATGGCTCTC | A31322Hi | +A*+C*+T*A*T*T*G*T*A*A*A*T*G*G*C*T*+C*+T*+C | 26028 |
| 306 | CACAAAGCTCTGATACTC | A31323Hi | +C*+A*+C*A*A*A*G*C*T*C*T*G*A*T*A*+C*+T*+C | 26214 |
| 307 | GACTTGTGTTCTTTCACAG | A31324Hi | +G*+A*+C*T*T*G*T*G*T*T*C*T*T*T*C*A*+C*+A*+G | 26674 |
| 308 | ATTAATGCCACTTATAGAT | A31325Hi | +A*+T*+T*A*A*T*G*C*C*A*C*T*T*A*T*A*+G*+A*+T | 27090 |
| 309 | ATAGGCTATACCTAACAC | A31326Hi | +A*+T*+A*G*G*C*T*A*T*A*C*C*T*A*A*+C*+A*+C | 27163 |
| | | | | 27199 |
| | | | | 27235 |
| | | | | 27253 |
| 310 | ACATAGGCTATACCTAAC | A31327Hi | +A*+C*+A*T*A*G*G*C*T*A*T*A*C*C*T*+A*+A*+C | 27237 |
| | | | | 27255 |
| 311 | GCCAAGCTAAGATGTCAC | A31328Hi | +G*+C*+C*A*A*G*C*T*A*A*G*A*T*G*T*+C*+A*+C | 27640 |
| 312 | CAGTGATGTCCACCTATA | A31329Hi | +C*+A*+G*T*G*A*T*G*T*C*C*A*C*C*T*+A*+T*+A | 27890 |
| 313 | GTAGGAGCTTCTTAGATG | A31330Hi | +G*+T*+A*G*G*A*G*C*T*T*C*T*T*A*G*+A*+T*+G | 28024 |
| 314 | GATATCTTGCCATGTACT | A31331Hi | +G*+A*+T*A*T*C*T*T*G*C*C*A*T*G*T*+A*+C*+T | 28745 |
| 315 | CAGGCTTTACCACTATTTC | A31332Hi | +C*+A*+G*G*C*T*T*T*A*C*C*A*C*T*A*T*+T*+T*+C | 29041 |
| 316 | TGAGTGGCTTCTACTAAG | A31333Hi | +T*+G*+A*G*T*G*G*C*T*T*C*T*A*C*T*+A*+A*+G | 29223 |
| 317 | TTTAAGCTCTTTGGTGTC | A31334Hi | +T*+T*+T*A*A*G*C*T*C*T*T*T*G*G*T*+G*+T*+C | 29441 |
| 318 | TTTGATGTACTATTCTTGA | A31335Hi | +T*+T*+T*G*A*T*G*T*A*C*T*A*T*T*C*T*+T*+G*+A | 29617 |
| 319 | GTTTCTCTCAAGGCCACAG | A31336Hi | +G*+T*+T*T*C*T*C*T*C*A*A*G*G*C*C*A*+C*+A*+G | 29800 |
| 320 | TGACCTTCTAAGCTGTAG | A31337Hi | +T*+G*+A*C*C*T*T*C*T*A*A*G*C*T*G*+T*+A*+G | 30601 |
| 321 | CCGTAACACTGCAGACAC | A31338Hi | +C*+C*+G*T*A*A*C*A*C*T*G*C*A*G*A*+C*+A*+C | 30732 |
| 322 | ACACCTCACTTCTAAGATA | A31339Hi | +A*+C*+A*C*C*T*C*A*C*T*T*C*T*A*A*G*+A*+T*+A | 31371 |
| 323 | GAAGTGGAATGTGTAGTTT | A31340Hi | +G*+A*+A*G*T*G*G*A*A*T*G*T*G*T*A*G*+T*+T*+T | 32126 |
| 324 | CCTTAAAGCCAAGCAGTGA | A31341Hi | +C*+C*+T*T*A*A*A*G*C*C*A*A*G*C*A*G*+T*+G*+A | 32843 |
| 325 | ACGTTTGAGAGGTTTATT | A31342Hi | +A*+C*+G*T*T*T*G*A*G*A*G*G*T*T*T*+A*+T*+T | 33123 |
| 326 | AAAGAGACAGTGACGACA | A31343Hi | +A*+A*+A*G*A*G*A*C*A*G*T*G*A*C*G*+A*+C*+A | 33252 |
| 327 | GAGTTTGTTGCTCTGATT | A31344Hi | +G*+A*+G*T*T*T*G*T*T*G*C*T*C*T*G*+A*+T*+T | 33530 |
| 328 | GGAGAGGCTGGATTATGTG | A31345Hi | +G*+G*+A*G*A*G*G*C*T*G*G*A*T*T*A*T*+G*+T*+G | 33566 |
| 329 | GATGACAGTGTTCTTGCAA | A31346Hi | +G*+A*+T*G*A*C*A*G*T*G*T*T*C*T*T*G*+C*+A*+A | 33654 |
| 330 | GATCATTAATATGGACAGA | A31347Hi | +G*+A*+T*C*A*T*T*A*A*T*A*T*G*G*A*C*+A*+G*+A | 33941 |
| 331 | GCTTGTTATCGCAGCCTGA | A31348Hi | +G*+C*+T*T*G*T*T*A*T*C*G*C*A*G*C*C*+T*+G*+A | 34241 |
| 332 | CAGCTTGTTATCGCAGCC | A31349Hi | +C*+A*+G*C*T*T*G*T*T*A*T*C*G*C*A*+G*+C*+C | 34244 |
| 333 | ACCAAATTCATCAGAGACT | A31350Hi | +A*+C*+C*A*A*A*T*T*C*A*T*C*A*G*A*G*+A*+C*+T | 34331 |
| 334 | CTCCTCTTTAATAAAGTTT | A31351Hi | +C*+T*+C*C*T*C*T*T*T*A*A*T*A*A*A*G*+T*+T*+T | 34695 |
| 335 | TAGTATCACTGTATGTCCA | A31352Hi | +T*+A*+G*T*A*T*C*A*C*T*G*T*A*T*G*T*+C*+C*+A | 34843 |
| 336 | GGTCATGTCAACTTGTTTC | A31353Hi | +G*+G*+T*C*A*T*G*T*C*A*A*C*T*T*G*T*+T*+T*+C | 35001 |
| 337 | TCAGAGTCGTTAACCTTA | A31354Hi | +T*+C*+A*G*A*G*T*C*G*T*T*A*A*C*C*+T*+T*+A | 35479 |
| 29 | TCGAATTTGCCATAGTT | A31355H | +T*+C*+G*A*+A*T*T*T*G*C*C*A*T*A*+G*+T*+T | 12273 |
| 29 | TCGAATTTGCCATAGTT | A31356H | +T*+C*+G*A*A*+T*T*T*G*C*C*A*T*A*+G*+T*+T | 12273 |
| 29 | TCGAATTTGCCATAGTT | A31357H | +T*+C*+G*A*A*T*+T*T*G*C*C*A*T*A*+G*+T*+T | 12273 |
| 29 | TCGAATTTGCCATAGTT | A31358H | +T*+C*+G*A*A*T*T*T*G*C*C*A*T*+A*+G*+T*+T | 12273 |
| 29 | TCGAATTTGCCATAGTT | A31359H | +T*+C*+G*A*A*T*T*T*G*C*C*A*+T*A*+G*+T*+T | 12273 |
| 29 | TCGAATTTGCCATAGTT | A31360H | +T*+C*+G*A*A*T*T*T*G*C*C*+A*T*A*+G*+T*+T | 12273 |
| 29 | TCGAATTTGCCATAGTT | A31361H | +T*+C*+G*A*A*T*T*T*G*+C*C*A*T*A*+G*+T*+T | 12273 |
| 30 | TCGAATTTGCCATAGT | A31362H | +T*+C*+G*A*+A*T*T*T*G*C*C*A*T*+A*+G*+T | 12274 |
| 30 | TCGAATTTGCCATAGT | A31363H | +T*+C*+G*A*A*+T*T*T*G*C*C*A*T*+A*+G*+T | 12274 |
| 30 | TCGAATTTGCCATAGT | A31364H | +T*+C*+G*A*A*T*+T*T*G*C*C*A*T*+A*+G*+T | 12274 |
| 30 | TCGAATTTGCCATAGT | A31365H | +T*+C*+G*A*A*T*T*T*G*C*C*A*+T*+A*+G*+T | 12274 |
| 30 | TCGAATTTGCCATAGT | A31366H | +T*+C*+G*A*A*T*T*T*G*C*C*+A*T*+A*+G*+T | 12274 |
| 30 | TCGAATTTGCCATAGT | A31367H | +T*+C*+G*+A*A*T*T*T*G*C*C*A*+T*+A*+G*+T | 12274 |
| 59 | GTAATGTCAACGGATC | A31368Hi | +G*+T*+A*A*+T*G*T*C*A*A*C*G*G*+A*+T*+C | 13706 |
| 59 | GTAATGTCAACGGATC | A31369Hi | +G*+T*+A*A*T*+G*T*C*A*A*C*G*G*+A*+T*+C | 13706 |
| 59 | GTAATGTCAACGGATC | A31370Hi | +G*+T*+A*A*T*G*+T*C*A*A*C*G*G*+A*+T*+C | 13706 |
| 59 | GTAATGTCAACGGATC | A31371Hi | +G*+T*+A*A*T*G*T*C*A*A*C*+G*G*+A*+T*+C | 13706 |
| 59 | GTAATGTCAACGGATC | A31372Hi | +G*+T*+A*A*T*G*T*C*A*A*+C*G*G*+A*+T*+C | 13706 |
| 59 | GTAATGTCAACGGATC | A31373Hi | +G*+T*+A*+A*T*G*T*C*A*A*C*G*+G*+A*+T*+C | 13706 |
| | | | | Position |
| | | | | on mRNA |
| | | Name of | | (NM_ |
| SEQ | | the LNA- | | 004895.4; |
| ID | Antisense Sequence | modified | Antisense Sequence | SEQ ID |
| NO. | 5′-3′ | ASO | 5′-3′ with PTO (*) and LNA (+) | NO. 2) |
| 338 | CCACAATCTCCGAATGTTA | A31270H ** | +C*+C*+A*C*A*A*T*C*T*C*C*G*A*A*T*G*+T*+T*+A | 16878 |
| 339 | CGTTTAGGCTATGTACTT | Neg1 | +C*+G*+T*T*T*A*G*G*C*T*A*T*G*T*A*+C*+T*+T | Control |
| 340 | TACGCGCGGTTGTTTA | R01002 | +T*+A*+C*G*C*G*C*G*G*T*T*G*T*+T*+T*+A | Control |
| 341 | GATCATTCGCGGACAAC | R01011 | +G*+A*+T*C*A*T*T*C*G*C*G*G*A*C*+A*+A*+C | Control |

The following Table 2 shows oligonucleotides hybridizing with murine NLRP3 mRNA:

TABLE 2
List of mouse NLRP3-specific ASOs hybridizing with mouse NLRP3 for example of SEQ ID NO. 3;
S5 is an antisense oligonucleotide representing a negative control which is not hybrid-
izing with NLRP3 of SEQ ID NO. 3 or SEQ ID NO. 4. An “M” after the ASO ID indicates a mouse
NLRP3-specific sequence that binds to an exonic region of the pre-mRNA and a “Mi” after the
ASO ID indicates a mouse NLRP3-specific sequence that binds to an intronic region of the
pre-mRNA. ** = exon spanning oligo, position depicted in Table 2 indicates positions
on mRNA of SEQ ID NO. 4 (RefSeq ID NM_145827) for exon spanning oligonucleotides. An “R”
after the ASO ID indicates a mouse NLRP3-specific with cross-reactivity to rat.

| | | | | Position on |
|---|---|---|---|---|
| | | | | pre-mRNA |
| | | | | (GRmCh38, |
| | | | | Chr11: |
| | | Name of | | 59539030- |
| SEQ | | the LNA- | | 59569495; |
| ID | Antisense Sequence | modified | Antisense Sequence | SEQ ID |
| NO. | 5′-3′ | ASO | 5′-3′ with PTO (*) and LNA (+) | NO. 3) |
| 342 | CTTGATCCAGACGTAT | A31001M | +C*+T*+T*G*A*T*C*C*A*G*A*C*G*+T*+A*+T | 3807 |
| 343 | AACGGACACTCGTCATC | A31002M | +A*+A*C*G*G*A*C*A*C*T*C*G*T*C*+A*+T*+C | 4083 |
| 344 | TTGCAACGGACACTCGT | A31003M | +T*+T*+G*C*A*A*C*G*G*A*C*A*C*T*+C*+G*+T | 4087 |
| 345 | CTCGCCTGTTGATCGCA | A31004M | +C*+T*+C*G*C*C*T*G*T*T*G*A*T*C*+G*+C*+A | 4305 |
| 346 | CTCGCCTGTTGATCGC | A31005M | +C*+T*+C*G*C*C*T*G*T*T*G*A*T*+C*+G*+C | 4306 |
| 347 | CTCGCCTGTTGATCG | A31006M | +C*+T*+C*G*C*C*T*G*T*T*G*A*+T*+C*+G | 4307 |
| 348 | TCTCGCCTGTTGATCG | A31007M | +T*+C*+T*C*G*C*C*T*G*T*T*G*A*+T*+C*+G | 4307 |
| 349 | AGACGCGCGTTCCTGT | A31008M | +A*+G*A*C*G*C*G*C*G*T*T*C*C*T*+G*+T | 9015 |
| 350 | CACCTAGACGCGCGTTC | A31009M | +C*+A*C*C*T*A*G*A*C*G*C*G*C*G*T*+T*+C | 9019 |
| 351 | CGTGTAGCGACTGTTGA | A31010M | +C*+G*+T*G*T*A*G*C*G*A*C*T*G*T*+T*+G*+A | 9048 |
| 352 | CGTGTAGCGACTGTTG | A31011M | +C*+G*+T*G*T*A*G*C*G*A*C*T*G*+T*+T*+G | 9049 |
| 353 | GCGTGTAGCGACTGTTG | A31012M | +G*+C*+G*T*G*T*A*G*C*G*A*C*T*G*+T*+T*+G | 9049 |
| 354 | GCGTGTAGCGACTGTT | A31013M | +G*+C*+G*T*G*T*A*G*C*G*A*C*T*+G*+T*+T | 9050 |
| 355 | TGCGTGTAGCGACTGTT | A31014M | +T*+G*+C*G*T*G*T*A*G*C*G*A*C*T*+G*+T*+T | 9050 |
| 356 | GGAGCTGCGTGTAGCGA | A31015M | +G*+G*A*G*C*T*G*C*G*T*G*T*A*G*C*+G*+A | 9055 |
| 357 | TAGTCCGGCCGATGGT | A31016M | +T*+A*+G*T*C*C*G*G*C*C*G*A*T*+G*+G*+T | 9122 |
| 358 | TTAGTCCGGCCGATGGT | A31017M | +T*+T*+A*G*T*C*C*G*G*C*C*G*A*T*+G*+G*+T | 9122 |
| 359 | ACCGGCCTCGTCGTTAT | A31018M | +A*+C*C*G*G*C*C*T*C*G*T*C*G*T*T*+A*+T | 9599 |
| 360 | CTACCGGCCTCGTCGTT | A31019M | +C*+T*+A*C*C*G*G*C*C*T*C*G*T*C*G*+T*+T | 9601 |
| 361 | CTACCGGCCTCGTCGT | A31020M | +C*+T*A*C*C*G*G*C*C*T*C*G*T*C*+G*+T | 9602 |
| 362 | AAGGCTACCGGCCTCGT | A31021M | +A*+A*G*G*C*T*A*C*C*G*G*C*C*T*+C*+G*+T | 9605 |
| 363 | TAGACGGCCGTAGTGGT | A31022M | +T*+A*+G*A*C*G*G*C*C*G*T*A*G*T*G*+G*+T | 9869 |
| 364 | TAGACGGCCGTAGTGG | A31023M | +T*+A*+G*A*C*G*G*C*C*G*T*A*G*+T*+G*+G | 9870 |
| 365 | GTAGACGGCCGTAGTGG | A31024M | +G*+T*+A*G*A*C*G*G*C*C*G*T*A*G*+T*+G*+G | 9870 |
| 366 | GTAGACGGCCGTAGTG | A31025M | +G*+T*+A*G*A*C*G*G*C*C*G*T*A*+G*+T*+G | 9871 |
| 367 | ACGTAGACGGCCGTAG | A31026M | +A*+C*+G*T*A*G*A*C*G*G*C*C*G*+T*+A*+G | 9873 |
| 368 | AGAAGACGTAGACGGC | A31027M | +A*+G*+A*A*G*A*C*G*T*A*G*A*C*+G*+G*+C | 9878 |
| 369 | AGCGGAGACGTCAGTCT | A31028M | +A*+G*+C*G*G*A*G*A*C*G*T*C*A*G*T*+C*+T | 10044 |
| 370 | GGACCTTCACGTCTCGG | A31029M | +G*+G*+A*C*C*T*T*C*A*C*G*T*C*T*+C*+G*+G | 10228 |
| 371 | TAGTAGGACCTTCACGT | A31030M | +T*+A*+G*T*A*G*G*A*C*C*T*T*C*A*+C*+G*+T | 10233 |
| 372 | TAGTAGGACCTTCACG | A31031M | +T*+A*+G*T*A*G*G*A*C*C*T*T*C*+A*+C*+G | 10234 |
| 373 | CTAGTAGGACCTTCACG | A31032M | +C*+T*+A*G*T*A*G*G*A*C*C*T*T*C*+A*+C*+G | 10234 |
| 374 | ACAACACGCGGATGTGA | A31033M | +A*+C*+A*A*C*A*C*G*C*G*G*A*T*G*+T*+G*+A | 19371 |
| 375 | GCACGTTTTGTTTCACG | A31034M | +G*+C*+A*C*G*T*T*T*T*G*T*T*T*C*+A*C*+G | 27157 |
| 376 | ACGTTATCGATGTGAGG | A31035M | +A*+C*+G*T*T*A*T*C*G*A*T*G*T*G*+A*+G*+G | 27405 |
| 377 | ACGTTATCGATGTGAG | A31036M | +A*+C*+G*T*T*A*T*C*G*A*T*G*T*+G*+A*+G | 27406 |
| 378 | TTCACGTTATCGATGT | A31037M | +T*+T*+C*A*C*G*T*T*A*T*C*G*A*+T*+G*+T | 27409 |
| 379 | TTTCACGTTATCGATGT | A31038M | +T*+T*+T*C*A*C*G*T*T*A*T*C*G*A*+T*+G*+T | 27409 |
| 380 | GTTTCACGTTATCGAT | A31039M | +G*+T*+T*T*C*A*C*G*T*T*A*T*C*+G*+A*+T | 27411 |
| 381 | AGTTTCACGTTATCGAT | A31040M | +A*+G*+T*T*T*C*A*C*G*T*T*A*T*C*+G*+A*+T | 27411 |
| 351 | CGTGTAGCGACTGTTGA | A31041M | +C*+G*+T*G*T*A*G*C*G*A*C*T*G*+T*+T*+G*+A | 9048 |
| 351 | CGTGTAGCGACTGTTGA | A31042M | +C*+G*+T*+G*T*A*G*C*G*A*C*T*G*T*+T*+G*+A | 9048 |
| 351 | CGTGTAGCGACTGTTGA | A31043M | +C*+G*T*G*T*A*G*C*G*A*C*T*G*T*+T*+G*+A | 9048 |
| 351 | CGTGTAGCGACTGTTGA | A31044M | +C*+G*+T*G*T*A*G*C*G*A*C*T*G*T*T*+G*+A | 9048 |
| 375 | GCACGTTTTGTTTCACG | A31045M | +G*+C*+A*+C*G*T*T*T*T*G*T*T*T*C*+A*C*+G | 27157 |
| 375 | GCACGTTTTGTTTCACG | A31046M | +G*+C*+A*C*G*T*T*T*T*G*T*T*T*+C*+A*C*+G | 27157 |
| 375 | GCACGTTTTGTTTCACG | A31047M | +G*C*+A*+C*G*T*T*T*T*G*T*T*T*C*A*+C*+G | 27157 |
| 376 | ACGTTATCGATGTGAGG | A31048M | +A*+C*+G*T*T*A*T*C*G*A*T*G*T*+G*+A*+G*+G | 27405 |
| 376 | ACGTTATCGATGTGAGG | A31049M | +A*+C*G*T*T*A*T*C*G*A*T*G*T*+G*+A*+G*+G | 27405 |
| 382 | ACGTGTACCTTACTGTA | A31050Mi | +A*+C*+G*T*G*T*A*C*C*T*T*A*C*T*+G*+T*+A | 4411 |
| 383 | CTACCTGCACGAGGAAT | A31051Mi | +C*+T*+A*C*C*T*G*C*A*C*G*A*G*G*+A*+A*+T | 4618 |
| 384 | GAACAATTACGGCTGC | A31052Mi | +G*+A*+A*+C*A*A*T*T*A*C*G*G*C*+T*+G*+C | 5367 |
| 385 | CCACATAGGTGCCGTAG | A31053Mi | +C*+C*+A*C*A*T*A*G*G*T*G*C*C*G*+T*+A*+G | 5517 |
| 386 | GCACGGATAGTAATAGG | A31054Mi | +G*+C*+A*+C*G*G*A*T*A*G*T*A*A*T*+A*+G*+G | 6279 |
| 387 | CTTACTATGACCGGCC | A31055Mi | +C*+T*+T*A*C*T*A*T*G*A*C*C*G*G*+C*+C | 6355 |
| 388 | TACTCTTACTATGACCG | A31056Mi | +T*+A*+C*T*C*T*T*A*C*T*A*T*G*A*+C*+C*+G | 6358 |
| 389 | GTGCAACCGCGAACCT | A31057Mi | +G*+T*+G*C*A*A*C*C*G*C*G*A*A*C*+C*+T | 6509 |
| 390 | CGTGCAACCGCGAACCT | A31058Mi | +C*+G*T*G*C*A*A*C*C*G*C*G*A*A*+C*+C*+T | 6509 |
| 391 | ACATTCGTGCAACCGC | A31059Mi | +A*+C*+A*T*T*C*G*T*G*C*A*A*C*+C*+G*+C | 6515 |
| 392 | AACATTCGTGCAACCGC | A31060Mi | +A*+A*+C*A*T*T*C*G*T*G*C*A*A*C*+C*+G*+C | 6515 |
| 393 | CTCAGTTGGATGTACGT | A31061Mi | +C*+T*+C*A*G*T*T*G*G*A*T*G*T*A*+C*+G*+T | 7132 |
| 394 | CTCAGTTGGATGTACG | A31062Mi | +C*+T*+C*A*G*T*T*G*G*A*T*G*T*+A*+C*+G | 7133 |
| 395 | CACTGCTCGAGTGTATC | A31063Mi | +C*+A*C*T*G*C*T*C*G*A*G*T*G*T*+A*+T*+C | 8170 |
| 396 | TGGTATGACCGGACAGA | A31064Mi | +T*+G*+G*T*A*T*G*A*C*C*G*G*A*C*+A*+G*+A | 8490 |
| 397 | ATAGAGCGAGCTGTCG | A31065Mi | +A*+T*+A*G*A*G*C*G*A*G*C*T*G*+T*+C*+G | 8756 |
| 398 | TATAGAGCGAGCTGTCG | A31066Mi | +T*+A*+T*A*G*A*G*C*G*A*G*C*T*G*+T*+C*+G | 8756 |
| 399 | AGGATTTACGAACAAC | A31067Mi | +A*+G*+G*A*T*T*T*A*C*G*A*A*C*+A*+A*+C | 8836 |
| 400 | TAAGCTGCTAGCGGACC | A31068Mi | +T*+A*+A*G*C*T*G*C*T*A*G*C*G*G*A*+C*+C | 8885 |
| 401 | GCATGGAAGCGATATTC | A31069Mi | +G*+C*+A*T*G*G*A*A*G*C*G*A*T*A*+T*+T*+C | 11021 |
| | | | | 13044 |
| 402 | TCTCAGAACGACGGAAT | A31070Mi | +T*+C*+T*C*A*G*A*A*C*G*A*C*G*G*+A*+A*+T | 11341 |
| 403 | GAGCATGTTTGCCGATA | A31071Mi | +G*+A*+G*C*A*T*G*T*T*T*G*C*C*G*+A*+T*+A | 12045 |
| 404 | CCGTTGGACTGGTATCA | A31072Mi | +C*+C*+G*T*T*G*G*A*C*T*G*G*T*A*+T*+C*+A | 13244 |
| 405 | TTCACGTATCAGCACA | A31073Mi | +T*+T*+C*A*C*G*T*A*T*C*A*G*C*+A*+C*+A | 13672 |
| 406 | GTGAACGTAATGGCAGG | A31074Mi | +G*+T*+G*A*A*C*G*T*A*A*T*G*G*C*+A*+G*+G | 13906 |
| 407 | TTAAAGACCGGTGTGCC | A31075Mi | +T*+T*+A*A*A*G*A*C*C*G*G*T*G*T*G*+C*+C | 14147 |
| 408 | ACCTCGAGTTTGGTTCA | A31076Mi | +A*+C*+C*T*C*G*A*G*T*T*T*G*G*T*T*+C*+A | 14378 |
| 409 | GACCGGTGATCTCATAA | A31077Mi | +G*+A*+C*C*G*G*T*G*A*T*C*T*C*A*+T*+A*+A | 15826 |
| 410 | GAACTGACCGGTGATC | A31078Mi | +G*+A*+A*C*T*G*A*C*C*G*G*T*G*A*+T*+C | 15832 |
| 411 | CCGTGCGTTAGGAGAAT | A31079Mi | +C*+C*+G*T*G*C*G*T*T*A*G*G*A*G*+A*+A*+T | 15893 |
| 412 | TCTTTCCGTGCGTTAG | A31080Mi | +T*+C*+T*T*T*C*C*G*T*G*C*G*T*+T*+A*+G | 15899 |
| 413 | TGAATGCACCAAGCTCG | A31081Mi | +T*+G*+A*A*T*G*C*A*C*C*A*A*G*C*+T*+C*+G | 16071 |
| 414 | CCGACCATATAATGCTA | A31082Mi | +C*+C*+G*+A*C*C*A*T*A*T*A*A*T*G*+C*T*+A | 16351 |
| 415 | AGCGTTCAGTAAGGACC | A31083Mi | +A*+G*+C*G*T*T*C*A*G*T*A*A*G*G*A*+C*+C | 17288 |
| 416 | TACGTTCCTACTAGAGC | A31084Mi | +T*+A*+C*G*T*T*C*C*T*A*C*T*A*G*+A*+G*+C | 17943 |
| 417 | ACGGTTGCATAGCTGGC | A31085Mi | +A*+C*+G*G*T*T*G*C*A*T*A*G*C*T*G*+G*+C | 18058 |
| 418 | TGTTACTGAGTACGACC | A31086Mi | +T*+G*+T*T*A*C*T*G*A*G*T*A*C*G*A*+C*+C | 20304 |
| 419 | AGCAATTTGGTAACGTC | A31087Mi | +A*+G*+C*A*A*T*T*T*G*G*T*A*A*C*+G*+T*+C | 20517 |
| 420 | CCGTGGCAGCACCTTAA | A31088Mi | +C*+C*+G*T*G*G*C*A*G*C*A*C*C*T*+T*+A*+A | 20545 |
| 421 | AACTGAGCGTAGTGAAG | A31089Mi | +A*+A*+C*T*G*A*G*C*G*T*A*G*T*G*+A*+A*+G | 22910 |
| 422 | TAACTTGCGGTATTATC | A31090Mi | +T*+A*+A*C*T*T*G*C*G*G*T*A*T*T*+A*+T*+C | 23548 |
| 423 | GTCTAACTTGCGGTAT | A31091Mi | +G*+T*C*T*A*A*C*T*T*G*C*G*G*+T*+A*+T | 23552 |
| 424 | CAATCCTCGTTGTTCC | A31092Mi | +C*+A*+A*T*C*C*T*C*G*T*T*G*T*+T*+C*+C | 23744 |
| 425 | TACGTCAGCAATCCTCG | A31093Mi | +T*+A*+C*G*T*C*A*G*C*A*A*T*C*C*+T*+C*+G | 23751 |
| 426 | AAGTTTGCTGTCCGGTA | A31094Mi | +A*+A*+G*T*T*T*G*C*T*G*T*C*C*G*+G*+T*+A | 23854 |
| 427 | CGAGAAGCGATGCTTAG | A31095Mi | +C*+G*+A*+G*A*A*G*C*G*A*T*G*C*T*+T*+A*+G | 24285 |
| 428 | GCTTGGATCCACGAGAG | A31096Mi | +G*+C*+T*T*G*G*A*T*C*C*A*C*G*+A*G*+A*+G | 25786 |
| 429 | CACTGCGAGCTGCATC | A31097Mi | +C*+A*C*T*G*C*G*A*G*C*T*G*C*+A*+T*+C | 25939 |
| 430 | CGCATATACAAAGTCAC | A31098M | +C*+G*+C*A*T*A*T*A*C*A*A*A*G*T*+C*+A*+C | 3705 |
| 431 | CTTGATCCAGACGTATG | A31099M | +C*+T*+T*G*A*T*C*C*A*G*A*C*G*T*+A*+T*+G | 3806 |
| 432 | TTGATCCAGACGTATG | A31100M | +T*+T*+G*A*T*C*C*A*G*A*C*G*T*+A*+T*+G | 3806 |
| 433 | GCTTGATCCAGACGTAT | A31101M | +G*+C*+T*T*G*A*T*C*C*A*G*A*C*G*+T*+A*+T | 3807 |
| 434 | CGGACACTCGTCATCTT | A31102M | +C*+G*+G*A*C*A*C*T*C*G*T*C*A*T*+C*+T*+T | 4081 |
| 435 | AGCTCGTTGGAGAAATA | A31103M | +A*+G*+C*T*C*G*T*T*G*G*A*G*A*A*+A*+T*+A | 9710 |
| 436 | CTGTAGAATCTCTCGCA | A31104M | +C*+T*+G*T*A*G*A*A*T*C*T*C*T*C*+G*+C*+A | 10097 |
| 437 | ATAGTACATAGCAGCGA | A31105M | +A*+T*+A*G*T*A*C*A*T*A*G*C*A*G*+C*+G*+A | 10143 |
| 438 | TAGTACATAGCAGCGA | A31106M | +T*+A*+G*T*A*C*A*T*A*G*C*A*G*+C*+G*+A | 10143 |
| 439 | CAAACTTGCCGTAATTT | A31107M | +C*+A*+A*A*C*T*T*G*C*C*G*T*A*A*+T*+T*+T | 10252 |
| 440 | TAGAACAGTTCCAGTTG | A31108M | +T*+A*+G*A*A*C*A*G*T*T*C*C*A*G*+T*+T*+G | 10442 |
| 441 | TGCTTCGTAGATAGAGG | A31109M | +T*+G*+C*T*T*C*G*T*A*G*A*T*A*G*+A*+G*+G | 26089 |
| 442 | CATTGCTTCGTAGATAG | A31110M | +C*+A*+T*T*G*C*T*T*C*G*T*A*G*A*+T*+A*+G | 26092 |
| 443 | CGCCAAGATCATTGTTG | A31111M | +C*+G*+C*C*A*A*G*A*T*C*A*T*T*G*+T*+T*+G | 26906 |
| 444 | GCACAGATCGCCAAGAT | A31112M | +G*+C*+A*C*A*G*A*T*C*G*C*C*A*A*+G*+A*+T | 26914 |
| 445 | CGTTTTGTTTCACGATT | A31113M | +C*+G*+T*T*T*T*G*T*T*T*C*A*C*G*+A*+T*+T | 27154 |
| 375 | GCACGTTTTGTTTCACG | A31114M | +G*+C*+A*C*G*T*T*T*T*G*T*T*T*C*+A*+C*+G | 27157 |
| 446 | GAAATCTCGAAGACTAT | A31115M | +G*+A*+A*A*T*C*T*C*G*A*A*G*A*C*+T*+A*+T | 27211 |
| 447 | CGTTATCGATGTGAGGA | A31116M | +C*+G*+T*T*A*T*C*G*A*T*G*T*G*A*+G*+G*+A | 27404 |
| 448 | GCAGTTTCACGTTATCG | A31117M | +G*+C*+A*G*T*T*T*C*A*C*G*T*T*A*+T*+C*+G | 27413 |
| 343 | AACGGACACTCGTCATC | A31118M | +A*+A*+C*G*G*A*C*A*C*T*C*G*T*C*+A*+T*+C | 4083 |
| 449 | GAGCCAGCTTGCAACG | A31119M | +G*+A*+G*C*C*A*G*C*T*T*G*C*A*+A*+C*+G | 4096 |
| 450 | GTCTCGCCTGTTGATCG | A31120MR | +G*+T*+C*T*C*G*C*C*T*G*T*T*G*A*+T*+C*+G | 4307 |
| 451 | GTCTCGCCTGTTGATC | A31121MR | +G*+T*+C*T*C*G*C*C*T*G*T*T*G*+A*+T*+C | 4308 |
| 452 | GTCGTCTGTACATCTTA | A31122M | +G*+T*+C*G*T*C*T*G*T*A*C*A*T*C*+T*+T*+A | 8968 |
| 453 | AGCGACTGTTGAGGTCC | A31123M | +A*+G*+C*G*A*C*T*G*T*T*G*A*G*G*+T*+C*+C | 9043 |
| 454 | AGTTGGAGCTGCGTGTA | A31124M | +A*+G*+T*T*G*G*A*G*C*T*G*C*G*T*+G*+T*+A | 9059 |
| 455 | GTCCAACATAATCTTCC | A31125MR | +G*+T*+C*C*A*A*C*A*T*A*A*T*C*T*+T*+C*+C | 9267 |
| 456 | GTCGTTATGAGCAGAGA | A31126M | +G*+T*+C*G*T*T*A*T*G*A*G*C*A*G*+A*+G*+A | 9590 |
| 359 | ACCGGCCTCGTCGTTAT | A31127M | +A*+C*+C*G*G*C*C*T*C*G*T*C*G*T*+T*+A*+T | 9599 |
| 457 | TACCGGCCTCGTCGTT | A31128M | +T*+A*+C*C*G*G*C*C*T*C*G*T*C*+G*+T*+T | 9601 |
| 458 | ACGTAGACGGCCGTAGT | A31129M | +A*+C*+G*T*A*G*A*C*G*G*C*C*G*T*+A*+G*+T | 9872 |
| 459 | CCGTGCTTCCGCAGATC | A31130M | +C*+C*+G*T*G*C*T*T*C*C*G*C*A*G*+A*+T*+C | 10019 |
| 460 | GCCGTGCTTCCGCAGAT | A31131M | +G*+C*+C*G*T*G*C*T*T*C*C*G*C*A*+G*+A*+T | 10020 |
| 369 | AGCGGAGACGTCAGTCT | A31132M | +A*+G*+C*G*G*A*G*A*C*G*T*C*A*G*+T*+C*+T | 10044 |
| 461 | CTCTCGCAGTCCACTTC | A31133M | +C*+T*+C*T*C*G*C*A*G*T*C*C*A*C*+T*+T*+C | 10088 |
| 462 | ATCTCTCGCAGTCCACT | A31134M | +A*+T*+C*T*C*T*C*G*C*A*G*T*C*C*+A*+C*+T | 10090 |
| 463 | GAACCAATGCGAGATCC | A31135MR | +G*+A*+A*C*C*A*A*T*G*C*G*A*G*A*+T*+C*+C | 19390 |
| 464 | AGTAAGGCCGGAATTC | A31137MR | +A*+G*+T*A*A*G*G*C*C*G*G*A*A*+T*+T*+C | 26017 |
| 465 | GGACTCGTGTCTGACCA | A31138M | +G*+G*+A*C*T*C*G*T*G*T*C*T*G*A*+C*+C*+A | 3686 |
| 466 | CGCGTTCCTGTCCTTGA | A31139M | +C*+G*+C*G*T*T*C*C*T*G*T*C*C*T*+T*+G*+A | 9009 |
| 467 | TAGACGCGCGTTCCTGT | A31140M | +T*+A*+G*A*C*G*C*G*C*G*T*T*C*C*+T*+G*+T | 9015 |
| 468 | TAGACGCGCGTTCCTG | A31141M | +T*+A*+G*A*C*G*C*G*C*G*T*T*C*+C*+T*+G | 9016 |
| 469 | CTCACCTAGACGCGCG | A31142M | +C*+T*+C*A*C*C*T*A*G*A*C*G*C*+G*+C*+G | 9022 |
| 470 | CTCTCACCTAGACGCG | A31143M | +C*+T*+C*T*C*A*C*C*T*A*G*A*C*+G*+C*+G | 9024 |
| 471 | GCTGCGTGTAGCGACTG | A31144M | +G*+C*+T*G*C*G*T*G*T*A*G*C*G*A*+C*+T*+G | 9052 |
| 472 | CGGCCGATGGTCAGGAG | A31145M | +C*+G*+G*C*C*G*A*T*G*G*T*C*A*G*+G*+A*+G | 9116 |
| 473 | CATTTTAGTCCGGCCGA | A31146M | +C*+A*+T*T*T*T*A*G*T*C*C*G*G*C*+C*+G*+A | 9126 |
| 474 | GCCTCGTCGTTATGAG | A31147M | +G*+C*+C*T*C*G*T*C*G*T*T*A*T*+G*+A*+G | 9596 |
| 475 | CAAGGCTACCGGCCTCG | A31148M | +C*+A*+A*G*G*C*T*A*C*C*G*G*C*C*+T*+C*+G | 9606 |
| 476 | CGGCCGTAGTGGTCTT | A31149M | +C*+G*+G*C*C*G*T*A*G*T*G*G*T*+C*+T*+T | 9866 |
| 477 | AGACGTAGACGGCCG | A31150M | +A*+G*+A*C*G*T*A*G*A*C*G*G*+C*+C*+G | 9876 |
| 478 | AGAAGACGTAGACGGCC | A31151M | +A*+G*+A*A*G*A*C*G*T*A*G*A*C*G*+G*+C*+C | 9877 |
| 479 | GCAGGCCGTGCTTCCGC | A31152M | +G*+C*+A*G*G*C*C*G*T*G*C*T*T*C*+C*+G*+C | 10024 |
| 480 | TCGGTTCAGAAGATCTG | A31153M | +T*+C*+G*G*T*T*C*A*G*A*A*G*A*T*+C*+T*+G | 10215 |
| 481 | GGTTGGTGCTTAGACTT | A31154M | +G*+G*+T*T*G*G*T*G*C*T*T*A*G*A*+C*+T*+T | 12790 |
| 482 | TGTCGAAGCAGCATTGA | A31155M | +T*+G*+T*C*G*A*A*G*C*A*G*C*A*T*+T*+G*+A | 16743 |
| 483 | CGCTTCTAAGGCACGTT | A31156M | +C*+G*+C*T*T*C*T*A*A*G*G*C*A*C*+G*+T*+T | 27167 |
| 484 | TCACGTTATCGATGTGA | A31157M | +T*+C*+A*C*G*T*T*A*T*C*G*A*T*G*+T*+G*+A | 27407 |
| | | | | Position on |
| | | Name of | | mRNA |
| SEQ | | the LNA- | | (NM_145827; |
| ID | Antisense Sequence | modified | Antisense Sequence | SEQ ID |
| NO. | 5′-3′ | ASO | 5′-3′ with PTO (*) and LNA (+) | NO. 4) |
| 485 | GCCGGAATTCACCAACC | A31136MR** | +G*+C*+C*G*G*A*A*T*T*C*A*C*C*A*+A*+C*+C | 26010 |
| 486 | TTATGTCCGGTTATTTC | S5 | +T*+T*+A*T*G*T*C*C*G*G*T*T*A*T*+T*+T*+C | Control |

The oligonucleotides of the present invention hybridize for example with mRNA and/or pre-mRNA of human NLRP3 of SEQ ID NO. 2 and SEQ ID NO 1, respectively, and/or of mouse NLRP3 of SEQ ID NO. 4 and SEQ ID NO 3, respectively. Such oligonucleotides are called NLRP3 antisense oligonucleotides. Oligonucleotides of the present invention, which are for example antisense oligonucleotides, are shown in Tables 1 and 2. The present invention further refers to oligonucleotides such as antisense oligonucleotides having 80 to 99%, 85 to 98%, 90 to 95 or 93% sequence homology to an oligonucleotide of Table 1 and/or Table 2. Such oligonucleotides still show an inhibitory activity of 70 to 100%, 80 to 90%, 70%, 75%80%, 85%, 90%, 95% or 100% compared to the oligonucleotide having the nucleic acid sequence shown in Table 1 or 2.

Each nucleotide of the sequence can be modified, wherein ASOs of the present invention preferably comprise a core of 6 to 8 unmodified nucleotides. ASOs of the present invention comprise for example one or more modified nucleotides, e.g., 1, 2, 3, 4 or 5 nucleotides at the 5′- and/or 3′-end of the oligonucleotide, i.e., on the 5′- and/or 3′-side of the core. The 5′- and 3′-end are modified identically or differently. If the 5′- and 3′-ends are modified identically the nucleotides are modified at the same positions counted from the 5′- and 3′-end (in each case starting the counting with 1 from the end), respectively, having the same modification for example LNA-modification. If the 5′- and 3′-ends are modified differently the position of the modified nucleotide and/or the type of modification at the 5′- and 3′-ends differ; the type of nucleotide modification is the same (e.g., LNA) or different. Modified nucleotides such as LNA-modified nucleotides need not to follow in a row, but may be separated by one or more unmodified nucleotides. In the following some modification patterns at the 5′- and 3′-end of the ASOs of the present invention are described, wherein an unmodified nucleotide is indicated by “_” and the figure refers to the number of modified nucleotides such as LNA-modified nucleotides in a row. The modified nucleotide(s) is/are at any position of the 5′- and/or 3′-end of the ASO as shown in the following Table 3:

TABLE 3

| LNA modification at the 5′- | LNA modification at the 3′- | |
|---|---|---|
| side of the core | side of the core | Abbreviation |
| 3 | 3 | 3 + 3 |
| 3 | 2 | 3 + 2 |
| 2 | 3 | 2 + 3 |
| 1_1 | 3 | 1_1 + 3 |
| 1_1 | 2 | 1_1 + 2 |
| 1_1 | 1_1 | 1_1 + 1_1 |
| 3 | 1_1 | 3 + 1_1 |
| 2 | 1_1 | 2 + 1_1 |
| 2 | 2 | 2 + 2 |
| 4 | 3 | 4 + 3 |
| 4 | 2 | 4 + 2 |
| 4 | 1_1 | 4 + 1_1 |
| 2_1 | 3 | 2_1 + 3 |
| 2_1 | 1_1 | 2_1 + 1_1 |
| 2_1 | 2 | 2_1 + 2 |
| 3 | 4 | 3 + 4 |
| 2 | 4 | 2 + 4 |
| 1_1 | 4 | 1_1 + 4 |
| 3 | 1_2 | 3 + 1_2 |
| 1_1 | 1_2 | 1_1 + 1_2 |
| 2 | 1_2 | 2 + 1_2 |

Typical modification patterns of each ASO of the present invention, comprising for example LNA-modified nucleotides, are shown for example in the following

TABLE 4

| Position of the modification | Position of the modification | |
|---|---|---|
| at the 5′-end (counted from | at the 3′-end (counted from | |
| the 5′-end starting with 1) | the 3′-end starting with 1) | Abbreviation |
| nucleotides 1 to 5 | nucleotides 1 to 5 | 5 + 5 |
| nucleotides 1 to 4 | nucleotides 1 to 4 | 4 + 4 |
| nucleotides 1 to 3 | nucleotides 1 to 3 | 3 + 3 |
| nucleotides 1 and 2 | nucleotides 1 and 2 | 2 + 2 |
| nucleotide 1 | nucleotide 1 | 1 + 1 |
| nucleotides 1 to 5 | nucleotides 1 to 4 | 5 + 4 |
| nucleotides 1 to 4 | nucleotides 1 to 3 | 4 + 3 |
| nucleotides 1 to 3 | nucleotides 1 and 2 | 3 + 2 |
| nucleotides 1 and 2 | nucleotide 1 | 2 + 1 |
| nucleotide 1 | nucleotides 1 to 5 | 1 + 5 |
| nucleotides 1 to 5 | nucleotides 1 to 3 | 5 + 3 |
| nucleotides 1 to 4 | nucleotides 1 and 2 | 4 + 2 |
| nucleotides 1 to 3 | nucleotide 1 | 3 + 1 |
| nucleotides 1 and 2 | nucleotides 1 to 5 | 2 + 5 |
| nucleotide 1 | nucleotides 1 to 4 | 1 + 4 |
| nucleotides 1 to 5 | nucleotides 1 and 2 | 5 + 2 |
| nucleotides 1 to 4 | nucleotide 1 | 4 + 1 |
| nucleotides 1 to 3 | nucleotides 1 to 5 | 3 + 5 |
| nucleotides 1 and 2 | nucleotides 1 to 4 | 2 + 4 |
| nucleotide 1 | nucleotides 1 to 3 | 1 + 3 |
| nucleotides 1 to 5 | nucleotide 1 | 5 + 1 |
| nucleotides 1 to 4 | nucleotides 1 to 5 | 4 + 5 |
| nucleotides 1 to 3 | nucleotides 1 to 4 | 3 + 4 |
| nucleotides 1 and 2 | nucleotides 1 to 3 | 2 + 3 |
| nucleotide 1 | nucleotides 1 and 2 | 1 + 2 |
| nucleotides 1 and 2 | nucleotides 1 and 3 | 2 + 1_1 |
| nucleotides 1 and 3 | nucleotides 1 to 3 | 1_1 + 3 |
| nucleotides 1 and 3 | nucleotides 1 and 3 | 1_1 + 1_1 |
| nucleotides 1 to 3 | nucleotides 1 and 3 | 3 + 1_1 |
| nucleotides 1 to 4 | nucleotides 1 and 3 | 4 + 1_1 |
| nucleotides 1, 2 and 4 | nucleotides 1 to 3 | 2_1 + 3 |
| nucleotides 1, 2 and 4 | nucleotides 1 and 3 | 2_1 + 1_1 |
| nucleotides 1, 2 and 4 | nucleotides 1 and 2 | 2_1 + 2 |
| nucleotides 1 and 3 | nucleotides 1 to 4 | 1_1 + 4 |
| nucleotides 1 to 3 | nucleotides 1, 2 and 4 | 3 + 1_2 |
| nucleotides 1 and 3 | nucleotides 1, 2 and 4 | 1_1 + 1_2 |
| nucleotides 1 and 2 | nucleotides 1, 2 and 4 | 2 + 1_2 |

which indicates specific positions of the LNA modifications at the 5′- and 3′-end of each ASO:

An oligonucleotide of the present invention further or alternatively hybridizes for example with the NLRP3 nucleic acid sequence of SEQ ID NO. 1 and SEQ ID NO. 2 (human) or SEQ ID NO. 3 and SEQ ID NO. 4 (murine) in a hybridizing active area. A hybridizing active area is an area on the NLRP3 pre-mRNA of SEQ ID NO. 1 (human) or SEQ ID NO. 3 (mouse), wherein binding of an oligonucleotide most likely leads to potent knockdown of the NLRP3 expression. An oligonucleotide of the present invention hybridizes for example within these positions or overlaps with a terminal position.

The human NLRP3 oligonucleotides of the present invention hybridize for example with hybridizing active areas of human NLRP3 pre-mRNA for example of SEQ ID NO. 1. Hybridizing active areas of SEQ ID NO. 1 are listed for example in the following

TABLE 5

| Region of | First position on | |
|---|---|---|
| SEQ ID NO. 1 | SEQ ID NO. 1 | SEQ ID NO. |
| 5280-5779 | | |
| A31001H | 5280 | 5 |
| A31002H | 5298 | 6 |
| A31003H | 5300 | 7 |
| A31004H | 5394 | 8 |
| A31128H | 5407 | 112 |
| 5780-6279 | | |
| A31005H | 5861 | 9 |
| A31006H | 5890 | 10 |
| A31007H | 5898 | 11 |
| A31008H | 6094 | 12 |
| A31009H | 6148 | 13 |
| A31010H | 6155 | 14 |
| A31011H | 6156 | 15 |
| A31095H | 6154 | 87 |
| A31129H | 5873 | 113 |
| A31130H | 5897 | 114 |
| A31131H | 5902 | 115 |
| A31132H | 6097 | 116 |
| A31133H | 6144 | 117 |
| A31225H | 5872 | 209 |
| A31226H | 5935 | 210 |
| A31227H | 6013 | 211 |
| A31228H | 6012 | 212 |
| A31229H | 6086 | 213 |
| A31230H | 6081 | 214 |
| A31231H | 6091 | 215 |
| 6280-6779 | | |
| A31290Hi | 6630 | 273 |
| 6780-7279 | | |
| A31167Hi | 7195 | 151 |
| 7780-8279 | | |
| A31049Hi | 7906 | 53 |
| A31168Hi | 7905 | 152 |
| A31169Hi | 8158 | 153 |
| A31291Hi | 7914 | 274 |
| 8280-8779 | | |
| A31292Hi | 8621 | 275 |
| A31293Hi | 8664 | 276 |
| 8780-9279 | | |
| A31170Hi | 8871 | 154 |
| 9280-9779 | | |
| A31171Hi | 9654 | 155 |
| A31294Hi | 9563 | 277 |
| A31295Hi | 9619 | 278 |
| A31296Hi | 9629 | 279 |
| 9780-10279 | | |
| A31050Hi | 9849 | 54 |
| A31051Hi | 9863 | 55 |
| A31172Hi | 9854 | 156 |
| A31173Hi | 9867 | 157 |
| A31297Hi | 10160 | 280 |
| A31298Hi | 10236 | 281 |
| 10280-10779 | | |
| A31012H | 10370 | 16 |
| A31013H | 10379 | 17 |
| A31014H | 10450 | 18 |
| A31052Hi | 10682 | 56 |
| A31096H | 10446 | 88 |
| A31119Hi | 10684 | 103 |
| A31174Hi | 10573 | 158 |
| A31175Hi | 10680 | 159 |
| A31176Hi | 10688 | 160 |
| A31177Hi | 10767 | 161 |
| A31232H | 10370 | 216 |
| A31233H | 10447 | 217 |
| A31299Hi | 10297 | 282 |
| 10780-11279 | | |
| A31015H | 11076 | 19 |
| A31016H | 11077 | 20 |
| A31017H | 11081 | 21 |
| A31018H | 11160 | 22 |
| A31053Hi | 10894 | 57 |
| A31054Hi | 10896 | 58 |
| A31097H | 11005 | 89 |
| A31098H | 11072 | 90 |
| A31099H | 11080 | 91 |
| A31100H | 11083 | 92 |
| A31101H | 11087 | 93 |
| A31102H | 11090 | 94 |
| A31120Hi | 10888 | 104 |
| A31121Hi | 10899 | 105 |
| A31134H | 11094 | 118 |
| A31135H | 11142 | 119 |
| A31136H | 11150 | 120 |
| A31137H | 11156 | 121 |
| A31138H | 11161 | 122 |
| A31178Hi | 10826 | 162 |
| A31179Hi | 10887 | 163 |
| A31180Hi | 10900 | 164 |
| A31181Hi | 10928 | 165 |
| A31234H | 11009 | 218 |
| A31235H | 11066 | 219 |
| A31236H | 11071 | 220 |
| A31237H | 11074 | 221 |
| A31238H | 11079 | 222 |
| A31239H | 11071 | 223 |
| A31240H | 11078 | 224 |
| A31241H | 11080 | 225 |
| A31242H | 11102 | 226 |
| A31243H | 11149 | 227 |
| A31300Hi | 10818 | 283 |
| 11280-11779 | | |
| A31019H | 11522 | 23 |
| A31020H | 11523 | 24 |
| A31103H | 11526 | 95 |
| A31139H | 11489 | 123 |
| A31140H | 11507 | 124 |
| A31141H | 11523 | 125 |
| A31142H | 11543 | 126 |
| A31143H | 11671 | 127 |
| A31144H | 11697 | 128 |
| A31244H | 11329 | 228 |
| A31245H | 11338 | 229 |
| A31246H | 11350 | 230 |
| A31247H | 11338 | 231 |
| A31248H | 11351 | 232 |
| A31249H | 11503 | 233 |
| A31250H | 11526 | 234 |
| A31251H | 11504 | 235 |
| A31252H | 11541 | 236 |
| A31253H | 11521 | 237 |
| A31254H | 11620 | 238 |
| A31255H | 11662 | 239 |
| A31256H | 11678 | 240 |
| 11780-12279 | | |
| A31021H | 11903 | 25 |
| A31022H | 12205 | 26 |
| A31023H | 12228 | 27 |
| A31024H | 12249 | 28 |
| A31025H | 12273 | 29 |
| A31026H | 12274 | 30 |
| A31081H | 12273 | 29 |
| A31082H | 12273 | 29 |
| A31083H | 12273 | 29 |
| A31084H | 12273 | 29 |
| A31085H | 12274 | 30 |
| A31086H | 12274 | 30 |
| A31087H | 12274 | 30 |
| A31088H | 12274 | 30 |
| A31089H | 12274 | 85 |
| A31090H | 12274 | 85 |
| A31091H | 12274 | 85 |
| A31092H | 12275 | 86 |
| A31093H | 12275 | 86 |
| A31094H | 12275 | 86 |
| A31145H | 11787 | 129 |
| A31146H | 11903 | 130 |
| A31147H | 11907 | 131 |
| A31148H | 12124 | 132 |
| A31149H | 12247 | 133 |
| A31257H | 11819 | 241 |
| A31258H | 12064 | 242 |
| A31259H | 12068 | 243 |
| A31260H | 12163 | 244 |
| A31261H | 12165 | 245 |
| A31262H | 12169 | 246 |
| A31263H | 12272 | 247 |
| A31355H | 12273 | 29 |
| A31356H | 12273 | 29 |
| A31357H | 12273 | 29 |
| A31358H | 12273 | 29 |
| A31359H | 12273 | 29 |
| A31360H | 12273 | 29 |
| A31361H | 12274 | 29 |
| A31362H | 12274 | 30 |
| A31363H | 12274 | 30 |
| A31364H | 12274 | 30 |
| A31365H | 12274 | 30 |
| A31366H | 12274 | 30 |
| A31367H | 12274 | 30 |
| 12280-12779 | | |
| A31027H | 12475 | 31 |
| A31104H | 12477 | 96 |
| A31150H | 12498 | 134 |
| A31264H | 12473 | 248 |
| A31265H | 12473 | 249 |
| A31266H | 12498 | 250 |
| A31267H | 12628 | 251 |
| A31301Hi | 12776 | 284 |
| 12780-13279 | | |
| A31302Hi | 13059 | 285 |
| 13280-13779 | | |
| A31055Hi | 13706 | 59 |
| A31109Hi | 13706 | 59 |
| A31110Hi | 13706 | 59 |
| A31111Hi | 13706 | 59 |
| A31112Hi | 13706 | 59 |
| A31113Hi | 13706 | 101 |
| A31114Hi | 13706 | 101 |
| A31115Hi | 13706 | 101 |
| A31116Hi | 13706 | 101 |
| A31117Hi | 13707 | 102 |
| A31118Hi | 13707 | 102 |
| A31182Hi | 13701 | 166 |
| A31183Hi | 13748 | 167 |
| A31303Hi | 13473 | 286 |
| A31304Hi | 13482 | 287 |
| A31305Hi | 13494 | 288 |
| A31306Hi | 13750 | 289 |
| A31368Hi | 13706 | 59 |
| A31369Hi | 13706 | 59 |
| A31370Hi | 13706 | 59 |
| A31371Hi | 13706 | 59 |
| A31372Hi | 13706 | 59 |
| A31373Hi | 13706 | 59 |
| 13780-14279 | | |
| A31056Hi | 14070 | 60 |
| A31057Hi | 14184 | 61 |
| A31184Hi | 13843 | 168 |
| A31185Hi | 13926 | 169 |
| A31186Hi | 14071 | 170 |
| A31187Hi | 14081 | 171 |
| A31188Hi | 14266 | 172 |
| A31307Hi | 13900 | 290 |
| 14280-14779 | | |
| A31058Hi | 14401 | 62 |
| A31059Hi | 14403 | 63 |
| A31122Hi | 14401 | 106 |
| A31123Hi | 14402 | 107 |
| 15280-15779 | | |
| A31308Hi | 15571 | 291 |
| A31309Hi | 15588 | 292 |
| 16280-16779 | | |
| A31060Hi | 16512 | 64 |
| 16780-17279 | | |
| A31028H | 16795 | 32 |
| A31029H | 16846 | 33 |
| A31030H | 16875 | 34 |
| A31105H | 16873 | 97 |
| A31151H | 16874 | 135 |
| A31189Hi | 16921 | 173 |
| A31268H | 16843 | 252 |
| A31269H | 16877 | 253 |
| A31310Hi | 16964 | 293 |
| 17780-18279 | | |
| A31311Hi | 18120 | 294 |
| 18280-18779 | | |
| A31061Hi | 18501 | 65 |
| A31124Hi | 18502 | 108 |
| A31190Hi | 18502 | 174 |
| A31191Hi | 18654 | 175 |
| A31192Hi | 18775 | 176 |
| A31312Hi | 18559 | 295 |
| 18780-19279 | | |
| A31193Hi | 18893 | 177 |
| 19280-19779 | | |
| A31062Hi | 19318 | 66 |
| 20280-20779 | | |
| A31063Hi | 20629 | 67 |
| A31194Hi | 20418 | 178 |
| A31195Hi | 20719 | 179 |
| 20780-21279 | | |
| A31031H | 21266 | 35 |
| A31032H | 21279 | 36 |
| A31152H | 21253 | 136 |
| A31153H | 21256 | 137 |
| A31196Hi | 20984 | 180 |
| A31271H | 21264 | 254 |
| A31272H | 21276 | 255 |
| A31273H | 21266 | 256 |
| A31274H | 21278 | 257 |
| 21280-21779 | | |
| A31033H | 21343 | 37 |
| A31154H | 21352 | 138 |
| A31197Hi | 21442 | 181 |
| A31275H | 21390 | 258 |
| 22280-22779 | | |
| A31064Hi | 22478 | 68 |
| A31065Hi | 22594 | 69 |
| A31198Hi | 22476 | 182 |
| A31199Hi | 22478 | 183 |
| A31200Hi | 22544 | 184 |
| 22780-23279 | | |
| A31034H | 23225 | 38 |
| A31106H | 23222 | 98 |
| 23280-23779 | | |
| A31201Hi | 23557 | 185 |
| A31313Hi | 23568 | 296 |
| 23780-24279 | | |
| A31314Hi | 23845 | 297 |
| A31315Hi | 23964 | 298 |
| A31316Hi | 24162 | 299 |
| A31317Hi | 24175 | 300 |
| 24280-24779 | | |
| A31202Hi | 24672 | 186 |
| A31318Hi | 24450 | 301 |
| 24780-25279 | | |
| A31066Hi | 24910 | 70 |
| A31067Hi | 24945 | 71 |
| A31203Hi | 24910 | 187 |
| A31319Hi | 25010 | 302 |
| A31320Hi | 25035 | 303 |
| 25280-25779 | | |
| A31068Hi | 25310 | 72 |
| A31204Hi | 25308 | 188 |
| 25780-26279 | | |
| A31321Hi | 25951 | 304 |
| A31322Hi | 26028 | 305 |
| A31323Hi | 26214 | 306 |
| 26280-26779 | | |
| A31324Hi | 26674 | 307 |
| 26780-27279 | | |
| A31325Hi | 27090 | 308 |
| A31326Hi | 27163 | 309 |
| A31327Hi | 27237 | 310 |
| 27280-27779 | | |
| A31328Hi | 27640 | 311 |
| 27780-28279 | | |
| A31205Hi | 28147 | 189 |
| A31329Hi | 27890 | 312 |
| A31330Hi | 28024 | 313 |
| 28280-28779 | | |
| A31206Hi | 28750 | 190 |
| A31331Hi | 28745 | 314 |
| 28780-29279 | | |
| A31207Hi | 28958 | 191 |
| A31332Hi | 29041 | 315 |
| A31333Hi | 29223 | 316 |
| 29280-29779 | | |
| A31069Hi | 29495 | 73 |
| A31070Hi | 29545 | 74 |
| A31208Hi | 29430 | 192 |
| A31209Hi | 29648 | 193 |
| A31334Hi | 29441 | 317 |
| A31335Hi | 29617 | 318 |
| 29780-30279 | | |
| A31336Hi | 29800 | 319 |
| 30280-30779 | | |
| A31210Hi | 30615 | 194 |
| A31337Hi | 30601 | 320 |
| A31338Hi | 30732 | 321 |
| 30780-31279 | | |
| A31035H | 31124 | 39 |
| A31036H | 31180 | 40 |
| A31155H | 31175 | 139 |
| A31156H | 31187 | 140 |
| A31157H | 31259 | 141 |
| A31276H | 31170 | 259 |
| A31277H | 31174 | 260 |
| A31278H | 31178 | 261 |
| A31279H | 31204 | 262 |
| A31280H | 31170 | 263 |
| A31283H | 31187 | 266 |
| A31284H | 31205 | 267 |
| 31280-31779 | | |
| A31211Hi | 31637 | 195 |
| A31212Hi | 31657 | 196 |
| A31339Hi | 31371 | 322 |
| 31780-32279 | | |
| A31037H | 31803 | 41 |
| A31038H | 31804 | 42 |
| A31071Hi | 32254 | 75 |
| A31072Hi | 32262 | 76 |
| A31125Hi | 32263 | 109 |
| A31158H | 31796 | 142 |
| A31159H | 31802 | 143 |
| A31160H | 31862 | 144 |
| A31213Hi | 32216 | 197 |
| A31214Hi | 32244 | 198 |
| A31215Hi | 32262 | 199 |
| A31281H | 31793 | 264 |
| A31282H | 31797 | 265 |
| A31285H | 31850 | 268 |
| A31286H | 31796 | 269 |
| A31340Hi | 32126 | 323 |
| 32780-33279 | | |
| A31216Hi | 33199 | 200 |
| A31341Hi | 32843 | 324 |
| A31342Hi | 33123 | 325 |
| A31343Hi | 33252 | 326 |
| 33280-33779 | | |
| A31073Hi | 33476 | 77 |
| A31074Hi | 33477 | 78 |
| A31126Hi | 33475 | 110 |
| A31127Hi | 33482 | 111 |
| A31217Hi | 33483 | 201 |
| A31218Hi | 33506 | 202 |
| A31219Hi | 33588 | 203 |
| A31344Hi | 33530 | 327 |
| A31345Hi | 33566 | 328 |
| A31346Hi | 33654 | 329 |
| 33780-34279 | | |
| A31347Hi | 33941 | 330 |
| A31348Hi | 34241 | 331 |
| A31349Hi | 34244 | 332 |
| 34280-34779 | | |
| A31075Hi | 34555 | 79 |
| A31220Hi | 34727 | 204 |
| A31350Hi | 34331 | 333 |
| A31351Hi | 34695 | 334 |
| 34780-35279 | | |
| A31076Hi | 35277 | 80 |
| A31077Hi | 35279 | 81 |
| A31221Hi | 35278 | 205 |
| A31222Hi | 35279 | 206 |
| A31352Hi | 34843 | 335 |
| A31353Hi | 35001 | 336 |
| 35280-35779 | | |
| A31039H | 35572 | 43 |
| A31040H | 35616 | 44 |
| A31041H | 35665 | 45 |
| A31042H | 35710 | 46 |
| A31043H | 35754 | 47 |
| A31044H | 35758 | 48 |
| A31045H | 35774 | 49 |
| A31046H | 35775 | 50 |
| A31078Hi | 35284 | 82 |
| A31079Hi | 35330 | 83 |
| A31080Hi | 35346 | 84 |
| A31107H | 35620 | 99 |
| A31108H | 35778 | 100 |
| A31161H | 35706 | 145 |
| A31162H | 35718 | 146 |
| A31163H | 35769 | 147 |
| A31223Hi | 35347 | 207 |
| A31224Hi | 35370 | 208 |
| A31287H | 35583 | 270 |
| A31288H | 35584 | 271 |
| A31354Hi | 35479 | 337 |
| 35780-36279 | | |
| A31047H | 35781 | 51 |
| A31048H | 35879 | 52 |
| A31164H | 35782 | 148 |
| A31165H | 35784 | 149 |
| A31166H | 35874 | 150 |
| A31289H | 35906 | 272 |

as well as the oligonucleotides hybridizing in these areas:

Table 5 shows some hybridizing active regions of SEQ ID NO. 1 and human NLRP3 antisense oligonucleotides hybridizing in these regions.

The mouse NLRP3 oligonucleotides of the present invention hybridize with hybridizing active regions of NLRP3 pre-mRNA for example of SEQ ID NO. 3 Hybridizing active areas of SEQ ID NO. 3 are for example listed in the following

TABLE 6

| Region of | First position on | |
|---|---|---|
| SEQ ID NO. 3 | SEQ ID NO. 3 | SEQ ID NO. |
| 3686-4185 | | |
| A31001M | 3807 | 342 |
| A31002M | 4083 | 343 |
| A31003M | 4087 | 344 |
| A31098M | 3705 | 430 |
| A31099M | 3806 | 431 |
| A31100M | 3806 | 432 |
| A31101M | 3807 | 433 |
| A31102M | 4081 | 434 |
| A31118M | 4083 | 343 |
| A31119M | 4096 | 449 |
| A31138M | 3686 | 465 |
| 4186-4685 | | |
| A31004M | 4305 | 345 |
| A31005M | 4306 | 346 |
| A31006M | 4307 | 347 |
| A31007M | 4307 | 348 |
| A31050Mi | 4411 | 382 |
| A31051Mi | 4618 | 383 |
| A31120MR | 4307 | 450 |
| A31121MR | 4308 | 451 |
| 5186-5685 | | |
| A31052Mi | 5367 | 384 |
| A31053Mi | 5517 | 385 |
| 6186-6685 | | |
| A31054Mi | 6279 | 386 |
| A31055Mi | 6355 | 387 |
| A31056Mi | 6358 | 388 |
| A31057Mi | 6509 | 389 |
| A31058Mi | 6509 | 390 |
| A31059Mi | 6515 | 391 |
| A31060Mi | 6515 | 392 |
| 6686-7185 | | |
| A31061Mi | 7132 | 393 |
| A31062Mi | 7133 | 394 |
| 7686-8185 | | |
| A31063Mi | 8170 | 395 |
| 8186-8685 | | |
| A31064Mi | 8490 | 396 |
| 8686-9185 | | |
| A31008M | 9015 | 349 |
| A31009M | 9019 | 350 |
| A31010M | 9048 | 351 |
| A31011M | 9049 | 352 |
| A31012M | 9049 | 353 |
| A31013M | 9050 | 354 |
| A31014M | 9050 | 355 |
| A31015M | 9055 | 356 |
| A31016M | 9122 | 357 |
| A31017M | 9122 | 358 |
| A31041M | 9048 | 351 |
| A31042M | 9048 | 351 |
| A31043M | 9048 | 351 |
| A31044M | 9048 | 351 |
| A31065Mi | 8756 | 397 |
| A31066Mi | 8756 | 398 |
| A31067Mi | 8836 | 399 |
| A31068Mi | 8885 | 400 |
| A31122M | 8968 | 452 |
| A31123M | 9043 | 453 |
| A31124M | 9059 | 454 |
| A31139M | 9009 | 466 |
| A31140M | 9015 | 467 |
| A31141M | 9016 | 468 |
| A31142M | 9022 | 469 |
| A31143M | 9024 | 470 |
| A31144M | 9052 | 471 |
| A31145M | 9116 | 472 |
| A31146M | 9126 | 473 |
| 9186-9685 | | |
| A31018M | 9599 | 359 |
| A31019M | 9601 | 360 |
| A31020M | 9602 | 361 |
| A31021M | 9605 | 362 |
| A31125MR | 9267 | 455 |
| A31126M | 9590 | 456 |
| A31127M | 9599 | 359 |
| A31128M | 9601 | 457 |
| A31147M | 9596 | 474 |
| A31148M | 9606 | 475 |
| 9686-10185 | | |
| A31022M | 9869 | 363 |
| A31023M | 9870 | 364 |
| A31024M | 9870 | 365 |
| A31025M | 9871 | 366 |
| A31026M | 9873 | 367 |
| A31027M | 9878 | 368 |
| A31028M | 10044 | 369 |
| A31103M | 9710 | 435 |
| A31104M | 10097 | 436 |
| A31105M | 10143 | 437 |
| A31106M | 10143 | 438 |
| A31129M | 9872 | 458 |
| A31130M | 10019 | 459 |
| A31131M | 10020 | 460 |
| A31132M | 10044 | 369 |
| A31133M | 10088 | 461 |
| A31134M | 10090 | 462 |
| A31149M | 9866 | 476 |
| A31150M | 9876 | 477 |
| A31151M | 9877 | 478 |
| A31152M | 10024 | 479 |
| 10186-10685 | | |
| A31029M | 10228 | 370 |
| A31030M | 10233 | 371 |
| A31031M | 10234 | 372 |
| A31032M | 10234 | 373 |
| A31107M | 10252 | 439 |
| A31108M | 10442 | 440 |
| A31153M | 10215 | 480 |
| 10686-11185 | | |
| A31069Mi | 11021 | 401 |
| 11186-11685 | | |
| A31070Mi | 11341 | 402 |
| 11686-12185 | | |
| A31071Mi | 12045 | 403 |
| 12686-13185 | | |
| A31154M | 12790 | 481 |
| 13186-13685 | | |
| A31072Mi | 13244 | 404 |
| A31073Mi | 13672 | 405 |
| 13686-14185 | | |
| A31074Mi | 13906 | 406 |
| A31075Mi | 14147 | 407 |
| 14186-14685 | | |
| A31076Mi | 14378 | 408 |
| 15686-16185 | | |
| A31077Mi | 15826 | 409 |
| A31078Mi | 15832 | 410 |
| A31079Mi | 15893 | 411 |
| A31080Mi | 15899 | 412 |
| A31081Mi | 16071 | 413 |
| 16186-16685 | | |
| A31082Mi | 16351 | 414 |
| 16686-17185 | | |
| A31155M | 16743 | 482 |
| 17186-17685 | | |
| A31083Mi | 17288 | 415 |
| 17686-18185 | | |
| A31084Mi | 17943 | 416 |
| A31085Mi | 18058 | 417 |
| 19186-19685 | | |
| A31033M | 19371 | 374 |
| A31135MR | 19390 | 463 |
| 20186-20685 | | |
| A31086Mi | 20304 | 418 |
| A31087Mi | 20517 | 419 |
| A31088Mi | 20545 | 420 |
| 22686-23185 | | |
| A31089Mi | 22910 | 421 |
| 23186-23685 | | |
| A31090Mi | 23548 | 422 |
| A31091Mi | 23552 | 423 |
| 23686-24185 | | |
| A31092Mi | 23744 | 424 |
| A31093Mi | 23751 | 425 |
| A31094Mi | 23854 | 426 |
| 24186-24685 | | |
| A31095Mi | 24285 | 427 |
| 25686-26185 | | |
| A31096Mi | 25786 | 428 |
| A31097Mi | 25939 | 429 |
| A31109M | 26089 | 441 |
| A31110M | 26092 | 442 |
| A31137MR | 26017 | 464 |
| 26686-27185 | | |
| A31034M | 27157 | 375 |
| A31045M | 27157 | 375 |
| A31046M | 27157 | 375 |
| A31047M | 27157 | 375 |
| A31111M | 26906 | 443 |
| A31112M | 26914 | 444 |
| A31113M | 27154 | 445 |
| A31114M | 27157 | 375 |
| A31156M | 27167 | 483 |
| 27186-27685 | | |
| A31035M | 27405 | 376 |
| A31036M | 27406 | 377 |
| A31037M | 27409 | 378 |
| A31038M | 27409 | 379 |
| A31039M | 27411 | 380 |
| A31040M | 27411 | 381 |
| A31048M | 27405 | 376 |
| A31049M | 27405 | 376 |
| A31115M | 27211 | 446 |
| A31116M | 27404 | 447 |
| A31117M | 27413 | 448 |
| A31157M | 27407 | 484 |

as well as the oligonucleotides hybridizing in these areas:

Table 6 shows some hybridizing active regions of SEQ ID NO. 3 and mouse NLRP3 antisense oligonucleotides hybridizing in these regions.

The oligonucleotide of the present invention inhibits for example at least about 30%, 35%, 40%, 45%, 50%, 55%, 60%, 65%, 70%, 75%, 80%, 85%, 90%, 92%, 94%, 95%, 96%, 97%, 98%, 99% or 100% of NLRP3 such as the, e.g., human or murine, NLRP3 expression compared to an untreated control. The oligonucleotide is for example administered via gymnotic delivery i.e., without a transfection reagent. The oligonucleotides of the present invention inhibit NLRP3 expression for example in a cell, tissue, organ, or a subject. The oligonucleotide of the present invention inhibits the expression of NLRP3 at a nanomolar or micromolar concentration for example in a concentration of 0.1, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900 or 950 nM, or 1, 10 or 100 μM.

The oligonucleotide of the present invention is used for example in a concentration of 1, 3, 5, 9, 10, 15, 27, 30, 40, 50, 75, 82, 100, 250, 300, 500, or 740 nM, or 1, 2.2, 3, 5, 6.6 or 10 μM.

An oligonucleotide of the present invention hybridizes for example with at least one exon and/or intron of SEQ ID NO. 1 and optionally with the mRNA of SEQ ID NO. 2. The oligonucleotide hybridizes for example with 2, 3, 4 or 5 exons and/or with 2, 3, 4 or 5 introns.

In addition, the present invention refers to a pharmaceutical composition comprising an oligonucleotide of the present invention and a pharmaceutically acceptable carrier, excipient and/or dilutant. The pharmaceutical composition further comprises for example another active agent for example selected from the group consisting of an oligonucleotide, an antibody, a small molecule, a polypeptide, a lipid, sugar and a combination thereof.

The oligonucleotide or the pharmaceutical composition of the present invention is for example for use in a method of preventing and/or treating a disorder. The disorder is for example characterized by an NLRP3 imbalance, i.e., the NLRP3 level is increased in comparison to the level in a normal, healthy cell, tissue, organ or subject. The NLRP3 level can be measured by any standard method such as immunohistochemistry, western blot, quantitative real time PCR, HHPLC, FPLC or QuantiGene assay known to a person skilled in the art.

A disorder treatable by an oligonucleotide of the present invention or a pharmaceutical composition comprising such oligonucleotide is for example selected from the group consisting of an inflammatory or autoimmune disorder, a neurological disorder, cardiovascular or metabolic disorder, renal disorder, liver disorder lung disorder, skin disorder, ocular disorder, disorder of the gastro-intestinal tract, joint inflammation, organ transplantation, fibrotic disorder and a combination thereof. Furthermore, the disorder is a hyperproliferative disorder such as a cancer. A cancer is for example selected from the group consisting of breast cancer, lung cancer, malignant melanoma, lymphoma, skin cancer, bone cancer, prostate cancer, liver cancer, brain cancer, cancer of the larynx, gall bladder, pancreas, testicular, rectum, parathyroid, thyroid, adrenal, neural tissue, head and neck, colon, stomach, bronchi, kidneys, basal cell carcinoma, squamous cell carcinoma, metastatic skin carcinoma, osteo sarcoma, Ewing's sarcoma, reticulum cell sarcoma, liposarcoma, myeloma, giant cell tumor, small-cell lung tumor, islet cell tumor, primary brain tumor, meningioma, acute and chronic lymphocytic and granulocytic tumors, acute and chronic myeloid leukemia, hairy-cell tumor, adenoma, hyperplasia, medullary carcinoma, intestinal ganglioneuromas, Wilm's tumor, seminoma, ovarian tumor, leiomyomater tumor, cervical dysplasia, retinoblastoma, soft tissue sarcoma, malignant carcinoid, topical skin lesion, rhabdomyosarcoma, Kaposi's sarcoma, osteogenic sarcoma, malignant hypercalcemia, renal cell tumor, polycythermia vera, adenocarcinoma, anaplastic astrocytoma, glioblastoma multiforma, leukemia, epidermoid carcinoma and a combination thereof.

In more detail such disorder is for example selected from the group consisting of Alzheimer's disease, multiple sclerosis, autoimmune encephalitis, stroke, traumatic brain injury, atherosclerosis, nonalcoholic fatty liver disease, nonalcoholic steatohepatitis, hypertension, myocardial infarction, acute kidney injury, ischemia reperfusion injury, chronic kidney diseases, crystal-induced nephropathies, glomerulonephritis, silicosis, asthma, allergic airway inflammation, inflammatory bowel disease, colitis ulcerosa, osteoarthritis, rheumatoid arthritis, juvenile idiopathic arthritis, transplantation of kidney, lung, liver and/or heart, fibrotic disorder of kidney, lung, liver and/or heart, hyperinflammation following influenza infection, graft-versus-host disease, interstitial cystitis, uveitis, sinusitis, periodontal disease, myelodysplastic syndrome, cryopyrin-associated periodic syndromes, gout, obesity-induced inflammation, insulin resistance, type 1 and type 2 diabetes, contact hypersensitivity, psoriasis, alopecia and a combination thereof.

An oligonucleotide or a pharmaceutical composition of the present invention is administered locally or systemically for example orally, sublingually, nasally, subcutaneously, intravenously, intraperitoneally, intramuscularly, intratumoral, intrathecal, intraventricular, transdermal, rectal, intraarticular, intraocular, intravitreal, subconjunctival, retro bulbar, intra nasal, intracameral, intratracheal, intrapleural, per inhalation, intraurethral and/or intra vesical. In addition, an oligonucleotide or a pharmaceutical composition of the present invention is for example used in an ex vivo treatment of a transplant.

According to the present invention, one or more oligonucleotides of the present invention can be administered together, at the same time point for example in a pharmaceutical composition or separately, or on staggered intervals. Alternatively, one or more oligonucleotides of the present invention are for example administered together with another active agent such as another oligonucleotide (i.e., not being part of the present invention), an antibody, a small molecule, a polypeptide, a statin, a vaccine, an adjuvant, a chemotherapeutic agent, a cytotoxic agent, an allergen, an antibiotic, a siRNA molecule, a TLR antagonist, an activated cell, a cell therapy product, a peptide, a polypeptide, a protein, a gene therapy vector, and/or a co-stimulatory molecule (e.g., a cytokine, a chemokine, a protein ligand, a trans-activating factor, a peptide or peptide comprising modified amino acid and/or a therapeutic mRNA), at the same time point for example in a pharmaceutical composition or separately, or on staggered intervals. The other active agent interacts or inhibits the same target and/or a different target than the oligonucleotide of the present invention, wherein interacting means that the active agent has an indirect effect on the target. Another active agent interacts or inhibits for example a target selected from the group consisting of NLRP3, CD39, CD73, IL-1β, IL-1 receptor, IL-1R accessory protein, IL-18, IL-18 receptor, ASC, NLRC4, AIM2, Caspase-1, RIPK3, Gasdermin D, MLKL, TLR4, Caspase-8, P2X7, NFκB, RORγt, TGF-β, IL-21, IL-17, IL-22, IL-23, IL-6, TNF-α, CCR6, CCL20, STAT3, MMP-1, MMP-8, ADAMTS-5, HMG-CoA, Myd-88, HMGB-1, ROS, TAK-1, Chop, FPR1, LIMCH1, caspase inhibitor and a combination thereof.

Further, one or more of the oligonucleotides of the present invention are connected, e.g., via a chemical bond for example based on a linker. The chemical bond such as a linker is for example cleavable or non-cleavable.

The present invention further refers to a kit comprising an oligonucleotide and/or a pharmaceutical composition of the present invention and an instruction manual. The oligonucleotide and/or the pharmaceutical composition of the kit is for example for use in preventing and/or treating a disease caused by an imbalanced NLRP3 expression. Diseases based on such imbalanced NLRP3 expression are mentioned above. Alternatively, the kit is for example a diagnostic kit for the determination of the NLRP3 level in a sample. The sample is for example blood, saliva, urine, liquor or smear.

A subject of the present invention is for example a mammalian, a bird or a fish, wherein the mammalian is for example a human, horse, cow, cat, dog, or rabbit.

### EXAMPLES

The following examples illustrate different embodiments of the present invention, but the invention is not limited to these examples. The following experiments are performed on cells endogenously expressing NLRP3 either in an unstimulated state or after stimulation with for example LPS, i.e., the cells do not represent an artificial system comprising transfected reporter constructs. Such artificial systems generally show a higher degree of inhibition and lower IC₅₀ values than endogenous systems which are closer to therapeutically relevant in vivo systems. Further, in the following experiments no transfecting agent is used, i.e., gymnotic delivery is performed. Transfecting agents are known to increase the activity of an oligonucleotide which influences the IC₅₀ value (see for example Zhang et al., Gene Therapy, 2011, 18, 326-333; Stanton et al., Nucleic Acid Therapeutics, Vol. 22, No. 5, 2012). As artificial systems using a transfecting agent are hard or impossible to translate into therapeutic approaches and no transfection formulation has been approved so far for oligonucleotides, the following experiments are performed without any transfecting agent.

### Example 1: Design of Mouse and Human NLRP3-Specific Antisense Oligonucleotides (ASOs)

For the design of human ASOs with specificity for exonic regions within the human NLRP3 gene the NRLP3 mRNA of SEQ ID NO. 2 (RefSeq ID NM_004895.4) was used. For ASOs with specificity for intronic regions within the human NLRP3 gene the NLRP3 pre-mRNA of SEQ ID NO. 1 (GRCh38, Chr1: 247412861-247452403) as annotated in FASTA format (visible range) downloaded from https://www.ncbi.nim.nih.gov/nuccore/NM 004895.4 was used. An “H” after the ASO ID indicates a human NLRP3-specific sequence that binds to an exonic region of the pre-mRNA and a “Hi” after the ASO ID indicates a human NLRP3-specific sequence that binds to an intronic region of the pre-mRNA. 16, 17, 18 and 19 mers were designed according to in house criteria.

For the design of mouse ASOs with specificity for exonic regions within the mouse NLRP3 gene the NRLP3 mRNA of SEQ ID NO. 4 (RefSeq ID NM_145827) was used. For ASOs with specificity for intronic regions within the mouse NLRP3 gene the NLRP3 pre-mRNA of SEQ ID NO. 3 (GRmCh38, Chr11: 59539030-59569495) as annotated in FASTA format (visible range) downloaded from https://www.ncbi.nlm.nih.gov/nuccore/NM_145827) was used. An “M” after the ASO ID indicates a mouse NLRP3-specific sequence that binds to an exonic region of the pre-mRNA and a “Mi” after the ASO ID indicates a mouse NLRP3-specific sequence that binds to an intronic region of the pre-mRNA. 15, 16 and 17 mers were designed according to in house criteria.

Neg1 (SEQ ID NO. 339; described in WO2014154843 A1), S5 (SEQ ID NO. 486), R01002 (SEQ ID NO. 340) and R01011 (SEQ ID NO. 341) were used as non-targeting control oligonucleotides in some experiments (Table 1 and Table 2).

### Example 2: First Screening—Single Concentration Efficacy Screen for Human NLRP3 Antisense Oligonucleotides in THP-1 Cells

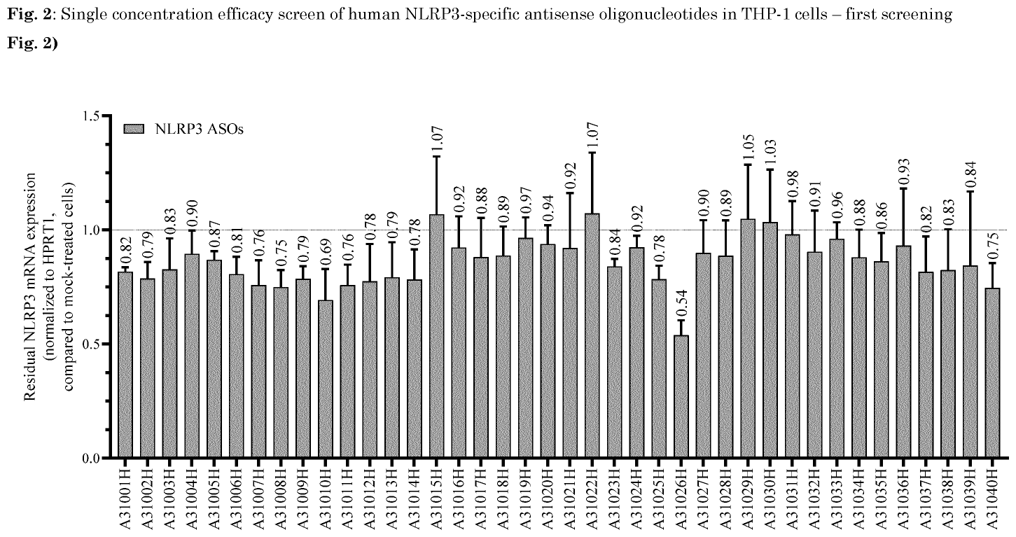

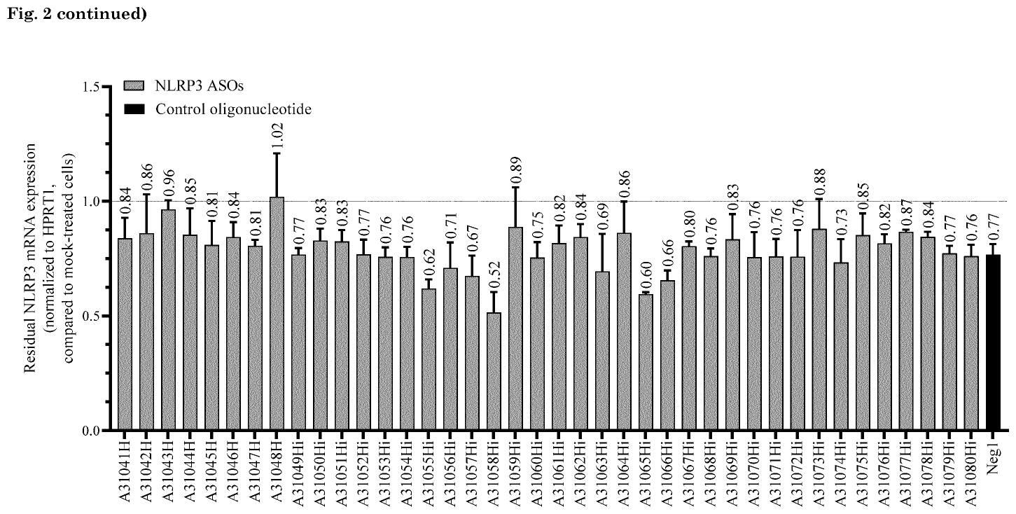

Knockdown efficacy of human NLRP3-specific ASOs were tested in human THP-1 cells (human monocytic cell line derived from an acute monocytic leukemia patient). The cells were treated with the respective human NLRP3-specific ASO or control oligonucleotide Neg1 (SEQ ID NO. 339) at a concentration of 10 μM. Mock-treated cells (no antisense oligonucleotide) were cultured without the addition of oligonucleotides (untreated control). After three days treatment, cells were lyzed. Human HPRT1 and human NLRP3 mRNA expression was measured using the QuantiGene Singleplex assay (ThermoFisher) and the NLRP3 expression values were normalized to HPRT1 values. Residual NLRP3 mRNA expression relative to mock-treated cells (set as 1) is shown as mean and SD in FIG. 2.

Treatment of THP-1 cells with the A31058Hi (SEQ ID NO. 62), A31026H (SEQ ID NO. 30) and A31065Hi (SEQ ID NO. 69) ASOs resulted in a target inhibition of >40% (represented by a residual NLRP3 mRNA expression of <0.6 as compared to mock-treated cells) (FIG. 2).

### Example 3: First Screening—Single Concentration Efficacy Screen for Human NLRP3 Antisense Oligonucleotides in U87MG Cells

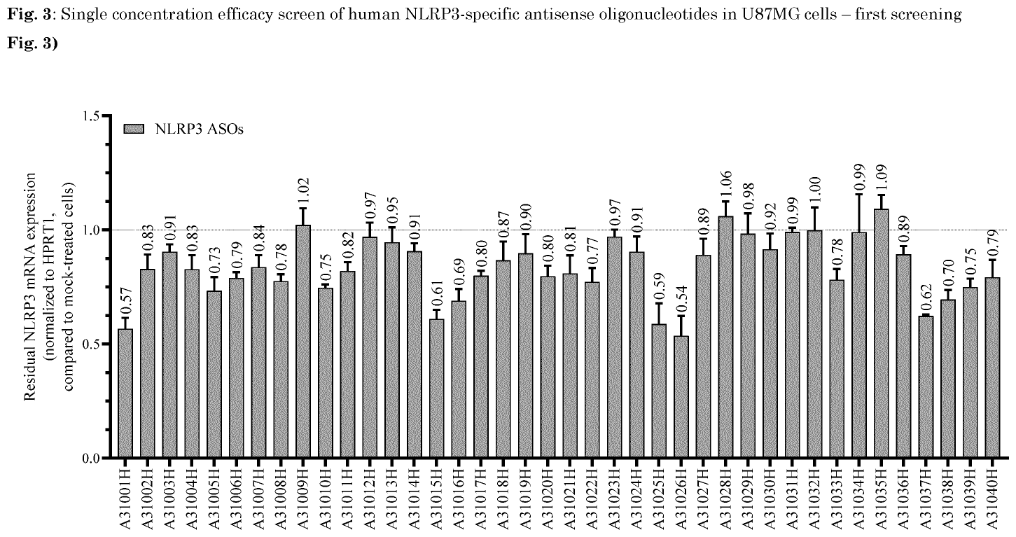

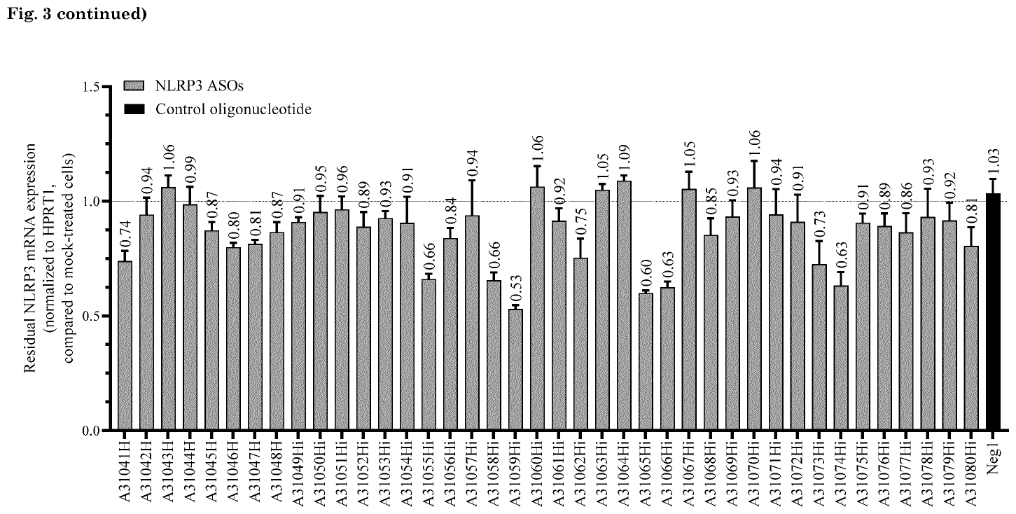

Knockdown efficacy of human NLRP3-specific ASOs were tested in human U87MG cells (human primary glioblastoma cell line). The cells were treated with the respective human NLRP3-specific ASO or control oligonucleotide Neg1 at a concentration of 10 μM. Mock-treated cells (no antisense oligonucleotide) were cultured without the addition of oligonucleotides (untreated control). After three days treatment, cells were lyzed. Human HPRT1 and human NLRP3 mRNA expression was measured using the QuantiGene Singleplex assay (ThermoFisher) and the NLRP3 expression values were normalized to HPRT1 values. Residual NLRP3 mRNA expression relative to mock-treated cells (set as 1) is shown as mean and SD in FIG. 3.

Treatment of U87-MG cells with the A31059Hi (SEQ ID NO. 63), A31026H (SEQ ID NO. 30), A31001H (SEQ ID NO. 5) and A31025H (SEQ ID NO. 29) ASOs resulted in a target inhibition of >40% (represented by a residual NLRP3 mRNA expression of <0.6 as compared to mock-treated cells) (FIG. 3).

### Example 4: First Screening—Single Concentration Efficacy Screen for Human NLRP3 Antisense Oligonucleotides in PBMC-Derived Macrophages

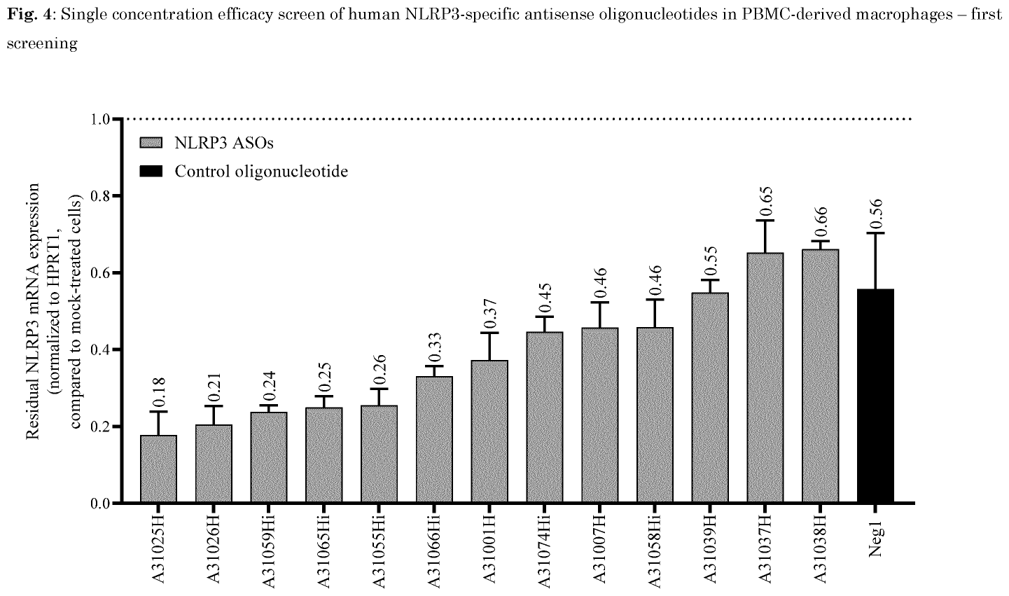

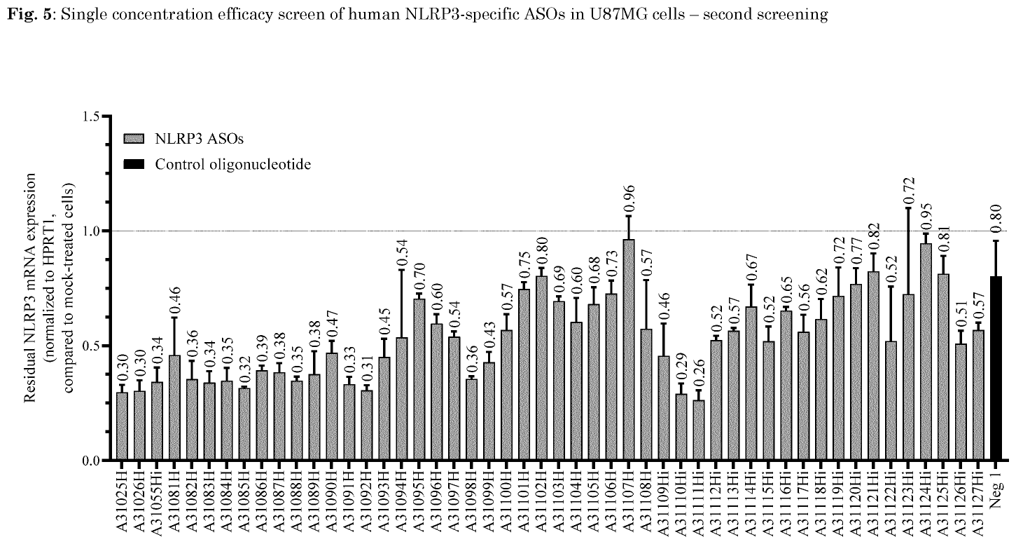

Knockdown efficacy of selected human NLRP3-specific ASOs from the first screening round were tested in human peripheral blood mononuclear cell (PBMC)-derived macrophages. PBMC were seeded in 96-well flat bottom plate and after two hours, non-adherent cells were washed away and the adherent cells (predominantly monocytes) were cultured in the presence of 20 ng/ml hGM-CSF and 50 ng/ml hIL-4. Cells were treated with the respective human NLRP3-specific ASO or control oligonucleotide Neg1 at a concentration of 10 μM. Mock-treated cells (no antisense oligonucleotide) were cultured without the addition of oligonucleotides (untreated control). After three days treatment, the supernatant and loose cells were removed and replaced by fresh medium containing 20 ng/ml hGM-CSF, 50 ng/ml hIL-4 and the respective human NLRP3-specific ASO or control oligonucleotide Neg1 at a concentration of 10 μM. On day 6, cells were lyzed. Human HPRT1 and human NLRP3 mRNA expression was measured using the QuantiGene Singleplex assay (ThermoFisher) and the NLRP3 expression values were normalized to HPRT1 values. Residual NLRP3 mRNA expression relative to mock-treated cells (set as 1) is shown as mean and SD in FIG. 4.

Treatment of PBMC-derived macrophages with the A31025H (SEQ ID NO. 29), A31026H (SEQ ID NO. 30), A31059Hi (SEQ ID NO. 63), A31065Hi (SEQ ID NO. 69) and A31055Hi (SEQ ID NO. 59) ASOs resulted in a target inhibition of >70% (represented by a residual NLRP3 mRNA expression of <0.3 as compared to mock-treated cells) (FIG. 4).

### Example 5: Second Screening—Single Concentration Efficacy Screen of NLRP3-Specific ASOs in U87MG Cells

Knockdown efficacy of human NLRP3-specific ASOs was tested in human U87MG cells in a second screening round. The cells were treated with the respective human NLRP3-specific ASO or control oligonucleotide Neg1 (SEQ ID NO. 339) at a concentration of 10 μM. Mock-treated cells (no antisense oligonucleotide) were cultured without the addition of oligonucleotides (untreated control). After three days treatment, cells were lyzed. Human HPRT1 and human NLRP3 mRNA expression was measured using the QuantiGene Singleplex assay (ThermoFisher) and the NLRP3 expression values were normalized to HPRT1 values. Residual NLRP3 mRNA expression relative to mock-treated cells (set as 1) is shown as mean and SD in FIG. 5.

Treatment of U87-MG cells with the A31111Hi (SEQ ID NO. 59), A31110Hi (SEQ ID NO. 59), A31025H (SEQ ID NO. 29), A31026H (SEQ ID NO. 30), A31092H (SEQ ID NO. 86), A31085H (SEQ ID NO. 30), A31091H (SEQ ID NO. 85) and A31083H (SEQ ID NO. 29) ASOs resulted in a target inhibition of >66% (represented by a residual NLRP3 mRNA expression of <0.34 as compared to mock-treated cells) (FIG. 5).

### Example 6: Second Screening—Single Concentration Efficacy Screen of Human NLRP3-Specific ASOs in PBMC-Derived Macrophages

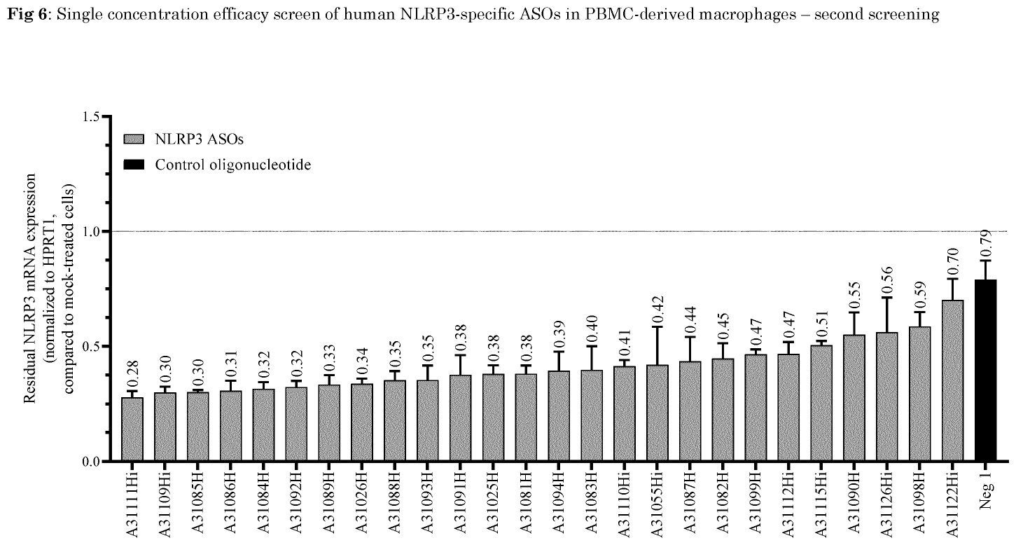

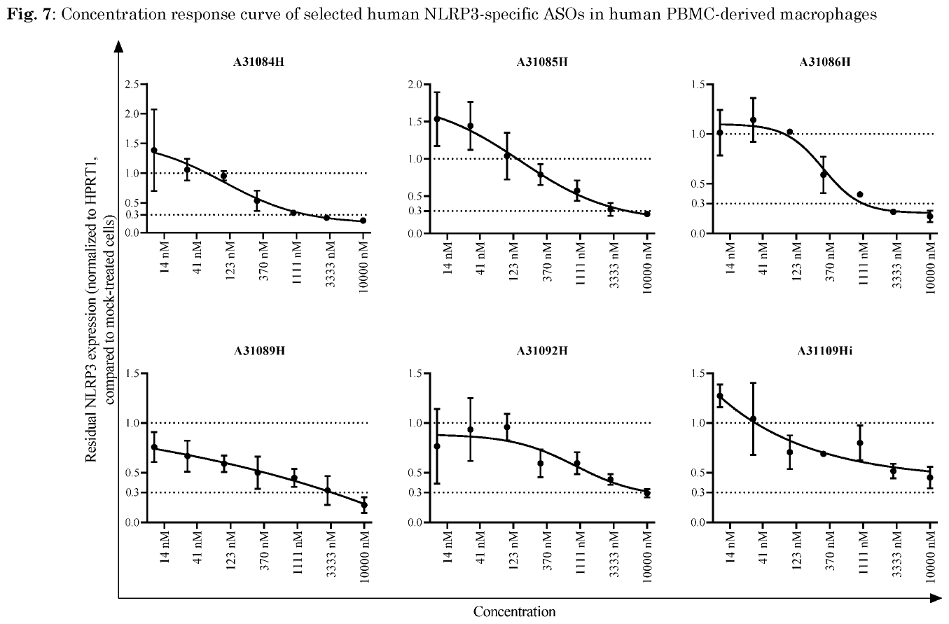

Knockdown efficacy of selected human NLRP3-specific ASOs from the second screening round were tested in PBMC-derived macrophages. Plastic adherent PBMC were treated with 10 μM of the respective human NLRP3-specific ASO or control oligonucleotide Neg1 (SEQ ID NO. 339) in the presence of 20 ng/ml hGM-CSF and 50 ng/ml hIL-4. Mock-treated cells (no antisense oligonucleotide) were cultured without the addition of oligonucleotides (untreated control). After three days treatment, cells were lyzed. Human HPRT1 and human NLRP3 mRNA expression was measured using the QuantiGene Singleplex assay (ThermoFisher) and the NLRP3 expression values were normalized to HPRT1 values. Residual NLRP3 mRNA expression relative to mock-treated cells (set as 1) is shown as mean and SD in FIG. 6.

Treatment of PBMC-derived macrophages with the A31111Hi (SEQ ID NO. 59), A31109Hi (SEQ ID NO. 59), A31085H (SEQ ID NO. 30), A31086H (SEQ ID NO. 30), A31084H (SEQ ID NO. 29), A31092H (SEQ ID NO. 86), A31089H (SEQ ID NO. 85) and A31026H (SEQ ID NO. 30) ASOs resulted in a target inhibition of at least >66% (represented by a residual NLRP3 mRNA expression of <0.34 as compared to mock-treated cells) (FIG. 6).

### Example 7: Investigation of the Concentration-Dependent Target Knockdown by Selected Human NLRP3-Specific ASOs in Human PBMC-Derived Macrophages

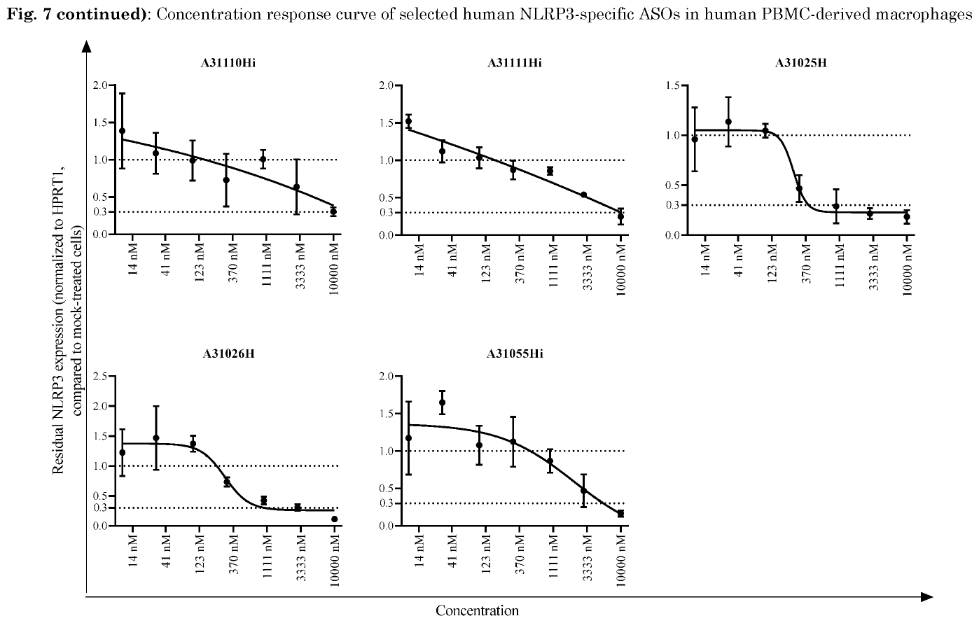

The concentration-dependent knockdown of NLRP3 mRNA expression by selected human NLRP3-specific ASOs from the first and second screening round was investigated on mRNA level in human PBMC-derived macrophages and the respective IC₅₀ values were calculated. Plastic adherent PBMC were cultured in the presence of 20 ng/ml hGM-CSF and 50 ng/ml hIL-4 and treated for three days with the respective ASO at the following concentrations: 10000 nM, 3333 nM, 1111 nM, 370 nM, 123 nM, 41 nM and 14 nM. Mock-treated cells (no antisense oligonucleotide) were cultured without the addition of oligonucleotides. After three days, the cell culture supernatant and loose cells were removed and replaced by fresh medium containing 40 ng/ml hGMCSF and 100 ng/ml hIL-4 and antisense oligonucleotides at a final concentration ranging from 14 nM to 10 μM. On day six, cells were lyzed. Human HPRT1 and human NLRP3 mRNA expression was measured using the QuantiGene Singleplex assay (ThermoFisher) and the NLRP3 expression values were normalized to HPRT1 values. Residual NLRP3 mRNA expression relative to mock-treated cells (set as 1) is shown as mean and SD in FIG. 7 and Table 7. The half maximal inhibitory concentration (IC₅₀) values of the concentration response curve for selected candidates are indicated in the Table 8. All ASOs concentration-dependently inhibited the expression of NLRP3 mRNA.

TABLE 7
Concentration-dependent inhibition of NRLP3 mRNA expression in PBMC-derived
macrophages by selected NLRP3-specific ASOs after six days treatment.

| | | | Residual NLRP3 | | | | Residual NLRP3 |
|---|---|---|---|---|---|---|---|
| | | | mRNA expression | | | | mRNA expression |
| | | | compared to | | | | compared to |
| | | | mock-treated cells | | | | mock-treated cells |
| ASO ID | Concentration | | (set as 1) | ASO ID | Concentration | | (set as 1) |
| A31025H | 10 | μM | 0.18 | A31089H | 10 | μM | 0.17 |
| (SEQ | 3.3 | μM | 0.22 | (SEQ ID | 3.3 | μM | 0.32 |
| IDNO. 29) | 1.1 | μM | 0.29 | NO. 85) | 1.1 | μM | 0.45 |
| | 370 | nM | 0.47 | | 370 | nM | 0.50 |
| | 123 | nM | 1.05 | | 123 | nM | 0.59 |
| | 41 | nM | 1.14 | | 41 | nM | 0.67 |
| | 14 | nM | 0.96 | | 14 | nM | 0.76 |
| A31026H | 10 | μM | 0.11 | A31092H | 10 | μM | 0.29 |
| (SEQ ID | 3.3 | μM | 0.31 | (SEQ ID | 3.3 | μM | 0.43 |
| NO. 30) | 1.1 | μM | 0.42 | NO. 86) | 1.1 | μM | 0.60 |
| | 370 | nM | 0.73 | | 370 | nM | 0.59 |
| | 123 | nM | 1.37 | | 123 | nM | 0.96 |
| | 41 | nM | 1.47 | | 41 | nM | 0.93 |
| | 14 | nM | 1.22 | | 14 | nM | 0.77 |
| A31055Hi | 10 | μM | 0.17 | A31109Hi | 10 | μM | 0.45 |
| (SEQ ID | 3.3 | μM | 0.47 | (SEQ ID | 3.3 | μM | 0.52 |
| NO. 59) | 1.1 | μM | 0.87 | NO. 59 | 1.1 | μM | 0.80 |
| | 370 | nM | 1.12 | | 370 | nM | 0.69 |
| | 123 | nM | 1.08 | | 123 | nM | 0.71 |
| | 41 | nM | 1.65 | | 41 | nM | 1.04 |
| | 14 | nM | 1.17 | | 14 | nM | 1.27 |
| A31084H | 10 | μM | 0.20 | A31110Hi | 10 | μM | 0.30 |
| (SEQ ID | 3.3 | μM | 0.25 | (SEQ ID | 3.3 | μM | 0.64 |
| NO. 29) | 1.1 | μM | 0.33 | NO. 59) | 1.1 | μM | 1.01 |
| | 370 | nM | 0.54 | | 370 | nM | 0.73 |
| | 123 | nM | 0.95 | | 123 | nM | 0.99 |
| | 41 | nM | 1.06 | | 41 | nM | 1.09 |
| | 14 | nM | 1.39 | | 14 | nM | 1.39 |
| A31085H | 10 | μM | 0.26 | A31111Hi | 10 | μM | 0.25 |
| (SEQ ID | 3.3 | μM | 0.32 | (SEQ ID | 3.3 | μM | 0.54 |
| NO. 30) | 1.1 | μM | 0.57 | NO. 59) | 1.1 | μM | 0.86 |
| | 370 | nM | 0.79 | | 370 | nM | 0.87 |
| | 123 | nM | 1.04 | | 123 | nM | 1.03 |
| | 41 | nM | 1.44 | | 41 | nM | 1.12 |
| | 14 | nM | 1.53 | | 14 | nM | 1.52 |

| | | | |
|---|---|---|---|
| A31086H | 10 | μM | 0.17 |
| (SEQ ID | 3.3 | μM | 0.22 |
| NO. 30) | 1.1 | μM | 0.39 |
| | 370 | nM | 0.59 |
| | 123 | nM | 1.02 |
| | 41 | nM | 1.14 |
| | 14 | nM | 1.01 |

TABLE 8
Half maximal inhibitory concentration (IC₅₀) values and R values
of selected human NLRP3-specific ASOs after six days treatment.

| ASO ID | IC₅₀ values (nM) | R squared |
|---|---|---|
| A31025H | 248.50 | 0.86 |
| A31026H | 281.00 | 0.82 |
| A31055Hi | 2190.00 | 0.71 |
| A31084H | 100.90 | 0.76 |
| A31085H | 154.40 | 0.85 |
| A31086H | 298.00 | 0.89 |
| A31092H | 949.90 | 0.55 |

### Example 8: Third Screening—Single Concentration Efficacy Screen for Human NLRP3-Specific Antisense Oligonucleotides in THP-1-Derived Macrophages

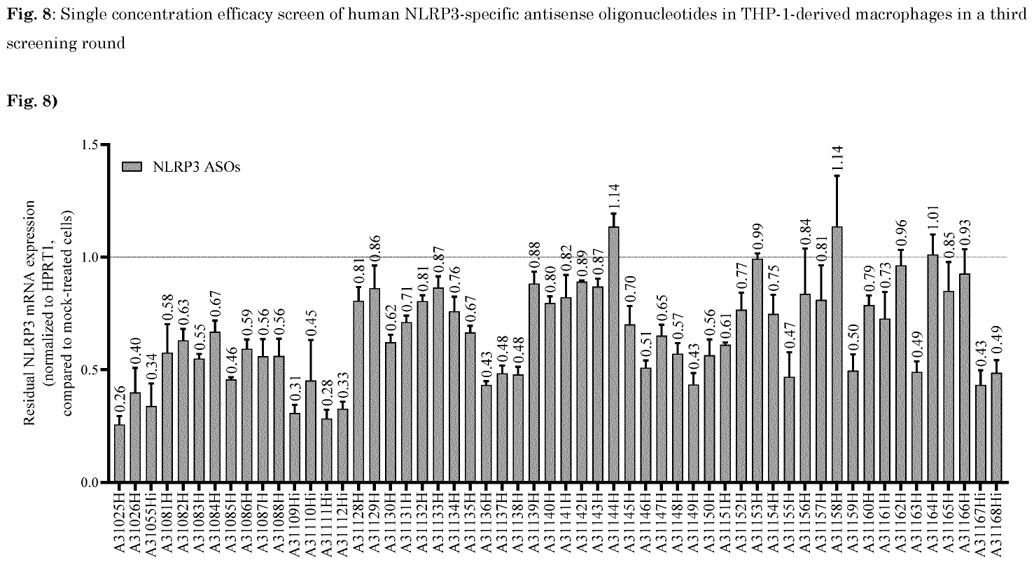

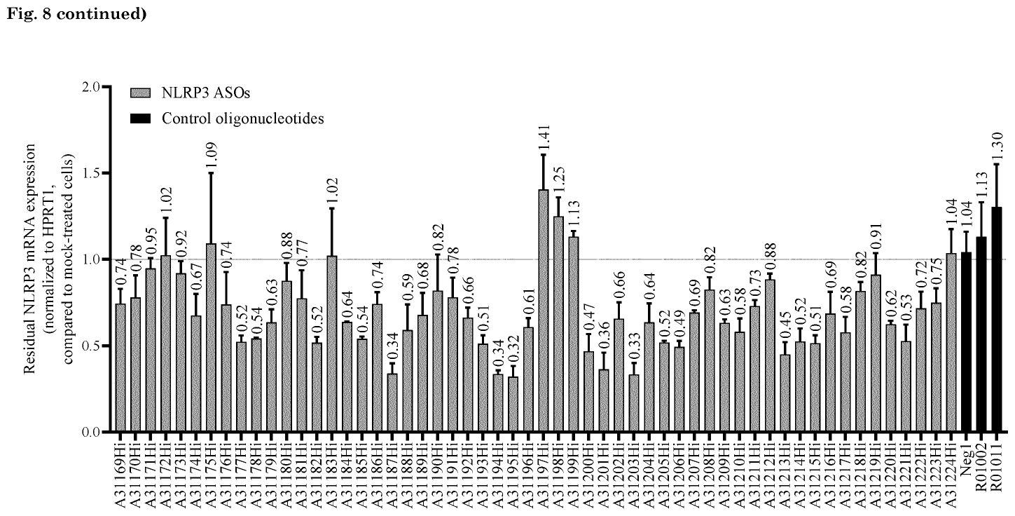

Knockdown efficacy of human NLRP3-specific ASOs were tested in human THP-1-derived macrophages in a third screening round. THP-1 cells were differentiated into macrophages by addition of 10 nM PMA. The respective NLRP3-specific ASOs or control oligonucleotides (Neg1 (SEQ ID NO. 339), R01002 (SEQ ID NO. 340) and R01011 (SEQ ID NO. 341)) were added at 5 μM final concentration. Mock-treated cells (no antisense oligonucleotide) were cultured without the addition of oligonucleotides (untreated control). After three days treatment, cells were lyzed. Human HPRT1 and human NLRP3 mRNA expression was measured using the QuantiGene Singleplex assay (ThermoFisher) and the NLRP3 expression values were normalized to HPRT1 values. Residual NLRP3 mRNA expression relative to mock-treated cells (set as 1) is shown as mean and SD in FIG. 8. Treatment of THP-1-derived macrophages with the A31025H (SEQ ID NO. 29), A31111Hi (SEQ ID NO. 59), A31109Hi (SEQ ID NO. 59), A31195Hi (SEQ ID NO. 179), A31112Hi (SEQ ID NO. 59), A31203Hi (SEQ ID NO. 187), A31194Hi (SEQ ID NO. 178), A31187Hi (SEQ ID NO. 171) and A31055Hi (SEQ ID NO. 59) ASOs resulted in a target inhibition of >66% (represented by a residual NLRP3 mRNA expression of <0.34 as compared to mock-treated cells) (FIG. 8).

### Example 9: Third Screening—Single Concentration Efficacy Screen of Human NLRP3-Specific ASOs in Human PBMC-Derived Macrophages

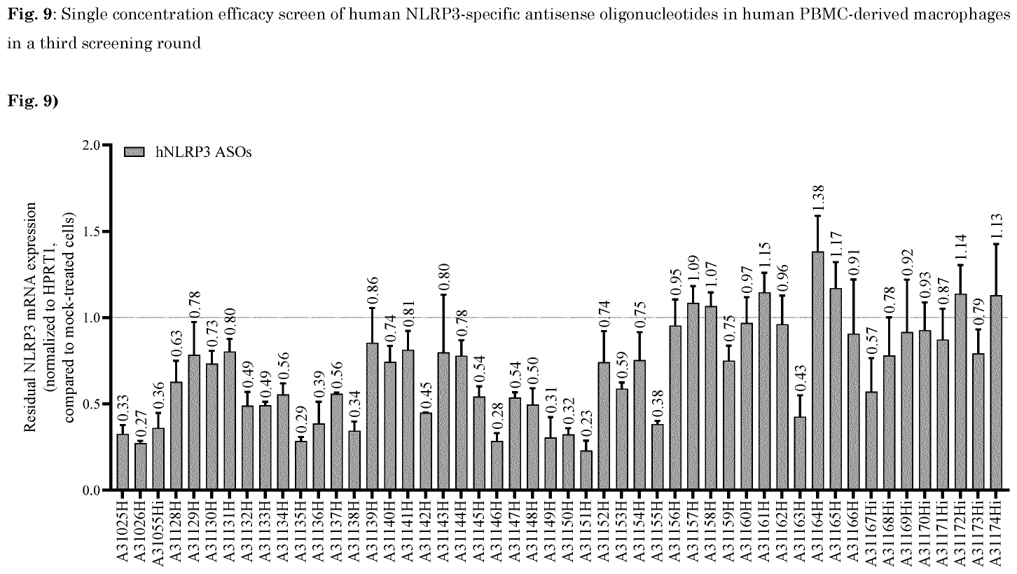

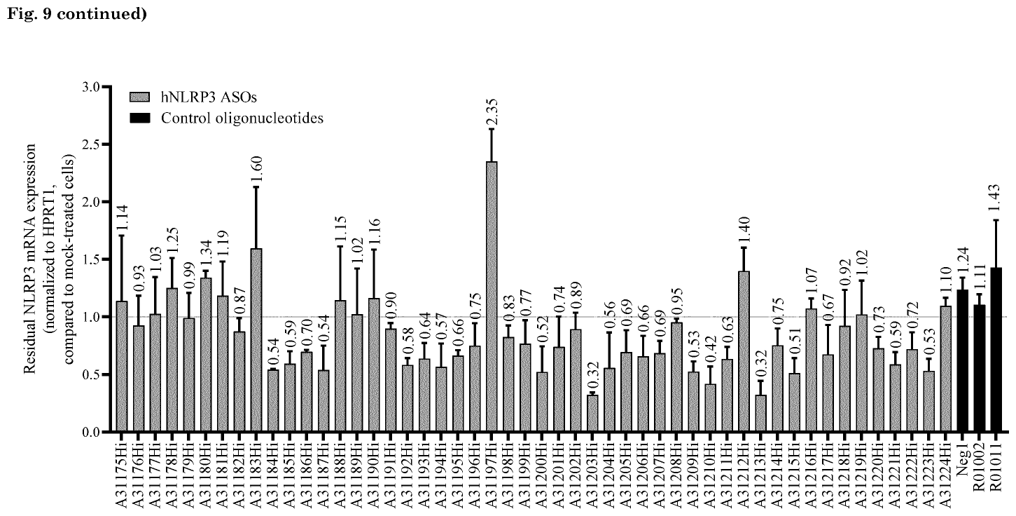

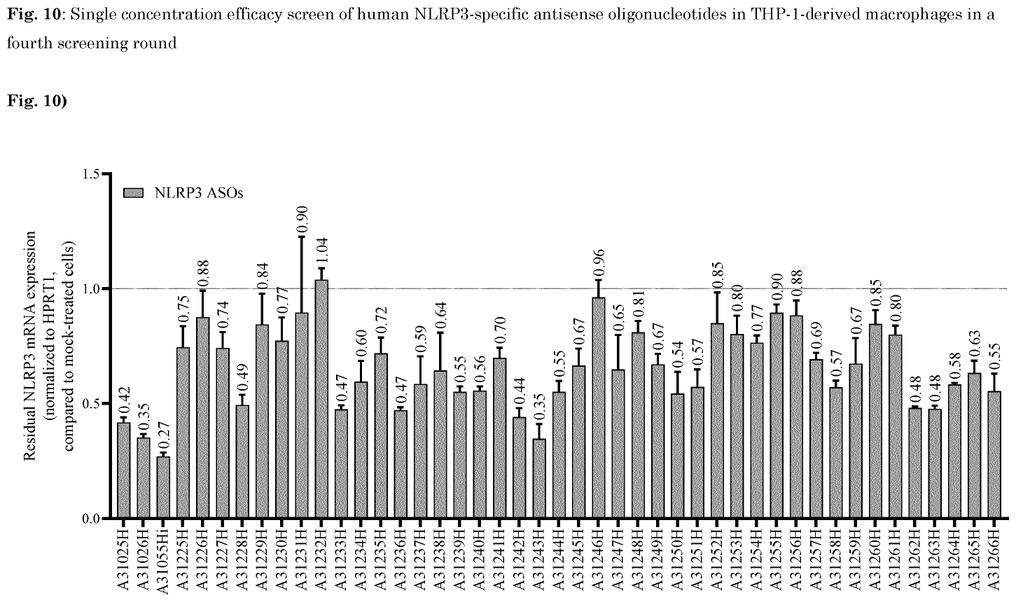

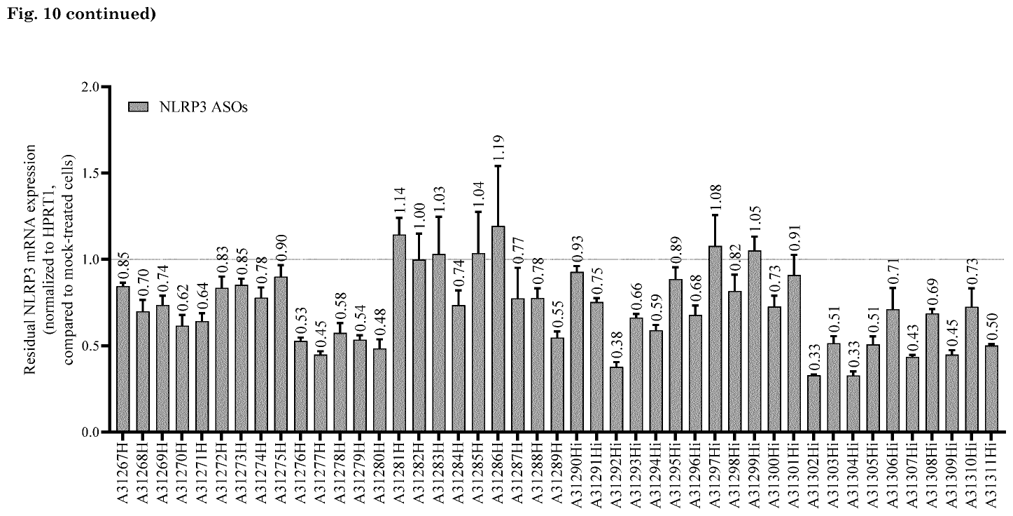

Knockdown efficacy of human NLRP3-specific ASOs was tested in PBMC-derived macrophages in a third screening round. Adherent PBMC cells were cultured in the presence of 20 ng/ml hGM-CSF and 50 ng/ml hIL-4. Cells were treated with the respective human NLRP3-specific ASO or control oligonucleotides (Neg1 (SEQ ID NO. 339), R01002 (SEQ ID NO. 340) and R01011 (SEQ ID NO. 341)) at a concentration of 5 μM. Mock-treated cells (no antisense oligonucleotide) were cultured without the addition of oligonucleotides. After three days treatment, the supernatant and loose cells were removed and replaced by fresh medium containing 20 ng/ml hGM-CSF, 50 ng/ml hIL-4 and the respective human NLRP3-specific ASO or control oligonucleotide at a concentration of 5 μM. On day six, cells were lyzed. Human HPRT1 and human NLRP3 mRNA expression was measured using the QuantiGene Singleplex assay (ThermoFisher) and the NLRP3 expression values were normalized to HPRT1 values. Residual NLRP3 mRNA expression relative to mock-treated cells (set as 1) is shown as mean and SD in FIG. 9.

Treatment of PBMC-derived macrophages with the A31151H (SEQ ID NO. 135), A31026H (SEQ ID NO. 30), A31146H (SEQ ID NO. 130), A31135H (SEQ ID NO. 119), A31149H (SEQ ID NO. 133), A31213Hi (SEQ ID NO. 197), A31203Hi (SEQ ID NO. 187), A31150H (SEQ ID NO. 134) and A31025H (SEQ ID NO. 29) ASOs resulted in a target inhibition of >67% (represented by a residual NLRP3 mRNA expression of <0.33 as compared to mock-treated cells (untreated control)) (FIG. 9).

### Example 10: Fourth Screening—Single Concentration Efficacy Screen for Human NLRP3-Specific Antisense Oligonucleotides in THP-1-Derived Macrophages

Knockdown efficacy of human NLRP3-specific ASOs was tested in human THP-1-derived macrophages in a fourth screening round. THP-1 cells were differentiated into macrophages by addition of 10 nM PMA. The respective NLRP3-specific ASOs or control oligonucleotides (Neg1 (SEQ ID NO. 339), R01002 (SEQ ID NO. 340) and R01011 (SEQ ID NO. 341)) were added at 5 μM final concentration. Mock-treated cells (no antisense oligonucleotide) were cultured without the addition of oligonucleotides (untreated control). After three days treatment, cells were lyzed. Human HPRT1 and human NLRP3 mRNA expression was measured using the QuantiGene Singleplex assay (ThermoFisher) and the NLRP3 expression values were normalized to HPRT1 values. Residual NLRP3 mRNA expression relative to mock-treated cells (set as 1) is shown as mean and SD in FIG. 10.

Treatment of THP-1-derived macrophages with the A31352Hi (SEQ ID NO. 335), A31055Hi (SEQ ID NO. 59), A31314Hi (SEQ ID NO. 297), A31317Hi (SEQ ID NO. 300), A31332Hi (SEQ ID NO. 315), A31324Hi (SEQ ID NO. 307), A31304Hi (SEQ ID NO. 287) and A31302Hi (SEQ ID NO. 285) ASOs resulted in a target inhibition of >67% (represented by a residual NLRP3 mRNA expression of <0.33 as compared to mock-treated cells) (FIG. 10).

### Example 11: Fourth Screening—Single Concentration Efficacy Screen of Human NLRP3-Specific ASOs in PBMC-Derived Macrophages

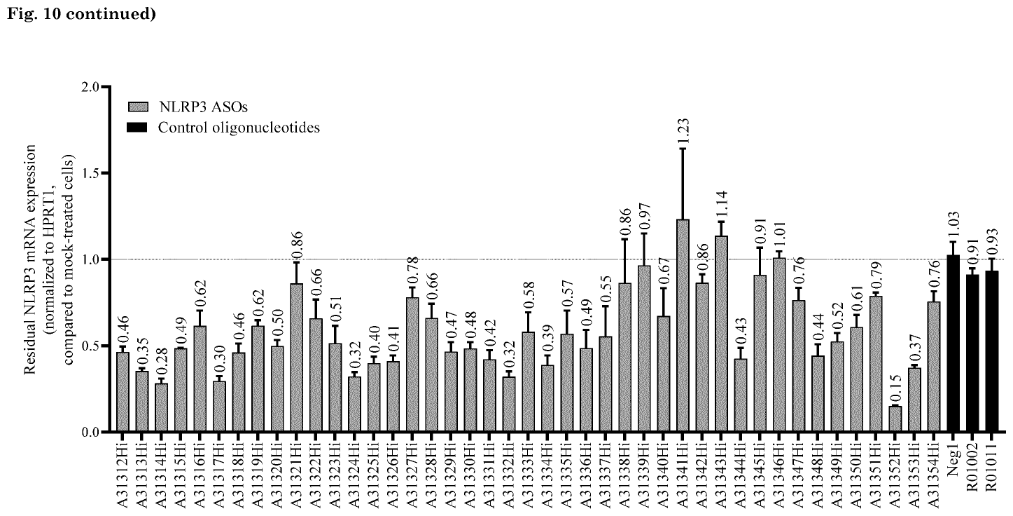

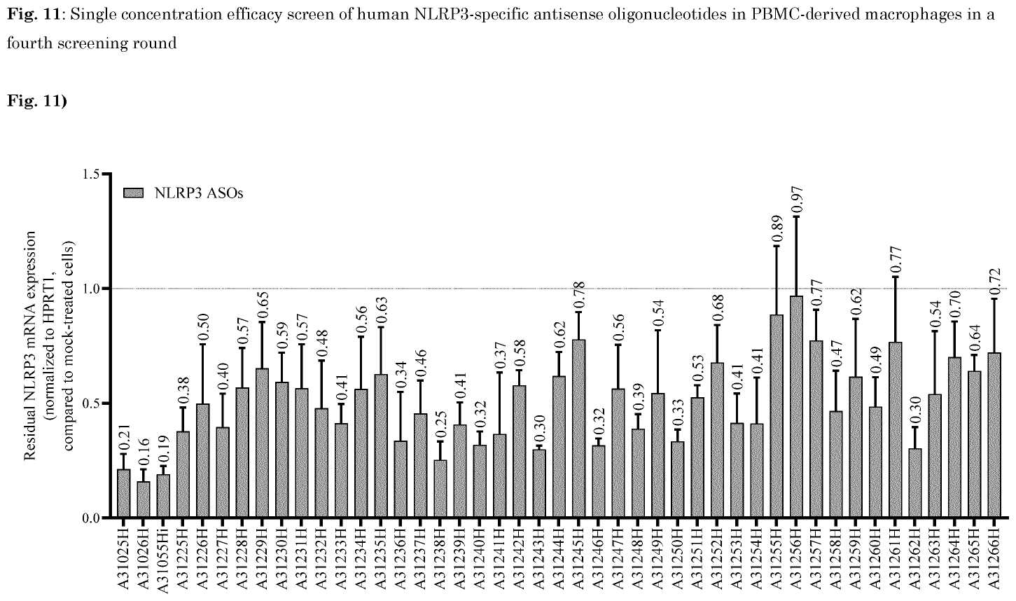

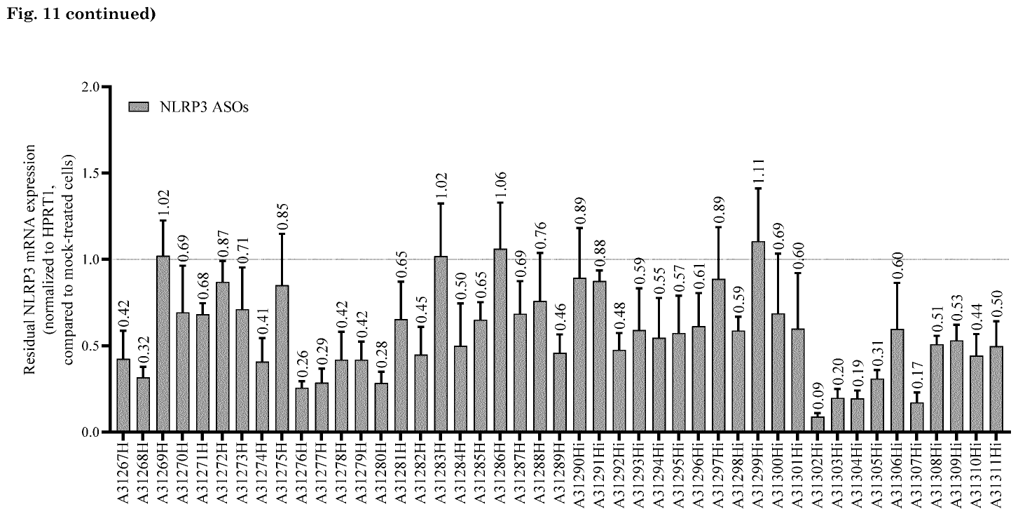

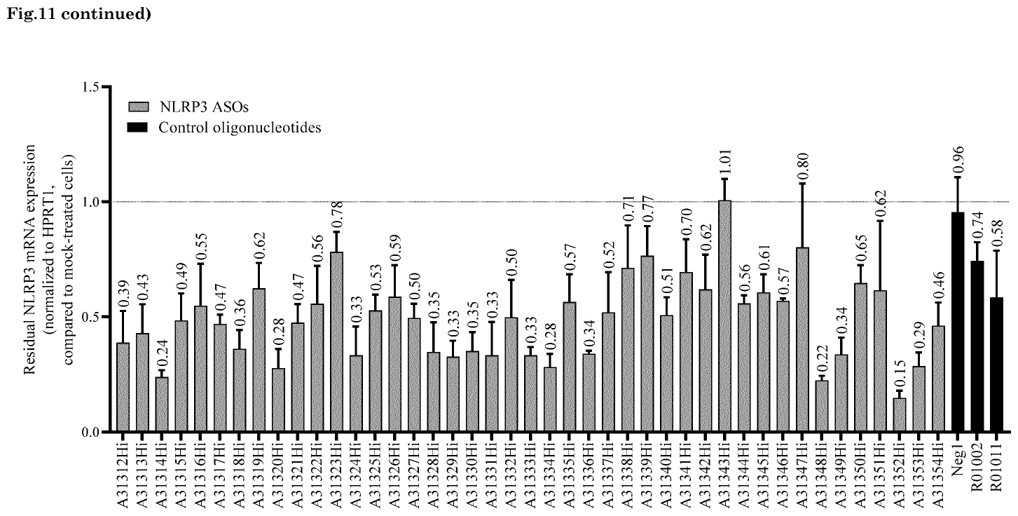

Knockdown efficacy of human NLRP3-specific ASOs was tested in PBMC-derived macrophages in a fourth screening round. Adherent PBMC cells were cultured in the presence of 20 ng/ml hGM-CSF and 50 ng/ml hIL-4. Cells were treated with the respective human NLRP3-specific ASO or control oligonucleotides (Neg1 (SEQ ID NO. 339), R01002 (SEQ ID NO. 340) and R01011 (SEQ ID NO. 341)) at a concentration of 5 μM. Mock-treated cells (no antisense oligonucleotide) were cultured without the addition of oligonucleotides (untreated control). After three days treatment, the supernatant and loose cells were removed and replaced by fresh medium containing 20 ng/ml hGM-CSF, 50 ng/ml hIL-4 and the respective human NLRP3-specific ASO or control oligonucleotide at a concentration of 5 μM. On day six, cells were lyzed. Human HPRT1 and human NLRP3 mRNA expression was measured using the QuantiGene Singleplex assay (ThermoFisher) and the NLRP3 expression values were normalized to HPRT1 values. Residual NLRP3 mRNA expression relative to mock-treated cells (set as 1) is shown as mean and SD in FIG. 11.

Treatment of PBMC-derived macrophages with the A31302Hi (SEQ ID NO. 285), A31352Hi (SEQ ID NO. 335), A31026H (SEQ ID NO. 30), A31307Hi (SEQ ID NO. 290), A31055Hi (SEQ ID NO. 59), A31304Hi (SEQ ID NO. 287) and A31303Hi (SEQ ID NO. 286) ASOs resulted in a target inhibition of >80% (represented by a residual NLRP3 mRNA expression of <0.2 as compared to mock-treated cells) (FIG. 11).

### Example 12: Investigation of the Concentration-Dependent Target Knockdown by Selected Human NLRP3-Specific ASOs in Human THP-1-Derived Macrophages

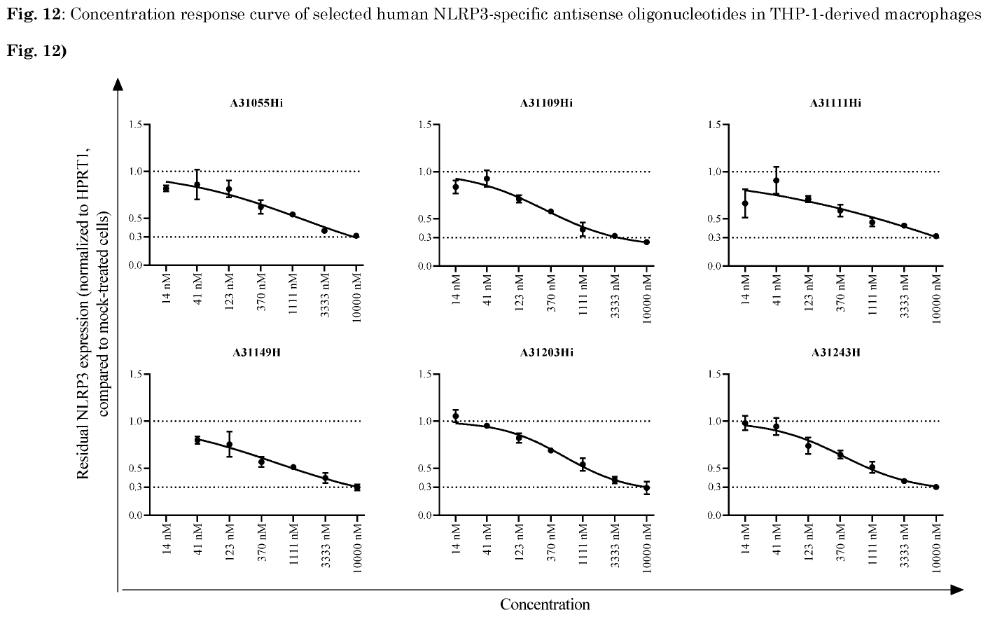

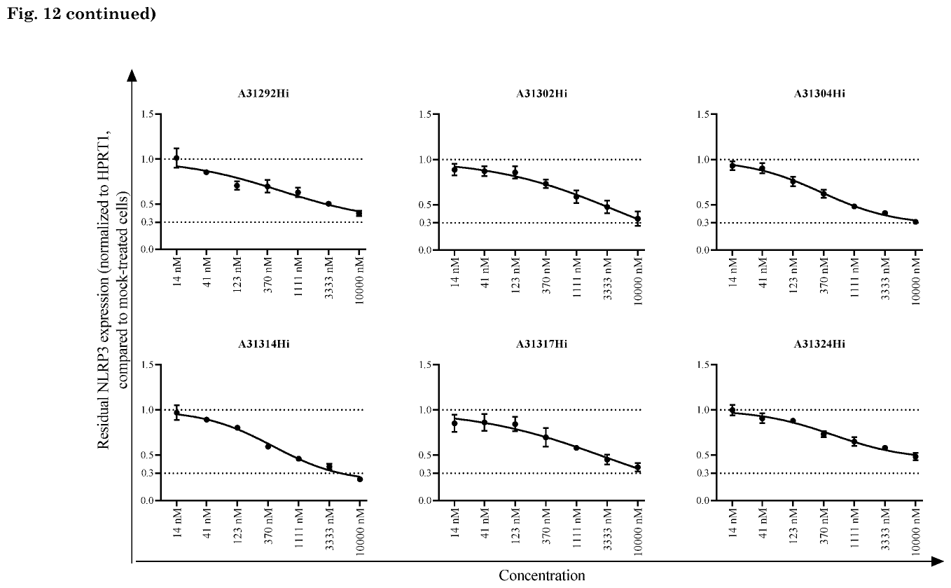

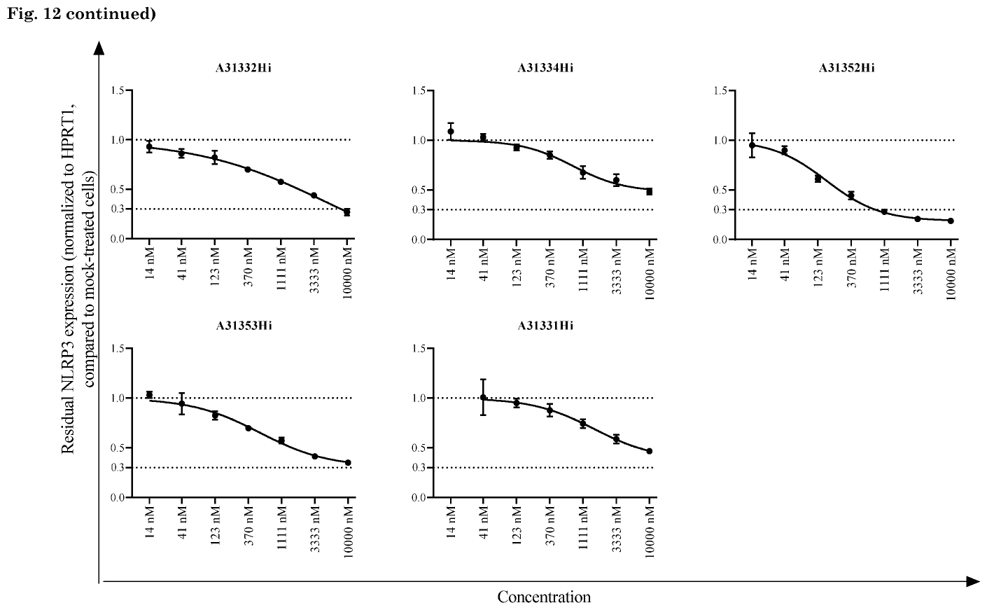

The concentration-dependent knockdown of NLRP3 mRNA expression by selected human NLRP3-specific ASOs from the first, second, third and fourth screening round was investigated on mRNA level in human THP-1-derived macrophages and the respective IC₅₀ values were calculated. THP-1 cells differentiated to macrophages in the presence of 10 nM PMA were treated for three days with the respective ASO at the following concentrations: 10000 nM, 3333 nM, 1111 nM, 370 nM, 123 nM, 41 nM and 14 nM. Mock-treated cells (no antisense oligonucleotide) were cultured without the addition of oligonucleotides. After three days, cells were lyzed. Human HPRT1 and human NLRP3 mRNA expression was measured using the QuantiGene Singleplex assay (ThermoFisher) and the NLRP3 expression values were normalized to HPRT1 values. Residual NLRP3 mRNA expression relative to mock-treated cells (set as 1) is shown as mean and SD in FIG. 12 and Table 9. The half maximal inhibitory concentration (IC₅₀) values of the dose response curve for selected candidates are indicated in the Table 10. All ASOs dose-dependently inhibited the expression of NLRP3 mRNA.

TABLE 9
Concentration-dependent inhibition of NRLP3 mRNA expression in THP-1-derived
macrophages by selected NLRP3-specific ASOs after three days treatment.

| | | | Residual NLRP3 | | | | Residual NLRP3 |
|---|---|---|---|---|---|---|---|
| | | | mRNA expression | | | | mRNA expression |
| | | | compared to | | | | compared to |
| | | | mock-treated cells | | | | mock-treated cells |
| ASO ID | Concentration | | (set as 1) | ASO ID | Concentration | | (set as 1) |
| A31055Hi | 14 | nM | 0.82 | A31314Hi | 14 | nM | 0.97 |
| (SEQ ID | 41 | nM | 0.86 | (SEQ ID | 41 | nM | 0.89 |
| NO. 59) | 123 | nM | 0.81 | NO. 297) | 123 | nM | 0.80 |
| | 370 | nM | 0.62 | | 370 | nM | 0.59 |
| | 1.1 | μM | 0.54 | | 1.1 | μM | 0.46 |
| | 3.3 | μM | 0.37 | | 3.3 | μM | 0.37 |
| | 10 | μM | 0.31 | | 10 | μM | 0.23 |
| A31109Hi | 14 | nM | 0.84 | A31317Hi | 14 | nM | 0.85 |
| (SEQ ID | 41 | nM | 0.93 | (SEQ ID | 41 | nM | 0.86 |
| NO. 59) | 123 | nM | 0.71 | NO. 300) | 123 | nM | 0.84 |
| | 370 | nM | 0.58 | | 370 | nM | 0.70 |
| | 1.1 | μM | 0.39 | | 1.1 | μM | 0.58 |
| | 3.3 | μM | 0.32 | | 3.3 | μM | 0.45 |
| | 10 | μM | 0.25 | | 10 | μM | 0.37 |
| A31111Hi | 14 | nM | 0.66 | A31324Hi | 14 | nM | 1.00 |
| (SEQ ID | 41 | nM | 0.91 | (SEQ ID | 41 | nM | 0.91 |
| NO. 59) | 123 | nM | 0.71 | NO. 307) | 123 | nM | 0.88 |
| | 370 | nM | 0.59 | | 370 | nM | 0.73 |
| | 1.1 | μM | 0.46 | | 1.1 | μM | 0.65 |
| | 3.3 | μM | 0.43 | | 3.3 | μM | 0.58 |
| | 10 | μM | 0.32 | | 10 | μM | 0.48 |
| A31149H | 14 | nM | — | A31332Hi | 14 | nM | 0.93 |
| (SEQ ID | 41 | nM | 0.80 | (SEQ ID | 41 | nM | 0.86 |
| NO. 133) | 123 | nM | 0.76 | NO. 315) | 123 | nM | 0.82 |
| | 370 | nM | 0.57 | | 370 | nM | 0.70 |
| | 1.1 | μM | 0.51 | | 1.1 | μM | 0.58 |
| | 3.3 | μM | 0.40 | | 3.3 | μM | 0.44 |
| | 10 | μM | 0.30 | | 10 | μM | 0.27 |
| A31203Hi | 14 | nM | 1.05 | A31334Hi | 14 | nM | 1.09 |
| (SEQ ID | 41 | nM | 0.95 | (SEQ ID | 41 | nM | 1.03 |
| NO. 187) | 123 | nM | 0.82 | NO. 317) | 123 | nM | 0.93 |
| | 370 | nM | 0.69 | | 370 | nM | 0.85 |
| | 1.1 | μM | 0.54 | | 1.1 | μM | 0.67 |
| | 3.3 | μM | 0.38 | | 3.3 | μM | 0.60 |
| | 10 | μM | 0.29 | | 10 | μM | 0.48 |
| A31243H | 14 | nM | 0.98 | A31352Hi | 14 | nM | 0.95 |
| (SEQ ID | 41 | nM | 0.94 | (SEQ ID | 41 | nM | 0.90 |
| NO. 227) | 123 | nM | 0.74 | NO. 335) | 123 | nM | 0.61 |
| | 370 | nM | 0.65 | | 370 | nM | 0.44 |
| | 1.1 | μM | 0.51 | | 1.1 | μM | 0.28 |
| | 3.3 | μM | 0.37 | | 3.3 | μM | 0.21 |
| | 10 | μM | 0.30 | | 10 | μM | 0.19 |
| A31292Hi | 14 | nM | 1.01 | A31353Hi | 14 | nM | 1.03 |
| (SEQ ID | 41 | nM | 0.86 | (SEQ ID | 41 | nM | 0.94 |
| NO. 275) | 123 | nM | 0.71 | NO. 336) | 123 | nM | 0.82 |
| | 370 | nM | 0.70 | | 370 | nM | 0.70 |
| | 1.1 | μM | 0.63 | | 1.1 | μM | 0.57 |
| | 3.3 | μM | 0.51 | | 3.3 | μM | 0.41 |
| | 10 | μM | 0.40 | | 10 | μM | 0.35 |
| A31302Hi | 14 | nM | 0.89 | A31331Hi | 14 | nM | — |
| (SEQ ID | 41 | nM | 0.87 | (SEQ ID | 41 | nM | 1.01 |
| NO. 285) | 123 | nM | 0.86 | NO. 314) | 123 | nM | 0.95 |
| | 370 | nM | 0.73 | | 370 | nM | 0.88 |
| | 1.1 | μM | 0.59 | | 1.1 | μM | 0.74 |
| | 3.3 | μM | 0.48 | | 3.3 | μM | 0.59 |
| | 10 | μM | 0.35 | | 10 | μM | 0.47 |

| | | | |
|---|---|---|---|
| A31304Hi | 14 | nM | 0.93 |
| (SEQ ID | 41 | nM | 0.90 |
| NO. 287) | 123 | nM | 0.76 |
| | 370 | nM | 0.62 |
| | 1.1 | μM | 0.48 |
| | 3.3 | μM | 0.41 |
| | 10 | μM | 0.31 |

TABLE 10
Half maximal inhibitory concentration (IC₅₀) values and R values
of selected human NLRP3-specific ASOs after three days treatment.

| ASO | IC50 (nM) | R squared |
|---|---|---|
| A31352Hi (SEQ ID NO. 335) | 163.3 | 0.97 |
| A31314Hi (SEQ ID NO. 297) | 439.9 | 0.98 |
| A31109Hi (SEQ ID NO. 59) | 297.3 | 0.94 |
| A31332Hi (SEQ ID NO. 315) | 4925 | 0.98 |
| A31203Hi (SEQ ID NO. 187) | 589.7 | 0.96 |
| A31149H (SEQ ID NO. 133) | 604.6 | 0.90 |
| A31243H (SEQ ID NO. 227) | 411 | 0.94 |
| A31304Hi (SEQ ID NO. 287) | 342.7 | 0.97 |
| A31055Hi (SEQ ID NO. 59) | 1449 | 0.88 |
| A31111Hi (SEQ ID NO. 59) | — | 0.70 |
| A31302Hi (SEQ ID NO. 285) | — | 0.92 |
| A31353Hi (SEQ ID NO. 336) | 538 | 0.96 |
| A31317Hi (SEQ ID NO. 300) | 2972 | 0.88 |
| A31292Hi (SEQ ID NO. 275) | 599.7 | 0.87 |
| A31331Hi (SEQ ID NO. 314) | 1595 | 0.88 |
| A31334Hi (SEQ ID NO. 317) | 798.1 | 0.92 |
| A31324Hi (SEQ ID NO. 307) | 515.3 | 0.95 |

### Example 13: Efficacy of Selected Human NLRP3 Antisense Oligonucleotides in Human Microglial HMC3 Cells

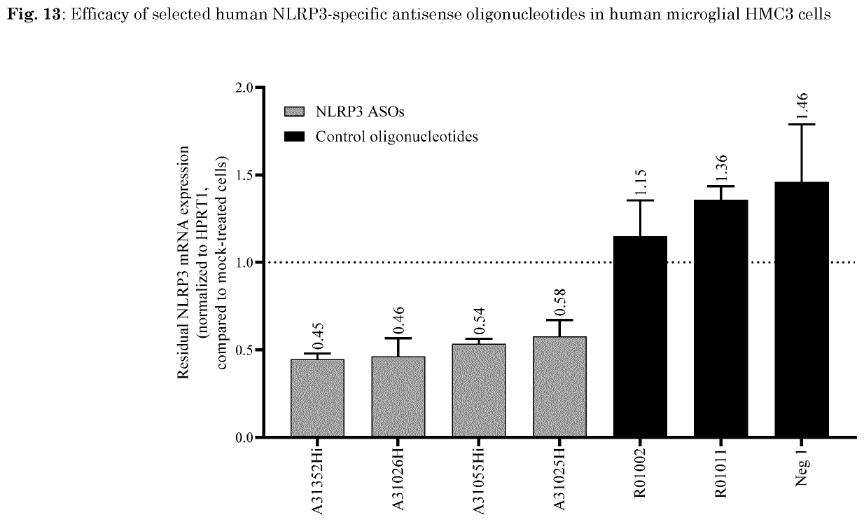

Knockdown efficacy of human NLRP3-specific ASOs selected from previous screening rounds were tested in human HMC3 cells (human microglial clone 3 cell line). The cells were treated with the respective human NLRP3-specific ASO or control oligonucleotides R01002 (SEQ ID NO. 340), R01011 (SEQ ID NO. 341) or Neg1 (SEQ ID NO. 339) at a concentration of 5 μM. Mock-treated cells (no antisense oligonucleotide) were cultured without the addition of oligonucleotides (untreated control). After three days treatment, cells were lyzed. Human HPRT1 and human NLRP3 mRNA expression was measured using the QuantiGene Singleplex assay (ThermoFisher) and the NLRP3 expression values were normalized to HPRT1 values. Residual NLRP3 mRNA expression relative to mock-treated cells (set as 1) is shown as mean and SD in FIG. 13.

Treatment of HMC3 cells with the A31352Hi (SEQ ID NO. 335), A31026H (SEQ ID NO. 30), A31055Hi (SEQ ID NO. 59) and A31025H (SEQ ID NO. 29) ASOs resulted in a target inhibition of >40% (represented by a residual NLRP3 mRNA expression of <0.6 as compared to mock-treated cells) (FIG. 13).

### Example 14: Fifth Screening—Single Concentration Efficacy Screen for Human NLRP3-Specific Antisense Oligonucleotides in THP-1-Derived Macrophages

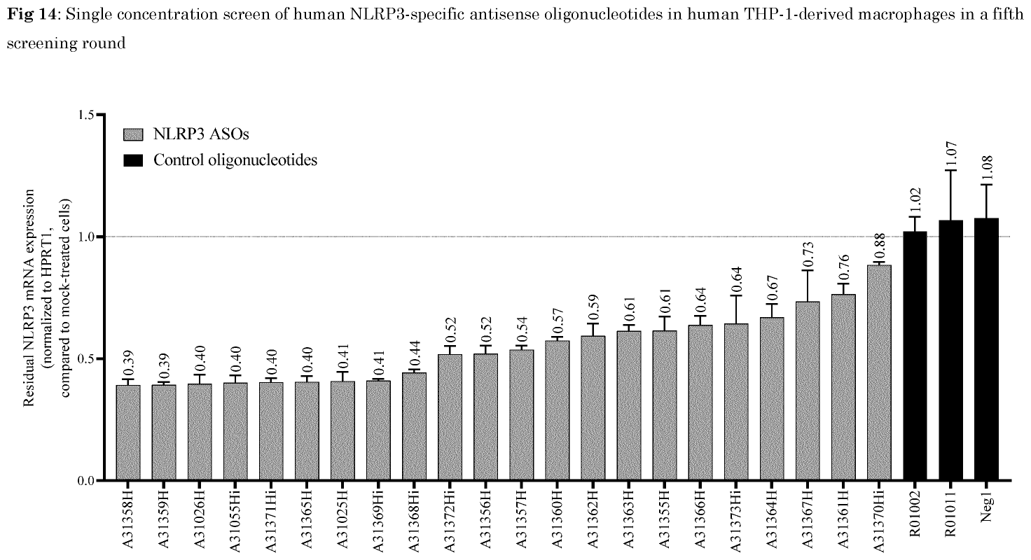

Knockdown efficacy of human NLRP3-specific ASOs was tested in human THP-1-derived macrophages in a fifth screening round. THP-1 cells were differentiated into macrophages by addition of 10 nM PMA. The respective NLRP3-specific ASOs or control oligonucleotides (Neg1 (SEQ ID NO. 339), R01002 (SEQ ID NO. 340) and R01011 (SEQ ID NO. 341)) were added at 5 μM final concentration. Mock-treated cells (no antisense oligonucleotide) were cultured without the addition of oligonucleotides. After three days treatment, cells were lyzed. Human HPRT1 and human NLRP3 mRNA expression was measured using the QuantiGene Singleplex assay (ThermoFisher) and the NLRP3 expression values were normalized to HPRT1 values. Residual NLRP3 mRNA expression relative to mock-treated cells (set as 1) is shown as mean and SD in FIG. 14.

Treatment of THP-1-derived macrophages with the A31358H (SEQ ID NO. 29), A31359H (SEQ ID NO. 29) and A31026H (SEQ ID NO. 30) ASOs resulted in a target inhibition of >60% (represented by a residual NLRP3 mRNA expression of <0.4 as compared to mock-treated cells) (FIG. 14).

### Example 15: Fifth Screening—Single Concentration Efficacy Screen of Human NLRP3-Specific ASOs in PBMC-Derived Macrophages

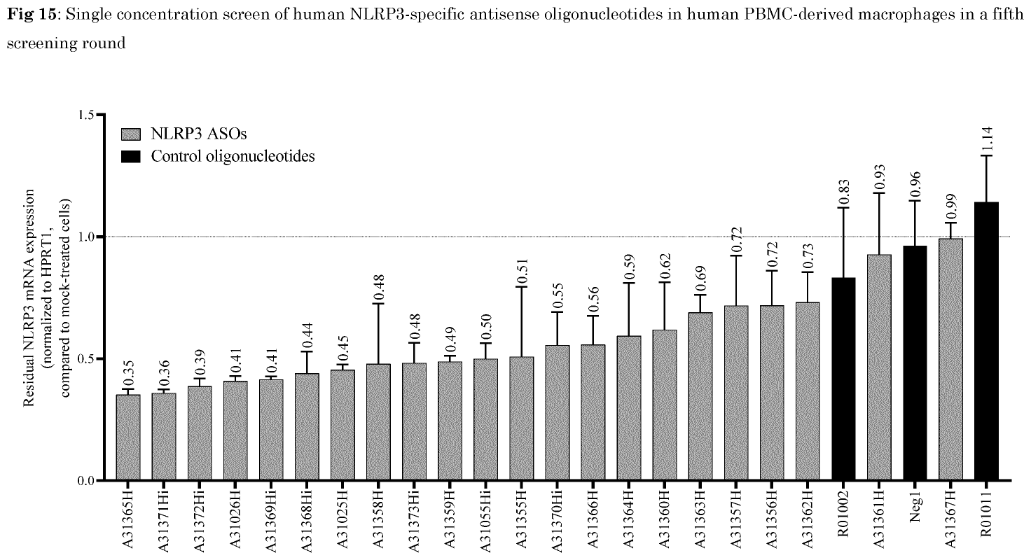

Knockdown efficacy of human NLRP3-specific ASOs was tested in PBMC-derived macrophages in a fifth screening round. Adherent PBMCs were cultured in the presence of 20 ng/ml hGM-CSF and 50 ng/ml hIL-4. Cells were treated with the respective human NLRP3-specific ASO or control oligonucleotides (Neg1 (SEQ ID NO. 339), R01002 (SEQ ID NO. 340) and R01011 (SEQ ID NO. 341)) at a concentration of 5 μM. Mock-treated cells (no antisense oligonucleotide) were cultured without the addition of oligonucleotides (untreated control). After three days treatment, the supernatant and loose cells were removed and replaced by fresh medium containing 20 ng/ml hGM-CSF, 50 ng/ml hIL-4 and the respective human NLRP3-specific ASO or control oligonucleotide at a concentration of 5 μM. On day six, cells were lyzed. Human HPRT1 and human NLRP3 mRNA expression was measured using the QuantiGene Singleplex assay (ThermoFisher) and the NLRP3 expression values were normalized to HPRT1 values. Residual NLRP3 mRNA expression relative to mock-treated cells (set as 1) is shown as mean and SD in FIG. 15.

Treatment of PBMC-derived macrophages with the A31365H (SEQ ID NO. 30), A31371Hi (SEQ ID NO. 59) and A31372Hi (SEQ ID NO. 59) ASOs resulted in a target inhibition of >60% (represented by a residual NLRP3 mRNA expression of <0.4 as compared to mock-treated cells) (FIG. 15).

### Example 16: NLRP3 mRNA Knockdown Prevents Cleavage of Pro-IL-1β and Secretion of Mature IL-1β in Human THP-1-Derived Macrophages

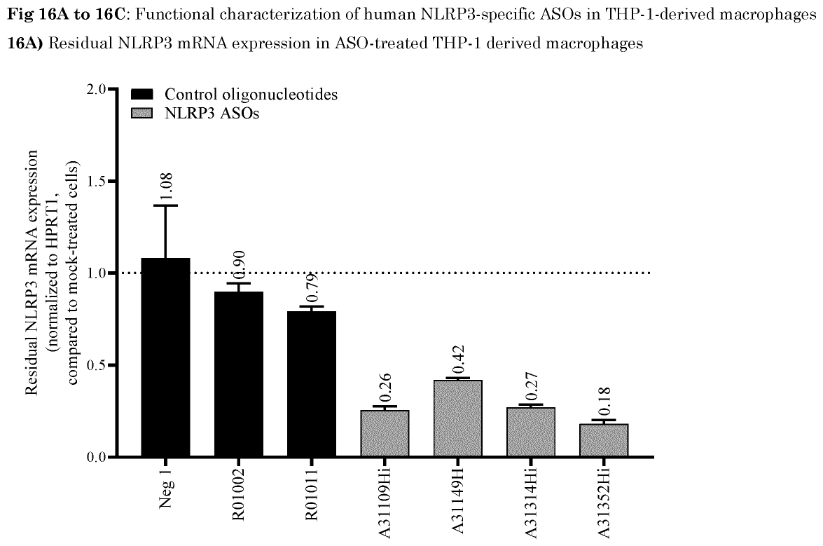

In order to further investigate the target knockdown efficacy of selected human NLRP3-specific ASOs at the mRNA level, THP-1 cells were treated with the NLRP3-specific ASOs A31109Hi (SEQ ID NO. 59), A31149H (SEQ ID NO. 133), A31314Hi (SEQ ID NO. 297) or A31352Hi (SEQ ID NO. 335) or the control oligonucleotide Neg1 (SEQ ID NO. 339), R01002 (SEQ ID NO. 340) or R01011 (SEQ ID NO. 341), respectively, at a concentration of 5 μM. Mock-treated cells (no antisense oligonucleotide) were cultured without the addition of oligonucleotides (untreated control). After three days treatment, medium was replaced by fresh medium containing 5 nM Phorbol 12-myristate 13-acetate (PMA) and the respective human NLRP3-specific ASO or control oligonucleotide at a concentration of 5 μM. After six days treatment, cell culture supernatant was replaced by serum free medium containing 10 μg/ml LPS for 4h (signal 1) and 5 mM ATP for 30 min (signal 2) in order to induce IL-1l production and activate the inflammasome complex. Afterwards the cells were lyzed. Human HPRT1 and human NLRP3 mRNA expression was measured using the QuantiGene Singleplex assay (ThermoFisher) and the NLRP3 expression values were normalized to HPRT1 values. Residual NLRP3 mRNA expression relative to mock-treated cells (set as 1) is shown as mean and SD in FIG. 16A. Treatment of THP-1-derived macrophages with the selected NLRP3-specific ASOs resulted in a target inhibition of >58% (represented by a residual NLRP3 mRNA expression of <0.42 as compared to mock-treated cells) (FIG. 16A).

In order to further investigate the effect of selected human NLRP3-specific ASOs in the inflammasome activation, Caspase-1 activity was assessed by Caspase-Glo® 1 Inflammasome Assay in cell lysate of THP-1-derived macrophages after six days ASO treatment. Data are shown in FIG. 16B as mean and SD. ASO treatment with the selected NLRP3-specific ASOs resulted in a caspase-1 activity inhibition of >65% (represented by a caspase activity <0.35 as compared to mock treated cells set as 1) (FIG. 16B).

As part of the inflammasome complex, NLRP3 is required for cleavage of pro-IL-1β into its mature and secreted form. As a proof-of-concept it was aimed to demonstrate that IL-1β secretion by THP-1-derived macrophages can be inhibited through treatment with NLRP3 specific antisense oligonucleotides. Pro-IL1β and mature IL-1β protein expression was analyzed in cell lysates and cell culture supernatants of THP-1-derived macrophages by western blot after six days ASO treatment (FIG. 16C). Mature IL-1β was only present in the supernatants of either mock-treated cells or cells treated with the control oligonucleotides, but not in cells treated with NLRP3-specific A31109Hi (SEQ ID NO. 59), A31149H (SEQ ID NO. 133), A31314Hi (SEQ ID NO. 297) or A31352Hi (SEQ ID NO. 335) (FIG. 16C). These results demonstrated that inhibition of NLRP3 on mRNA level with antisense oligonucleotides effectively prevented the cleavage of pro-Il-1β in THP-1-derived macrophages.

### Example 17: First Screening—Single Concentration Efficacy Screen for Mouse NLRP3-Specific Antisense Oligonucleotides in 4T1 Cells

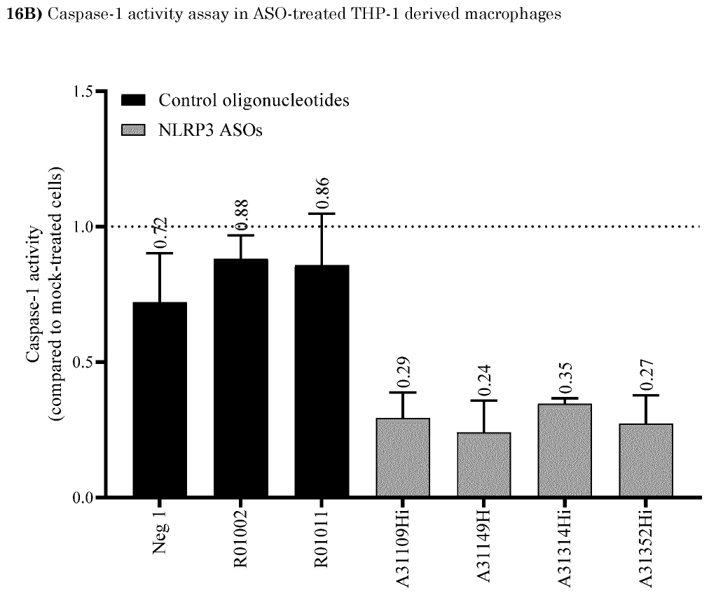

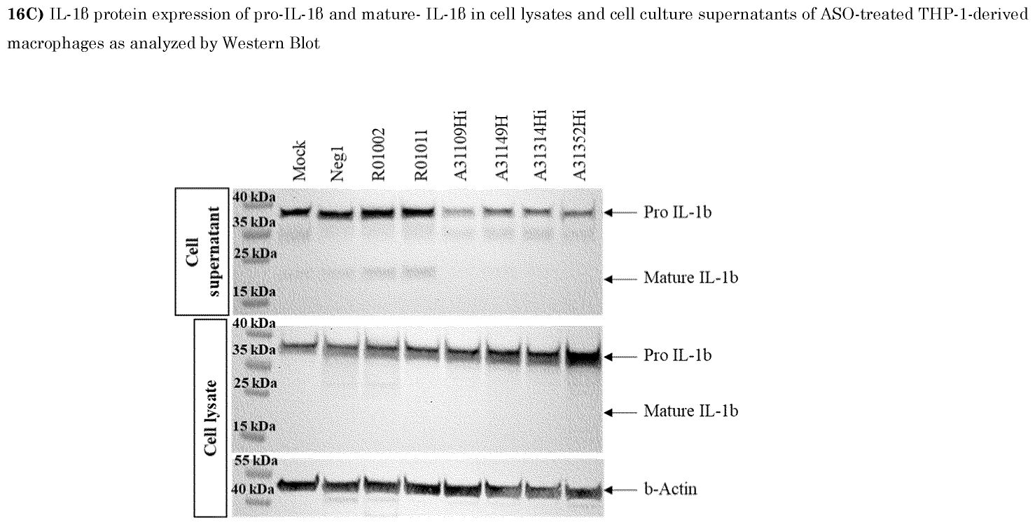

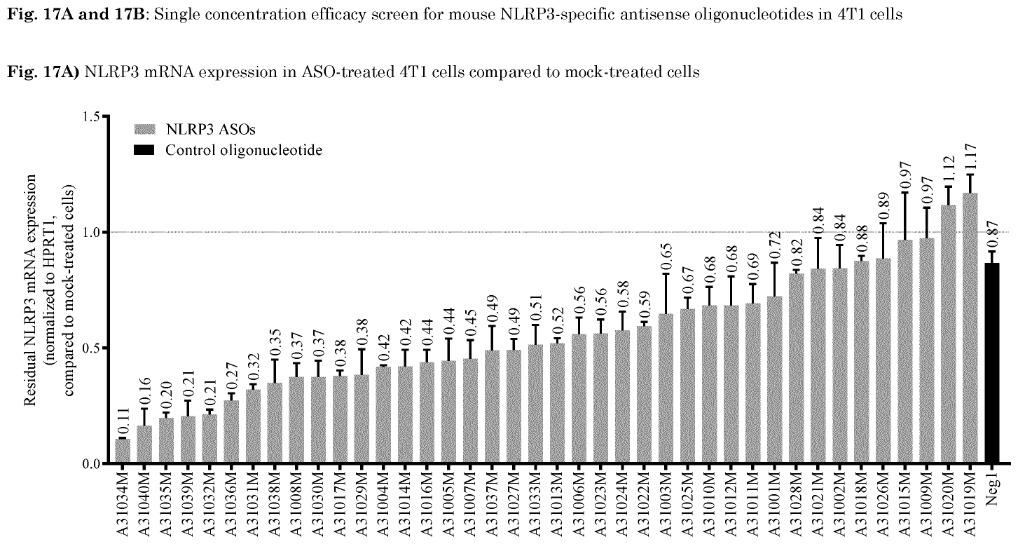

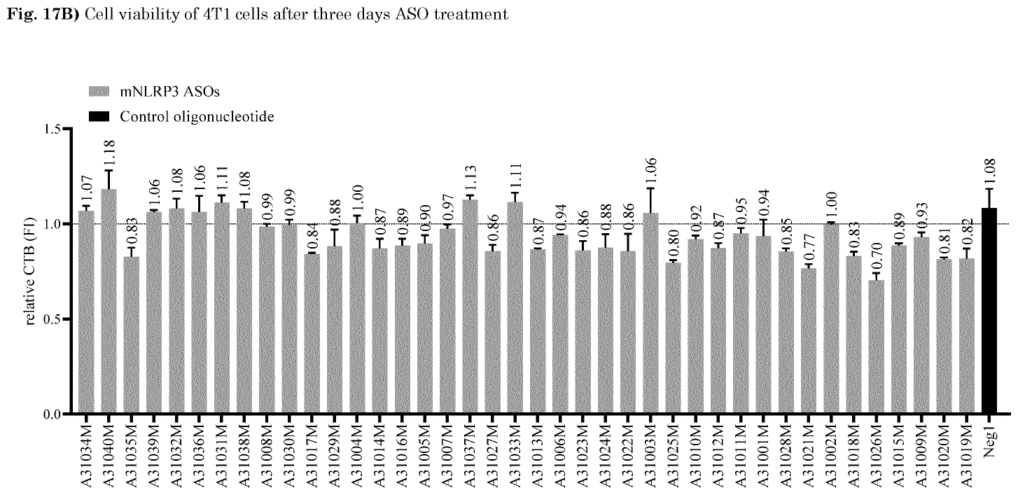

Knockdown efficacy of mouse NLRP3-specific ASOs was tested in mouse 4T1 cells (breast cancer cell line derived from the mammary gland tissue of a mouse). The cells were treated with the respective mouse NLRP3-specific ASO or control oligonucleotide Neg1 (SEQ ID NO. 339) at a concentration of 10 μM. As unstimulated 4T1 cells did not express NLRP3 to a sufficient extent, LPS was added to the cell culture at a final concentration of 1 μg/ml. After three days treatment, cells were lyzed. Mouse HPRT1 and mouse NLRP3 mRNA expression was measured using the QuantiGene Singleplex assay (ThermoFisher) and the NLRP3 expression values were normalized to HPRT1 values. Residual NLRP3 mRNA expression relative to mock-treated cells (set as 1) is shown as mean and SD in FIG. 17A.

Treatment of 4T1 cells with the A31034M (SEQ ID NO. 375), A31040M (SEQ ID NO. 381) and A31035M (SEQ ID NO. 376) ASOs resulted in a target inhibition of ≥80% (represented by a residual NLRP3 mRNA expression of <0.2 as compared to mock-treated cells) (FIG. 17A).

Cell viability of 4T1 cells after three days ASO treatment was also determined by Cell Titer Blue assay. Absolute Fluorescence Intensity (FI) normalized to mock-treated cells (“no antisense oligonucleotides” set as 1) is shown in FIG. 17B. Treatment of 4T1 cells with mouse NLRP3-specific ASOs for three days has no effect on cell viability (FIG. 17B).

### Example 18: First Screening—Single Concentration Efficacy Screen for Mouse NLRP3-Specific Antisense Oligonucleotides in Raw246.7 Cells

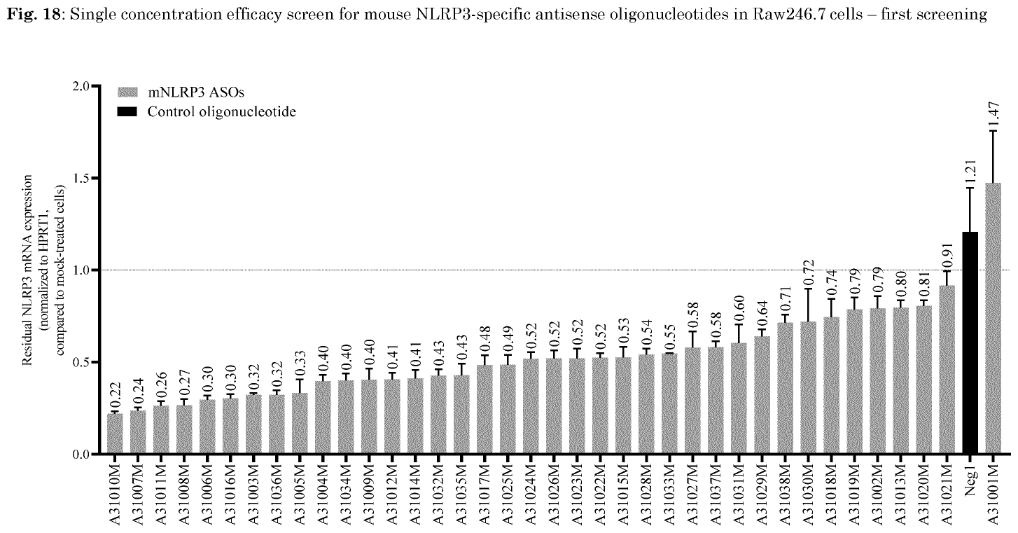

Knockdown efficacy of mouse NLRP3-specific ASOs was tested in mouse Raw246.7 cells (murine macrophage from blood). The cells were treated with the respective mouse NLRP3-specific ASO or control oligonucleotide Neg1 (SEQ ID NO. 339) at a concentration of 10 μM. After three days treatment, cells were lyzed. Mouse HPRT1 and mouse NLRP3 mRNA expression was measured using the QuantiGene Singleplex assay (ThermoFisher) and the NLRP3 expression values were normalized to HPRT1 values. Residual NLRP3 mRNA expression relative to mock-treated cells (set as 1) is shown as mean and SD in FIG. 18.

Treatment of Raw246.7 cells with the A31010M (SEQ ID NO. 351), A31007M (SEQ ID NO. 348), A31011M (SEQ ID NO. 352) and A31008 (SEQ ID NO. 349) ASOs resulted in a target inhibition of >70% (represented by a residual NLRP3 mRNA expression of <0.3 as compared to mock-treated cells) (FIG. 18).

### Example 20: Second Screening—Single Concentration Efficacy Screen for Mouse NLRP3-Specific Antisense Oligonucleotides in Raw246.7 Cells

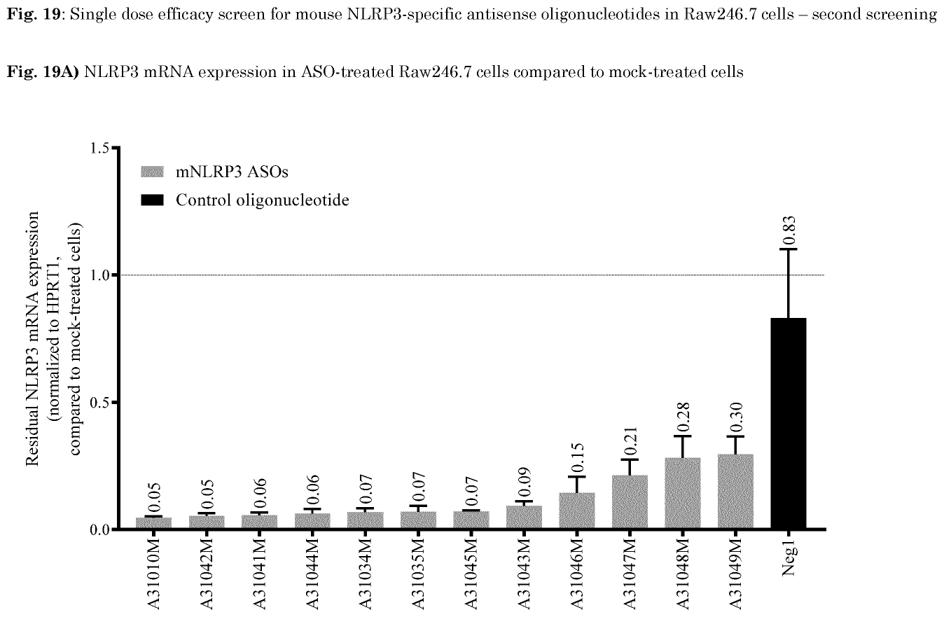

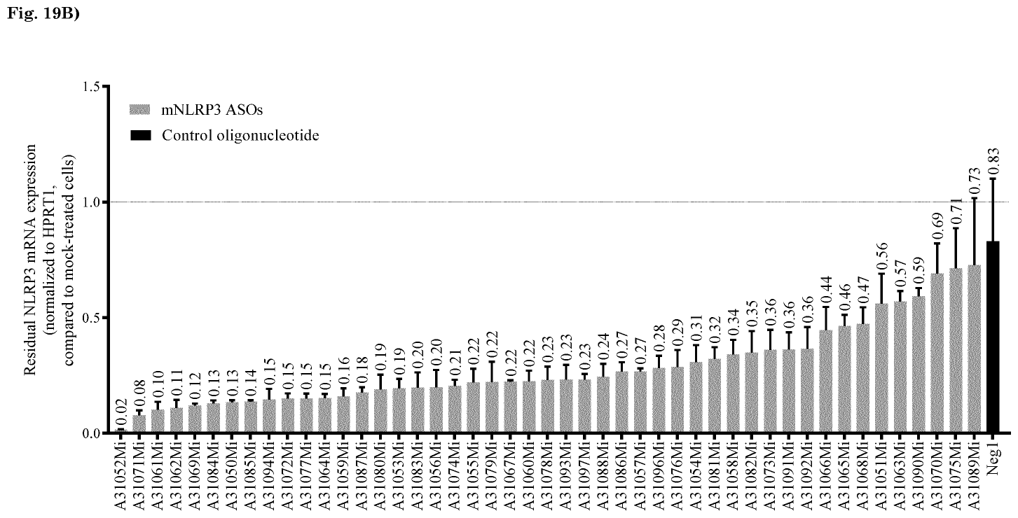

Knockdown efficacy of mouse NLRP3-specific ASOs was tested in mouse Raw246.7 cells in a second screening round. Raw246.7 cells were treated with the respective mouse NLRP3-specific ASO or control oligonucleotide Neg1 (SEQ ID NO. 339) at a concentration of 10 μM. After three days treatment, cells were lyzed. Mouse HPRT1 and mouse NLRP3 mRNA expression was measured using the QuantiGene Singleplex assay (ThermoFisher) and the NLRP3 expression values were normalized to HPRT1 values. Residual NLRP3 mRNA expression relative to mock-treated cells (set as 1) is shown as mean and SD in FIG. 19A and FIG. 19B

Treatment of Raw246.7 cells with A31010M (SEQ ID NO. 351), A31042M (SEQ ID NO. 351), A31041M (SEQ ID NO. 351), A31044M (SEQ ID NO. 351), A31034M (SEQ ID NO. 375), A31035M (SEQ ID NO. 376), A31045M (SEQ ID NO. 375) and A31043M (SEQ ID NO. 351) ASOs had a knockdown efficacy of >90% (represented by a residual NLRP3 mRNA expression of <0.1 as compared to mock treated cells) in Raw246.7 cells after three days treatment (FIG. 19A). Among the antisense oligonucleotides that target the introns of the NLRP3 pre-mRNA, 18 caused a target knockdown of >80% (represented by a residual NLRP3 mRNA expression of <0.2) with two antisense oligonucleotides (A31052Mi (SEQ ID NO. 384) and A31071Mi (SEQ ID NO. 403)) inhibiting target mRNA expression by >90% (FIG. 19B)

### Example 20: Second Screening—Single Concentration Efficacy Screen for Mouse NLRP3-Specific Antisense Oligonucleotides in 4T1 Cells

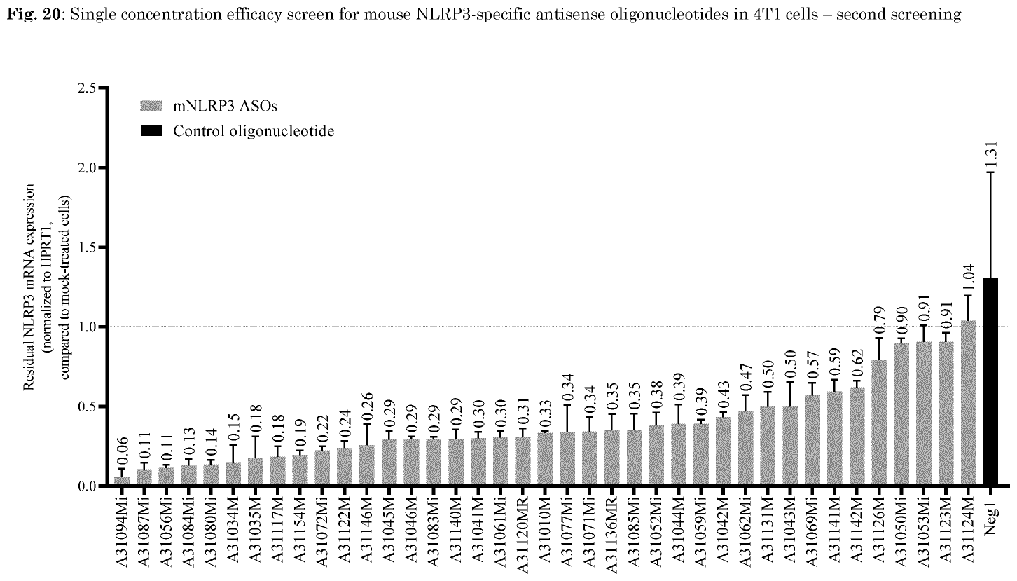

Knockdown efficacy of the most potent antisense oligonucleotides in Raw246.7 cells from the second screening round were further screened in 4T1 cells. 4T1 cells were treated with the respective antisense oligonucleotides or control oligonucleotide (Neg1, SEQ ID NO. 339) at a final concentration of 10 μM. To induce NLRP3 mRNA expression, cells were simultaneously treated with 1 μg/ml LPS. After three days, cells were lysed and mouse HPRT1 as well as mouse NLRP3 mRNA expression was measured using the QuantiGene RNA Singleplex assay. NLRP3 mRNA expression values were normalized to expression of the housekeeping gene HPRT1. Residual NLRP3-mRNA expression relative to mock-treated cells (“no antisense oligonucleotides” set as 1) is shown as mean and SD as shown in FIG. 20.

Treatment of 4T1 cells with the A31094Mi (SEQ ID NO. 426), A31087Mi (SEQ ID NO. 419), A31056Mi (SEQ ID NO. 388), A31084Mi (SEQ ID NO. 416) and A31080Mi (SEQ ID NO. 412) ASOs resulted in a target inhibition of >85% (represented by a residual NLRP3 mRNA expression of <0.15 as compared to mock-treated cells) (FIG. 20).

### Example 21: Concentration Response Curve of Selected Mouse NLRP3-Specific Antisense Oligonucleotides in Raw246.7 Cells

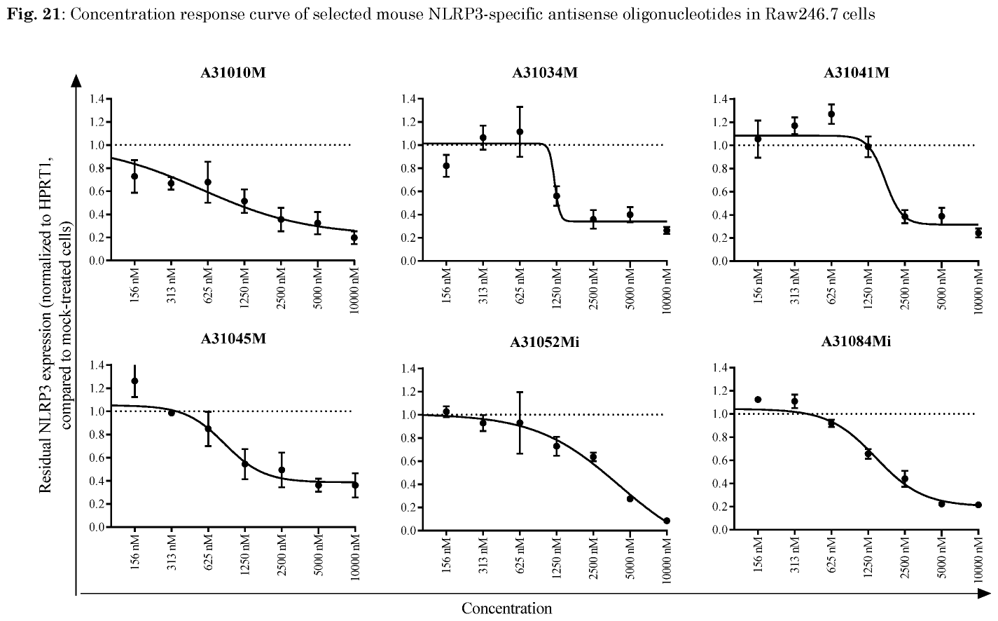

Selected antisense oligonucleotides from the first and second screening rounds which showed no signs of cellular toxicity in vitro were selected for IC₅₀ determination. Raw246.7 cells were treated with the respective mouse NLRP3-specific ASO at the following concentrations: 10000 nM, 5000 nM, 2500 nM, 1250 nM, 325 nM, 313 nM and 156 nM. After three days, cells were lysed and mouse HPRT1 as well as mouse NLRP3 mRNA expression was measured using the QuantiGene RNA Singleplex assay (FIG. 21). NLRP3 mRNA expression values were normalized to expression of the housekeeping gene HPRT1. Residual NLRP3 mRNA expression relative to mock-treated cells (“no antisense oligonucleotide” set as 1) is shown as mean and SD in FIG. 21 and Table 11. The half maximal inhibitory concentration (IC₅₀) values of the concentration response curve for selected candidates are indicated in the Table 12. All ASOs concentration-dependently inhibited the expression of NLRP3 mRNA with two candidates having an IC₅₀ value in nanomolar range.

TABLE 11
Concentration-dependent inhibition of NRLP3 mRNA expression in Raw246.7
cells by selected NLRP3-specific ASOs after 3 days treatment.

| | | | Residual NLRP3 | | | | Residual NLRP3 |
|---|---|---|---|---|---|---|---|
| | | | mRNA expression | | | | mRNA expression |
| | | | compared to | | | | compared to |
| | | | mock-treated cells | | | | mock-treated cells |
| ASO ID | Concentration | | (set as 1) | ASO ID | Concentration | | (set as 1) |
| A31010M | 10 | μM | 0.20 | A31084Mi | 10 | μM | 0.21 |
| (SEQ ID | 5 | μM | 0.32 | (SEQ ID | 5 | μM | 0.22 |
| NO. 351) | 2.5 | μM | 0.36 | NO. 416) | 2.5 | μM | 0.44 |
| | 1.25 | μM | 0.51 | | 1.25 | μM | 0.65 |
| | 625 | nM | 0.68 | | 625 | nM | 0.92 |
| | 313 | nM | 0.67 | | 313 | nM | 1.11 |
| | 156 | nM | 0.73 | | 157 | nM | 1.12 |
| A31034M | 10 | μM | 0.26 | A31087Mi | 10 | μM | 0.15 |
| (SEQ ID | 5 | μM | 0.40 | (SEQ ID | 5 | μM | 0.20 |
| NO. 375) | 2.5 | μM | 0.36 | NO. 419) | 2.5 | μM | 0.25 |
| | 1.25 | μM | 0.56 | | 1.25 | μM | 0.67 |
| | 625 | nM | 1.12 | | 625 | nM | 0.94 |
| | 313 | nM | 1.07 | | 313 | nM | 0.97 |
| | 157 | nM | 0.82 | | 157 | nM | 1.33 |
| A31041M | 10 | μM | 0.24 | A31094Mi | 10 | μM | 0.22 |
| (SEQ ID | 5 | μM | 0.39 | (SEQ ID | 5 | μM | 0.33 |
| NO. 351) | 2.5 | μM | 0.38 | NO. 426) | 2.5 | μM | 0.46 |
| | 1.25 | μM | 0.99 | | 1.25 | μM | 0.78 |
| | 625 | nM | 1.27 | | 625 | nM | 0.94 |
| | 313 | nM | 1.17 | | 313 | nM | 0.95 |
| | 157 | nM | 1.05 | | 157 | nM | 0.95 |
| A31045M | 10 | μM | 0.36 | A31117M | 10 | μM | 0.28 |
| (SEQ ID | 5 | μM | 0.36 | (SEQ ID | 5 | μM | 0.28 |
| NO. 375) | 2.5 | μM | 0.49 | NO. 448) | 2.5 | μM | 0.40 |
| | 1.25 | μM | 0.54 | | 1.25 | μM | 0.65 |
| | 625 | nM | 0.85 | | 625 | nM | 0.85 |
| | 313 | nM | 0.98 | | 313 | nM | 0.78 |
| | 157 | nM | 1.26 | | 157 | nM | 0.87 |

| | | | |
|---|---|---|---|
| A31052Mi | 10 | μM | 0.08 |
| (SEQ ID | 5 | μM | 0.27 |
| NO. 384) | 2.5 | μM | 0.64 |
| | 1.25 | μM | 0.73 |
| | 625 | nM | 0.93 |
| | 313 | nM | 0.93 |
| | 157 | nM | 1.03 |

TABLE 12
Half maximal inhibitory concentration (IC₅₀) values and R values
of selected mouse NLRP3-specific ASOs after three days treatment.

| Antisense oligonucleotide | IC₅₀ [nM] | R squared |
|---|---|---|
| A31010M (SEQ ID NO. 351) | 560.9 | 0.76 |
| A31034M (SEQ ID NO. 375) | ~1206 | 0.75 |
| A31041M (SEQ ID NO. 351) | 1721 | 0.79 |
| A31045M (SEQ ID NO. 375) | 851.7 | 0.83 |
| A31052Mi (SEQ ID NO. 384) | 4039 | 0.89 |
| A31084Mi (SEQ ID NO. 416) | 1446 | 0.93 |
| A31087Mi (SEQ ID NO. 419) | 1327 | 0.85 |
| A31094Mi (SEQ ID NO. 426) | 1824 | 0.94 |
| A31117M (SEQ ID NO. 448) | 1498 | 0.90 |

### Example 22: Murine NLRP3-Specific Antisense Oligonucleotides Prevent Cleavage of Pro-IL-1β in Mouse Bone Marrow Derived Macrophages (BMDM)

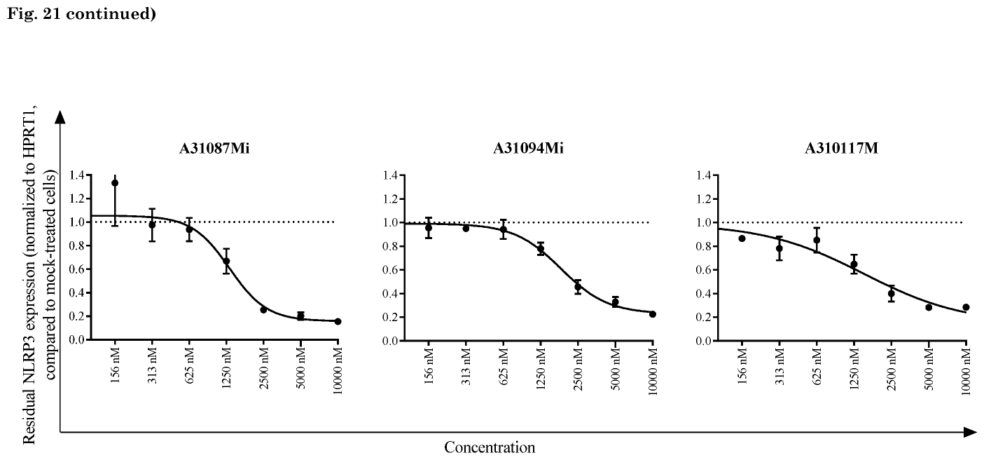

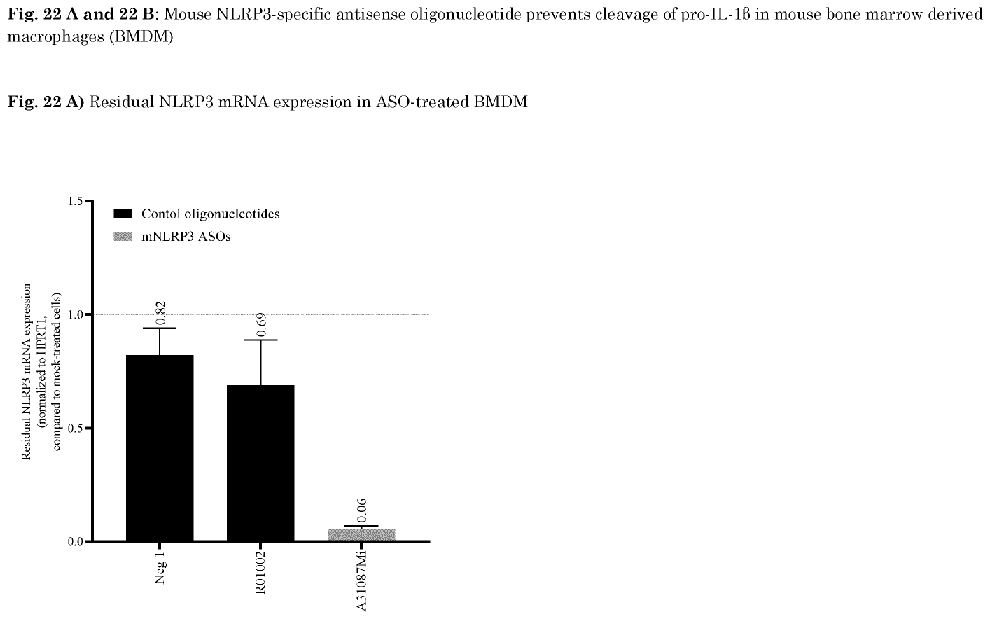

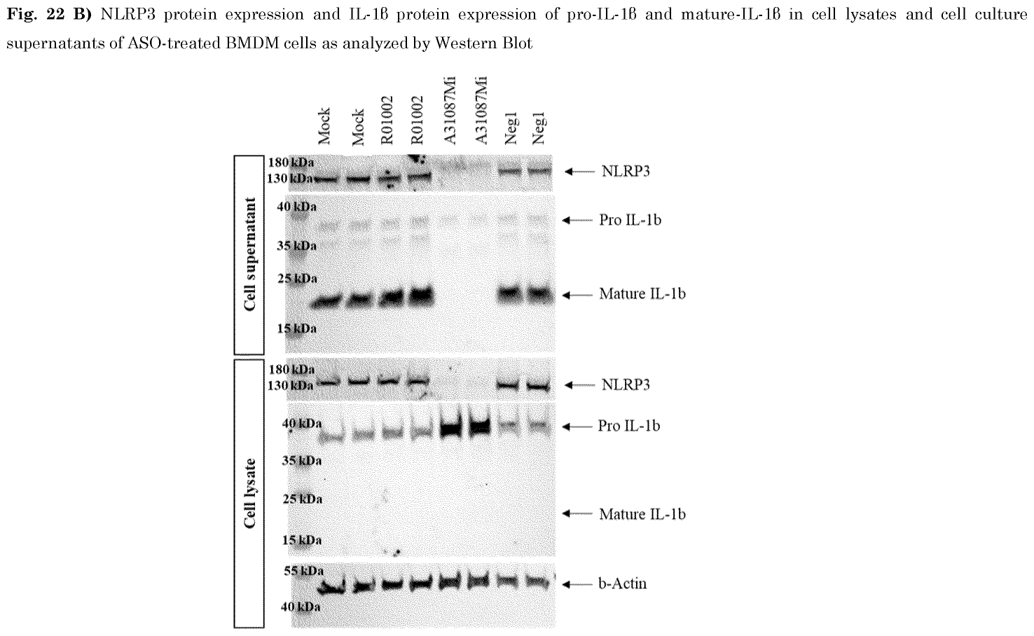

Knockdown efficacy of the selected mouse NLRP3-specific ASOs A31087Mi (SEQ ID NO. 419) was further screened in bone marrow derived macrophages (BMDM). For that, murine bone marrow cells were differentiated into BMDM for seven days in the presence of 50 ng/ml M-CSF. During the last four days of the BMDM differentiation protocol, cells were treated with the respective mouse NLRP3-specific ASOs A31087Mi or control oligonucleotide (Neg1 (SEQ ID NO. 339) or R01002 (SEQ ID NO. 340) at a concentration of 10 μM. After seven days treatment, cell culture supernatant was replaced by serum free medium containing 200 ng/ml LPS for 4h (signal 1) and 5 mM ATP for 30 min (signal 2) in order to induce IL-1β production and activate the inflammasome complex. Afterwards the cells were lyzed. Mouse HPRT1 and mouse NLRP3 mRNA expression was measured using the QuantiGene Singleplex assay (ThermoFisher) and the NLRP3 expression values were normalized to HPRT1 values. Residual NLRP3 mRNA expression relative to mock-treated cells (set as 1) is shown as mean and SD in FIG. 22A. Treatment of BMDM with the selected NLRP3-specific ASOs resulted in a target inhibition of ≥94% (represented by a residual NLRP3 mRNA expression of ≤0.06 as compared to mock-treated cells) (FIG. 22A).

As part of the inflammasome complex, NLRP3 is required for cleavage of pro-IL-1β into its mature and secreted form. As a proof-of-concept it was aimed to demonstrate that IL-1β secretion by primary immune cells can be inhibited through treatment with NLRP3 specific antisense oligonucleotides. NLRP3, pro-IL1β and mature IL-1β protein expression was analyzed by Western Blot in cell lysates and cell culture supernatants of BMDM after six days ASO treatment (FIG. 22B). NLRP3 could be detected in the cell supernatant and cell lysates of mock-treated cells and cells treated with the control oligonucleotides, but not in cells treated with NLRP3-specific ASO A31087 Mi (SEQ ID NO. 419) (FIG. 22B). As the transcription and translation of pro-IL-1β is independent of NLRP3, pro-IL-1β could be detected in the cell lysates under all treatment conditions (cells treated with NLRP3-specific ASOs, control oligonucleotides or mock-treated cells (no ASO) (FIG. 22B). In contrast, mature IL-1β was only present in the supernatants of either mock-treated cells (no ASO, untreated control) or cells that were treated with the control oligonucleotides, but not in cells treated with NLRP3-specific ASOs A31087Mi (SEQ ID NO. 419) (FIG. 22B). These results demonstrate that inhibition of NLRP3 on mRNA level with antisense oligonucleotides effectively prevents the cleavage of pro-Il-1β in BMM.

### Example 23: Efficacy of Selected Mouse NLRP3 Antisense Oligonucleotides in Raw246.7 Cells

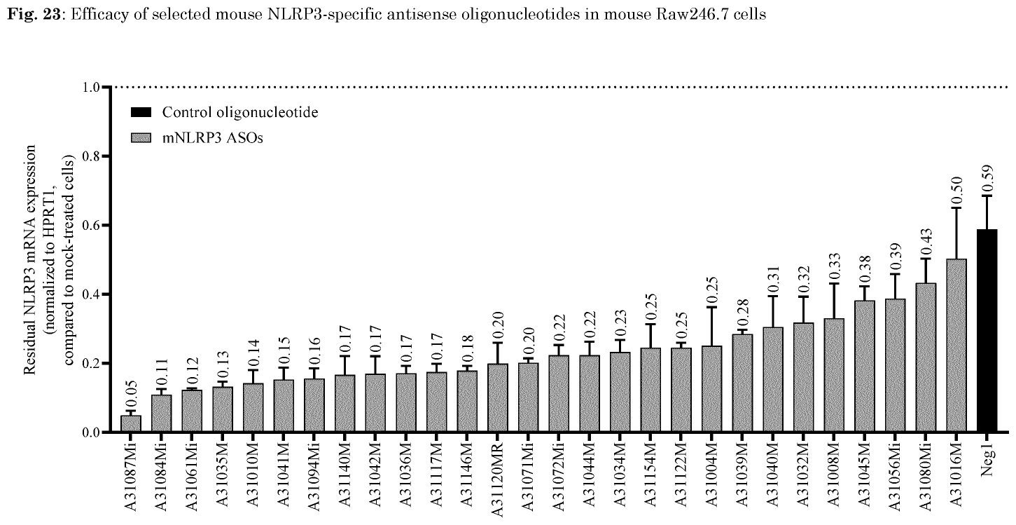

Knockdown efficacy of selected mouse NLRP3-specific ASOs was further analyzed in Raw246.7 cells. Raw246.7 cells were treated with the respective antisense oligonucleotides or control oligo (Neg1) at a final concentration of 5 μM. After three days, cells were lysed and mouse HPRT1 as well as mouse NLRP3 mRNA expression was measured using the QuantiGene RNA Singleplex assay. NLRP3 mRNA expression values were normalized to the expression of the housekeeping gene HPRT1. Residual NLRP3-mRNA expression relative to mock-treated cells (“no antisense oligonucleotides” set as 1, untreated control) is shown as mean and SD as shown in FIG. 23.

Treatment of Raw246.7 cells with the A31087Mi (SEQ ID NO. 419), A31084Mi (SEQ ID NO. 416), A31061Mi (SEQ ID NO. 393), A31035M (SEQ ID NO. 376), A31010M (SEQ ID NO. 351) and A31041M (SEQ ID NO. 351) ASOs resulted in a target inhibition of ≥85% (represented by a residual NLRP3 mRNA expression of ≤0.15 as compared to mock-treated cells) (FIG. 23).

### Example 24: Efficacy of Selected Mouse NLRP3 Antisense Oligonucleotides in Mouse Microglial IMG Cells

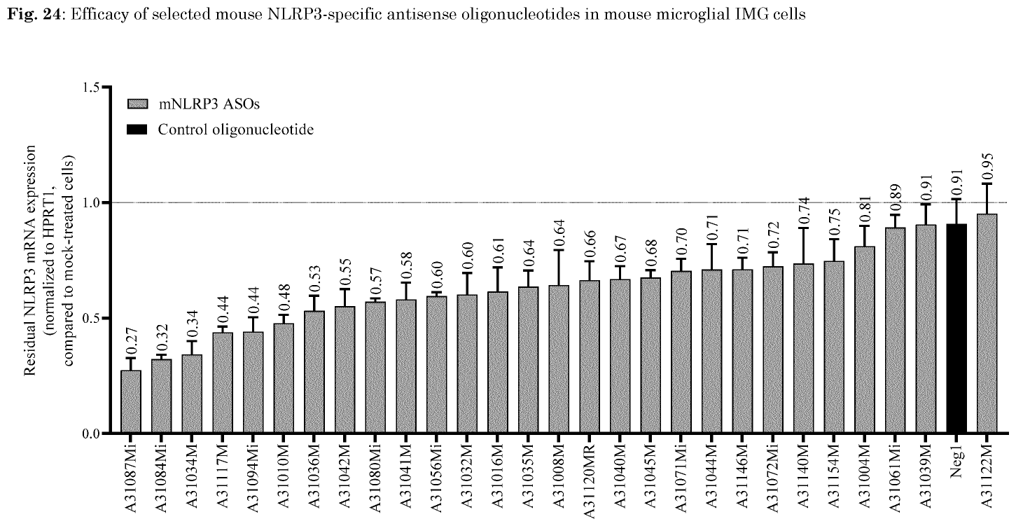

Knockdown efficacy of selected mouse NLRP3-specific ASOs was further assessed in IMG cells (microglial cell line isolated from the brains of adult mice). IMG cells were treated with the respective antisense oligonucleotides or control oligo (Neg1) at a final concentration of 5 μM. After three days, cells were lysed and mouse HPRT1 as well as mouse NLRP3 mRNA expression was measured using the QuantiGene RNA Singleplex assay. NLRP3 mRNA expression values were normalized to expression of the housekeeping gene HPRT1. Residual NLRP3-mRNA expression relative to mock-treated cells (“no ASO” set as 1, untreated control) is shown as mean and SD as shown in FIG. 2. Treatment of IMG cells with the A31087Mi (SEQ ID NO. 419), A31084Mi (SEQ ID NO. 416) and A31034M (SEQ ID NO. 375) ASOs resulted in a target inhibition of ≥66% (represented by a residual NLRP3 mRNA expression of <0.34 as compared to mock-treated cells) (FIG. 24).

1. Oligonucleotide comprising a sequence selected from the group consisting of SEQ ID NO. 59, SEQ ID NO. 335, SEQ ID NO. 297, and SEQ ID NO. 133 comprising at least one modified nucleotide, hybridizing with a nucleic acid sequence of a NLR family pyrin domain containing 3 (NLRP3) of human SEQ ID NO. 1 and/or human SEQ ID NO. 2 resulting in a reduction of the level of NLRP3, NLRP3 mRNA, NLRP3 pre-mRNA or a combination thereof.

2. The oligonucleotide of claim 1, wherein the modified nucleotide is selected from the group consisting of a bridged nucleic acid such as LNA, cET, ENA, 2′Fluoro modified nucleotide, 2′O-Methyl modified nucleotide, a 2 O-Methoxy modified nucleotide, a FANA and a combination thereof.

3. The oligonucleotide of claim 1 or 2, wherein the reduction of the level of NLRP3, NLRP3 mRNA, NLRP3 pre-mRNA or a combination thereof is 30 to 99% compared to an untreated control.

4. The oligonucleotide of any one of claims 1 to 3, wherein the oligonucleotide comprises a modification selected from the group consisting of
(A31109Hi; SEQ ID NO. 59)
+G*+T*+A*A*T*G*T*C*A*A*C*G*G*A*+T*+C,
(A31055Hi, SEQ ID NO. 59)
+G*+T*+A*A*T*G*T*C*A*A*C*G*G*+A*+T*+C,
(A31110Hi, SEQ ID NO. 59)
+G*+T*A*A*T*G*T*C*A*A*C*G*G*+A*+T*+C,
(A31111Hi, SEQ ID NO. 59)
+G*+T*+A*+A*T*G*T*C*A*A*C*G*G*+A*+T*+C,
(A31112Hi, SEQ ID NO. 59)
+G*T*+A*A*T*G*T*C*A*A*C*G*G*+A*+T*+C,
(A31368Hi, SEQ ID NO. 59)
+G*+T*+A*A*+T*G*T*C*A*A*C*G*G*+A*+T*+C,
(A31369Hi, SEQ ID NO. 59)
+G*+T*+A*A*T*+G*T*C*A*A*C*G*G*+A*+T*+C,
(A31370Hi, SEQ ID NO. 59)
+G*+T*+A*A*T*G*+T*C*A*A*C*G*G*+A*+T*+C,
(A31371Hi, SEQ ID NO. 59)
+G*+T*+A*A*T*G*T*C*A*A*C*+G*G*+A*+T*+C,
(A31372Hi, SEQ ID NO. 59)
+G*+T*+A*A*T*G*T*C*A*A*+C*G*G*+A*+T*+C,
(A31373Hi, SEQ ID NO. 59)
+G*+T*+A*+A*T*G*T*C*A*A*C*G*+G*+A*+T*+C,
(A31149H, SEQ ID NO. 133)
+G*+A*C*T*G*T*C*A*C*G*T*C*T*C*+G*+G*+C,
(A31314Hi, SEQ ID NO. 297)
+C*+A*+T*A*G*T*T*C*T*C*T*G*C*A*A*C*+A*+G*+G,
(A31352Hi, SEQ ID NO. 335)
+T*+A*+G*T*A*T*C*A*C*T*G*T*A*T*G*T*+C*+C*+A,
and a combination thereof, wherein + indicates an LNA nucleotide and * indicates a phosphorothioate (PTO) linkage between the nucleotides.

5. The oligonucleotide of any one of claims 1 to 4, wherein the oligonucleotide comprises the modification selected from the group consisting of
(A31109Hi; SEQ ID NO. 59)
+G*+T*+A*A*T*G*T*C*A*A*C*G*G*A*+T*+C,
(A31149H, SEQ ID NO. 133)
+G*+A*C*T*G*T*C*A*C*G*T*C*T*C*+G*+G*+C,
(A31314Hi, SEQ ID NO. 297)
+C*+A*+T*A*G*T*T*C*T*C*T*G*C*A*A*C*+A*+G*+G,
(A31352Hi, SEQ ID NO. 335)
+T*+A*+G*T*A*T*C*A*C*T*G*T*A*T*G*T*+C*+C*+A,
and a combination thereof, wherein + indicates an LNA nucleotide and * indicates a phosphorothioate (PTO) linkage between the nucleotides.

6. The oligonucleotide of any one of claims 1 to 5, wherein the oligonucleotide hybridizes with at least one exon or intron of SEQ ID NO. 1 and/or with the mRNA of SEQ ID NO. 2.

7. The oligonucleotide of any one of claims 1 to 6, wherein the oligonucleotide inhibits the expression of NLRP3, NLRP3 mRNA, NLRP3 pre-mRNA or a combination thereof at a nanomolar or micromolar concentration.

8. Pharmaceutical composition comprising an oligonucleotide of any one of claims 1 to 7 and a pharmaceutically acceptable carrier, excipient, dilutant or a combination thereof.

9. The pharmaceutical composition of claim 8, further comprising another active agent selected from the group consisting of an oligonucleotide, an antibody, a small molecule, a polypeptide, a lipid, a sugar and a combination thereof.

10. The pharmaceutical composition of claim 9, wherein the oligonucleotide and the other active agent inhibit the same target or a different target.

11. The pharmaceutical composition of claim 10, wherein the other active agent modulates the target selected from the group consisting of NLRP3, CD39, CD73, IL-1β, IL-1 receptor, IL-1R accessory protein, IL-18, IL-18 receptor, ASC, NLRC4, AIM2, Caspase-1, RIPK3, Gasdermin D, MLKL, TLR4, Caspase-8, P2X7, NFκB, RORγt, TGF-β, IL-21, IL-17, IL-22, IL-23, IL-6, TNF-α, CCR6, CCL20, STAT3, MMP-1, MMP-8, ADAMTS-5, HMG-CoA, Myd-88, HMGB-1, ROS, TAK-1, Chop, FPR1, LIMCH1, caspase inhibitor and a combination thereof.

12. The oligonucleotide of any one of claims 1 to 7 or the pharmaceutical composition of any one of claims 8 to 11 for use in a method of preventing and/or treating a disorder, where an NLRP3 imbalance is involved.

13. The oligonucleotide or the pharmaceutical composition for use according to claim 12, wherein the disorder is selected from the group consisting of a hyperproliferative disorder such as cancer, an inflammatory or autoimmune disorder, neurodegenerative disease, a neurological disorder, cardiovascular, metabolic disorder, renal disorder, liver disorder, lung disorder, skin disorder, ocular disorder, disorder of the gastro-intestinal tract, joint inflammation, organ transplantation, fibrotic disorder and a combination thereof.

14. The oligonucleotide or the pharmaceutical composition for use according to claim 12 or 13, wherein the disorder is selected from the group consisting of Alzheimer's disease, multiple sclerosis, autoimmune encephalitis, stroke, traumatic brain injury, atherosclerosis, nonalcoholic fatty liver disease, nonalcoholic steatohepatitis, hypertension, myocardial infarction, acute kidney injury, ischemia reperfusion injury, chronic kidney diseases, crystal-induced nephropathies, glomerulonephritis, silicosis, asthma, allergic airway inflammation, inflammatory bowel disease, colitis ulcerosa, osteoarthritis, rheumatoid arthritis, juvenile idiopathic arthritis, transplantation of kidney, lung, liver and/or heart, fibrotic disorder of kidney, lung, liver and/or heart, hyperinflammation following influenza infection, graft-versus-host disease, interstitial cystitis, uveitis, sinusitis, periodontal disease, optic neuritis, myelodysplastic syndrome, cryopyrin-associated periodic syndromes (CAPS) including familial cold autoinflammatory syndrome (FCAS), the Muckle-Wells syndrome (MWS) and neonatal-onset multisystem inflammatory disease (NOMID), gout, obesity-induced inflammation, insulin resistance, type 1 and type 2 diabetes, contact hypersensitivity and a combination thereof, and/or a cancer selected from the group consisting of breast cancer, lung cancer, malignant melanoma, lymphoma, skin cancer, bone cancer, prostate cancer, liver cancer, brain cancer, cancer of the larynx, gall bladder, pancreas, testicular, rectum, parathyroid, thyroid, adrenal, neural tissue, head and neck, colon, stomach, bronchi, kidneys, basal cell carcinoma, squamous cell carcinoma, metastatic skin carcinoma, osteo sarcoma, Ewing's sarcoma, reticulum cell sarcoma, liposarcoma, myeloma, giant cell tumor, small-cell lung tumor, islet cell tumor, primary brain tumor, meningioma, acute and chronic lymphocytic and granulocytic tumors, acute and chronic myeloid leukemia, hairy-cell tumor, adenoma, hyperplasia, medullary carcinoma, intestinal ganglioneuromas, Wilm's tumor, seminoma, ovarian tumor, leiomyomater tumor, cervical dysplasia, retinoblastoma, soft tissue sarcoma, malignant carcinoid, topical skin lesion, rhabdomyosarcoma, Kaposi's sarcoma, osteogenic sarcoma, malignant hypercalcemia, renal cell tumor, polycythermia vera, adenocarcinoma, anaplastic astrocytoma, glioblastoma multiforma, leukemia, epidermoid carcinoma and a combination thereof.

15. The oligonucleotide or the pharmaceutical composition for use according to any one of claims 12 to 13, wherein the oligonucleotide or the composition is administered locally or systemically.
